(19) United States
(12) Patent Application Publication
(10) Pub. No.: US 2026/0167750 A1
(43) Pub. Date: Jun. 18, 2026

(54) BIO-RENEWABLE POLYMERS AND USES THEREOF

(71) Applicant: The Regents of the University of California, Oakland, CA (US)

(72) Inventors: Brett A. HELMS, Oakland, CA (US); Jeremy DEMARTEAU, Oakland, CA (US); Alexander EPSTEIN, Oakland, CA (US); Kristin A. CEDER-PERSSON, Oakland, CA (US); Robert W. HAUSHALTER, Oakland, CA (US); Jay D. KEASLING, Oakland, CA (US); Eric DAILING, Oakland, CA (US)

(21) Appl. No.: 18/997,488

(22) PCT Filed: Jul. 20, 2023
(86) PCT No.: PCT/US2023/028298

§ 371 Date: Jan. 21, 2026

Related U.S. Application Data
(60) Provisional application No. 63/390,962, filed on Jul. 20, 2022.

(51) Int. Cl. C08F 126/02; C07D 309/36; C08J 11/10

(52) U.S. Cl. CPC C08F 126/02; C07D 309/36; C08J 11/105

(57) ABSTRACT

Novel bio-based polymers are described that may be chemically recycled to monomer at end of life. The polymers may be manufactured as fiber, molded parts, film, coatings, etc. that are more efficiently recycled than commodity plastics. Specific embodiments exhibit performance advantages arising from the biofunctionalization of monomers over related designs where such functionality is absent.

### CROSS-REFERENCE TO RELATED APPLICATIONS

This application claims the benefit of and priority to U.S. Provisional Application 63/390,962, filed on Jul. 20, 2022, which is hereby incorporated by reference.

### STATEMENT AS TO RIGHTS TO INVENTIONS MADE UNDER FEDERALLY SPONSORED RESEARCH AND DEVELOPMENT

This disclosure was made with government support under DE-AC02-05CH11231 awarded by the United Stated Department of Energy. The government has certain rights in the invention.

### FIELD

The present disclosure is in the field of bio-renewable circular polymers.

### BACKGROUND

Over 260M tons of plastic waste are generated annually. That amount is projected to double by 2030. Of this waste ˜52M tons/year is incinerated for energy recovery and ˜100M tons/year is landfilled, predominantly in poor communities that will bear the burden of its toxic legacy. Marine ecosystems are similarly affected by leakage of ˜50M tons/year into the world's oceans. Despite growing public awareness and concern over the impacts of plastic waste on global health, only ˜40M tons/year is recycled. Current recycling practices are based on mechanical grinding, which degrades the performance and value of the recovered materials. These practices have failed to stimulate collection and re-use of materials from waste plastics. Instead, to meet growing demand, producers have increased manufacturing capacity. Notably, the EPA recorded that CO₂ emissions from petrochemicals increased by ˜43% between 1990-2019, from 21.6 MMT CO₂e to 30.8 MMT CO₂e, driven by a 2-fold increase in ethylene production for polyethylene resin production. This rise is likely to continue unless new chemical processes and materials that bring greater circularity to polymer life cycles are discovered and developed.

Chemical depolymerization of condensation polymers for monomer recovery is most often conducted by solvolysis, acidolysis, or ring-closing depolymerization in the presence of a catalyst. Monomer composition, temperature, chemical environment, and catalyst all are relevant to whether the original monomers are recovered in the process. Polyethylene terephthalate and nylon-6 are notable examples of commodity plastics comprising condensation polymers that can be chemically recycled to reusable monomers. Polyamides other than nylon-6, however, are not efficiently recycled to monomer: ring-closing depolymerization is less favorable for larger ring monomers and amide bonds in nylon-m,n (where m and n refer to the number of carbons in the diamine and diacid monomers) are slow to solvolyze or hydrolyze. For polyurethanes, chemical recycling to reusable monomers is also problematic. Typically, in the synthesis of polyurethanes, isocyanate monomers are partially hydrolyzed to produce a pre-polymer with urea linkages, prior to mixing with polyols, many of which are based on hydroxy end-capped polyesters and polyethers, to produce polyurethane. Upon solvolysis or acidolysis, the urethane bonds are cleaved, however, the urea bonds remain intact. This produces a complex mixture of amine-terminated polyureas as well as polyols (or small molecules, if the polyol is unstable). If the polyol is stable during depolymerization, it may be recovered for reuse. None of the other compounds, other than the polyols, are reused in secondary polyurethane production. Thus, for many important condensation polymers, circularity remains out of reach, or is otherwise practiced with low efficiency. It remains a challenge to design novel condensation polymers for efficient circularity in chemical recycling of end-of-life plastics to their original monomers.

Certain embodiments of polydiketoenamines (PDK) have recently been shown to be circular, deconstructing in strong aqueous acid to the original monomers in a nearly lossless and low-intensity manner—even from mixed-waste streams. Pigments, additives, flame retardants, fillers, and fibers are dissociated from triketone and amine monomers using simple chemical separations, allowing in most cases all components to be re-used in circular manufacturing. A systems-level analysis of circularity in PDK recycling showed that GHG emissions of depolymerizing PDK to monomers and re-generating virgin-quality resin from them are lower than those tied to the primary production of commodity polymer resins such as PET, HDPE, and polyurethane (PU).

Prior art in polydiketoenamines has demonstrated that by introducing heteroatoms in the ring comprising a diketoenamine, the rate of diketoenamine hydrolysis slows by over two orders of magnitude. Leveraging the differentiated rates, mixed-polymer recycling becomes possible. For example, several PDK resins may be chemospecifically deconstructed to monomer in a prescribed sequence, e.g., by varying the depolymerization temperature. Sequential depolymerization processes may also conducted in the presence of metal, glass, and fiber without significantly affecting yield or quality. For comparison, chemospecific recycling of more than one polyester, polyamide, or polyurethane from a mixture is often difficult to achieve due to their similar rates of depolymerization.

From a manufacturing perspective, access to circular polymers and chemical recycling practices increases industrial materials efficiency, as secondary manufacturing with recycled feedstocks can be lower in cost and can be conducted with less carbon- and energy-intensity. There are growing concerns, however, over the sustainability of supply chains for primary production, e.g., if raw materials used to produce circular polymers rely on fossil resources. Bio-based polymers are defined as materials for which at least a portion of the polymer consists of material produced from renewable raw materials. Bio-based raw materials seeking to replace petroleum-derived counterparts are in some cases commercially viable, although subject to fluctuations in the market price of fossil resources, energy costs, taxes, and other externalities. A wide range of biological and hybrid processes have been developed to make bio-based substitutions for fossil resource-derived raw materials used in the production of commodity and specialty plastics. Yet, bio-renewable circularity in polymers remains a significant challenge, particularly for condensation polymers that are formulated with different monomers to access specific properties.

### SUMMARY

Lacking in the prior art has been the identification of PDK compositions that may be produced, in part or in whole, from bio-based raw materials. Many bio-based raw materials are functionalized in ways that would be difficult to replicate with chemical synthesis. That functionalization can offer a means to tailor the properties of PDK materials. Furthermore, many bio-based raw materials are chiral. If chiral monomers were used in the production of PDK resins, the resins would, in turn, be chiral. Chirality is known to influence the properties of many polymers, but has not been used to tailor the properties of PDK materials.

Also lacking in the prior art has been the identification of PDK compositions that undergo acidolysis when the polymers have a linear chain topology. There are no reports of monomer recovery after acidolysis of linear PDK polymers because the rate of diketoenamine hydrolysis is demonstrably too slow with PDK compositions based on prior art.

In one aspect the present disclosure pertains to a composition of polymers containing diketoenamine bonds that hydrolyze in aqueous acid due to the placement of heteroatoms at specific sites near the diketoenamine bond. Without the heteroatoms at those sites, depolymerization rates may be outside of the range of what is suitable for chemical recycling of materials and products comprising the polymer. After depolymerization, the contrasting properties of the dissociated polytopic triketone and amine monomers may enable their separation, recovery, and refinement for reuse in circular plastics manufacturing. Studies were performed that show bio-based raw materials may be used to synthesize polytopic triketone and amine monomers with heteroatoms at the appropriate sites necessary to accelerate diketoenamine hydrolysis and depolymerization rates. The use of bio-based raw materials also opens the door to functionalized and, in some examples, chiral monomers from which to prepare functionalized and, in some examples, chiral polymers containing diketoenamine bonds.

Some examples provide for a composition of polymers comprising hydrolyzable diketoenamine bonds. This composition allows the formulation of polymeric materials with a wide range of architectures and properties, controllable bio-based content, and further allows these materials to be recycled using thermal, chemical, or mechanical processes.

In examples described are polymers. For example, polydiketoenamines are a class of polymers provided. An example polymer has a formula according to Formula IA:

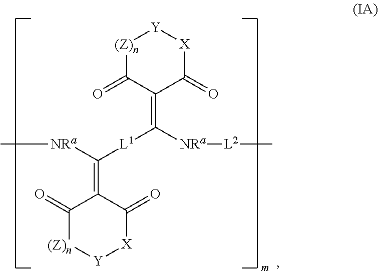

or a tautomer thereof, wherein:
subscript n is an integer ranging from 0 to 2;
L¹ is a linear, divalent linker moiety, and
L² is a linear, divalent linker moiety or a branched, trivalent linker moiety,
provided that at least one of L¹ and L² comprises one or more heteroatoms selected from the group consisting of O, S, Se, and P;
optionally, each subscript n is independently 0 or 1;
each X is independently selected from the group consisting of O, CR^{1a}R^{1b}, SiR^{1a}R^{1b}, NR^{1c}, S, Se, and PR^{1d};
each Z is independently selected from the group consisting of O, CR^{3a}R^{3b}, SiR^{3a}R^{3b}, NR^{3c}, S, Se, and PR^{3d};
each Y is independently selected from the group consisting of O, CR^{2a}R^{2b}, SiR^{2a}R^{2b}, NR^{2c}, S, Se, and PR^{2d}, or Y is CR^{2a}R^{2b} when X is O, SiR^{1a}R^{1b}, NR^{1c}, S, Se, or PR^{1d}, or Y is CR^{2a}R^{2b} when Z is O, SiR^{3a}R^{3b}, NR^{3c}, S, Se, or PR^{3d};
each R^{1a}, R^{1b}, R^{2a}, R^{2b}, R^{3a}, and R^{3b} is independently selected from the group consisting of hydrogen, C₁₋₂₀ alkyl, C₂₋₂₀ alkenyl, C₂₋₂₀ alkynyl, C₃₋₈ cycloalkyl, C₆₋₁₄ aryl, 3- to 12-membered heterocyclyl, and 5- to 12-membered heteroaryl, wherein each alkyl is optionally and independently substituted with C₃₋₈ cycloalkyl, C₆₋₁₄ aryl, 3- to 12-membered heterocyclyl, or 5- to 12-membered heteroaryl;
each R^{1c}, R^{2c}, and R^{3c} is independently selected from the group consisting of hydrogen and C₁₋₆ alkyl, wherein each alkyl is optionally substituted with C₃₋₈ cycloalkyl, C₆₋₁₄ aryl, 3- to 12-membered heterocyclyl, or 5- to 12-membered heteroaryl;
each R^{1d}, R^{2d}, and R^{3d} is independently selected from the group consisting of C₁₋₆ alkyl, C₃₋₈ cycloalkyl, and C₆₋₁₄ aryl, wherein each alkyl is optionally substituted with C₃₋₈ cycloalkyl, C₆₋₁₄ aryl, 3- to 12-membered heterocyclyl, or 5- to 12-membered heteroaryl; and
R^{2a} is optionally taken together with R^{1a} or R^{3a} to form a carbon-carbon double bond.

Optionally L¹ is a divalent hydrocarbon linker, optionally containing one or more heteroatoms selected from the group consisting of O, N, S, Se, and P. Optionally, L² is a divalent hydrocarbon linker, optionally containing one or more heteroatoms selected from the group consisting of O, N, S, Se, and P, or L² is a trivalent hydrocarbon linker containing one or more heteroatoms selected from the group consisting of O, N, S, Se, and P.

Optionally, L¹ is —CH₂OCH₂— or —CH₂OCH₂CH₂OCH₂—. Optionally, L² is C₁₋₂₀ alkylene. Optionally, L² is:

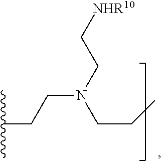

and R¹⁰ is H or a branching polydiketoenamine moiety.

In some examples, L² is:

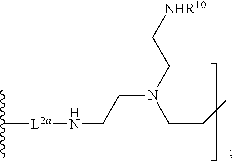

R¹⁰ is H or a branching polydiketoenamine moiety, and
L²a is an oxygen-containing divalent polymer. Optionally, L^{2a} is a poly(tetrahydrofuran) moiety or a poly(ethylene glycol) moiety.

Optionally, L¹ is C₁₋₂₀ alkylene.

Optionally, L² is —(CH₂CH₂O)ₚCH₂CH₂— or —(CH₂)₃O(CH₂)₄O(CH₂)₃—.

In some examples, L² is:

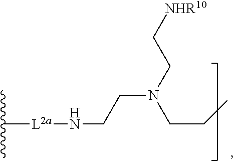

wherein R¹⁰ is H or a branching polydiketoenamine moiety, and
L²a is an oxygen-containing divalent polymer. Optionally, L^{2a} is a poly(tetrahydrofuran) moiety or a poly(ethylene glycol) moiety.

In some examples, X is O, Y is CR^{2a}R^{2b}, subscript n is 1, and Z is CR^{3a}R^{3b}.

Optionally, R^{2a} and R^{3a} are taken together to form a carbon-carbon double bond, R^{2b} is C₁₋₆ alkyl, and R^{3b} is hydrogen. Optionally, R^{2a} and R^{3a} are hydrogen and R^{2b} and R^{3b} are independently C₁₋₆ alkyl. Optionally, R^{2a} and R^{2b} are independently C₁₋₆ alkyl and R^{3a} and R^{3b} are hydrogen.

In some examples, composites are provided, such as a composition comprising a poly diketoenamine according any of the examples described herein and one or materials selected from the group consisting of an additional polymer, a filler material, a substrate material, a flame-retardant, and a pigment. Optionally, one or more additional polymers are selected from the group consisting of a polyurethane, a polyurea, an epoxy, a phenolic resin, a polyolefin, a silicone, a rubber, a polyacrylate, a polymethacrylate, a polycyanoacrylate, a polyester, a polycarbonate, a polyimide, a polyamide, a vitrimer, a poly(vinylogous amide), a poly(vinylogous urethane), and a thermoplastic elastomer. Optionally, the filler material is selected from the group consisting of woven or non-woven carbon fibers, woven or non-woven polyaramid fibers, woven or non-woven glass fibers, carbon black, carbon nanotubes, graphene, diamondoids, aluminum, steel, stainless steel, iron, zinc, titanium, liquid metals, silicon carbide, boron nitride, metal oxide, metal pnictides, metal chalcogenides, metal halides, transition metal dichalcogenides, metal alloys, MXenes, vitrimers, zeolites, metal-organic frameworks, covalent organic frameworks, alumina, silica, silicate clays, and combinations thereof. Optionally, the silicate clay is selected from the group consisting of laponite, sumecton, monomorillonite, sodium fluorohectorite, sodium tetrasilicic mica, and combinations thereof. Optionally, the substrate material is selected from the group consisting of plastic, metal, ceramic, glass, composite, wood, and combinations thereof. Optionally, the flame-retardant is selected from the group consisting of a brominated compound, a chlorinated compound, a nitrogen-containing compound, a phosphorous-containing compound, a metal oxide such (e.g., antimony trioxide), a hydrated metal oxide (e.g., a hydrated aluminum oxide or hydrated magnesium oxide), or a combination thereof. Optionally, the composite may be provided as an extruded solid.

In some examples, polymers described herein and composites described herein are Provided as a population of fibers having an average diameter, width, or thickness ranging from about 0.5 nm to about 1.0 mm and an average length ranging from about 5 nm to about 5000 meters. In examples, the average diameter, width, or thickness may be from 0.5 nm to 1 nm, from 1 nm to 5 nm, from 5 nm to 10 nm, from 10 nm to 50 nm, from 50 nm to 100 nm, from 100 nm to 500 nm, from 500 nm to 1 μm, from 1 μm to 5 μm, from 5 μm to 10 μm, from 10 μm to 50 m, from 50 μm to 100 μm, from 100 μm to 500 μm, or from 500 μm to 1 μmm. In examples, the average length may be from 5 nm to 10 nm, from 10 nm to 50 nm, from 50 nm to 100 nm, from 100 nm to 500 nm, from 500 nm to 1 μm, from 1 μm to 5 μm, from 5 μm to 10 μm, from 10 μm to 50 μm, from 50 μm to 100 μm, from 100 μm to 500 μm, from 500 μm to 1 μmm, from 1 μmm to 5 mm from 5 mm to 1 cm, from 1 cm to 5 cm, from 5 cm to 10 cm, from 10 cm to 50 cm, from 50 cm to 1 m, from 1 m to 5 m, from 5 m to 10 m, from 10 m to 50 m, from 50 m to 100 m, from 100 m to 500 m, from 500 m to 1000 m, or from 1000 m to 5000 m.

In some examples, polymers described herein and composites described herein are Provided as a porous material, such as having pore sizes ranging from about 0.5 nm to about 5000 nm. In examples, the pore sizes may be from 0.5 nm to 1 nm, from 1 nm to 5 nm, from 5 nm to 10 nm, from 10 nm to 50 nm, from 50 nm to 100 nm, from 100 nm to 500 nm, from 500 nm to 1 μm, or from 1 μm to 5 μm. In some examples, such porous materials may comprise a sorbent or a membrane or a foam. Optionally, the foam has a density ranging from about 0.1 pounds per cubic foot to about 10 pounds per cubic foot, such as from 0.1 pounds per cubic foot to 0.5 pounds per cubic foot, from 0.5 pounds per cubic foot to 1 pound per cubic foot, from 1 pound per cubic foot to 5 pounds per cubic foot, or from 5 pounds per cubic foot to 10 pounds per cubic foot.

Optionally, polymers described herein and composites described herein may be provided as a suspension or in a solvent. In some examples, the polymers described herein and composites may be present in an amount ranging from about 0.01% to about 80% on a per weight basis with respect to the solvent, such as from 0.01% to 0.05%, from 0.05% to 0.10%, from 0.10% to 0.50%, from 0.50% to 1.0%, from 1.0% to 5.0%, from 5.0% to 10%, from 10% to 20%, from 20% to 40%, from 40% to 60%, or from 60% to 80%.

Optionally, polymers described herein and composites described herein may comprise or be configured as a conductive material or an insulating material.

Methods are also provided herein, such as methods for recycling a polydiketoenamines. An example, method comprising combining a polydiketoenamine described herein with an acid or a base or a combination thereof to depolymerize the polydiketoenamine. Optionally, the polydiketoenamines is provided as a composite, as described herein. Optionally, the acid is selected from the group consisting of HCl, H₂SO₄, H₃PO₄, p-toluenesulfonic acid, methane sulfonic acid, trifluoroacetic acid, and trifluoromethanesulfonic acid. Optionally, the base is an amine base.

In examples, other compositions are provided, such as those having a formula of:

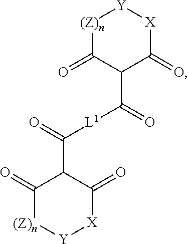

or a tautomer thereof, wherein:
L¹ is a linear, divalent linker moiety comprising one or more heteroatoms selected from the group consisting of O, N, S, Se, and P;
each subscript n is independently 0 or 1;
each X is independently selected from the group consisting of O, CR^{1a}R^{1b}, SiR^{1a}R^{1b}, NR^{1c}, S, Se, and PR^{1d};
each Z is independently selected from the group consisting of O, CR^{3a}R^{3b}, SiR^{3a}R^{3b}, NR^{3c}, S, Se, and PR^{3d};
each Y is independently selected from the group consisting of O, CR^{2a}R^{2b}, SiR^{2a}R^{2b}, NR^{2c}, S, Se, and PR^{2d}, or Y is CR^{2a}R^{2b} when X is O, SiR^{1a}R^{1b}, NR^{1c}, S, Se, or PR^{1d}, or Y is CR^{2a}R^{2b} when Z is O, SiR^{3a}R^{3b}, NR^{3c}, S, Se, or PR^{3d};
each R^{1a}, R^{1b}, R^{2a}, R^{2b}, R^{3a}, and R^{3b} is independently selected from the group consisting of hydrogen, C₁₋₂₀ alkyl, C₂₋₂₀ alkenyl, C₂₋₂₀ alkynyl, C₃₋₈ cycloalkyl, C₆₋₁₄ aryl, 3- to 12-membered heterocyclyl, and 5- to 12-membered heteroaryl, wherein each alkyl is optionally and independently substituted with C₃₋₈ cycloalkyl, C₆₋₁₄ aryl, 3- to 12-membered heterocyclyl, or 5- to 12-membered heteroaryl;
each R^{1c}, R^{2c}, and R^{3c} is independently selected from the group consisting of hydrogen and C₁₋₆ alkyl, wherein each alkyl is optionally substituted with C₃₋₈ cycloalkyl, C₆₋₁₄ aryl, 3- to 12-membered heterocyclyl, or 5- to 12-membered heteroaryl;
each R^{1d}, R^{2d}, and R^{3d} is independently selected from the group consisting of C₁₋₆ alkyl, C₃₋₈ cycloalkyl, and C₆₋₁₄ aryl, wherein each alkyl is optionally substituted with C₃₋₈ cycloalkyl, C₆₋₁₄ aryl, 3- to 12-membered heterocyclyl, or 5- to 12-membered heteroaryl; and
R^{2a} is optionally taken together with R^{1a} or R^{3a} to form a carbon-carbon double bond.

Optionally, L¹ is —CH₂OCH₂— or —CH₂OCH₂CH₂OCH₂—. Optionally, R^{2a} and R^{3a} are taken together to form a carbon-carbon double bond, R^{2b} is C₁₋₆ alkyl, and R^{3b} is hydrogen. Optionally, R^{2a} and R^{3a} are hydrogen and R^{2b} and R^{3b} are independently C₁₋₆ alkyl. Optionally, R^{2a} and R^{2b} are independently C₁₋₆ alkyl and R^{3a} and R^{3b} are hydrogen.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 provides a plot showing activation barrier for diketoenamine hydrolysis as it varies with O-atom placement on the triketone monomer.

FIG. 2 provides a plot showing activation barrier for diketoenamine hydrolysis as it varies with O-atom placement on the amine monomer.

FIG. 3 provides a plot showing activation barrier for diketoenamine hydrolysis as it varies with N-atom placement on the amine monomer.

FIG. 4 provides a single crystal X-ray structure for a ditopic triketone monomer, which is derived from triacetic acid lactone and suberic acid.

FIG. 5 provides a single crystal X-ray structure fora ditopic triketone monomer, which is derived from triacetic acid lactone and sebacic acid.

FIG. 6 provides a single crystal X-ray structure for a triketone monomer, which is derived from triacetic acid lactone and dodecanedioic acid.

FIG. 7 provides a schematic overview of bio-Renewable circularity in polydiketoenamines.

FIG. 8 provides images of compression molding of polydiketoenamine networks from resin powders.

FIG. 9 provides a plot showing glass transition temperatures for polydiketoenamine networks derived from triacetic acid lactone.

FIG. 10 provides a plot showing odd-even effects in the density of polydiketoenamine networks derived from triacetic acid lactone.

FIG. 11 provides a plot showing odd-even effects in the tensile elastic modulus of polydiketoenamine networks derived from triacetic acid lactone.

FIG. 12 provides a plot showing storage modulus and loss modulus of elastomeric poly diketoenamines derived from polytetrahydrofuran, whose chain ends are functionalized with tris(2-aminoethyl)amine.

FIG. 13 provides a plot showing storage modulus and loss modulus of carbon-black reinforced polydiketoenamine rubbers derived from polytetrahydrofuran, whose chain ends are functionalized with tris(2-aminoethyl)amine.

FIG. 14 provides a plot showing stress relaxation of elastomeric polydiketoenamines derived from polytetrahydrofuran, whose chain ends are functionalized with tris(2-aminoethyl)amine.

FIG. 15 provides a plot showing stress relaxation of carbon-black reinforced polydiketoenamine rubbers derived from polytetrahydrofuran, whose chain ends are functionalized with tris(2-aminoethyl)amine.

FIG. 16 provides a plot showing creep resistance of elastomeric polydiketoenamines derived from polytetrahydrofuran, whose chain ends are functionalized with tris(2-aminoethyl)amine.

FIG. 17 provides a plot showing creep resistance of carbon-black reinforced polydiketoenamine rubbers derived from polytetrahydrofuran, whose chain ends are functionalized with tris(2-aminoethyl)amine.

FIG. 18 provides an overview of triketone monomer recovery from chemically recycled polydiketoenamine networks derived from triacetic acid lactone.

FIG. 19 provides an overview of triketone monomer recovery from chemically recycled polydiketoenamine networks derived from 0-keto-6-lactones.

FIG. 20 provides an overview of chemical depolymerization of polydiketoenamines with linear polymer architectures.

FIG. 21 provides data showing NMR spectra of a triketone monomer recovered from chemically recycled polydiketoenamines with linear polymer architectures.

FIG. 22 provides images showing chemical depolymerization of elastomeric polydiketoenamines and carbon-reinforced polydiketoenamine rubbers derived from polytetrahydrofuran, whose chain ends are functionalized with tris(2-aminoethyl)amine.

FIG. 23 provides images showing triketone monomer recovery from elastomeric polydiketoenamines and carbon-reinforced polydiketoenamine rubbers derived from polytetrahydrofuran, whose chain ends are functionalized with tris(2-aminoethyl)amine.

FIG. 24 provides images showing comparison of the depolymerization rates for two different elastomeric polydiketoenamines derived from polytetrahydrofuran: (1) incomplete depolymerization in ˜24 h observed for a polydiketoenamine elastomer prepared using TREN and a commercially available polytetrahydrofuran, whose chain ends are functionalized with an amine, and (2) complete depolymerization in ˜24 h of a polydiketoenamine elastomer prepared using a polytetrahydrofuran, whose chain ends are functionalized with tris(2-aminoethyl)amine.

FIG. 25 provides a synthetic scheme for preparation of a first example monomer.

FIG. 26 provides a synthetic scheme for preparation of a second example monomer.

FIG. 27 provides a synthetic scheme for preparation of a third example monomer.

FIG. 28 provides a synthetic scheme for preparation of a fourth example monomer.

FIG. 29 provides a synthetic scheme for preparation of a fifth example monomer.

FIG. 30 provides a synthetic scheme for preparation of an example monomer intermediate.

FIG. 31 provides a synthetic scheme for preparation of an example monomer intermediate.

FIG. 32 provides a synthetic scheme for preparation of an example monomer intermediate.

FIG. 33 provides a synthetic scheme for preparation of a sixth example monomer.

FIG. 34 provides a synthetic scheme for preparation of a seventh example monomer.

FIG. 35 provides a synthetic scheme for preparation of an eighth example monomer.

FIG. 36 provides a synthetic scheme for preparation of an example PDK Network.

FIG. 37 provides a synthetic scheme for preparation of an example chiral PDK Network.

FIG. 38 provides a synthetic scheme for preparation of a first example linear polydiketonenamine.

FIG. 39 provides a synthetic scheme for preparation of a second example linear polydiketonenamine.

FIG. 40 provides a synthetic scheme for preparation of a third example linear polydiketonenamine.

FIG. 41 provides a synthetic scheme for preparation of an example monomer intermediate.

FIG. 42 provides a synthetic scheme for preparation of an example monomer intermediate.

FIG. 43 provides a synthetic scheme for preparation of an example PDK elastomer.

FIG. 44 provides a synthetic scheme for depolymerization of an example PDK Network.

FIG. 45 provides a synthetic scheme for depolymerization of an example chiral bio-based PDK Network.

FIG. 46 provides a synthetic scheme for preparation of an example PDK elastomer with incomplete depolymerization in strong acid.

FIG. 47 depicts examples of commercial products that incorporate crosslinked elastomeric components that are challenging to recover and recycle (Panel A); schematics of monomer and corresponding polymer network structure for PDK-multivalent and PDK-monovalent (Panel B); monomer structures for multivalent soft segment: poly(tetrahydrofuran)-bis-tris-2(aminoethyl)amine (pTHF-bis-TREN); triketone: 2,2′-decanedioylbis(5,5-dimethylcyclohexane-1,3-dione) (TK-10); monovalent soft segment: poly(tetrahydrofuran)-diamine (pTHF-diamine); TREN: tris-2(aminoethyl)amine (Panel C); stress-strain plots for PDK-multivalent (strain at break=104%, tensile strength at break=1.14 MPa, toughness=78.5 MJ m⁻³) and PDK-monovalent (strain at break=268%, tensile strength at break=0.257 MPa, toughness=49.9 MJ m⁻³) (Panel D).

FIG. 48 provides data showing frequency sweep (Panel A), amplitude sweep (Panel B), and stress relaxation measurements (Panel C) for PDK-multivalent elastomers, as well as frequency sweep (Panel D), amplitude sweep (Panel E), and stress relaxation measurements (Panel E) for PDK-monovalent elastomers.

FIG. 49 provides data showing frequency sweep (Panel A), amplitude sweep (Panel B), and stress relaxation measurements (Panel C) for PDK-multivalent containing 0.5 wt % carbon black, as well as frequency sweep (Panel D), amplitude sweep (Panel E), and stress relaxation measurements (Panel F) for PDK-monovalent containing 0.5 wt % carbon black.

FIG. 50 provides data showing PDK-multivalent elastomer creep, showing exceptional creep resistance at all temperatures (Panel A), PDK-monovalent elastomer creep, showing high susceptibility to creep at all temperatures (Panel B), strain rate (dγ/dt) vs temperature for PDK-multivalent and PDK-monovalent elastomers (Panel C), PDK-multivalent carbon-reinforced (0.5 wt %) rubber creep, showing exceptional creep resistance at all temperatures (Panel D), PDK-monovalent carbon-reinforced (0.5 wt %) creep, showing improved creep resistance at all temperatures (Panel E), and strain rate (dγ/dt) vs temperature for PDK-multivalent and PDK-monovalent carbon-reinforced (0.5 wt %) elastomers (Panel F).

FIG. 51 provides polymerization and depolymerization schemes for elastomers and photographs of chemical depolymerization of elastomers with and without carbon black.

FIG. 52 provides computational reaction coordinates for acid-catalyzed diketoenamine hydrolysis.

FIG. 53 provides photographs of elastomer samples before and after reprocessing.

FIG. 54 provides DSC traces of PDK-multivalent elastomers without carbon black (Panel A), and with 0.5 wt % carbon black (Panel B).

FIG. 55 provides DSC traces of PDK-monovalent elastomers: without carbon black (Panel A), and with 0.5 wt % carbon black (Panel B).

FIG. 56 provides ATR-FTIR spectra of PDK-multivalent elastomers: without carbon black (Panel A), and with 0.5 wt % carbon black (Panel B).

FIG. 57 provides ATR-FTIR spectra of PDK-monovalent elastomers: without carbon black (Panel A), and with 0.5 wt % carbon black (Panel B).

FIG. 58 provides ¹H NMR spectra of recycled and pristine TK-10 (Panel A) and MALDI mass spectra of recycled and pristine pTHF-bis-TREN (Panel B).

FIG. 59 depicts chemical structures of small-molecule analogues of PDK-multivalent or PDK-monovalent using acyl dimedone and n-butylamine (top series) or N,N-dimethylaminoethylamine (bottom series).

FIG. 60 provides an illustration relating to how varying the amine spacing in circular polydiketoenamines tunes their depolymerization rate.

FIG. 61 illustrates variation of the diketoenamine hydrolysis rate with increasing amine spacing.

FIG. 62 provides plots showing decomposition of the energy barrier in the distortion-interaction model.

FIG. 63 illustrates calculated and observed hydrolysis free energy barriers.

FIG. 64 illustrates C₂ and C₃ PDK formulations hydrolysis.

FIG. 65 depicts an example procedure for identifying the lowest energy conformers of the addition transition state.

FIG. 66 provides data showing hydrolysis kinetics of an example elastomer at 60, 70 and 75° C.

FIG. 67 provides data showing hydrolysis kinetics of an example elastomer at 20, 30, and 40° C.

FIG. 68 provides data showing hydrolysis kinetics of an example elastomer at 41, 50, and 60° C.

FIG. 69 provides data showing hydrolysis kinetics of an example elastomer at 65, 70 and 80° C.

FIG. 70 provides data showing hydrolysis kinetics of an example elastomer at 60, 70 and 80° C.

FIG. 71 provides data showing a ¹H NMR of pristine and chemically recycled triketone monomer from C₂ triamine PDK recycling after 24 h.

FIG. 72 provides data showing ¹H NMR of pristine and chemically recycled triketone monomer from C₃ triamine PDK recycling after 96 h.

FIG. 73 illustrates a reaction mechanism for the acid-catalyzed hydrolysis of an example elastomer.

FIG. 74 illustrates biorenewable circularity in PDK plastics derived from triacetic acid lactone (TAL).

FIG. 75 illustrates recycling of TAL-PDK formulations.

FIG. 76 depicts biosynthesis of triacetic acid lactone (bioTAL) and biorenewable TAL-PDK characterization.

FIG. 77 provides an overview of systems analysis of the production of bioTAL.

FIG. 78 provides single-crystal XRD for a variety of compounds.

FIG. 79 provides single-crystal XRD for a variety of compounds.

FIG. 80 provides data showing DSC of a variety of compounds.

FIG. 81 provides data showing solid-state ¹³C NMR spectra of different compounds.

FIG. 82 provides data showing solid-state ¹³C NMR spectra of different compounds.

FIG. 83 provides data showing solid-state ¹³C NMR spectra of different compounds.

FIG. 84 provides data showing solid-state ¹³C NMR spectra of different compounds.

FIG. 85 provides data showing solid-state ¹³C NMR spectra of different compounds.

FIG. 86 provides data showing solid-state ¹³C NMR spectra of different compounds.

FIG. 87 provides an overview of processing of TAL-PDK resins into solid bar samples.

FIG. 88 provides data showing DSC of a variety of compounds.

FIG. 89 provides data showing TGA of a variety of compounds and powder and pressed compounds.

FIG. 90 provides DMA of a variety of compounds.

FIG. 91 provides data showing a ¹H NMR of a compound recovered from depolymerized resin (top) and the original monomer (bottom).

FIG. 92 provides data showing a ¹H NMR of a compound recovered from depolymerized resin (top) and the original monomer (bottom).

FIG. 93 provides data showing a ¹H NMR of a compound recovered from depolymerized resin (top) and the original monomer (bottom).

FIG. 94 provides data showing a ¹H NMR of a compound recovered from depolymerized resin (top) and the original monomer (bottom).

FIG. 95 provides data showing a ¹H NMR of a compound recovered from depolymerized resin (top) and the original monomer (bottom).

FIG. 96 provides data showing ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 97. provides data showing ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 98 provides data showing ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 99 provides data showing ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 100 provides data showing ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 101 provides data showing a ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 102 provides data showing a ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 103 provides data showing a ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 104 provides data showing a ¹H NMR spectra of a purified compound recovered from depolymerized compression-molded plastics (top), crude material recovered from depolymerized compression-molded plastics (middle), and original monomer (bottom). Notable impurities caused by parasitic side reactions are identified by asterisks (*).

FIG. 105 provides data showing ESI-MS spectra of crude material recovered from depolymerized compression-molded plastics (top) and the original monomer (bottom). The prevalent peak corresponding to a decarboxylated subproduct is identified.

FIG. 106 provides data showing ESI-MS spectra of crude material recovered from depolymerized compression-molded plastics (top) and the original monomer (bottom). The prevalent peak corresponding to a decarboxylated subproduct is identified.

FIG. 107 provides data showing ESI-MS spectra of crude material recovered from depolymerized compression-molded plastics (top) and the original monomer (bottom). The prevalent peak corresponding to a decarboxylated subproduct is identified.

FIG. 108 provides data showing ESI-MS spectra of crude material recovered from depolymerized compression-molded plastics (top) and the original monomer (bottom). The prevalent peak corresponding to a decarboxylated subproduct is identified.

FIG. 109 provides data showing ESI-MS spectra of crude material recovered from depolymerized compression-molded plastics (top) and the original monomer (bottom). The prevalent peak corresponding to a decarboxylated subproduct is identified.

FIG. 110 provides a mechanistic hypothesis of TAL-PDK degradation leading to pyrone-triketone monomer.

FIG. 111 depicts 1-L fed batch fermentation of TAL production with E. coli JBEI-3695 pBbA5A-BktB.

FIG. 112 provides dual-wavelength optical densities of eluent acquired during TAL purification by column chromatography.

### DETAILED DESCRIPTION

Before the present disclosure is described in detail, it is to be understood that, unless otherwise indicated, this disclosure is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although, any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The present disclosure relates to a composition of polymers comprising diketoenamine bonds optionally with heteroatoms placed at specific sites near the diketoenamine bond. This composition allows the formulation of polymeric materials with bio-renewable monomers and bio-advantaged properties, and further allows these polymers to be recycled using mechanical, thermal, and chemical processes.

Polydiketoenamine polymers bearing heteroatoms at specific locations undergo depolymerization at rates that are orders of magnitude higher than if those heteroatoms are placed elsewhere, or are not present at all. This opens the door, for the first time, to chemical recycling of PDK resins with linear chain architectures and significantly widens the scope of polymer compositions. With respect to the latter, heteroatom placement can be controlled through chemical synthesis or biosynthesis. Embodiments are identified where biosynthesized chemicals used in monomer and polymer synthesis offer performance advantages that would be difficult to replicate using chemically synthesized analogues. Embodiments along those lines therefore have the potential to be high performance, highly recyclable, and bio-renewable.

In some examples, the disclosed composition comprises a polymer. Without limitation, examples include those comprising a polymer, or polymer network, which may have at least one unit of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI) and/or (XXXII), or a mixture thereof.

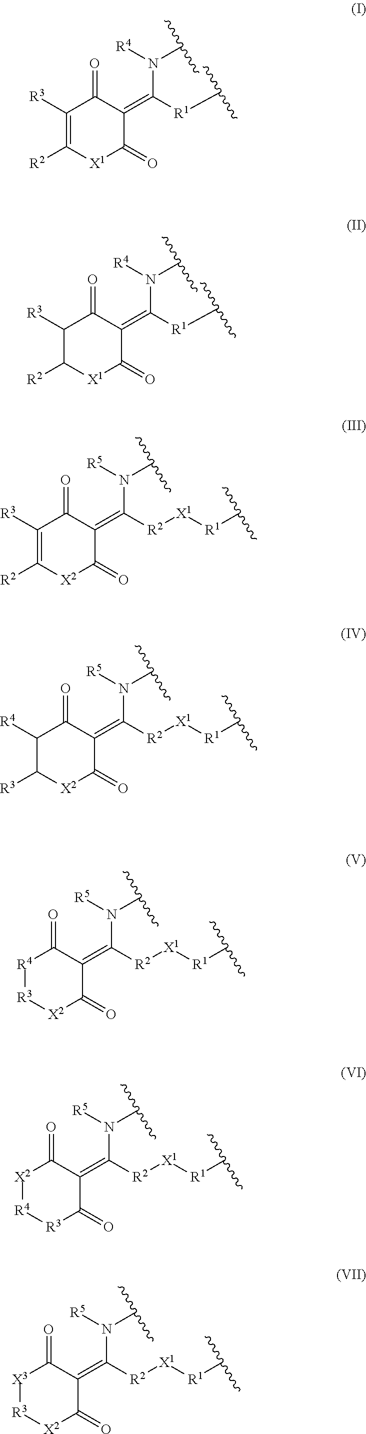

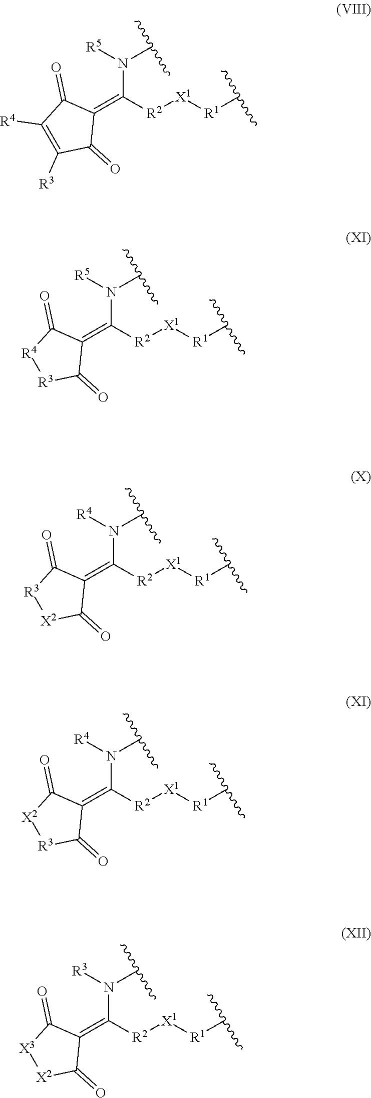

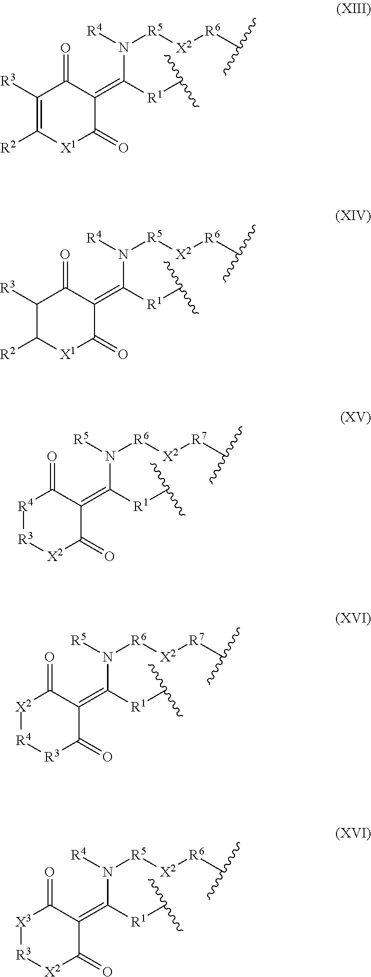

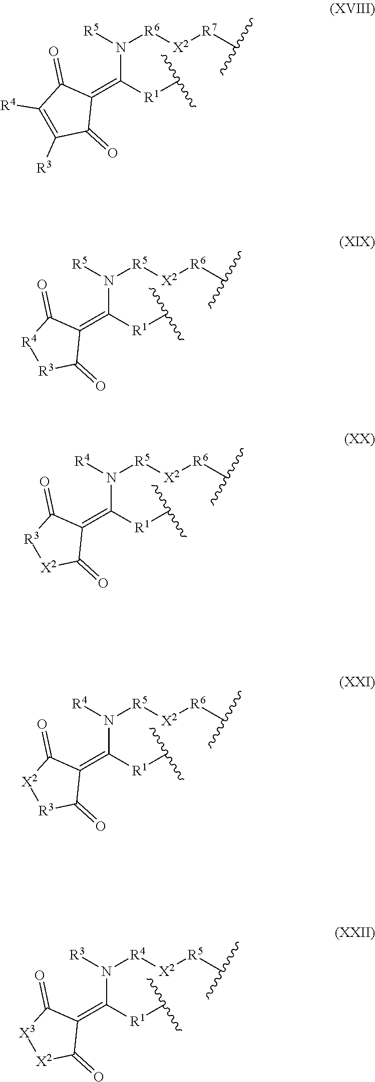

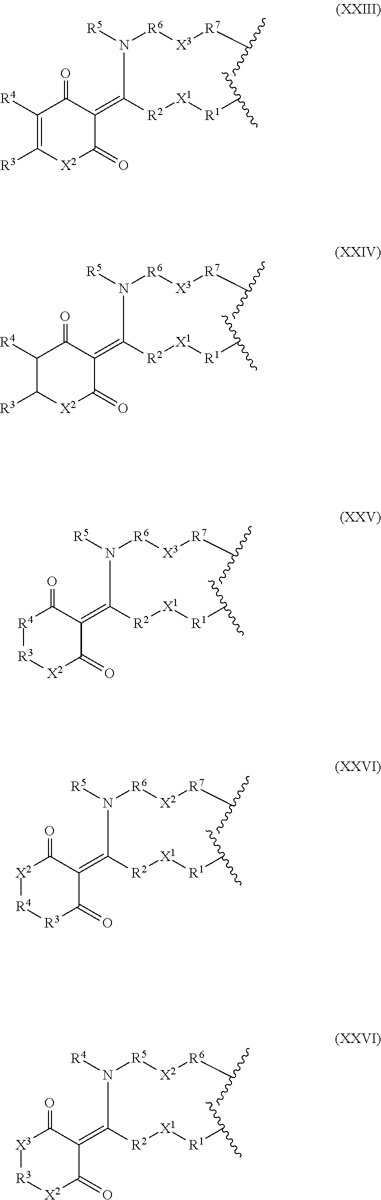

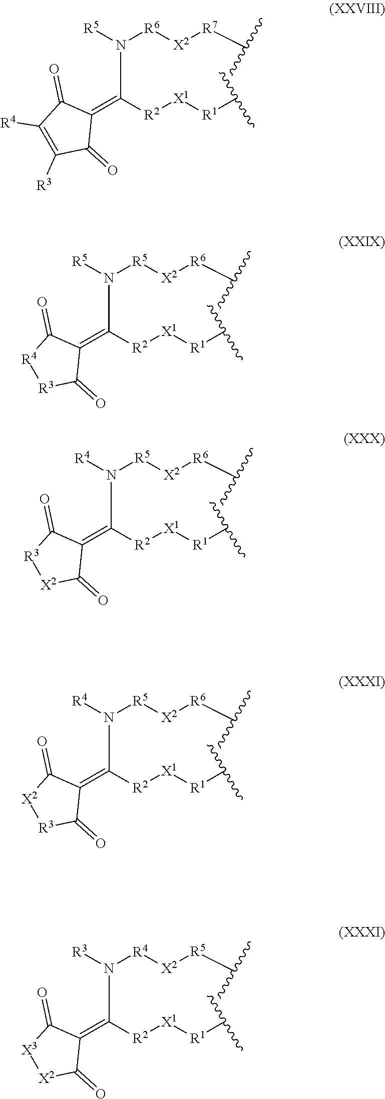

In some examples, the polymer, or polymer network, may optionally be obtained by connecting a first compound to a second Compound. In examples the first compound may comprise at least two functional groups selected from group (A), (B), (C), (D), (E), (F), (G), (H), (I), (J), (K), (L), (M), (N), (O), (P), (Q), (R), (S), (T), (U), (V), (W), (X), (Y) and/or (Z), or a mixture thereof.

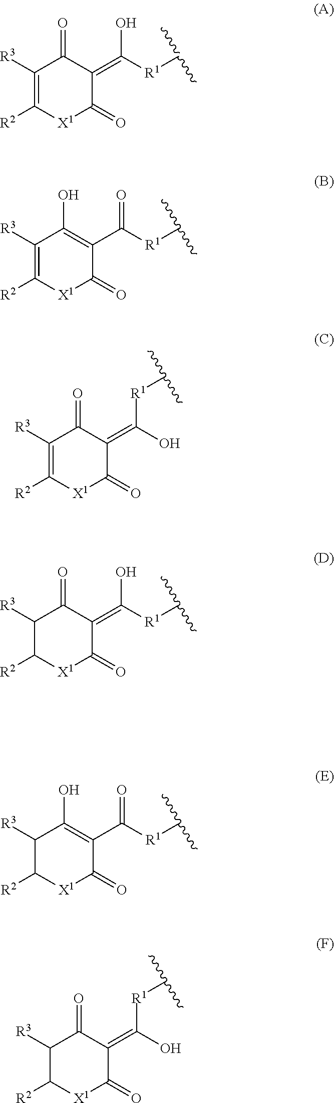

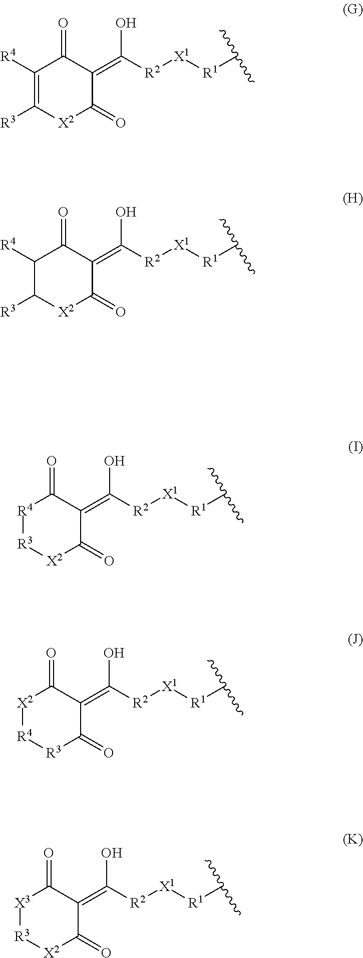

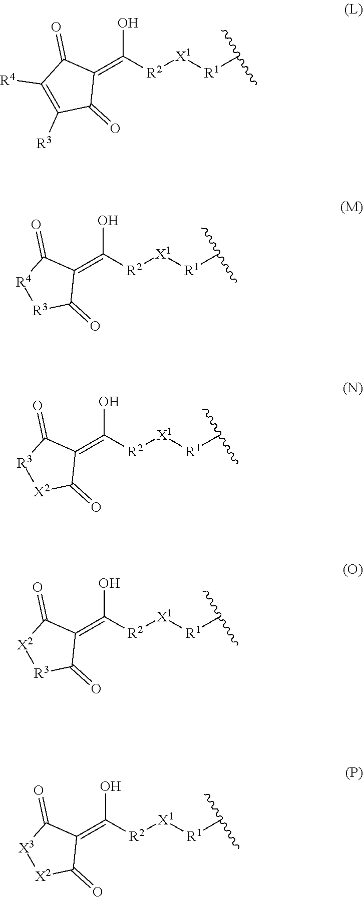

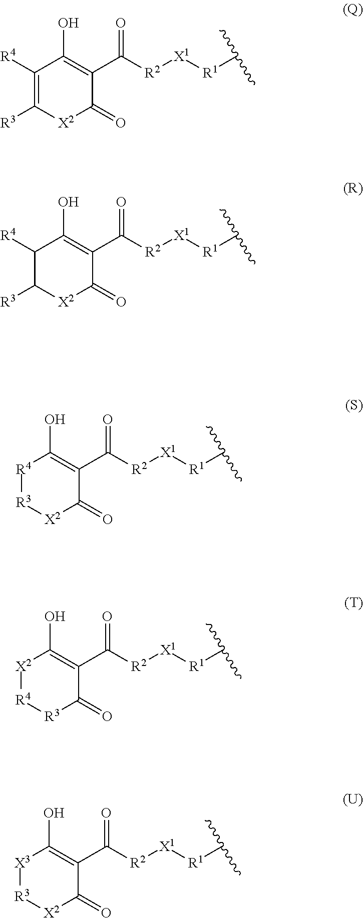

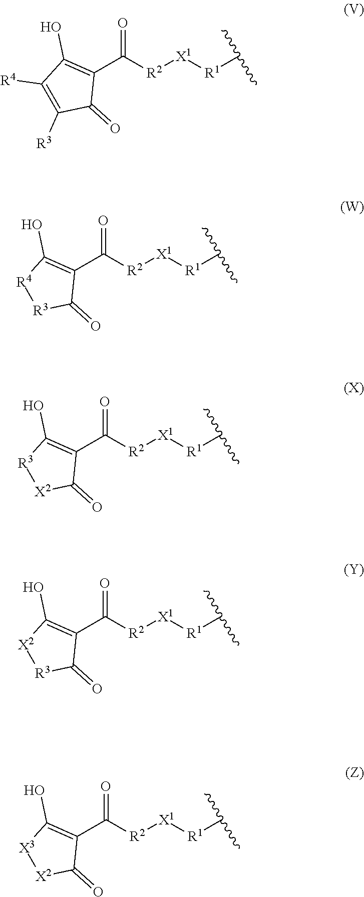

In some examples, the second compound may optionally have at least two amine functional groups of the type —NH₂, —NHR⁴, —NH₃⁺ and/or —NHR⁴R⁵⁺ groups, or optionally at least two functional groups that generates —NH₂, —NHR⁴, —NH₃⁺ and/or —NHR⁴R⁵⁺ in situ, or a mixture thereof.

In some examples, the amine on the second compound may be (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, (C₃₋₈)cycloalkyl(C₁₋₂₀)alkyl, hetero(C₁₋₂₀)alkyl, heterocyclyl, heterocyclyl(C₁₋₂₀)alkyl, heteroaryl, and/or heteroaryl(C₁₋₂₀)alkyl.

In some examples, the (C₁₋₂₀)alkyl, (C₂₋₂₀)alkenyl, (C₂₋₂₀)alkynyl, (C₃₋₈)cycloalkyl(C₁₋₂₀)alkyl, (C₆₋₁₂)aryl(C₁₋₂₀)alkyl, heterocyclyl(C₁₋₂₀)alkyl, and/or heteroaryl(C₁₋₂₀)alkyl optionally comprises one or more heteroatoms in the alkyl, alkenyl, alkynyl moiety, the heteroatoms being each independently a C, Si, chalcogenide (such as O, S, or Se), or a pnictide (such as N, or P).

In some examples, at least one carbon atom or heteroatom of the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀) alkyl, heterocyclyl(C₁₋₂₀)alkyl, and/or heteroaryl(C₁₋₂₀)alkyl may be oxidized to form at least one C═O, C═S, N═O, N═S, S═O or S(O)₂.

In some examples, the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, and/or heteroaryl(C₁₋₂₀)alkyl may be unsubstituted or substituted with one or more Z¹.

In some examples, each Z¹ may be independently selected from the group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁶R⁷, —NO₂, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴. Optionally, the ratio R may be less than, or equal to 1 (R≤1), where R may be described as:

$$R = \frac{\sum {F}_{Y} {N}_{Y}}{\sum {F}_{Z} {N}_{Z}} \leq 1.$$

In some examples, R¹ is optionally selected from the group consisting of (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, (C₃₋₈)cycloalkyl(C₁₋₂₀)alkyl, hetero(C₁₋₂₀)alkyl, heterocyclyl, heterocyclyl(C₁₋₂₀)alkyl, heteroaryl, and/or heteroaryl(C₁₋₂₀)alkyl.

In some examples, the (C₁₋₂₀)alkyl, (C₂₋₂₀)alkenyl, (C₂₋₂₀)alkynyl, (C₃₋₈)cycloalkyl(C₁₋₂₀)alkyl, (C₆₋₁₂)aryl(C₁₋₂₀)alkyl, heterocyclyl(C₁₋₂₀)alkyl, and/or heteroaryl(C₁₋₂₀)alkyl, optionally comprises one or more heteroatoms in the alkyl, alkenyl, alkynyl moiety, each heteroatom may be independently a C, Si, chalcogenide (such as O, S, or Se), or a pnictide (such as N, or P).

In some examples, at least one carbon atom or heteroatom of the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀) alkyl, heterocyclyl(C₁₋₂₀)alkyl, and/or heteroaryl(C₁₋₂₀)alkyl may be oxidized to form at least one C═O, C═S, N═O, N═S, S═O or S(O)₂

In some examples, the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, and/or heteroaryl(C₁₋₂₀)alkyl may be unsubstituted or substituted with one or more Z¹.

In some examples, each Z¹ may be independently selected from the group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁶R⁷, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, R² may be selected from the group consisting of (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, (C₃₋₈)cycloalkyl(C₁₋₂₀)alkyl, hetero(C₁₋₂₀)alkyl, heterocyclyl, heterocyclyl(C₁₋₂₀)alkyl, heteroaryl, and/or heteroaryl(C₁₋₂₀)alkyl.

In some examples, the (C₁₋₂₀)alkyl, (C₂₋₂₀)alkenyl, (C₂₋₂₀)alkynyl, (C₃₋₈)cycloalkyl(C₁₋₂₀)alkyl, (C₆₋₁₂)aryl(C₁₋₂₀)alkyl, heterocyclyl(C₁₋₂₀)alkyl, and heteroaryl(C₁₋₂₀)alkyl, optionally comprises one or more heteroatoms in the alkyl, alkenyl, alkynyl moiety, each heteroatom may be independently a C, Si, chalcogenide (such as O, S, or Se), or a pnictide (such as N, or P).

In some examples, at least one carbon atom or heteroatom of the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀) alkyl, heterocyclyl(C₁₋₂₀)alkyl, and/or heteroaryl(C₁₋₂₀)alkyl may be oxidized to form at least one C═O, C═S, N═O, N═S, S═O or S(O)₂.

In some examples, the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, and/or heteroaryl(C₁₋₂₀)alkyl may be unsubstituted or substituted with one or more Z¹.

In some examples, each Z¹ may be independently selected from the group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁶R⁷, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, R² and R³ may be directly bonded together to form a, 5 membered cycloalkyl, heterocyclyl, or heteroaryl.

In some examples, R² and R³ may be bonded together with a linker X¹ to form a 6, 7, or 8 membered cycloalkyl, heterocyclyl, or heteroaryl.

In some examples, X¹ within the cycloalkyl, heterocyclyl, or heteroaryl may be independently selected from the group consisting of C, Si, chalcogenide (such as O, S, or Se), or a pnictide (such as N, or P).

In some examples, each heterocyclyl or heteroaryl may be independently substituted with one or more Z². Optionally, each Z² may be independently selected from the following group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁵R⁶, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, R² and R¹ may be directly bonded together to form a, 5 membered cycloalkyl, heterocyclyl, or heteroaryl.

In some examples, R² and R¹ may be bonded together with a linker X¹ to form a 6, 7, or 8 membered cycloalkyl, heterocyclyl, or heteroaryl.

In some examples, X¹ within the cycloalkyl, heterocyclyl, or heteroaryl may be independently selected from the group consisting of C, Si, chalcogenide (such as O, S, or Se), or a pnictide (such as N, or P).

In some examples, X¹ and R² may be directly bonded together to form a 5, 6, 7, or 8 membered heterocyclyl, or heteroaryl.

In some examples, X¹ and R³ may be directly bonded together to form a 5, 6, 7, or 8 membered cycloalkyl, heterocyclyl, or heteroaryl.

In some examples, each heterocyclyl or heteroaryl may be substituted with one or more Z² where each Z² may be independently selected from the following group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁵R⁶, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, R³ may be selected from the group consisting of (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, (C₃₋₈)cycloalkyl(C₁₋₂₀)alkyl, hetero(C₁₋₂₀)alkyl, heterocyclyl, heterocyclyl(C₁₋₂₀)alkyl, heteroaryl, and/or heteroaryl(C₁₋₂₀)alkyl.

In some examples, the (C₁₋₂₀)alkyl, (C₂₋₂₀)alkenyl, (C₂₋₂₀)alkynyl, (C₃₋₈)cycloalkyl(C₁₋₂₀)alkyl, (C₆₋₁₂)aryl(C₁₋₂₀)alkyl, heterocyclyl(C₁₋₂₀)alkyl, and heteroaryl(C₁₋₂₀)alkyl, optionally comprises one or more heteroatoms in the alkyl, alkenyl, alkynyl moiety, each heteroatom may be independently a C, Si, chalcogenide (such as O, S, or Se), or a pnictide (such as N or P).

In some examples, at least one carbon atom or heteroatom of the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀) alkyl, heterocyclyl(C₁₋₂₀)alkyl, and heteroaryl(C₁₋₂₀)alkyl may be oxidized to form at least one C═O, C═S, N═O, N═S, S═O or S(O)₂.

In some examples, the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, and heteroaryl(C₁₋₂₀)alkyl may be unsubstituted or substituted with one or more Z¹.

In some examples, each Z¹ may be independently selected from the group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁶R⁷, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, R³ and R¹ may be directly bonded together to form a 5 membered cycloalkyl, heterocyclyl, or heteroaryl.

In some examples, R³ and R¹ may be bonded together with a linker X¹ to form a 6, 7, or 8 membered cycloalkyl, heterocyclyl, or heteroaryl.

In some examples, X¹ within the cycloalkyl, heterocyclyl, or heteroaryl may be independently selected from the group consisting of C, Si, chalcogenide (such as O, S, or Se), or a pnictide (such as N or P).

In some examples, each heterocyclyl or heteroaryl may be substituted with one or more Z², where each Z² may be independently selected from the following group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁵R⁶, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, R³ may be linked to R¹ with a linker X¹ to form a 4, 5, 6, or 7 membered cycloalkyl, heterocyclyl, or heteroaryl.

In some examples, X¹ within the cycloalkyl, heterocyclyl, or heteroaryl may be independently selected from the group consisting of C, Si, chalcogenide (such as O, S, or Se), or a pnictide (such as N or P).

In some examples, each heterocyclyl or heteroaryl may be substituted with one or more Z² where each Z² may be independently selected from the following group consisting of halogen, (C₁₋₂₀)alkyl, (C₂₋₂₀)alkenyl, (C₂₋₂₀)alkynyl, (C₆₋₁₂)aryl, (C₃₋₈) cycloalkyl, (C₆₋₁₂)aryl(C₁₋₂₀)alkyl, hetero(C₁₋₂₀)alkyl, heterocyclyl, heteroaryl, (C₃₋₈)cycloalkyl(C₁₋₂₀)alkyl, heterocyclyl(C₁₋₂₀)alkyl, heteroaryl(C₁₋₂₀)alkyl, halo(C₁₋₂₀)alkyl, halo(C₁₋₂₀)alkyloxy, —OR⁵, —SR⁵, —S(O)R⁴, —S(O)₂R⁴, —SO₂NR⁵R⁶, nitro, —NR⁵C(O)R⁴, —NR⁵S(O)₂R⁴, —NR⁵C(O)NR⁶R⁷, NR⁶R⁷, cyano, —CO₂R⁵, —C(O)NR⁶R⁷, and/or —C(O)R⁴.

In some examples, each R⁴ may be independently selected from the group consisting of hydrogen, C(₁₋₂₀) alkyl, C(₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl.

In some examples, the (C₁₋₂₀)alkyl, (C₂₋₂₀)alkenyl, (C₂₋₂₀)alkynyl, (C₆₋₁₂)aryl(C₁₋₂₀)alkyl, heterocyclyl(C₁₋₂₀)alkyl, or heteroaryl(C₁₋₂₀)alkyl, optionally comprises one or more heteroatoms in the alkyl, alkenyl or alkynyl moiety, the heteroatoms being each independently selected from O, S and N. Optionally, at least one carbon atom or heteroatom of the (C₁₋₂₀)alkyl, (C₂₋₂₀) alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl heterocyclyl, heteroaryl, heterocyclyl(C₁₋₂₀)alkyl, and/or heteroaryl(C₁₋₂₀)alkyl may be oxidized to form at least one C═O, C═S, N═O, N═S, S═O or S(O)₂.

In some examples, each R⁵ may be independently selected from the group consisting of hydrogen, (C₁₋₂₀) alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl.

In some examples, the (C₁₋₂₀)alkyl, (C₂₋₂₀)alkenyl, (C₂₋₂₀)alkynyl, (C₆₋₁₂)aryl(C₁₋₂₀)alkyl, heterocyclyl(C₁₋₂₀)alkyl, or heteroaryl(C₁₋₂₀)alkyl optionally comprise one or more heteroatoms in the alkyl, alkenyl or alkynyl moiety, the heteroatoms being each independently selected from O, S and N. Optionally, at least one carbon atom or heteroatom of the (C₁₋₂₀)alkyl, (C₂₋₂₀) alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl may be oxidized to form at least one C═O, C═S, N═O, N═S, S═O or S(O)₂.

In some examples, each R⁶ and R⁷ may be independently selected from the group consisting of hydrogen, (C₁₋₂₀)alkyl, (C₂₋₂₀)alkenyl, (C₂₋₂₀) alkynyl, (C₆₋₁₂) aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl.

In some examples, the (C₁₋₂₀)alkyl, (C₂₋₂₀)alkenyl, (C₂₋₂₀)alkynyl, (C₃₋₈)cycloalkyl(C₁₋₂₀)alkyl, (C₆₋₁₂)aryl(C₁₋₂₀)alkyl, heterocyclyl(C₁₋₂₀)alkyl, and/or heteroaryl(C₁₋₂₀)alkyl, optionally comprises one or more heteroatoms in the alkyl, alkenyl, alkynyl moiety. Optionally, each heteroatom may be independently a C, Si, chalcogenide (such as O, S, or Se), or a pnictide (such as N, or P).

In some examples, at least one carbon atom or heteroatom of the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl may be oxidized to form at least one C═O, C═S, N═O, N═S, S═O or S(O)₂.

In some examples, R⁶ and R⁷ together with the atom to which they are attached form a 5-, 6-, or 7-membered heterocyclyl.

In some examples, R⁸ may be hydrogen or is optionally selected from the group consisting of (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl.

In some examples, the (C₁₋₂₀)alkyl, (C₂₋₂₀)alkenyl, (C₂₋₂₀)alkynyl, (C₃₋₈)cycloalkyl(C₁₋₂₀)alkyl, (C₆₋₁₂)aryl(C₁₋₂₀)alkyl, heterocyclyl(C₁₋₂₀)alkyl, and/or heteroaryl(C₁₋₂₀)alkyl, optionally comprises one or more heteroatoms in the alkyl, alkenyl, alkynyl moiety, the heteroatoms being each independently a C, Si, chalcogenide (such as O, S, or Se), or a pnictide (such as N, or P).

In some examples, at least one carbon atom or heteroatom of the (C₁₋₂₀)alkyl, (C₂₋₂₀)alkenyl, (C₂₋₂₀)alkynyl, (C₆₋₁₂)aryl, (C₃₋₈)cycloalkyl, (C₆₋₁₂)aryl(C₁₋₂₀)alkyl, hetero(C₁₋₂₀)alkyl, heterocyclyl, heteroaryl, heterocyclyl(C₁₋₂₀)alkyl, and/or heteroaryl(C₁₋₂₀)alkyl may be oxidized to form at least one C═O, C═S, N═O, N═S, S═O or S(O)₂.

In some examples, A composition comprising a polymer, or polymer network, having at least one unit of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI) and/or (XXXII), or a mixture thereof. Optionally, the mean bio-based content may be at least 10%, e.g., about 1%, about 2%, about 3%, about 4%, about 5%, about 6%, about 7%, about 8%, about 9%, and/or about 10%.

In some examples, this disclosure describes a composition comprising a polymer, or polymer network, having at least one unit of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI) and/or (XXXII), or a mixture thereof. Optionally, at least one of the units may be chiral.

In some examples, this disclosure describes a method for synthesizing a polymer, or polymer network, from one or more precursors in one or more solvents, the method comprising: dissolving, dispersing, or suspending one or more precursors individually in the same solvent, or individually in different and/or separate solvents, optionally with one or more surfactants; optionally heating the solvent or one or more solvents of the different and/or separate solvents; and mixing the solvent and/or solvents comprising the one or more precursors together to form a polymer.

In some examples, this disclosure describes a method for synthesizing a polymer, or polymer network, by melting one or more solid precursors, the method comprising: melting one or more precursors together to form a polymer, where at least one precursor is optionally solid prior to melting; optionally mixing the one or more precursors that may be solid prior to, during, and/or subsequent to the melting step, or the precursors which may be solid is optionally first melted individually then mixed together to form a polymer; where the melting of the one or more precursors is optionally in a single or twin-screw compound extrusion device.

In some examples, this disclosure describes a method for synthesizing a polymer, or polymer network, from one or more precursors using mechanical grinding, the method comprising: mixing one or more the precursors together in a shaking or rotating chamber to form a polymer. Optionally, the shaking or rotating chamber may be a ball mill. Optionally, the shaking or rotating chamber may contain a grinding medium. Optionally, the grinding medium comprises of one or several sizes of spheres and/or rods made of metallic, composite, ceramic and/or polymer materials. Optionally, the precursors are dissolved in a solvent prior to mechanical grinding in the rotating chamber, also optionally called ball mill. Optionally, the precursors may be mixed together in a solvent during mechanical grinding. Optionally, if one or more precursors are solids, precursors may be melted together before mixing. Optionally, the duration of mixing of precursors within the shaking or rotating chamber, optionally with the grinding medium, may be used to control the extent of polymerization. Optionally, the duration of mixing of the precursors within the shaking or rotating chamber may be used to control polymer properties. Optionally, the polymer properties may include the glass transition temperature (T_{g}), polymer solubility, modulus, tensile strength, polymer color, polymer toughness, and/or polymer rigidity.

In some examples, this disclosure describes a polymer alloy comprising a mixture of two or more polymers. Optionally, at least of the two or more polymers comprises one or more of the following: polyurethane, polyurea, epoxy, phenolic resin, polyolefin, silicone, rubber, polyacrylate, polymethacrylate, polycyanoacrylate, polyester, polycarbonate, polyimide, polyamide, vitrimer, poly(vinylogous amide), poly(vinylogous urethane), and/or thermoplastic elastomers.

In some examples, this disclosure describes a method of obtaining a polymer alloy using one or more solvents, the method comprising: mixing one or more polymers together in one or more solvents to form a polymer alloy optionally having the composition as described herein.

In some examples, this disclosure describes a method of obtaining a polymer alloy by compound extrusion, the method comprising: melting one or more polymers together to form a polymer alloy; wherein optionally the mixing takes place in a compound single or twin screw extruder.

In some examples, this disclosure describes a method of obtaining a polymer alloy by mechanical grinding, the method comprising: mixing one or more polymers together in a shaking or rotating chamber to form a polymer alloy; wherein the shaking or rotating chamber is optionally a ball mill; wherein the shaking or rotating chamber optionally contains a grinding medium; wherein the grinding medium optionally comprises of one or more, or several, metallic or ceramic spheres and/or rods; wherein one or more of the polymers are optionally dissolved in a solvent or melted together prior to mixing in the rotating chamber; wherein the duration of mixing within the shaking or rotating chamber may be used to control the properties of the polymer alloy formed.

In some examples, this disclosure describes a composite material comprising a polymer and a filler material; wherein the filler material may have a unit having the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI) and/or (XXXII), or a mixture thereof, or a polymer alloy as described herein, or one or several described in other embodiments.

In some examples, the filler material is optionally flame-retardant materials, woven or non-woven carbon fibers, woven or non-woven polyaramid fibers, woven or non-woven glass fibers, carbon black, carbon nanotubes, graphene, diamondoids, aluminum, steel, stainless steel, iron, zinc, titanium, liquid metals, silicon carbide, boron nitride, metal oxide, metal pnictides, metal chalcogenides, metal halides, transition metal dichalcogenides, metal alloys, MXenes, vitrimers, zeolites, metal-organic frameworks, covalent organic frameworks, alumina, silica, and/or silicate clays.

In some examples, flame-retardant materials may be a brominated compound, a chlorinated compound, a nitrogen-containing compound, a phosphorous-containing compound, a hydrated metal oxide such as hydrated aluminum oxide or hydrated magnesium oxide, a metal oxide such as antimony trioxide.

In some examples, silicate clays may be laponite, sumecton, monomorillonite (also known as bentonite), sodium fluorohectorite, and/or sodium tetrasilicic mica.

In some examples, composite material optionally comprises a coloring agent, also called a dye or pigment.

In some examples, this disclosure describes an adhesive material, comprising: a polymer, and a polymer alloy and/or a composite material; wherein the polymer alloy may be as described herein; wherein the composite material is optionally as described herein.

In some examples, this disclosure describes a method for extruding a polymer, the method comprising: processing, such as extruding, one or more polymers using a single or dual screw melt extrusion apparatus; wherein one of the polymers is optionally a polymer alloy as described herein; wherein one of the polymers is optionally a composite material as described herein; wherein one of the polymers is optionally an adhesive material as described herein.

In some examples, this disclosure describes a method for shaping a polymer into a pellet, the method comprising: processing or extruding a polymer that may be first extruded as described herein; wherein the polymer is optionally of the composition as described herein; wherein the polymer is optionally a polymer alloy as described herein; wherein the polymer is optionally a composite as described herein; wherein the polymer is optionally an adhesive as described herein.

In some examples, a polymer fiber may have a diameter, width or thickness, or average thereof, ranging from about 0.5 nm to about 1.0 mm; e.g., about 0.5 nm, about 0.6 nm, about 0.7 nm, about 0.8 nm, about 0.9 nm, about 1.0 nm, about 1 nm to about 50 nm, about 50 nm to about 150 nm, about 150 to about 500 nm, about 500 nm to about 1000 nm, about 1000 nm to about 10000 nm, about 10000 nm to about 50000 nm, and/or about 50000 nm to about 1.0 mm. In some examples a polymer fiber may have a length ranging from about 5 nm to up to about 5000 m; e.g., about 0.5 nm to about 1.0 nm, about 1.0 nm to about 5.0 nm, about 5.0 nm to about 10 nm, about to 10 nm to about 50 nm, about 50 nm to about 100 nm, about 100 nm to about 500 nm, about 500 nm to about 1000 nm, about 1000 nm to about 5000 nm, about 5000 nm to about 50000 nm, about 50000 nm to about 100000 nm, about 100000 nm to about 500000 nm, about 500000 nm to about 1 m, about 1 m to about 5 m, about 5 m to about 50 m, about 50 m to about 100 m, about 100 m to about 1000 m, and/or about 1000 m to about 5000 m. In some examples, the polymer composition is optionally as described herein; wherein the polymer is optionally a polymer alloy as described herein; wherein the polymer is optionally a composite as described herein; wherein the polymer is optionally an adhesive as described herein.

In some examples, this disclosure describes a porous material comprising a polymer and having one or more pores with pore sizes ranging from about 0.5 nm to about 5000 nm, e.g., about 0.5 nm to about 1.0 nm, about 1.0 nm to about 5.0 nm, about 5.0 nm to about 10 nm, about to 10 nm to about 50 nm, about 50 nm to about 100 nm, about 100 nm to about 500 nm, about 500 nm to about 1000 nm, about 1000 to about 2000 nm, about 2000 to about 3000 nm, about 3000 to about 4000 nm, and/or about 4000 to about 5000 nm. In some examples, the porous material (optionally a sorbent) may be modified to bind small molecules; wherein the porous material (optionally a sorbent) may bind small molecules without modification; wherein the porous material (optionally called a membrane) may allow specific molecules, ions, solids, gases and/or liquids to transport into and/or through the porous material; wherein the polymer is optionally of the composition as described herein; wherein the polymer is optionally a polymer alloy as described herein; wherein the polymer is optionally a composite as described herein; wherein the polymer is optionally an adhesive as described herein.

In some examples, this disclosure describes a foam comprising a polymer, a polymer alloy, a composite, an adhesive, a porous material, and/or a polymer fiber that is optionally combined with one or several additives; wherein the foam may have a density of from about 0.1 to about 10 pounds per cubic foot (PCF), e.g., about 0.1 PFC, about 0.2 PFC, about 0.3 PFC, about 0.4 PFC, about 0.5 PFC, about 0.6 PFC, about 0.7 PFC, about 0.8 PFC, about 0.9 PFC, about 1.0 PFC, about 2 PFC, about 3 PFC, about 4 PFC, about 5 PFC, about 6 PFC, about 7 PFC, about 8 PFC, about 9 PFC, and/or about 10 PFC. In some examples, the polymer is optionally of the composition as described herein. Optionally, the polymer alloy is as described herein. Optionally, the composite material is as described herein. Optionally, the adhesive material is as described herein. Optionally, the polymer fiber is as described herein. Optionally, the porous material is as described herein. Optionally, the additive is a blowing agent, a surfactant, a plasticizer, a coloring agent (also called a dye, also called a pigment), a flame retardant, a catalyst, a polymer, a poly-alcohol (also called a polyol), PTFE, and/or a polyolefin wax.

In some examples, this disclosure describes a method whereby a foam may be synthesized, the method comprising: mixing a first compound(s) and a second compound(s), as described herein, with one or more additives to form a polymer; wherein the first compound(s) and the second compound(s) optionally have the ratio R as described herein; wherein the additives optionally comprise one or more polymer alloys, composite material, adhesive material, or any other composition as described herein.

In some embodiments, this disclosure describes an emulsion comprising a suspension of a material in a solvent, where the material is optionally a polymer, a polymer alloy, a composite, and/or an adhesive that is optionally combined with one or several additives; wherein the emulsion may optionally have a solids content from about 0.01 to about 80% on a per weight basis with respect to the solvent; e.g., about 0.01%, about 0.02%, about 0.03%, about 0.04%, about 0.05%, about 0.06%, about 0.07%, about 0.08%, about 0.09%, about 0.1%, about 0.1%, about 0.2%, about 0.3%, about 0.4%, about 0.5%, about 0.6%, about 0.7%, about 0.8%, about 0.9%, about 1%, about 1% to about 5%, about 5% to about 10%, about 20%, about 30%, about 40%, about 50%, about 60%, about 70%, and/or about 80%.

In some embodiments, the polymer is optionally of the composition (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI) and/or (XXXII), or a mixture thereof; wherein the polymer alloy is optionally as described herein; wherein the composite is optionally as described herein; wherein the adhesive is optionally as described herein; wherein the additives may optionally include, a blowing agent, a surfactant, a plasticizer, a coloring agent (also called a dye, also called a pigment), a flame retardant, a catalyst, a polymer, a poly-alcohol (also called a polyol), PTFE, and/or a polyolefin wax; wherein the solvent is optionally water, an alcohol, and/or an organic solvent.

In some embodiments, this disclosure describes a conductive material that may be capable of conducting photons (light), phonons, electrons, holes, spin, ions, excitons, and/or acoustic waves (sound), the conductive material comprising a polymer, and optionally a porous material, a polymer fiber, a polymer alloy, an adhesive material, a composite material, and/or a foam that is optionally combined with one or several additives; wherein the polymer is optionally of the composition as described herein; wherein the polymer alloy is optionally as described herein; wherein the composite material is optionally as described herein; wherein the adhesive material is optionally as described herein; wherein the adhesive may be specifically formulated to maintain integrity when bonding two or more substrates with different coefficients of thermal expansion; wherein the additives optionally includes electrical and/or chemical dopants added to control the conductivity of the conductive material.

In some embodiments, this disclosure describes an insulating material that may have low conductivity to photons (light), phonons, electrons, holes, spin, ions, excitons, and/or acoustic waves (sound); the insulating material optionally comprising a polymer, and optionally a porous material, a polymer fiber, a polymer alloy, an adhesive material, a composite material, and/or a foam that is optionally combined with one or several additives; wherein the polymer is optionally of the composition as described herein; wherein the polymer alloy is optionally as described herein; wherein the composite material is optionally as described herein; wherein the adhesive material is optionally as described herein; wherein the additives may optionally include additives added to control the conductivity of the insulating material.

In some embodiments, this disclosure describes a method for recycling a polymer or mixture of polymers, the method comprising: depolymerizing a polymer or mixture of polymers with an excess of amine containing at least one of the type R⁸—NH₂, R⁸—NHR⁴, R⁸—NH₃⁺ and/or R⁸—NHR⁴R⁵⁺ groups, or at least one functional group that generates R⁸—NH₂, R⁸—NHR⁴, R⁸—NH₃⁺ and/or R⁸—NHR⁴R⁵⁺.

In some embodiments, the polymer or mixture of polymers may be depolymerized by hydrolysis in the presence an acid or a mixture of acids selected from, but not limited to, HCl, H₂SO₄, H₃PO₄, p-toluenesulfonic acid, methane sulfonic acid, trifluoroacetic acid, and/or trifluoromethanesulfonic acid.

In some embodiments, the polymer or mixture of polymers may be depolymerized by hydrolysis in the presence an acid or a mixture of acids selected from, but not limited to, HCl, H₂SO₄, H₃PO₄, p-toluenesulfonic acid, methane sulfonic acid, trifluoroacetic acid, and/or trifluoromethanesulfonic acid, alongside the presence of an amine containing at least one of the type R⁸—NH₂, R⁸—NHR⁴, R⁸—NH₃⁺ and/or R⁸—NHR⁴R⁵⁺ groups, or at least one functional group that generates R⁸—NH₂, R⁸—NHR⁴, R⁸—NH₃⁺ and/or R⁸—NHR⁴R⁵⁺.

In some embodiments, the polymer or mixture of polymers optionally contains at least one polymer of the composition (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI) and/or (XXXII), or other polymers described herein or in the appended claims, or any mixture of these; and/or the polymer or mixture of polymers optionally comprises at least one polymer alloy as described herein; and/or the polymer or mixture of polymers optionally comprises at least one composite as described herein; and/or the polymer or mixture of polymers optionally comprises at least one adhesive as described herein.

Computational Design of Polydiketoenamines with Controlled Rates of Acidolysis and Aminolysis

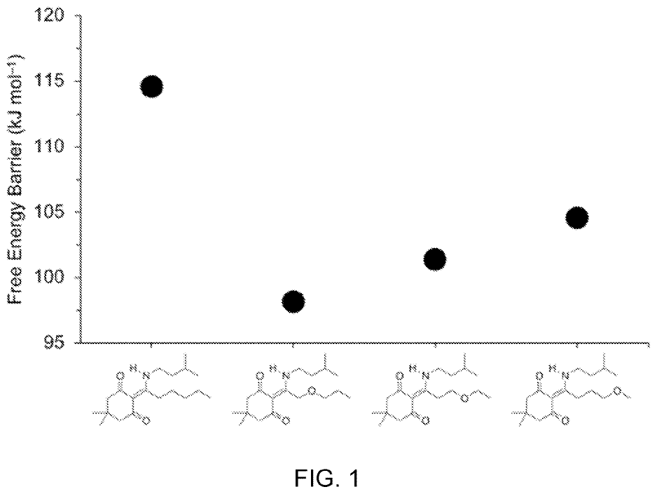

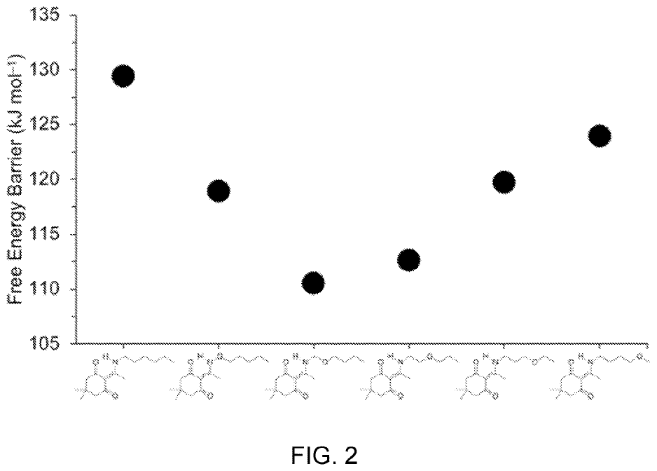

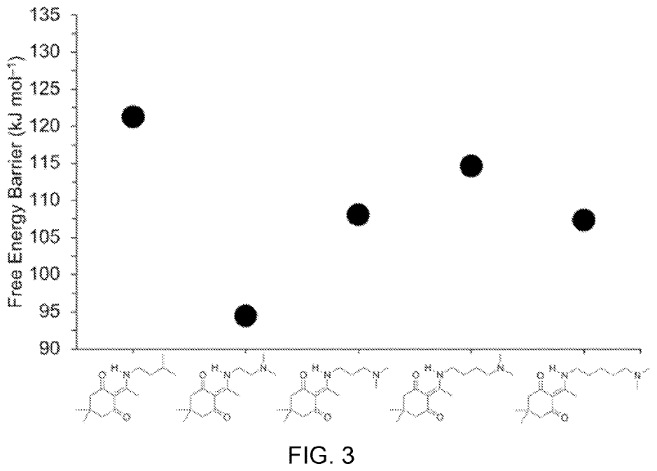

In some examples, the free energy barriers were calculated for acidolysis and aminolysis of poly diketoenamines with varied placement of the O-atom on the amine monomer and triketone monomer and varied placement of the N-atom on the amine monomer. Other examples show modelled polydiketoenamine acidolysis and aminolysis as the addition of H₂O to a small molecule model of the diketoenamine bonding motif used in polymer. For each molecule, a conformer search was performed to find the lowest-energy reactant and transition state conformers contributing to the reaction using CREST. Then hybrid density functional theory (hybrid-DFT) methods implemented in Gaussian16 were used to optimize the structures of the reactant and transition state and calculated the free energy barrier as the difference in free energy between the two structures (FIG. 1, FIG. 2, FIG. 3).

### Biosynthesis of Raw Materials for Producing Monomers

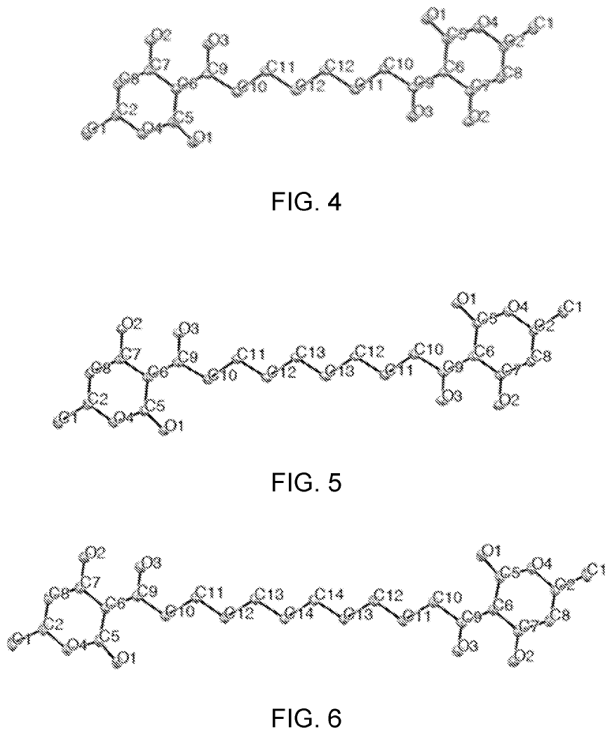

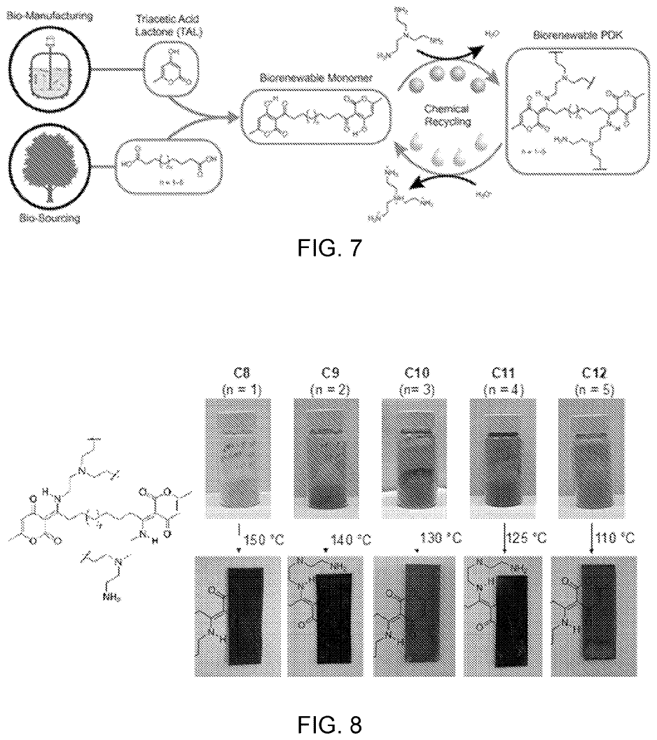

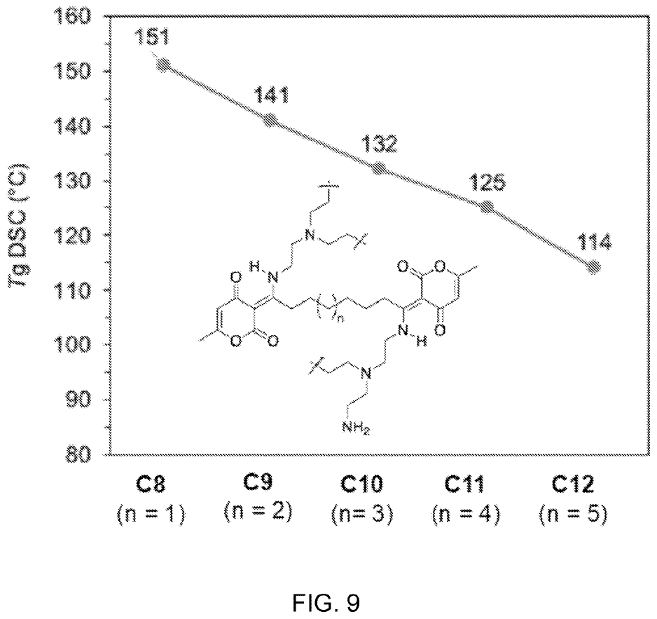

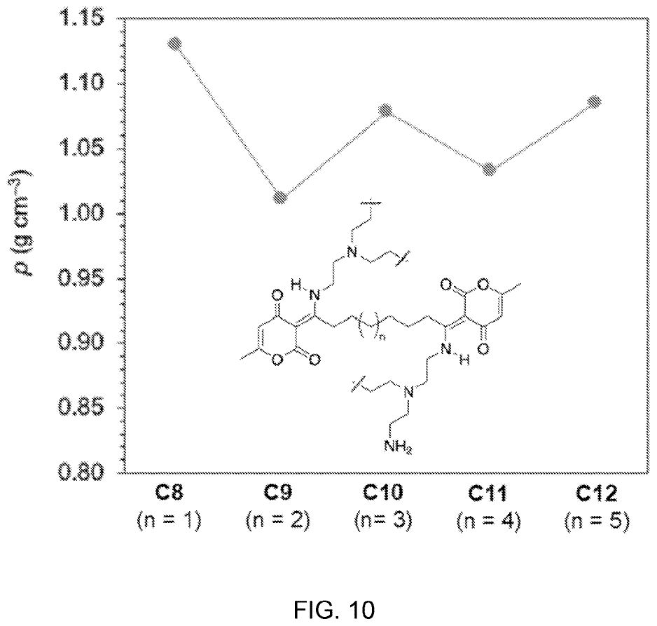

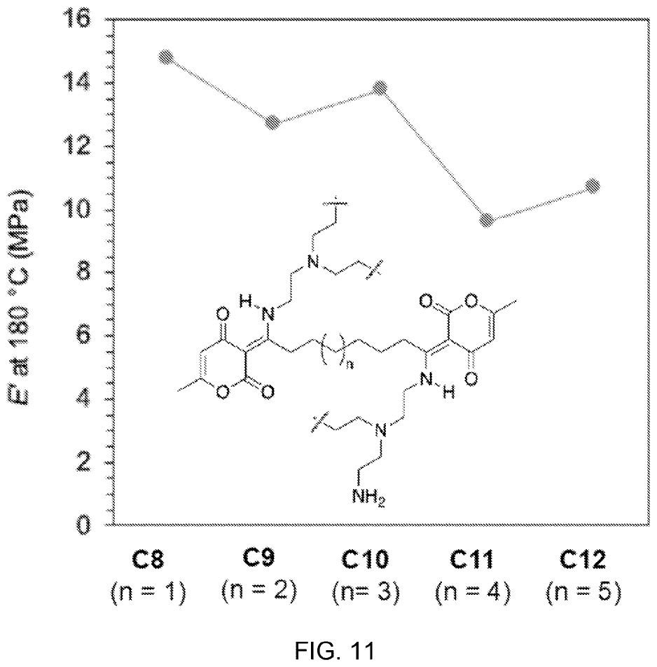

Bio-production of the simple polyketide, triacetic acid lactone (TAL), has been demonstrated, e.g., in Escherichia coli, Saccharomyces cerevisiae, and Yarrowia lipolytica. Bio-based TAL may be used in the synthesis of polytopic triketone monomers (FIG. 4, FIG. 5, FIG. 6), which in turn may be used to produce a composition of polymers with a plurality of diketoenamine bonds and high bio-content (FIG. 7, FIG. 8, FIG. 9, FIG. 10, FIG. 11).

Bio-production of polytopic organic acids, including dicarboxylic acids, has been demonstrated, e.g., through direct conversion of fatty acids, through biosynthesis in microorganisms, or through other means known. Certain organic diacids, such as sebacic acid, are commercially available as a bio-product derived from castor oil. Similarly, diglycolic acid is commercially available as a bio-product derived from glycolic acid. Bio-based sebacic acid and diglycolic acid may be used in the synthesis of polytopic triketone monomers, which in turn may be used to produce a composition of polymers with a plurality of diketoenamine bonds and high bio-content.

Bio-production of malonic acids and dialkyl malonate esters has been demonstrated and commercialized. Bio-based malonates may be used as bio-renewable raw materials for the production of a wide variety of β-diketones, including dimedone, Meldrum's acid, and barbituric acids. Bio-based β-diketones may be used in the synthesis of polytopic triketone monomers, which in turn may be used to produce a composition of polymers with a plurality of diketoenamine bonds and high bio-content.

Bio-production of acetone may be carried out by several known and commercialized processes, including a carbon-negative fermentation route from abundant, low-cost waste gas feedstocks, such as industrial emissions and syngas. Acetone may be used as a bio-renewable raw material for the production of mesityl oxide. Mesityl oxide may be used as a bio-renewable raw material for the production of dimedone, which in turn may be used to produce a composition of polymers with a plurality of diketoenamine bonds and high bio-content.

Bio-production of polyols is well established. Many of these polyols may be modified using processes that are industrialized or are known to produce bio-based amine-terminated molecules, including oximes, 1-aminomethyl, 2-aminoethyl, and 3-aminopropyl functionality. Bio-based small molecules or macromolecules featuring oximes, 1-aminomethyl, 2-aminoethyl and/or 3-aminopropyl functionality may be used to produce a composition of polymers with a plurality of diketoenamine bonds and high bio-content.

Bio-production of monomers used to synthesize bio-based polyethers has been demonstrated. Certain diols, such as 1,4-butane diol (BDO), are commercially available as a bio-product of microbial fermentation. BDO may be used in the synthesis of bio-based polytetrahydrofuran (PTHF), an important component in elastomer and reinforced rubber formulations. Similarly, 1,2-propane diol (also known as propylene glycol) is commercially available as a bio-product. Propylene glycol may be used in the synthesis of bio-based polypropylene glycol (PPG), which is likewise an important component in elastomer and reinforced rubber formulations. The chain ends of PTHF and PPG may be converted from alcohols to amines, optionally monotopic or polytopic, using processes that are industrialized or are known. Bio-based amine-modified PTHF and PPG may be used to produce a composition of polymers with a plurality of diketoenamine bonds and high bio-content.

### Chemical Synthesis of Triketone Monomers

In some examples, polytopic triketone monomers may be used to produce a composition of polymers with a plurality of diketoenamine bonds. Polytopic triketones may be prepared from polytopic carboxylic acids and a wide range of β-diketones, including aromatic and aliphatic β-keto-δ-lactones (BKDL), which may optionally be chiral. Direct condensation of polytopic carboxylic acids and β-diketones may employ a condensation agent, e.g., N,N′-dicyclohexylcarbodiimide (DCC), and in some examples also a catalyst, e.g., 4-(dimethylamino)pyridine (DMAP). Indirect condensation of pre-activated polytopic carboxylic acids, e.g., carboxylic acid halides, N-hydroxy-succinimidyl esters, N-imidazoyl esters, or tetrafluorophenyl esters, with β-diketones is also possible. In some examples, C-acylation producing the desired triketone is observed, while in other examples, O-acylation producing a mixed anhydride may be observed. If O-acylation is observed, a second step may be used to produce the desired triketone. This second step may be carried out with an O- to C-acyl transfer catalyst, e.g., 4-(dimethylamino)pyridine (DMAP). In some examples, elevated temperature may be needed to promote efficient O- to C-acyl transfer in the presence of the catalyst.

### Chemical Synthesis of Amine Monomers

In some examples, polytopic amine monomers may be used to produce a composition of polymers with a plurality of diketoenamine bonds. In some examples, it may be important for accelerating the rate of diketoenamine bond hydrolysis in strong aqueous acid to place a heteroatom in close proximity, e.g., within 4 atoms of the amine undergoing condensation with the triketone. Polytopic amine monomers optionally featuring heteroatoms within 4 atoms of the reactive amine in diketoenamine synthesis may be produced from small molecules or macromolecules, including linear or branched polymeric precursors having chain-end or mid-chain functionality. One aspect describes a multi-step synthesis of a polytopic amine monomer from a macromolecular α,ω-polyether diol and tris(2-aminoethyl)amine to yield a macromolecular crosslinker whose chain-end functionality features a heteroatom within 4 bonds of the reactive amine.

Chemical Synthesis of Polydiketoenamines with Linear Topology

In some examples, a composition of polymers with a plurality of diketoenamine bonds and with a linear topology may be prepared from ditopic triketone and amine monomers, and may produce water as a byproduct. The molar ratio of monomers used in the synthesis and the extent of reaction may be relevant to or dictate the degree of polymerization. In general, high molecular weight polymers may utilize a 1:1 molar ratio of monomers. It is demonstrated that the polymerization of polydiketoenamines using a 1:1 ratio of ditopic triketone and amine monomers by using a melt polymerization at elevated temperature. A multi-stage polymerization process is also demonstrated, where a low molecular weight pre-polymer may be first generated at a low temperature and a higher molecular weight polymer may be subsequently generated at a higher temperature with active removal of the aqueous byproduct in vacuo to increase the extent of reaction.

Chemical Synthesis of Polydiketoenamines with Network Topology

In some examples, a composition of polymers with a plurality of diketoenamine bonds and with a network topology may be prepared from ditopic triketone and polytopic amine monomers, and may produce water as a byproduct. The molar ratio of monomers used in the synthesis and the extent of reaction may be relevant to or dictate the degree of polymerization and gelation behavior. The conversion of polymers to insoluble gels, may occur through crosslinking reactions. In general, the crosslinking of growing polymers to form insoluble gels with a network topology may be conducted with either stoichiometric or non-stoichiometric ratios of participating crosslinkable functionality. Owing to the dynamic covalent character of the diketoenamine bond, the use of non-stoichiometric ratios of crosslinkable functionality may produce polydiketoenamine networks with tunable and useful thermomechanical properties. In some examples, non-stoichiometric triketone:amine ratios of 1:1.1 or larger may be useful for subsequent thermomechanical processing and for controlling the response of the material to mechanical deformation, e.g., as evidenced in the rate or extent of stress relaxation or in the resistance to creep. In general, a decrease in the crosslinking density may result in a decrease of the glass transition temperature of the polymer network. In some examples, crosslinked networks may be prepared by ball-milling, by polycondensation from solutions or dispersions of polytopic triketone and amine monomers, and/or by other methods known to one skilled in the art.

### Polydiketoenamine Formulation and Properties

In some examples, polydiketoenamine formulation may be used to tailor polymer properties. For example, elastomeric crosslinked polydiketoenamines form spontaneously from simple mixing of concentrated solutions of amine and triketone monomers. Either or both of the amine and triketone components may serve as the soft segment. The resulting polymer may be directly processed into molds, optionally at elevated temperature and pressure. Rheological properties including storage modulus, stress relaxation, and creep may be measured, e.g., by using a parallel plate rheometer. In some examples, elastomeric polydiketoenamine networks were formulated from a ditopic triketone monomer and an amine component with average functionality >2, with a total triketone:amine molar ratio of 1:1.1 or greater. Dissolving either or both components in a suitable organic solvent may be used to ensure homogeneous mixing prior to gelation, particularly if one or both components is a solid at ambient conditions. The inclusion of fillers and additives may be accomplished at this step, e.g., by dispersing the filler or additive in solution with either or both the triketone or amine prior to gelation.

In some examples, polymers with linear topology may be processed by compression molding, by injection molding, blow molding, extrusion pelletizing, extrusion molding, reactive injection molding, thermoforming, transfer molding, film blowing, laser sintering, from solutions and dispersions, and/or other processes known to one skilled in the art. In some examples, polymers with network topology may be processed by reactive injection molding, compression molding, extrusion molding, laser sintering, spin casting, and/or other processes known to one skilled in the art (FIG. 8). In some examples, polymers may be blended with other polymers, may be mixed with a variety of additives, including but not limited to stabilizers, lubricants, plasticizers, flame retardants, dyes, anti-oxidants, surfactants, and/or dispersing agents.

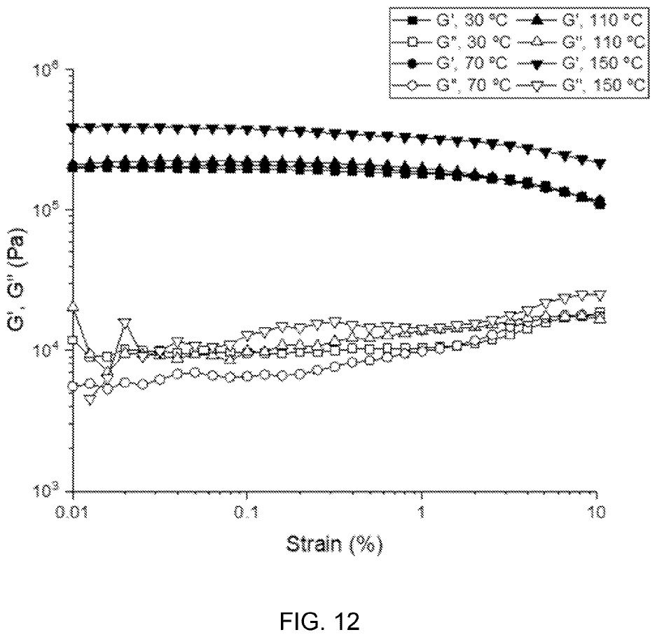

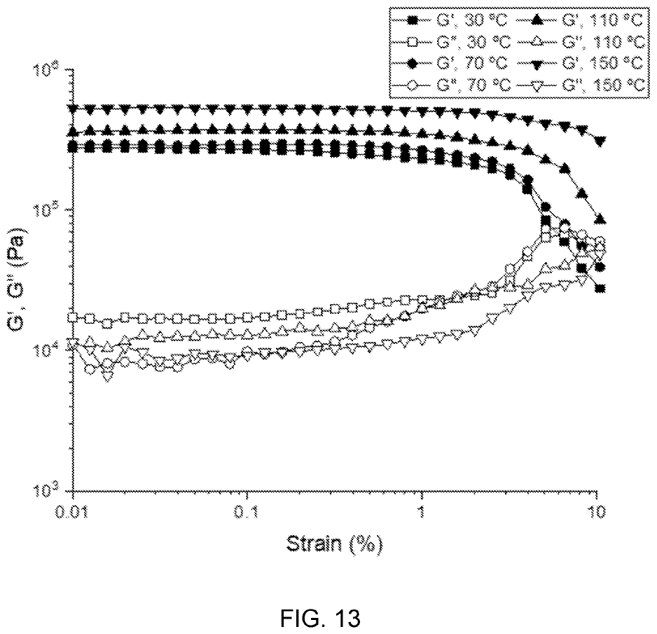

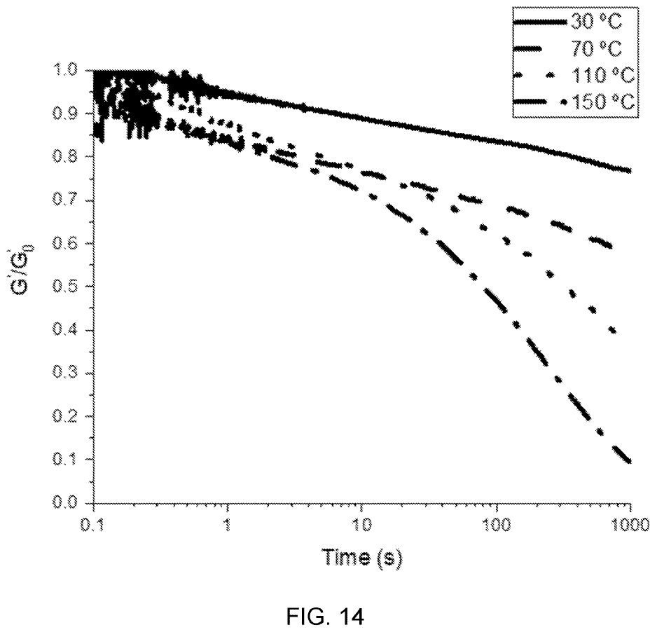

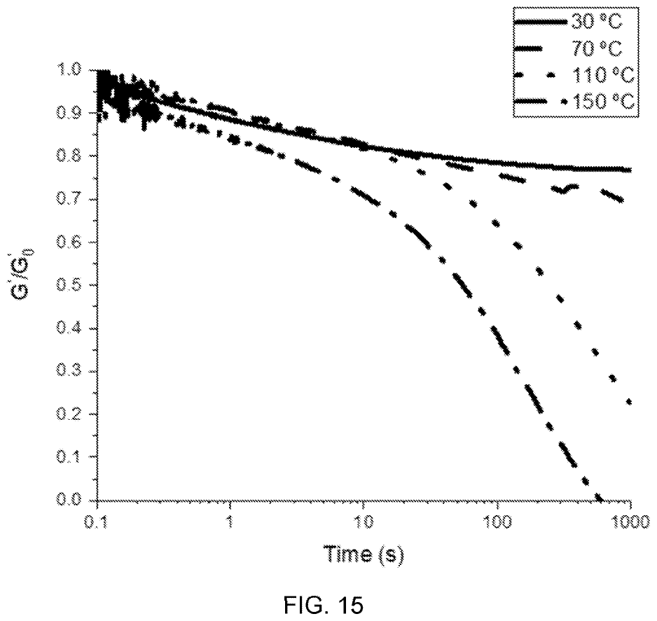

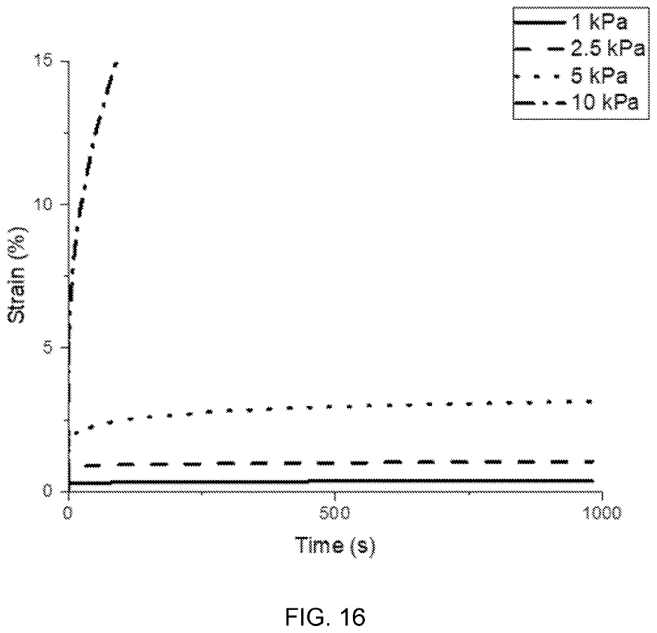

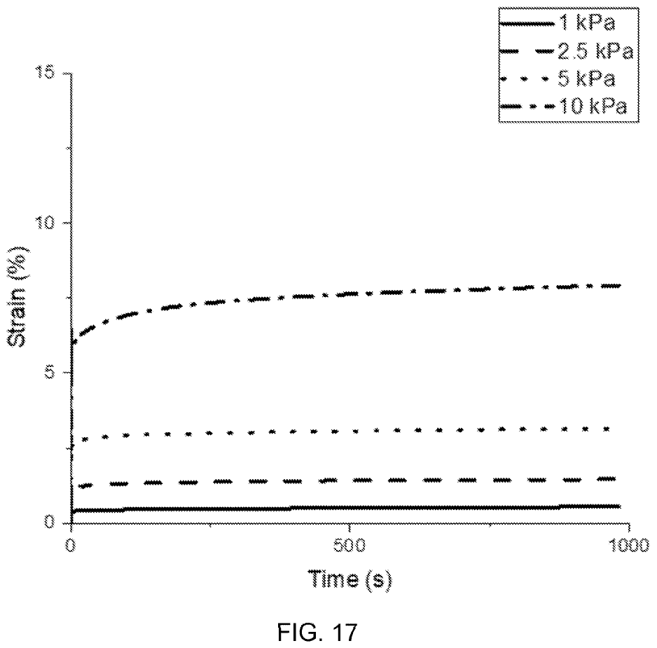

A variety of analytical methods may be applied to determine the properties of the materials, including but not limited to glass transition temperature using differential scanning calorimetry or dynamic mechanical analysis, thermal stability using thermogravimetric analysis, molecular weight and dispersity by size exclusion chromatography, tensile strength using tensiometer, flexural strength using dynamic mechanical analysis, compressive strength using rheometer, hardness using durometer, and/or fracture toughness by impact tests (FIG. 9, FIG. 10, FIG. 11, FIG. 12, FIG. 13, FIG. 14, FIG. 15, FIG. 16, FIG. 17).

In some examples, rheological characterization of elastomeric and composite rubber materials was performed using a parallel plate rheometer with 8 mm sample discs (FIG. 12, FIG. 13, FIG. 14, FIG. 15, FIG. 16, FIG. 17). Elastomer storage and loss moduli were measured via strain sweep from 0.01 to 10% strain at 10 rad/s and 30, 70, 110, or 150° C. Elastomer storage modulus was ≈200 kPa between 30-110° C., and exhibited temperature-induced stiffening to ≈400 kPa at 150° C. Adding 0.5% carbon black as a filler increased the storage modulus to ≈300 kPa at 30° C. and ≈550 kPa at 150° C. Formulations with and without carbon black underwent similar stress relaxation profiles over 1000 s at 5% strain, demonstrating the presence of exchangeable diketoenamine bonds. Both formulations underwent minimal creep over 1000 s at 30° C. and 1, 2.5 or 5 kPa stress. The formulation without carbon black exhibited significant creep at 10 kPa stress, while the inclusion of carbon black substantially reduced creep at the same stress.

### Polydiketoenamine Chemical Recycling to Monomer

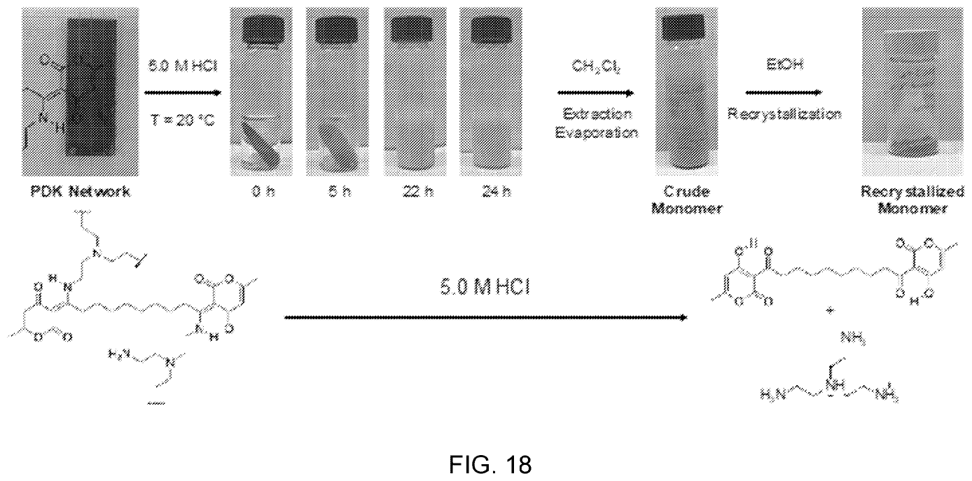

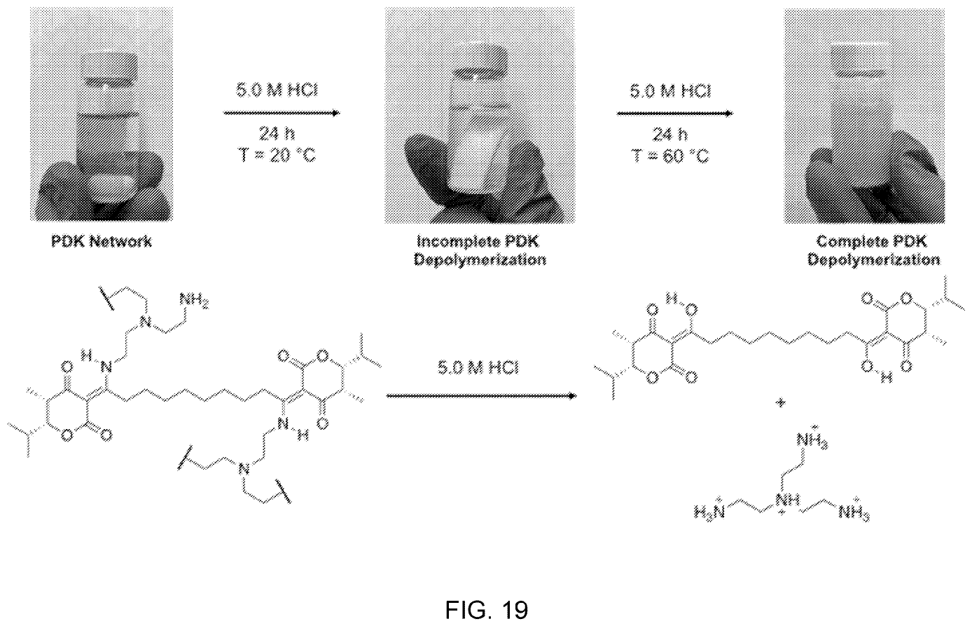

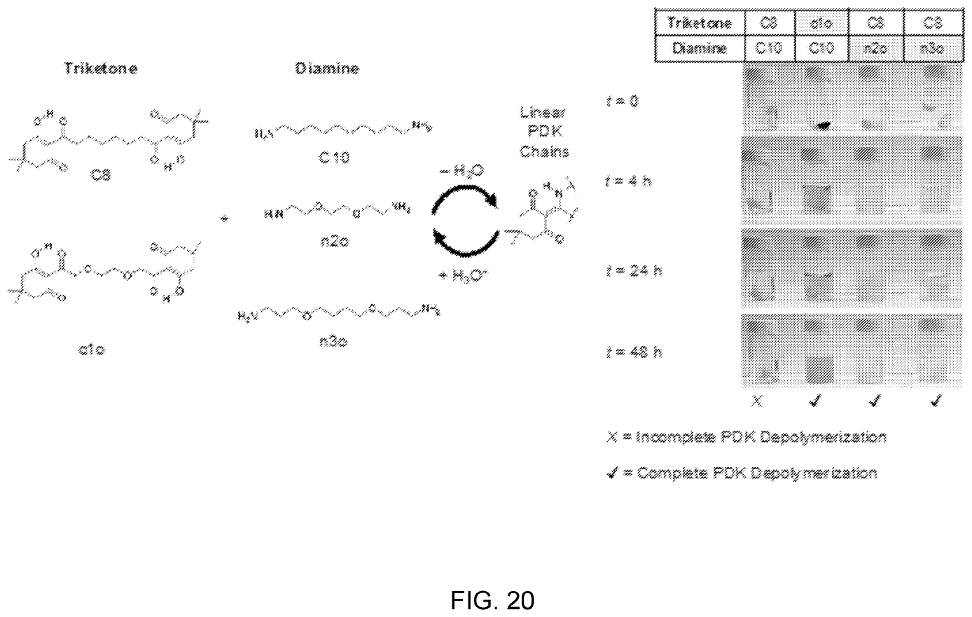

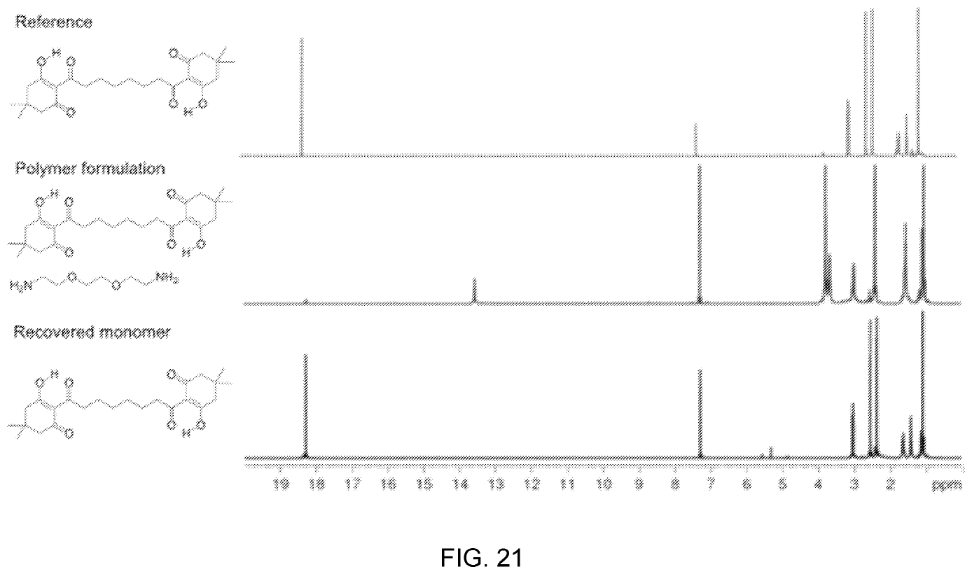

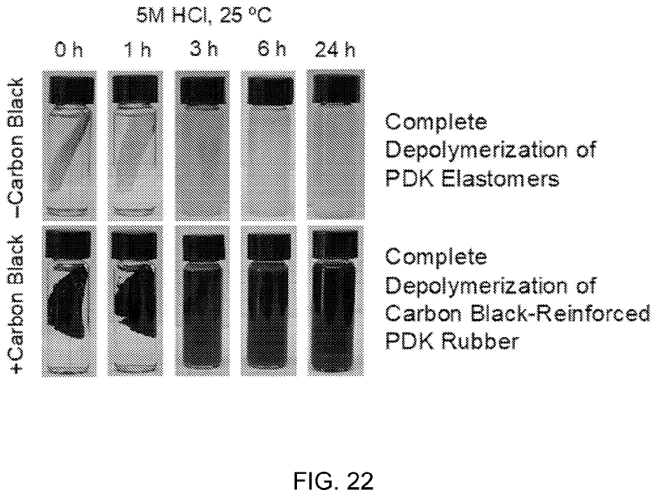

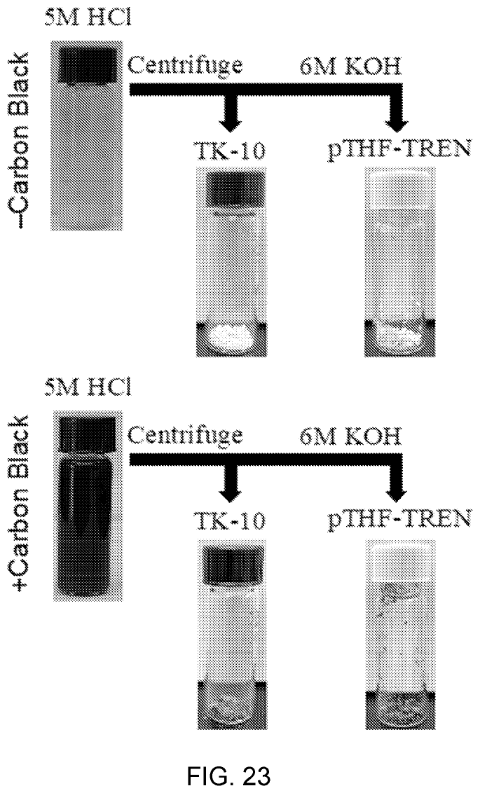

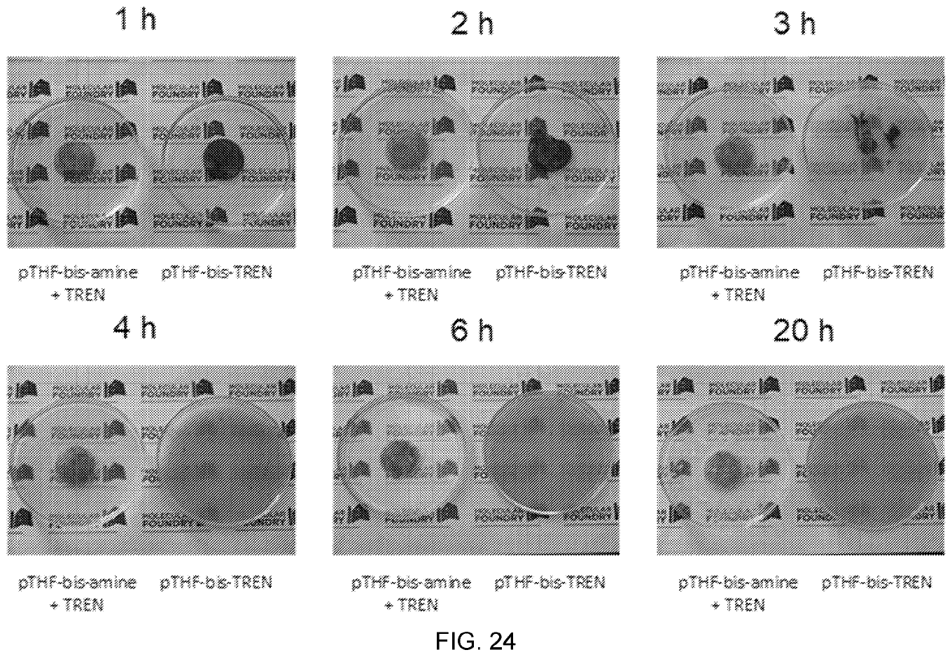

In some examples, polydiketoenamines may be deconstructed into triketone and amine monomers in strong aqueous acid (FIG. 18, FIG. 19, FIG. 20, FIG. 21, FIG. 22, FIG. 23, FIG. 24). The rate of depolymerization may be dependent on temperature, as well as placement of the heteroatoms near diketoenamine bond in the polymer. The contrasting properties of triketone and amine monomers in some embodiments allow for monomer separation and recovery through solid-liquid filtration. Monomer refinement to remove additives or impurities may also be undertaken, e.g., by recrystallization of a solid, by filtration of a liquid, and/or by liquid-liquid extractions. After acidolysis of the diketoenamine bonds, the liberated amine monomers may be ionized and a basic ion exchange resin may be used to regenerate the original amine monomer. Triketone and amine monomers are generally recovered in high purity and in high yields, varying from 75 to 99%, e.g., about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, and/or about 99%, provided the triketone and/or amine monomers feature a heteroatom within 4 bonds of the diketoenamine. Yield was determined gravimetrically. Purity of the recovered monomers was determined using ¹H NMR from the isolated dry powders.

Aspects of the disclosure may be further understood by reference to the following non-limiting examples.

### Example 1

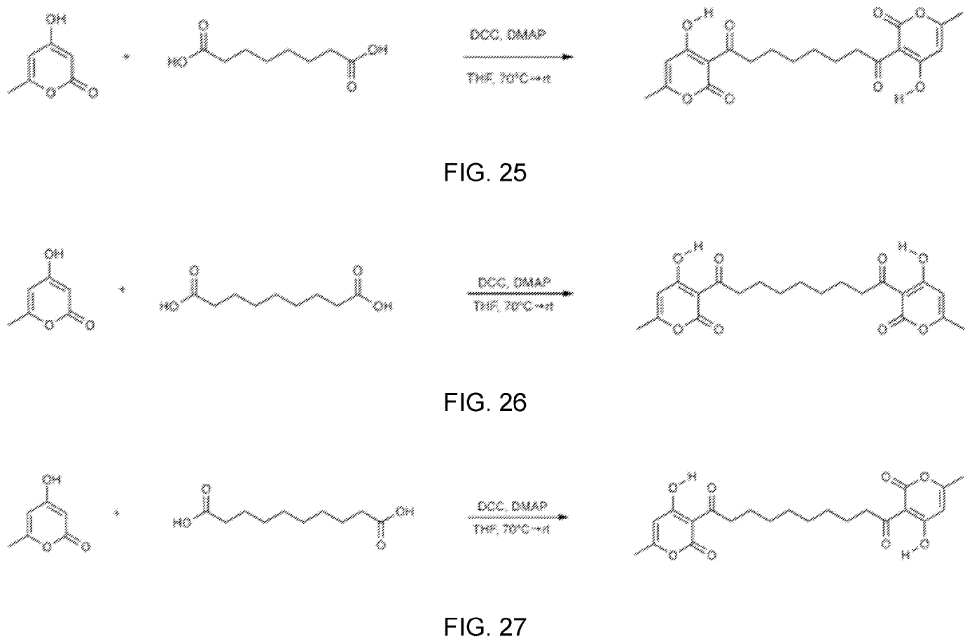

FIG. 25 provides a synthetic scheme for preparation of an Example monomer compound:

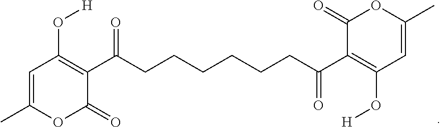

1,8-bis(4-hydroxy-6-methyl-2-oxo-2H-pyran-3-yl)octane-1,8-dione was synthesized by the following procedure. Triacetic acid lactone (80.1 mmol), suberic acid (38.2 mmol), and DMAP (114.9 mmol) were solubilized in tetrahydrofuran (200 mL) upon heating at 70° C. A separate solution of DCC (90.4 mmol) in tetrahydrofuran (50 mL) was added slowly to the reaction mixture. The reaction mixture gradually turned yellow, accompanied by the formation of a white precipitate. After the complete addition of DCC, the reaction was allowed to cool to room temperature and pursued overnight (24 h). The mixture was filtered and washed with CH₂Cl₂ until the solid became colorless. The filtrate was concentrated and the recovered product, a dark red oil, was dissolved in CH₂Cl₂ (400 mL) and extracted with 2 M HCl (2×150 mL). The organic phase was dried over MgSO₄ and concentrated, leaving the crude product as an orange paste. The crude product was recrystallized from ethanol to yield a light orange needles (6.00 g, 15.4 mmol, 40.2%). ¹H NMR (500 MHz, CDCl₃): δ 16.84 (s, 2H), 5.92 (s, 2H), 3.05-3.08 (t, J=7.4 Hz, 4H), 2.26 (s, 6H), 1.65-1.67 (m, 4H), 1.40-1.43 (m, 4H) ppm; ¹³C NMR (125 MHz, CDCl₃): δ 207.99, 181.38, 168.94, 161.11, 101.66, 99.61, 41.70, 29.13, 23.88, 20.81 ppm; ESI-MS: m/z for (C₂₀H₂₂O₈)Na⁺ ([M+Na]⁺) calculated 413.1207, found 413.1350; FT-IR: 3076, 2951, 2860, 1711, 1643, 1601, 1540, 1451, 1421, 1377, 1354, 1313, 1237, 1164, 1027, 992, 972, 920, 855, 801, 781, 766, 711, 638, 582, 502, 484, 400 cm⁻¹.

### Example 2

FIG. 26 provides a synthetic scheme for preparation of an Example monomer compound:

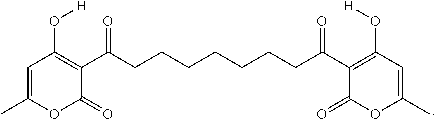

1,9-bis(4-hydroxy-6-methyl-2-oxo-2H-pyran-3-yl)nonane-1,9-dione was synthesized in an identical fashion to Example 1, except that azelaic acid was used in place of suberic acid. The crude product was co-recrystallized from ethanol and H₂O to yield a yellow powder (isolated yield: 42.9%). ¹H NMR (500 MHz, CDCl₃): δ 16.85 (s, 2H), 5.92 (s, 2H), 3.04-3.07 (t, J=7.4 Hz, 4H), 2.26 (s, 6H), 1.63-1.65 (m, 4H), 1.36-1.39 (m, 6H) ppm; ¹³C NMR (125 MHz, CDCl₃): δ 208.08, 181.39, 168.92, 161.10, 101.66, 99.62, 41.73, 29.37, 29.16, 24.01, 20.78 ppm; ESI-MS: m/z for (C₂₁H₂₄O₈)Na⁺ ([M+Na]⁺) calculated 427.1364, found 427.1519; FT-IR: 3087, 2934, 2854, 1707, 1639, 1604, 1548, 1450, 1234, 992, 924, 773, 713, 639, 567, 502, 401 cm⁻¹.

### Example 3

FIG. 27 provides a synthetic scheme for preparation of an Example monomer compound:

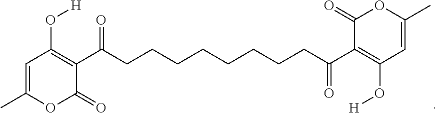

1,10-bis(4-hydroxy-6-methyl-2-oxo-2H-pyran-3-yl)decane-1,10-dione was synthesized in an identical fashion to Example 1, except that sebacic acid was used in place of suberic acid. The crude product was recrystallized from ethanol to yield a yellow-orange needles (isolated yield: 40.6%). ¹H NMR (500 MHz, CDCl₃): δ 16.87 (s, 2H), 5.92 (s, 2H), 3.04-3.07 (t, J=7.4 Hz, 4H), 2.26 (s, 6H), 1.61-1.67 (m, 4H), 1.34-1.38 (m, 8H) ppm; ¹³C NMR (125 MHz, CDCl₃): δ 208.15, 181.40, 168.92, 161.13, 101.68, 99.63, 41.78, 29.41, 29.28, 24.04, 20.81 ppm; ESI-MS: m/z for (C₂₂H₂₆O₈)Na⁺ ([M+Na]⁺) calculated 441.1520, found 441.1350; FT-IR: 3076, 2930, 2856, 1710, 1641, 1603, 1543, 1455, 1421, 1335, 1285, 1234, 1165, 1024, 992, 918, 853, 776, 710, 637, 506, 431, 400 cm⁻¹.

### Example 4

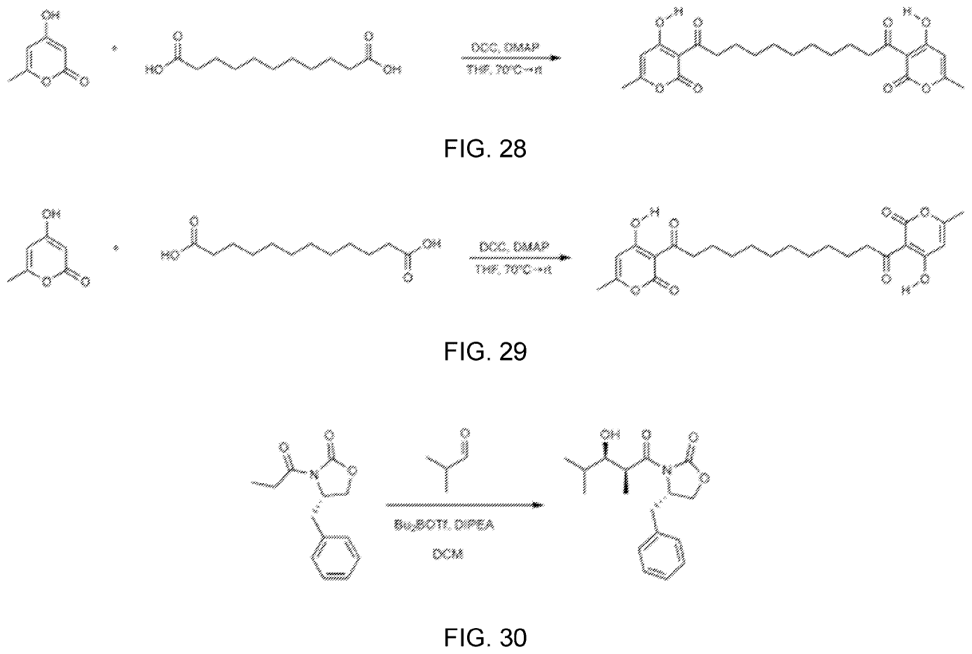

FIG. 28 provides a synthetic scheme for preparation of an Example monomer compound:

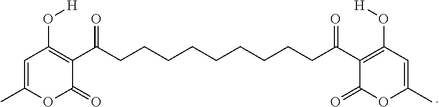

1,11-bis(4-hydroxy-6-methyl-2-oxo-2H-pyran-3-yl)undecane-1,11-dione was synthesized in an identical fashion to Example 1, except that 1,9-nonanedicarboxylic acid was used in place of suberic acid. The crude product was co-recrystallized from ethanol and H₂O to yield yellow granules (isolated yield: 47.9%). ¹H NMR (500 MHz, CDCl₃): δ 16.87 (s, 2H), 5.92 (d, J=0.65 Hz, 2H), 3.04-3.07 (t, J=7.4 Hz, 4H), 2.26 (d, J=0.55 Hz, 6H), 1.60-1.66 (m, 4H), 1.30-1.35 (m, 10H) ppm; ¹³C NMR (125 MHz, CDCl₃): δ 208.17, 181.39, 168.91, 161.12, 101.67, 99.61, 41.78, 29.52, 29.43, 29.31, 24.04, 20.80 ppm; ESI-MS: m/z for (C₂₃H₂₈O₈)Na⁺ ([M+Na]⁺) calculated 455.1676, found 455.1831; FT-IR: 3094, 2926, 2857, 1711, 1643, 1600, 1549, 1456, 1423, 1388, 1385 1238, 1187, 1171, 1025, 994, 972, 919, 846, 775, 709, 640, 590, 506, 447 cm⁻¹.

### Example 5

FIG. 29 provides a synthetic scheme for preparation of an Example monomer compound:

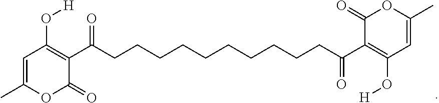

1,12-bis(4-hydroxy-6-methyl-2-oxo-2H-pyran-3-yl)dodecane-1,12-dione was synthesized in an identical fashion to Example 1, except that dodecanedioic acid was used in place of suberic acid. The crude product was recrystallized from ethanol to yield yellow needles (isolated yield: 62.1%). ¹H NMR (500 MHz, CDCl₃): δ 16.88 (s, 2H), 5.93 (d, J=0.65 Hz, 2H), 3.05-3.08 (t, J=7.4 Hz, 4H), 2.26 (d, J=0.5 Hz, 6H), 1.61-1.67 (m, 4H), 1.28-1.36 (m, 12H) ppm; ¹³C NMR (125 MHz, CDCl₃): δ 208.21, 181.41, 168.91, 161.14, 101.69, 99.63, 41.81, 29.57, 29.34, 24.07, 20.82 ppm; ESI-MS: m/z for (C₂₄H₃₀O₈)Na⁺ ([M+Na]⁺) calculated 469.1833, found 469.1974; FT-IR: 2923, 2852, 1715, 1643, 1606, 1550, 1452, 1422, 1350, 1318, 1269, 1238, 1166, 1031, 993, 967, 919, 852, 768, 711, 640, 504, 416 cm⁻¹.

### Example 6

FIG. 30 provides a synthetic scheme for preparation of an Example monomer compound:

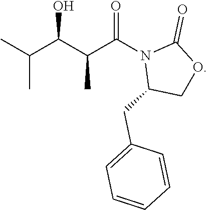 [SMILES: CC(C)[C@@H](O)[C@H](C)C(=O)N1C(=O)OC[C@@H]1Cc1ccccc1]

(5 S,6R)-6-isopropyl-5-methyldihydro-2H-pyran-2,4(3H)-dione was synthesized by the following procedure. To a stirred solution of (S)-4-benzyl-3-propionyloxazolidin-2-one (20.0 g, 86.7 mmol) in DCM (200 mL) under argon atmosphere at 0° C. was added Bu₂BOTf (1.0 M in DCM, 100 mL, 100 mmol) dropwise. Diisopropylethylamine (13.5 g, 104 mmol) was then slowly added to the reaction mixture. The reaction mixture was further stirred at 0° C. for 30 min before cooling to −78° C. Distilled propionaldehyde (7.19 g, 99.7 mmol) was slowly added to the reaction mixture under stirring. After stirring for another 1 h at −78° C., the reaction mixture was allowed to warm to 0° C. and was further stirred for an additional 1 h. At 0° C., the reaction was quenched by slow addition of 350 mL methanol and 100 mL phosphate buffer (1.0 M, pH 7), followed by the slow addition of a solution of 30 wt % H₂O₂ (125 mL) in methanol (150 mL). The reaction mixture was stirred for another 1 h before concentration in vacuo. The concentrated mixture was extracted by DCM (3×100 mL). The organic layers were combined, washed with saturated aqueous NaHCO₃ (100 mL) and brine (100 mL) before being dried over MgSO₄. The solvent was removed in vacuo to yield the crude product, which was purified by column chromatography (silica gel, 10-50% EtOAc in hexane) to give (S)-4-benzyl-3-((2S,3R)-3-hydroxy-2,4-dimethylpentanoyl)oxazolidin-2-one as colorless crystalline solids (96% isolated yield, R_{f}=0.46, 1:2 EtOAc/hexane); ¹H NMR (500 MHz, CDCl₃) δ 7.37-7.22 (m, 5H), 4.70 (m, 1H), 4.27-4.16 (m, 2H), 3.98 (dd, J=7.0, 2.8 Hz, 1H), 3.78 (qd, 7.2, 2.8 Hz, 1H), 3.26 (dd, J=13.5, 3.3 Hz, 1H), 3.01 (s, 1H), 2.82 (dd, J=13.4, 9.4 Hz, 1H), 1.74 (m, 1H), 1.26 (d, J=7.2 Hz, 3H), 1.05 (d, J=6.7 Hz, 3H), 0.93 (d, J=6.9 Hz, 3H) ppm; ¹³C NMR (126 MHz, CDCl₃) δ 177.61, 152.87, 135.06, 129.40, 128.88, 127.33, 76.57, 66.10, 55.10, 39.70, 37.63, 30.79, 19.17, 18.88, 10.02 ppm.

### Example 7

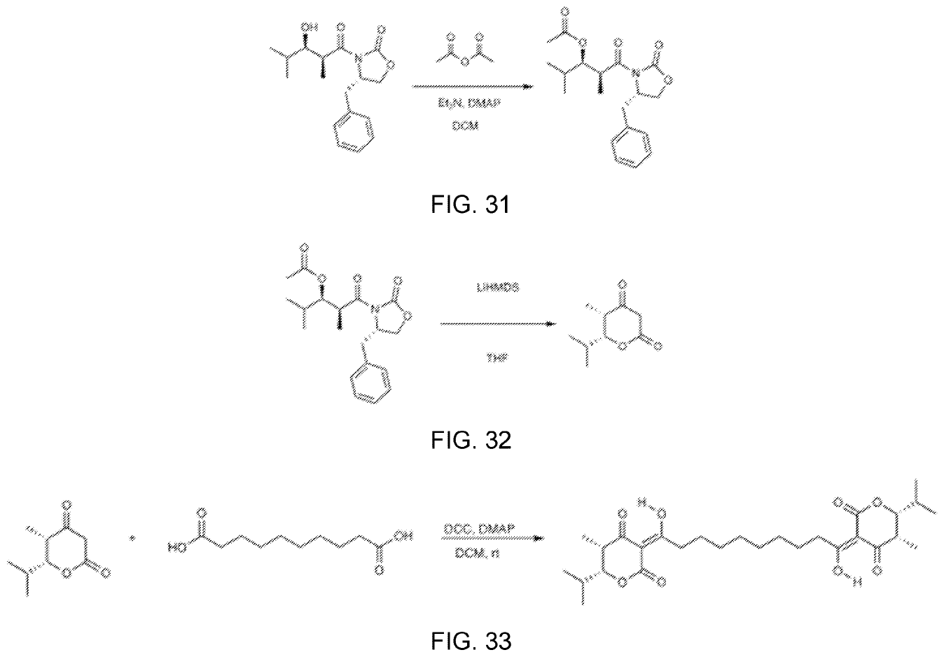

FIG. 31 provides a synthetic scheme for preparation of an Example monomer intermediate:

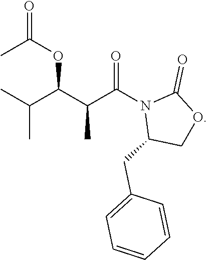 [SMILES: CC(=O)O[C@H](C(C)C)[C@H](C)C(=O)N1C(=O)OC[C@@H]1Cc1ccccc1]

(2S,3R)-1-((S)-4-benzyl-2-oxooxazolidin-3-yl)-2,4-dimethyl-1-oxopentan-3-yl acetate was synthesized by the following procedure. Et₃N (9.95 g, 98.4 mmol) was added to a stirred solution of (S)-4-benzyl-3-((2S,3R)-3-hydroxy-2,4-dimethylpentanoyl)oxazolidin-2-one (22.0 g, 75.7 mmol) in anhydrous DCM (200 mL), followed by the addition of freshly distilled acetic anhydride (9.27 g, 90.8 mmol). The solution mixture was cooled to 0° C., and a solution of DMAP (1.85 g, 15.1 mmol) in DCM (5 mL) was dropwise added. The reaction was allowed to stir at 0° C. for 30 min before warmed up to room temperature and further reacted for another 90 min. The reaction was then quenched by the addition of saturated aqueous NH₄Cl (200 mL) and the aqueous phase was separated and extracted by DCM (2×100 mL). All the organic layers were combined, washed with brine (100 mL) before being dried over Na₂SO₄. The solvent was removed in vacuo to yield the crude product, which was purified by column chromatography (silica gel, 5-30% EtOAc in hexane) to give (2S,3R)-1-((S)-4-benzyl-2-oxooxazolidin-3-yl)-2,4-dimethyl-1-oxopentan-3-yl acetate as colorless crystals (81% isolated yield, R_{f}=0.55, 1:3 EtOAc/hexane); ¹H NMR (500 MHz, CDCl₃) δ 7.37-7.08 (m, 5H), 4.90 (dd, J=9.3, 2.9 Hz, 1H), 4.47 (m, 1H), 4.27 (dd, J=8.4, 8.1 Hz, 1H), 4.21-4.03 (m, 2H), 3.26 (dd, J=13.4, 3.4 Hz, 1H), 2.75 (dd, J=13.3, 9.9 Hz, 1H), 2.03 (s, 3H), 1.90 (m, 1H), 1.18 (d, J=6.9 Hz, 3H), 0.99 (d, J=6.8 Hz, 3H), 0.88 (d, J=6.6 Hz, 3H) ppm; ¹³C NMR (126 MHz, CDCl₃) δ 174.63, 171.24, 153.88, 135.63, 129.57, 129.01, 127.37, 78.35, 66.52, 56.20, 39.49, 38.09, 29.64, 20.93, 19.30, 18.40, 9.18 ppm.

### Example 8

FIG. 32 provides a synthetic scheme for preparation of an Example monomer intermediate:

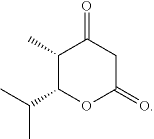

(5 S,6R)-6-isopropyl-5-methyldihydro-2H-pyran-2,4(3H)-dione was synthesized by the following procedure. (2S,3R)-1-((S)-4-benzyl-2-oxooxazolidin-3-yl)-2,4-dimethyl-1-oxopentan-3-yl acetate (3.13 g, 9.01 mmol) was dissolved in anhydrous THF (50 mL), and the solution was cooled to −78° C., followed by dropwise addition of a pre-cooled (−78° C.) solution of LiHMDS in THF (1.0 M, 50 mL) under vigorous stirring. The reaction was further allowed to stir for 1 h before quenched by a solution mixture of sat. NH₄Cl/H₂O/MeOH (v:v:v=1:1:1. 250 mL). EtOAc (150 mL) was added to the solution, and the aqueous phase was separated and acidified to pH<2 by HCl (2.0 M). The mixture was extracted by DCM (3×50 mL), and all the DCM layers were combined before being dried over Na₂SO₄. The solvent was removed in vacuo to yield the crude product, which was purified by column chromatography (silica gel, 10-60% EtOAc in hexane) to give (5S,6R)-6-isopropyl-5-methyldihydro-2H-pyran-2,4(3H)-dione as white solids (75% isolated yield, R_{f}=0.42, 1:1 EtOAc/hexane); ¹H NMR (500 MHz, CDCl₃) δ 5.21 (s, 1H, enol isomer), 4.17 (dd, J=9.8, 2.5 Hz, 1H), 3.60 (d, J=18.9 Hz, 1H), 3.39 (d, J=19.0 Hz, 1H), 2.70 (qd, J=7.5, 2.4 Hz, 1H), 2.02 (dt, J=9.9, 6.6 Hz, 1H), 1.14 (d, J=6.6 Hz, 3H), 1.13 (d, J=7.4 Hz, 3H), 0.93 (d, J=6.8 Hz, 3H) ppm; ¹³C NMR (126 MHz, CDCl₃) δ 204.51, 167.63, 83.84, 45.88, 43.55, 28.86, 19.67, 17.95, 9.43 ppm.

### Example 9

FIG. 33 provides a synthetic scheme for preparation of an Example monomer compound:

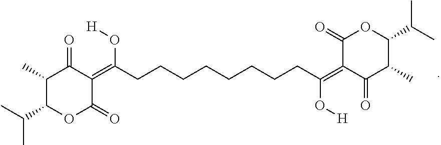 [SMILES: CC(C)[C@H]1OC(=O)/C(=C(/O)CCCCCCCC/C(O)=C2\C(=O)O[C@H](C(C)C)[C@H](C)C2=O)C(=O)[C@H]1C]

(6R,6′R)-3,3′-(1,10-dihydroxydecane-1,10-diylidene)bis(6-isopropyl-5-methyldihydro-2H-pyran-2,4(3H)-dione) was synthesized by the following procedure. (5S,6R)-6-isopropyl-5-methyldihydro-2H-pyran-2,4(3H)-dione (29.4 mmol), sebacic acid (14.3 mmol), and DMAP (42.9 mmol) were dissolved in 50 mL DCM under stirring at room temperature, followed by slow addition of a solution of DCC (34.3 mmol) in DCM (40 mL). The reaction was stirred for 24 h before filtered, the residue of which was washed by DCM (10 mL). The residue was washed by HCl (2.0 M) until the pH of the aqueous phase was less than 3, before being dried over MgSO₄. The solvent was removed in vacuo to yield the crude product, which was purified by column chromatography (silica gel, 5-40% EtOAc in DCM) to give (6R,6′R)-3,3′-(1,10-dihydroxydecane-1,10-diylidene)bis(6-isopropyl-5-methyldihydro-2H-pyran-2,4(3H)-dione) as a pale-yellow powder (isolated yield: 65%); ¹H NMR (500 MHz, CDCl₃) δ 18.17 (s, 1H), 17.85 (s, 1H), 16.21 (s, 1H), 16.14 (s, 1H), 4.04-3.78 (m, 2H), 3.15-2.88 (m, 4H), 2.78-2.50 (m, 2H), 2.03-1.86 (m, 2H), 1.74-1.52 (m, 4H), 1.42-1.25 (m, 8H), 1.19 (d, J=7.0 Hz, 3H), 1.11 (d, J=6.4 Hz, 3H), 1.09 (d, J=6.9 Hz, 3H), 0.97 (d, J=6.8 Hz, 3H), 0.88 (d, J=6.8 Hz, 3H) ppm; ¹³C NMR (125 MHz, CDCl₃): δ 205.66, 203.56, 199.47, 199.30, 198.30, 197.18, 194.92, 193.07, 175.03, 164.49, 164.41, 102.39, 101.84, 100.12, 99.03, 84.75, 83.68, 82.93, 81.68, 43.36, 43.12, 39.39, 38.93, 38.84, 38.52, 38.07, 36.32, 29.53, 29.48, 29.40, 29.37, 29.33, 29.27, 29.25, 29.22, 29.21, 29.04, 28.81, 28.61, 26.15, 26.11, 25.26, 24.84, 19.58, 19.55, 19.52, 17.80, 17.69, 14.80, 14.63, 10.64, 9.98, 9.70 ppm.

### Example 10

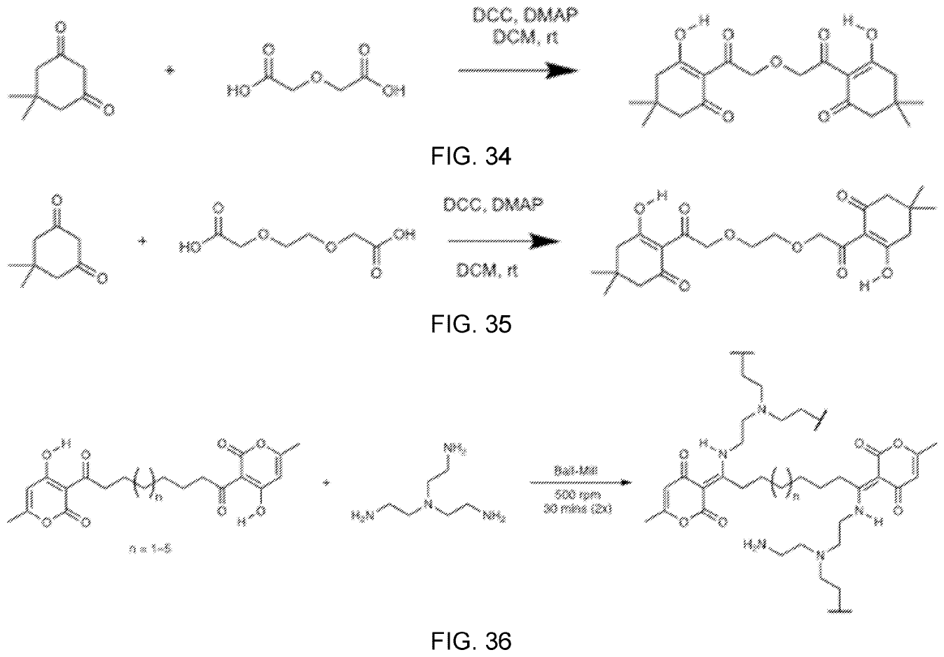

FIG. 34 provides a synthetic scheme for preparation of an Example monomer compound:

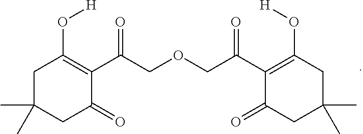

2,2′-(2,2′-oxybis(acetyl))bis(3-hydroxy-5,5-dimethylcyclohex-2-en-1-one) was synthesized by the following procedure. Dimedone (2.1 mmol), diglycolic acid (1 mmol), and DMAP (3 mmol) were dissolved in dichloromethane ([dimedone]=1.0 M) with stirring. A separate solution of DCC (2.4 mmol) in dichloromethane ([DCC]=1.0 M) was added slowly at room temperature to the reaction mixture. The yellow solution gradually turned strong orange, which was accompanied by the formation of a precipitate. The mixture was stirred at room temperature overnight (16 h), at which point the white N,N′-dicyclohexylurea precipitate was filtered. The precipitate was washed with DCM until colorless. The filtrate was collected and washed with 3% HCl until the pH of the aqueous phase was <3. The organic phase was separated, dried over MgSO₄, filtered and the solvent is removed under vacuum. The crude, yellow solid was solubilized in KOH (2.0 M) and the mixture stirred for 2 h, filtered, and precipitated in HCl (2.0 M). The solid was recrystallized in cyclohexane into a fine yellow powder. The solvent was filtrated and the yellow solid was dried under vacuum overnight (78% isolated yield). ¹H NMR (500 MHz, CDCl₃): δ 17.35 (s, 2H), 4.92 (s, 4H), 2.58 (s, 4H), 2.36 (s, 4H), 1.11 (s. 12H) ppm.

### Example 11

FIG. 35 provides a synthetic scheme for preparation of an Example monomer compound:

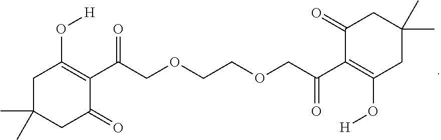

2,2′-(2,2′-(ethane-1,2-diylbis(oxy))bis(acetyl))bis(3-hydroxy-5,5-dimethylcyclohex-2-en-1-one) was synthesized in the same manner as Example 10 except that 3,3′-(ethane-1,2-diylbis(oxy))dipropanoic acid was used in place of diglycolic acid. The crude product was purified through column chromatography (DCM:EtOAc, 3:7). The solvent was evaporated and the pale-yellow solid was dried under vacuum overnight (42% isolated yield). ¹H NMR (500 MHz, CDCl₃): δ 17.45 (s, 2H), 4.86 (s, 4H), 2.57 (s, 4H), 2.35 (s, 4H), 1.10 (s, 12H) ppm. ¹³C NMR (125 MHz, CDCl₃): δ 202.6, 195.6, 195.2, 111.24, 75.33, 70.87, 51.9, 45.6, 31.1, 28.2 ppm.

### Example 12

FIG. 36 provides a synthetic scheme for preparation of an Example PDK Network derived from Triacetic Acid Lactone:

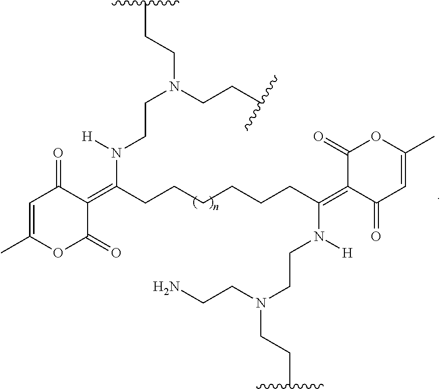

Polymer networks synthesized from TAL-derived ditopic triketone monomers and tris(2-aminoethyl)amine crosslinkers were synthesized using solventless ball-milling. The reactor in which the reactions were carried out was a zirconium-coated cylinder with an inner diameter of 4.5 cm and a height of 3.5 cm (reactor volume ˜50 mL). The same weight ratio of zirconium oxide ball bearings (5 mm diameter) to triketone monomer, with the ball bearings being 10 times the weight of triketone. The general procedure for all ball-milling reactions involved weighing out the appropriate amount of triketone monomer (2.0) and placing at the bottom of the ball mill, along with the ball bearings (20 g). To the triketone monomer was added tris(2-aminoethyl)amine (TREN) using a pre-calibrated micropipette such that the ratio of amine to triketone functional groups is 1.1 to 1. This was immediately followed by ball-milling the contents of the closed container for 30 min at 500 rpm with changes in spinning direction every 1 min. The reactor was opened to air and the reactor walls were scraped to bring together the reactants homogeneously. Ball-milling was resumed under identical conditions for 30 more minutes. The powders were recovered from the reactor and the residual water was removed under vacuum at 90° C. The polymers were characterized by DSC, DMA, and density measurements. ¹³C Solid State NMR and X-Ray Detection techniques were also used to determine the full conversion of the triketone into network. Glass Transition Temperature (determined via DSC)=151, 141, 132, 125 and 114° C., respectively for PDK networks, where n=1-5. Tensile Storage Modulus (determined via DMA)=2.78, 2.02, 2.06, 2.04 and 1.76 GPa respectively for PDK networks, where n=1-5 and Tensile Elastic Modulus (determined via DMA)=14.8, 12.7, 13.8, 9.6 and 10.7 MPa respectively for PDK networks, where n=1-5. Glass Transition Temperature (determined via tan δ in DMA)=155, 147, 136, 110 and 108° C. respectively for PDK networks, where n=1-5. Density measurements=1.129, 1.011, 1.078, 1.032 and 1.086 g mL⁻¹ respectively for PDK networks, where n=1-5.

### Example 13

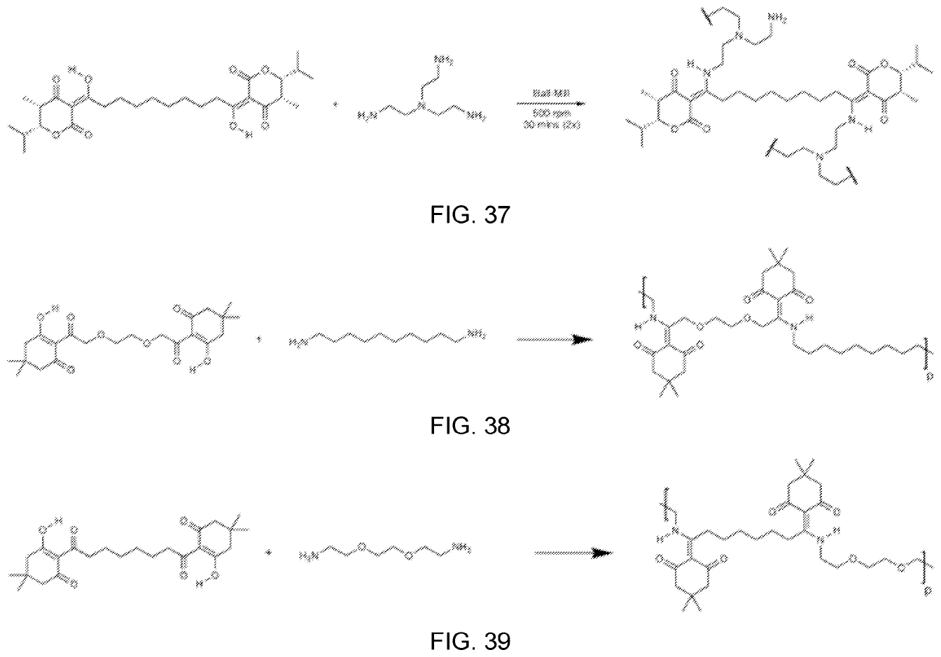

FIG. 37 provides a synthetic scheme for preparation of an Example chiral PDK Network:

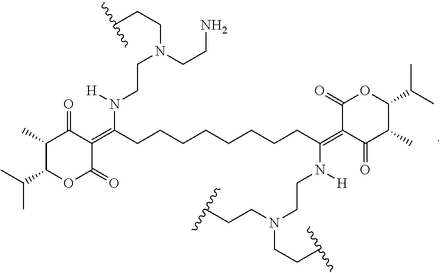 [SMILES: *CCN(CC*)CCN/C(CCCCCCCC/C(NCCN(CC*)CCN)=C1\C(=O)O[C@H](C(C)C)[C@H](C)C1=O)=C1/C(=O)O[C@H](C(C)C)[C@H](C)C1=O]

A chiral PDK network was synthesized using similar procedure to Example 12. The polymers were characterized by DSC and TGA. Glass Transition Temperature (determined via DSC)=72° C., Degradation temperature at 95% mass loss (determined via TGA)=210° C.

### Example 14

FIG. 38 provides a synthetic scheme for preparation of an Example linear polydiketonenamines from Oxo-Functionalized Triketone Monomers:

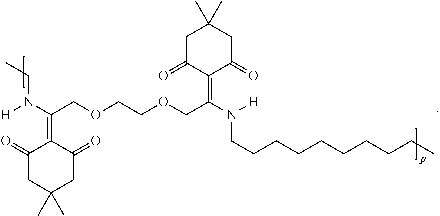

Linear poly(diketoenamine) bearing oxo-groups in the triketone monomer were prepared from 2,2′-(2,2′-(ethane-1,2-diylbis(oxy))bis(acetyl))bis(3-hydroxy-5,5-dimethylcyclohex-2-en-1-one) and 1,10-diaminodecane in a 1:1 stoichiometry. The reaction proceeded in the melt via a two-step process: first, the monomers were combined in a closed reactor with at 200 rpm and 150° C. for 2 h, then the water from the polycondensation reaction was removed under vacuum (<0.1 mbar) at 200 rpm and 200° C. for 2 h. The reactor was opened to air and cooled to room temperature. The polymer solidified and was subsequently solubilized in methanol and precipitated from water. The polymer was filtered and washed several times with fresh portions of water. The polymer was finally dried under vacuum line at 80° C. overnight and characterized using DSC and ¹H NMR. Glass Transition Temperature (DSC)=21° C. ¹H NMR (500 MHz, CDCl₃): δ 13.31 (s), 8.39 (s), 4.99-4.71 (m), 3.83-3.58 (m), 2.98-2.35 (m), 1.68-1.65 (m), 1.40-1.27 (m) 1.16-1.01 (m) ppm.

### Example 15

FIG. 39 provides a synthetic scheme for preparation of an Example linear polydiketonenamines from Oxo-Functionalized Amine Monomers:

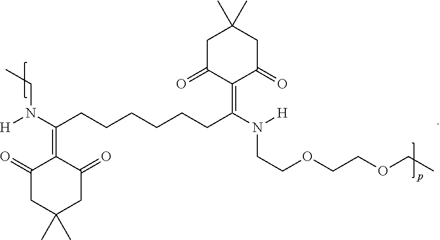

Linear poly(diketoenamine) bearing oxo-groups in the amine monomer was synthesized in the manner described in Example 14, using 1,8-bis(2-hydroxy-4,4-dimethyl-6-oxocyclohex-1-en-1-yl)octane-1,8-dione and 2,2′-(ethylenedioxy)bis(ethylamine) in a 1:1 stoichiometry. Glass Transition Temperature (DSC)=63° C., Degradation temperature at 90% mass loss (TGA)=354° C. Size Exclusion Chromatography (Solvent=DMF): M_{w}=77,700 g mol⁻¹, Mₙ=41,100 g mol⁻¹, Ð=1.89. ¹H NMR (500 MHz, CDCl₃): δ 13.55 (s), 3.78-3.76 (m), 3.66-3.63 (m), 2.99-2.98 (m), 2.37 (s), 1.55 (s), 1.03 (s) ppm.

### Example 16

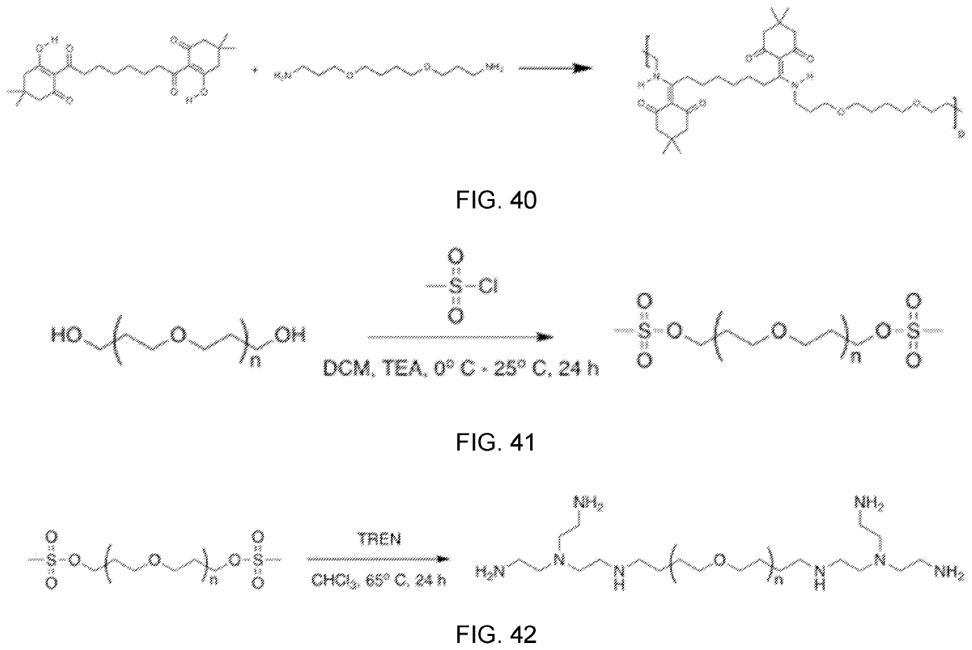

FIG. 40 provides a synthetic scheme for preparation of an Example linear polydiketonenamines from Oxo-Functionalized Amine Monomers:

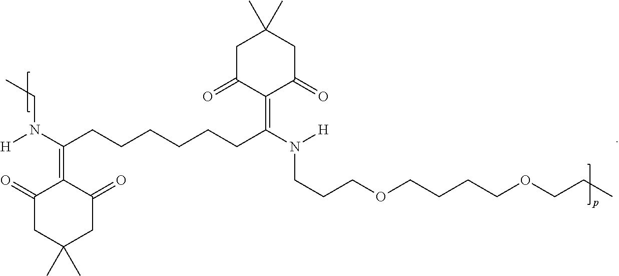

Linear poly(diketoenamine) bearing oxo-groups in the amine monomer was synthesized in the manner described in Example 14, using 1,8-bis(2-hydroxy-4,4-dimethyl-6-oxocyclohex-1-en-1-yl)octane-1,8-dione and 4,9-dioxa-1,12-dodecanediamine in a 1:1 stoichiometry. Glass Transition Temperature (DSC)=31° C., Degradation temperature at 90% mass loss (TGA)=357° C. Size Exclusion Chromatography (Solvent=DMF): M_{w}=45,900 g mol⁻¹, Mₙ=29,600 g mol⁻¹, Ð=1.55. ¹H NMR (500 MHz, CDCl₃): δ 13.55 (s), 3.59-3.53 (m), 3.00 (s), 2.44-2.42 (m), 1.97-1.95 (m), 1.64-1.56 (m), 1.19-1.05 (m) ppm.

### Example 17

FIG. 41 provides a synthetic scheme for preparation of an Example poly(tetrahydrofuran)-bis-O-mesylate intermediate.

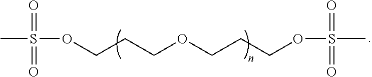

Poly(tetrahydrofuran)-bis-O-mesylate was synthesized by the following procedure. Poly(tetrahydrofuran) diol (Mₙ=2,000 g mol⁻¹) (30 g, 15 mmol) and triethylamine (10.6 g, 7 eq) were dissolved in dichloromethane while stirring. The reaction mixture was cooled to 0° C. and a separate solution of methanesulfonyl chloride (6 g, 3.5 eq) in dichloromethane was added dropwise. The reaction was allowed to proceed for 24 h while warming to room temperature. The resulting suspension was filtered to remove the triethylamine hydrochloride salt, and the organic fraction was washed 3 times with deionized water. The product was dried over magnesium sulfate and the solvent was removed under reduced pressure to yield pTHF-bis-O-mesylate as a waxy orange solid. ¹H NMR (500 MHz, CDCl₃): δ 4.23 (t, 4H, J=6.49 Hz), 3.38 (s, 144H), 2.98 (s, 6H), 1.81 (m, 4H, J=2.38 Hz), 1.59 (s, 144H). FT-IR (ATR, cm⁻¹): 2941, 2861, 2803, 1491, 1475, 1371, 1250, 1209, 1175, 1103, 995, 945, 815, 746, 562, 528, 448.

### Example 18

FIG. 42 provides a synthetic scheme for preparation of an Example poly(tetrahydrofuran)-bis-tris(2-aminoethyl)amine monomer intermediate:

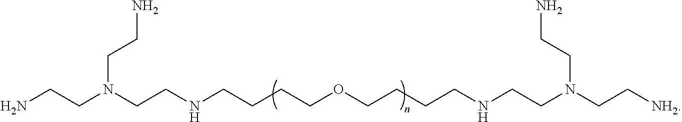

Poly(tetrahydrofuran)-bis-tris(2-aminoethyl)amine was synthesized by the following procedure. 20 g pTHF-bis-O-mesylate was dissolved in 120 mL chloroform and added dropwise to 35 g TREN (4 equivalents of NH₂:mesylate) at 65° C. under a dry N₂ stream. The reaction was allowed to proceed for 18 h. The crude product was dissolved in 200 mL CHCl₃, filtered, and stirred with basic ion exchange resin for 12 h to generate the free base. The resin was removed by filtration and the solvent was removed under reduced pressure to yield a yellow semisolid, which was then precipitated into cold DI water to recover a white solid. The product was washed twice with cold water and dried under reduced pressure to yield polyTHF-bis-TREN as a waxy white solid. ¹H NMR (500 MHz, CDCl₃): δ 3.40 (s, 144H), 2.76-2.52 (m, 28H J=16.8 Hz), 1.60 (s, 149H).). FT-IR (ATR, cm⁻¹): 3352, 2941, 2862, 2804, 1577, 1491, 1475, 1460, 1371, 1301, 1251, 1209, 1108, 996, 959, 869, 817, 746, 564. MALDI (reflector mode, dithranol+NaTFA): Mₙ=2,160 g mol⁻¹, Ð=1.18.

### Example 19

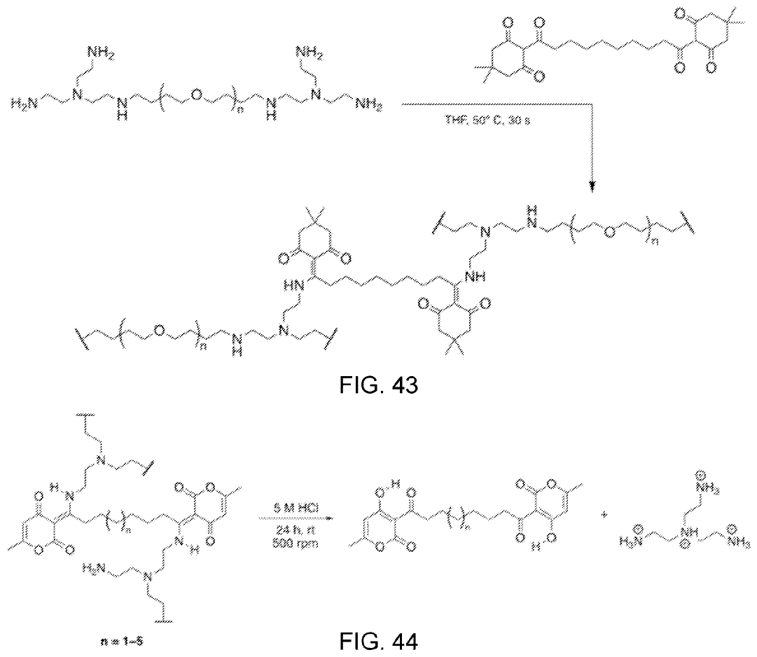

FIG. 43 provides a synthetic scheme for preparation of an Example PDK elastomers:

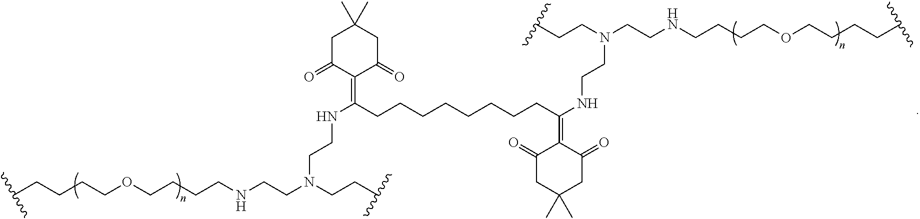

Crosslinked polydiketoenamine elastomers were prepared from pTHF-bis-TREN and 1,10-bis(2-hydroxy-4,4-dimethyl-6-oxocyclohex-1-en-1-yl)decane-1,10-dione. pTHF-bis-TREN (1 g) was dissolved in 1 g THF and heated to 50° C. Separately, 1,10-bis(2-hydroxy-4,4-dimethyl-6-oxocyclohex-1-en-1-yl)decane-1,10-dione (0.313 g, 1:1.3 triketone:amine) was dissolved in 0.313 mL THF at 50° C. The solutions were combined and stirred for approximately 30 s, upon which the solution rapidly formed a gel. To synthesize polydiketoenamine elastomers containing carbon black, a 0.5% w/v dispersion of carbon black in THF was first prepared, and the elastomer was synthesized using the above procedure using the THF solution of carbon black. The gel was dried under reduced pressure at 70° C. for 12 h.

### Example 20

FIG. 44 provides a synthetic scheme for depolymerization of an Example PDK Networks Derived from Triacetic Acid Lactone:

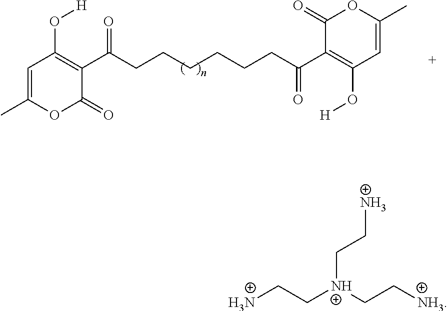

PDK resins were placed in 20-mL glass vials along with 5.0 M HCl (15 mL) and a magnetic stirrer. Depolymerization reactions were conducted over 24 h at room temperature while stirring at 500 rpm. Triketones were isolated by extraction with CH₂Cl₂ and evaporation of the organic phase. Percent triketone recovery was calculated by the following equation:

$$Percent TK Recovery = \frac{TK ( g )}{\frac{PDK ( g )}{1 + x - \frac{2 ( {MW}_{H 2 O} )}{{MW}_{TK}}}} * 100 \%$$

### Where:

TK (g) is the mass of recovered ditopic triketone monomer,
PDK (g) is the mass of the PDK to be depolymerized,
x is the mass ratio of TREN:Triketone used during PDK polymerization,
MW_{H2O} is the molecular weight of H₂O,
MW_{TK} is the molecular weight of the triketone,

The PDK network corresponding to n=1 (472 mg) was completely depolymerized in aqueous 5.0 M HCl (15 mL) over 24 h at room temperature, yielding a light brown suspension. The mixture was extracted with CH₂Cl₂ (20 mL), and the organic layer was evaporated under vacuum to give an orange-brown paste. The product was dried under vacuum at 80° C. to yield the ditopic triketone monomer where n=1 (yield=289 mg, 72.4%). The PDK network corresponding to n=2 (320 mg) was completely depolymerized in aqueous 5.0 M HCl (15 mL) over 24 h at room temperature, yielding a light brown suspension. The mixture was extracted with CH₂Cl₂ (20 mL), and the organic layer was evaporated under vacuum to give a dark red solid. The product was dried under vacuum at 80° C. to yield the ditopic triketone monomer where n=2 (yield=254 mg, 93.4%). The PDK network corresponding to n=3 (487 mg) was completely depolymerized in aqueous 5.0 M HCl (15 mL) over 24 h at room temperature, yielding a dark yellow suspension. The mixture was extracted with CH₂Cl₂ (20 mL), and the organic layer was evaporated under vacuum to give a yellow-orange paste. The product was dried under vacuum at 80° C. to yield the ditopic triketone monomer where n=3 (yield=376 mg, 90.3%). The PDK network corresponding to n=4 (303 mg) was completely depolymerized in aqueous 5.0 M HCl (15 mL) over 24 h at room temperature, yielding a dark orange suspension. The mixture was extracted with CH₂Cl₂ (20 mL), and the organic layer was evaporated under vacuum to give a dark red solid. The product was dried under vacuum at 80° C. to yield the ditopic triketone monomer where n=4 (yield=256 mg, 98.4%). The PDK network corresponding to n=5 (527 mg) was completely depolymerized in aqueous 5.0 M HCl (15 mL) over 24 h at room temperature, yielding a brown suspension. The mixture was extracted with CH₂Cl₂ (20 mL), and the organic layer was evaporated under vacuum to give a yellow-brown paste. The product was dried under vacuum at 80° C. to yield the ditopic triketone monomer where n=5 (yield=455 mg, 100%).

### Example 21

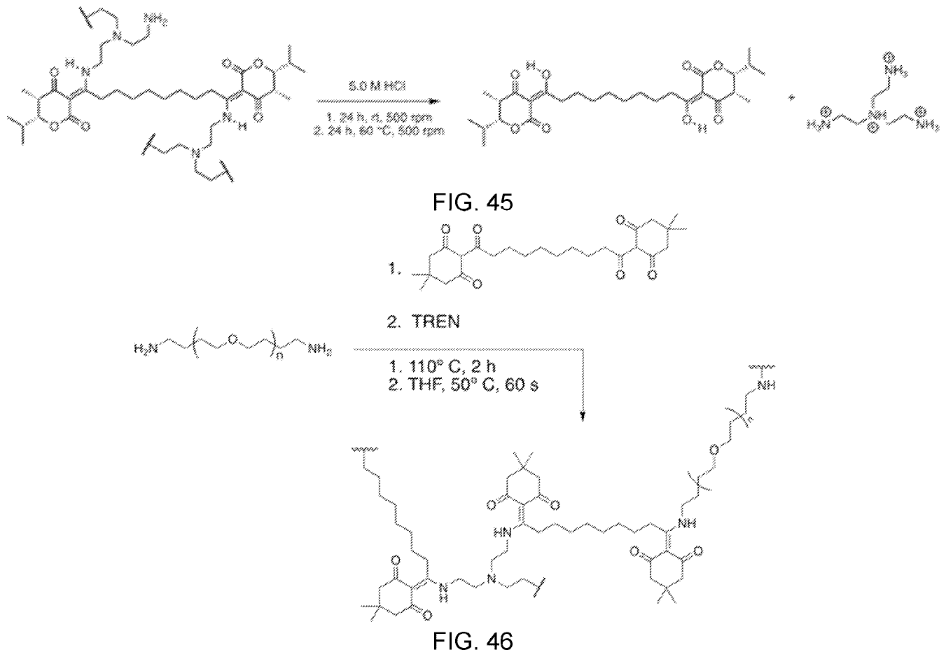

FIG. 45 provides a synthetic scheme for depolymerization of an Example Chiral Bio-Based PDK Network:

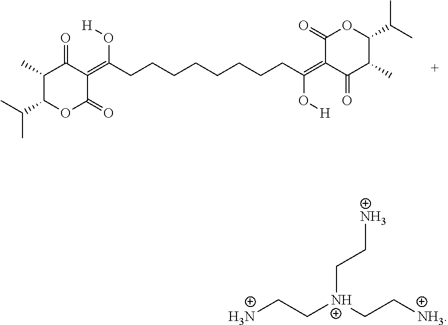

The chiral bio-based PDK network (506 mg) was placed in a 20-mL glass vial, along with 5.0 M HCl (15 mL) and a magnetic stirrer. Depolymerization reaction was conducted over 24 h at room temperature while stirring at 500 rpm, yielding a white PDK solid sample with partially hydrolyzed surface. Depolymerization reaction was further pursued over 24 h at 60° C. while stirring at 500 rpm, which resulted in complete depolymerization of the chiral bio-based PDK network. The chiral bio-based ditopic triketone monomer was isolated by extraction with CH₂Cl₂ and evaporation of the organic phase. The light brown powder product was dried under vacuum at 80° C. to yield the chiral bio-based ditopic triketone monomer (400 mg, yield=90%, purity=98%).

### Example 22

### Depolymerization of an Example PDK Elastomer Network.

PDK elastomers synthesized from bio-based 1,10-bis(2-hydroxy-4,4-dimethyl-6-oxocyclohex-1-en-1-yl)decane-1,10-dione and poly(tetrahydrofuran)-bis-TREN were incubated in a solution of 5 M HCl at room temperature with stirring. 1,10-bis(2-hydroxy-4,4-dimethyl-6-oxocyclohex-1-en-1-yl)decane-1,10-dione was recovered as a solid from the reaction mixture after centrifugation. The supernatant containing dissolved and ionized poly(tetrahydrofuran)-bis-TREN was reserved. The recovered 1,10-bis(2-hydroxy-4,4-dimethyl-6-oxocyclohex-1-en-1-yl)decane-1,10-dione was washed with DI water and dried under vacuum as the regenerated triketone monomer in 90% yield. The reserved aqueous solution containing the dissolved and ionized poly(tetrahydrofuran)-bis-TREN was basified with 6.0 M aqueous sodium hydroxide until the pH was 14, and water was removed under vacuum. The residual solids were redissolved in dichloromethane. Insoluble NaCl and NaOH were removed from the organic phase by filtration, and the solvent was removed under vacuum to yield poly(tetrahydrofuran)-bis-TREN as the regenerated free base in 44% yield.

### Example 23

FIG. 46 provides a synthetic scheme for preparation of an Example PDK elastomer with incomplete depolymerization in strong acid:

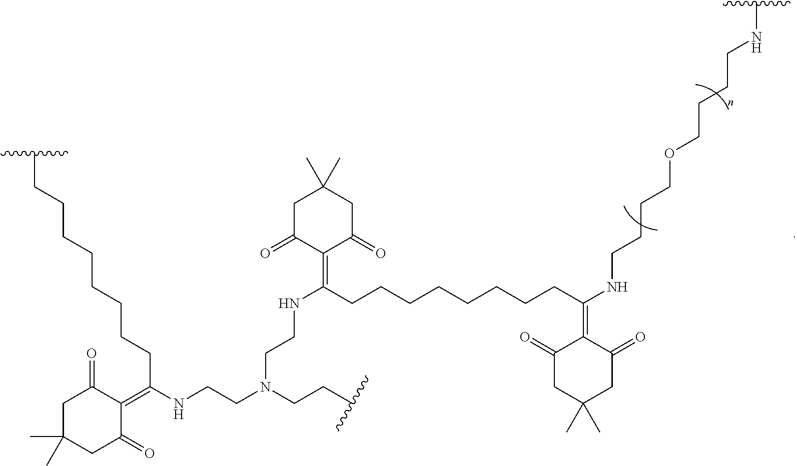

Crosslinked polydiketoenamine elastomers were prepared from bio-based 1,10-bis(2-hydroxy-4,4-dimethyl-6-oxocyclohex-1-en-1-yl)decane-1,10-dione, pTHF-bis-amine, and TREN. The pTHF-bis-amine (1 g) was combined with 1,10-bis(2-hydroxy-4,4-dimethyl-6-oxocyclohex-1-en-1-yl)decane-1,10-dione (0.313 g) without solvent and heated to 110° C. with stirring for 2 h. The mixture was then cooled to 50° C., and 1.3 mL THF was added. TREN (0.025 g) was added to the solution and stirred for approximately 60 s, upon which the solution rapidly formed a gel. The overall triketone:amine content was 1:1.3. The gel was dried under reduced pressure at 70° C. for 12 h. Samples of the elastomer were incubated in a solution of 5 M HCl at room temperature or at elevated temperature (60° C.) with stirring for up to 48 h, after which the depolymerization was found to be incomplete.

### Example 24

### Depolymerization of an Example PDK Reinforced Rubber.

PDK elastomer networks synthesized from bio-based 1,10-bis(2-hydroxy-4,4-dimethyl-6-oxocyclohex-1-en-1-yl)decane-1,10-dione and poly(tetrahydrofuran)-bis-TREN that were also reinforced with carbon black (0.5% w/w) during the synthesis were incubated in a solution of 5 M HCl at room temperature with stirring. 1,10-bis(2-hydroxy-4,4-dimethyl-6-oxocyclohex-1-en-1-yl)decane-1,10-dione was recovered as a solid from the reaction mixture after centrifugation. The supernatant containing dissolved and ionized poly(tetrahydrofuran)-bis-TREN was reserved. The recovered 1,10-bis(2-hydroxy-4,4-dimethyl-6-oxocyclohex-1-en-1-yl)decane-1,10-dione was washed with DI water and dried under vacuum as the regenerated triketone monomer in 79% yield. The reserved aqueous solution containing the dissolved and ionized poly(tetrahydrofuran)-bis-TREN was basified with 6.0 M aqueous sodium hydroxide until the pH was 14, and water was removed under vacuum. The residual solids were redissolved in dichloromethane. Insoluble NaCl and NaOH were removed from the organic phase by filtration, and the solvent was removed under vacuum to yield poly(tetrahydrofuran)-bis-TREN as the regenerated free base in 39% yield.

### Example 25—Uncoupling Short-Range Bond Exchange from Long-Range Viscoelastic Flow in Circular Polydiketoenamine Elastomers

Elastomers are widely used in textiles, foam, and rubber, yet are rarely recycled due to the difficulty in deconstructing polymer chains to reusable monomers. Introducing reversible bonds in these materials offers prospects for improving their circularity, however, concomitant bond exchange permits creep, which is undesirable. This example describes how to architect dynamic covalent polydiketoenamine (PDK) elastomers prepared from polyetheramine and triketone monomers, not only for energy-efficient circularity, but also outstanding creep resistance at high temperature. By appending polytopic crosslinking functionality at the chain ends of flexible polyetheramines, creep can be reduced from >200% to less than 1%, relative to monotopic controls, producing mechanically robust and stable elastomers and carbon-reinforced rubbers that are readily depolymerized to pure monomer in high yield. The multivalent chain end was also found valuable for ensuring complete PDK deconstruction. Mapping reaction coordinates in energy and space across a range of potential conformations reveals the underpinnings of this behavior, which involves preorganization of the transition state for diketoenamine bond acidolysis when a tertiary amine is also nearby.

Crosslinked elastomers and rubbers have broad commercial and industrial uses due to their thermal, chemical, and mechanical stability. This stability makes deconstructing them to their original monomers a persistent challenge, yet important for a circular plastics economy. Elastomer networks featuring reversible bonds open the door to chemical recycling, however, most fail to close the loop upon deconstruction-more often, chemical recycling returns fragments of the network, rather than monomers. Chemical recycling to monomer has been most successful when networks are crosslinked using dynamic covalent bonds that can be cleaved solvolytically. However, bond exchange reactions inherent to dynamic covalent elastomers also promote creep and the materials exhibit poor mechanical stability in load-bearing environments, particularly at elevated temperature. Controlling viscous flow to minimize or eliminate creep in future generations of circular elastomers therefore requires more careful consideration, not only of the reversible bond, but also the network architecture. Ideally, short-range bond-exchange kinetics and long-range viscoelastic flow can be uncoupled.

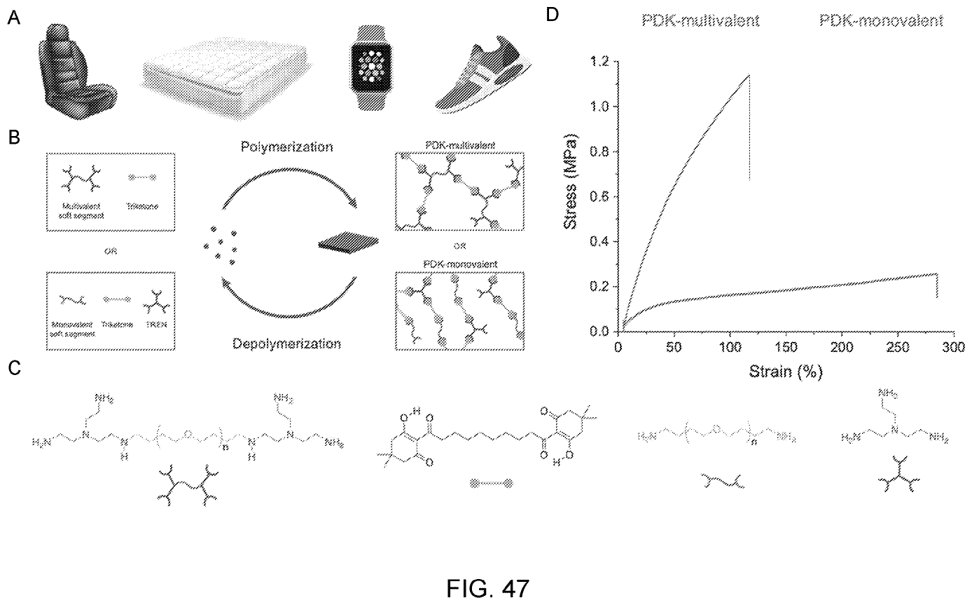

This example describes how to architect circular polydiketoenamine (PDK) elastomers and carbon black-reinforced PDK rubbers to resist creep by tailoring the valency at crosslinking sites associated with flexible polyetheramine segments within the network (FIG. 47). Notable in the designs, diketoenamine bonds retain their ability to participate in short-range bond exchange, enabling thermoforming during manufacturing and stress relaxation upon strain; however, viscoelastic flow over longer length scales is entropically disfavored due to the multiplicity of anchor points to the network, which render the elastomers exceptionally resistant to creep, even at high temperature. Interestingly, the end-group structure of the polyetheramine monomer was also found to be relevant to the rate of PDK depolymerization during chemical recycling: whereas monovalent polyetheramine monomers producing creep-susceptible PDK elastomers were slow to depolymerize, multivalent polyetheramine monomers producing creep-resistant PDK elastomers were completely depolymerized within 24 h. This enabled facile recovery of the multivalent polyetheramine and triketone monomers in high yields with high purity, permitting their reuse in subsequent manufacturing cycles. To understand this behavior, a theoretical framework was developed to explore reactive conformations along the reaction coordinate in PDK hydrolysis. In doing so, an important role played by a proximal ionized amine was identified, exclusive to the multivalent polyetheramine end-group, that pre-organizes the transition state associated with the rate-limiting step, lowering the standard free energy of activation (ΔG^{‡}) by 13 kJ mol⁻¹ for diketoenamine hydrolysis in strong acid. Transition-state pre-organization emerges with due importance in achieving high efficiency and low-carbon intensity in PDK chemical recycling.

This examples advances macromolecular design of elastomeric soft segments in chemically recyclable dynamic covalent thermosets that simultaneously enable mechanical stability and depolymerization to monomer. Given the vast chemical space available to both polyetheramine and triketone monomers, the examples described herein can be exploited for co-designing elastomers and rubbers on the basis of both performance and circularity in chemical recycling.

Results and Discussion. PDK resins are created from a wide variety of polytopic triketone and amine monomers via spontaneous polycondensation (e.g., click) reactions. Previously, navigation of monomer chemical space has produced rigid vitrimers, whose glass transition temperatures (T_{g}) of 70-150° C. prioritized their use to applications requiring strength and structural integrity in that working temperature range. By incorporating flexible amine monomers into the network, it was determined to be possible to explore new regions of chemical space to seek sub-ambient temperature T_{g} and impart elasticity to the network architecture. The circularity afforded by PDK materials can extend to a broader range of useful polymeric materials, particularly elastomers and reinforced rubbers (FIG. 47, panel A).

The manner in which flexible amine monomers are integrated into PDK networks is non-trivial, given the tendency of elastomers to creep in polymer networks crosslinked with dynamic covalent bonds. It was hypothesized that by increasing the valency of amine end-groups in the flexible amine monomer, it is possible for PDK networks to retain their ability to engage in short-range bond exchange reactions, however, long-range viscoelastic flow responsible for creep may be entropically disfavored due to the large number of anchor points of the amine monomer to the larger network architecture.

To test this hypothesis, flexible polyetheramine monomers with multivalent amine end-groups were designed. Specifically, the chain-ends of polytetrahydrofuran diol (pTHF-diol) were transformed to the corresponding mesylates prior to reaction with tris(2-aminoethylamine) (TREN) to obtain the target monomer, pTHF-bis-TREN. Crosslinked PDK elastomers were then prepared from pTHF-bis-TREN and a triketone monomer (TK-10) separately synthesized from dimedone and sebacic acid (FIG. 47, Panel B and Panel C). The amine-to-triketone molar ratio was 1.3:1, and solid samples of these multivalent PDK elastomers (PDK-multivalent) were obtained within 30 s of polymerization at 60° C. in THF. As a control, to understand the impact of PDK network architecture and monomer end-group structure on thermomechanical properties and efficiency of recycling circularity, crosslinked PDK elastomers (PDK-monovalent) were also prepared from pTHF-diamine (e.g., a flexible polyether amine monomer with monovalent amine end-groups), TREN as the crosslinker, and TK-10; the pTHF weight fraction was matched and total excess amine content to those featured in the PDK-multivalent elastomer networks. Polymerized samples were easily remolded into defined shapes by pressing in Teflon molds at 150° C. and 60 psi for 5 min (FIG. 53). Tensile stress-strain measurements were then performed as an initial investigation of mechanical properties (FIG. 47, Panel D). PDK-multivalent had a lower elongation at break relative to PDK-monovalent (104% vs. 268%), yet maintained significantly higher tensile strength (1.14 vs. 0.257 MPa) and toughness (78.5 MJ m⁻³ vs. 49.9 MJ m⁻³). These results suggested a strong dependence between network architecture and properties in PDK elastomers, motivating a deeper investigation into their rheological behavior.

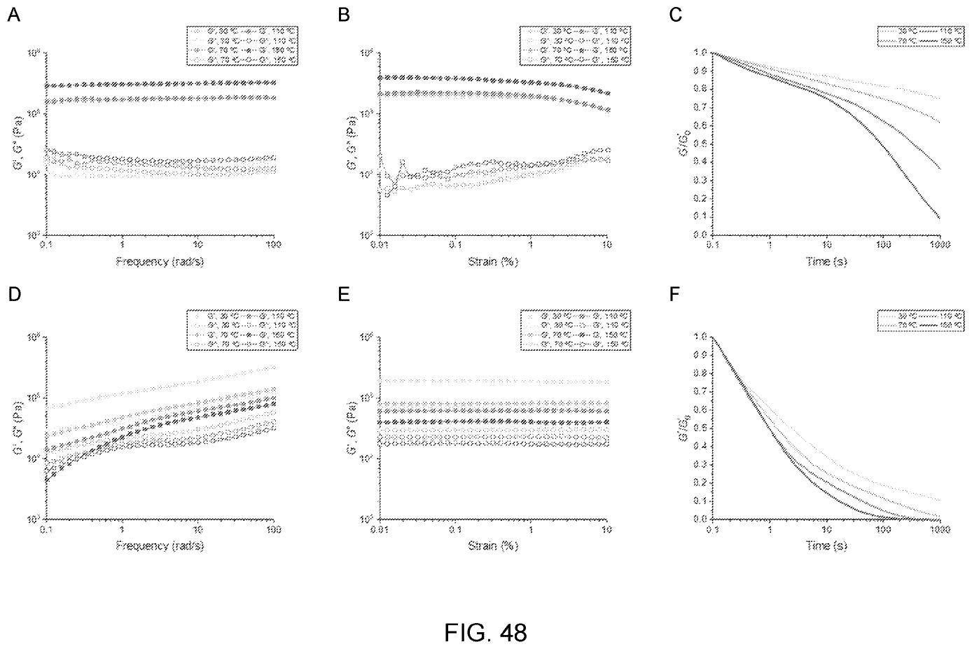

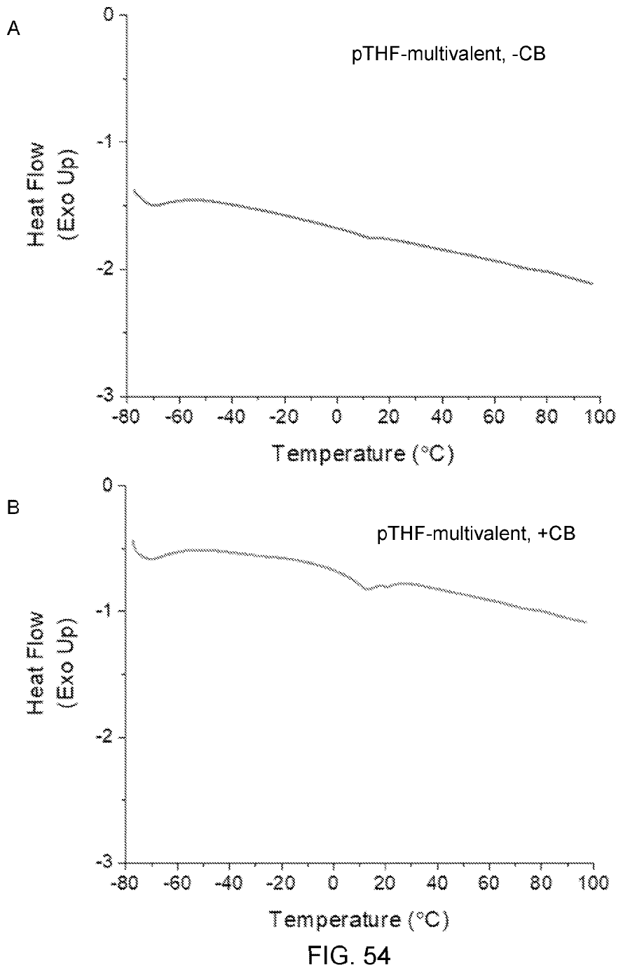

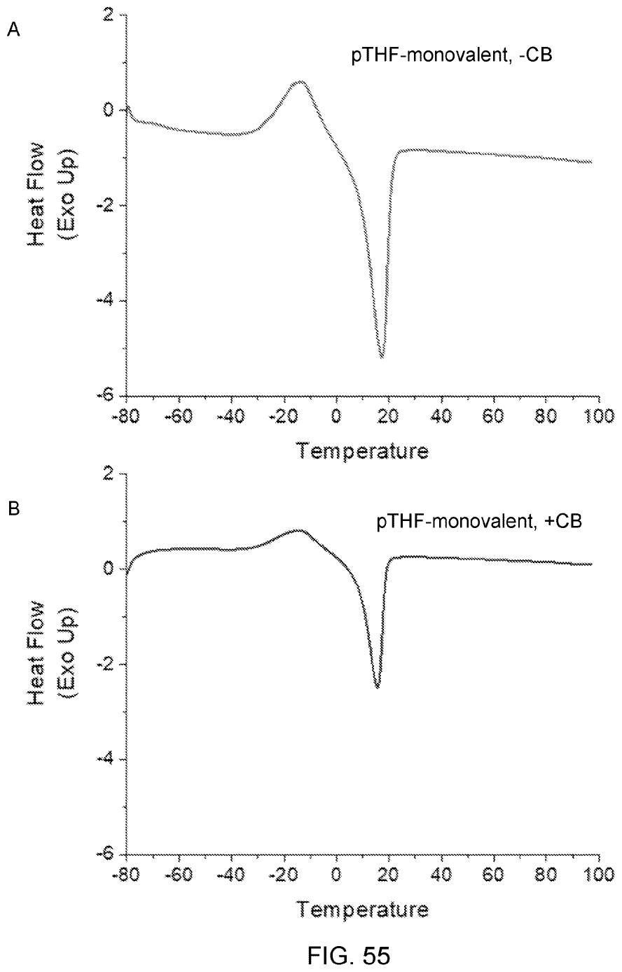

To test the second part of the hypothesis, pertaining to long-range viscoelastic flow, a series of rheology experiments were performed to reveal how amine monomer valency within the PDK network architecture may be relevant to or dictates the modulus and transient flow behavior of the materials (FIG. 48). To study the interactions between the network and reinforcing fillers, each PDK polycondensation was further carried out in the presence of 0.5 wt % carbon black, which produced black-pigmented carbon-reinforced PDK rubbers with essentially quantitative incorporation of the filler. Similar to the unfilled PDK formulations, the carbon-reinforced rubbers were amenable to thermoforming at 150° C. and 60 psi, producing samples that conformed to Teflon molds after 5 min of processing (FIG. 53). This confirmed part of the initial hypothesis in that short-range diketoenamine bond exchange remained feasible for all materials in this study. These experiments were carried out between 30-150° C., which is above the temperature of all thermal transitions in both PDK elastomers and rubbers, as observed by differential scanning calorimetry (DSC, FIG. 54 and FIG. 55).

Frequency sweep measurements quantifying the storage modulus (G′) and loss modulus (G″) for PDK-multivalent elastomers showed no crossover point in G′/G″ over the measured frequency range and minimal frequency-dependence on modulus (FIG. 48, Panel A). The modulus of PDK-multivalent elastomers was 200 kPa at 30° C. and increased slightly to 210 kPa at 110° C. Notably, at 150° C., the modulus nearly doubled to 390 kPa (FIG. 48, Panel B).

The observation of G′ increasing with temperature is consistent with polymer networks that engage in associative bond exchange. Moreover, this phenomenon comports with rubbery elasticity theory which states that the force (f) required to deform a network is related to temperature and the change in entropy with sample length (L) through the equation:

$$f = - T ( \frac{\partial S}{\partial L} ) \qquad ( 1 )$$

Since conformational entropy decreases when a polymer network is deformed, f becomes a positive quantity. Temperature (T) can be further to storage modulus (G) as:

$$G = {vk}_{B} T \qquad ( 2 )$$

where ν is network strands per unit volume and k_{B} is Boltzmann's constant, demonstrating the direct proportionality between G and T Surprisingly, however, the opposite trend was observed with PDK-monovalent elastomers, for which G decreased monotonically with temperature. Compared to the modulus for PDK-multivalent elastomers, the modulus for PDK-monovalent elastomers showed a strong frequency dependence, which indicates relatively shorter network relaxation times due to higher chain mobility (FIG. 48, Panel D). There is also noted a G/G″ crossover at 150° C., further confirming that at elevated temperature, chain mobility is high enough to permit long-range viscous flow.

Relevant to understanding this trend is the observation that the modulus for PDK-monovalent elastomers at 30° C. (190 kPa) (FIG. 48, Panel E) is comparable to that for PDK-multivalent elastomers (200 kPa) at the same temperature, despite PDK-monovalent elastomers containing less TREN as a crosslinker. This suggests that at relatively low temperature, non-covalent entanglements manifest in PDK-monovalent networks that increase the apparent crosslink density (ν). As temperature increases, excess amines can participate in diketoenamine bond exchange reactions to disentangle the linear segments within the network, allowing it to reach an equilibrium state with comparatively lower ν and thus a lower observed modulus. By contrast, PDK-multivalent elastomers have a higher density of covalent crosslinks by virtue of the pTHF-bis-TREN multivalent chain-end structure, which appears to dominate over any loss of non-covalent network entanglements associated with diketoenamine bond exchange.

Further evidence was observed that network reorganization is facile for PDK-monovalent elastomers by comparison to PDK-multivalent elastomers in the stress relaxation data for both (FIG. 48, Panel C and Panel F). Stress relaxation in PDK-multivalent elastomers did not follow a simple exponential decay, suggesting far more complex behavior associated with its relaxation than conventional models account for, e.g., extracting the activation energy (Arrhenius) or standard free energy of activation (Eyring) for bond exchange. Instead, the relative rates of relaxation between PDK-multivalent and PDK-monovalent elastomers were compared, with the characteristic time for PDK-monovalent to relax to a reduced modulus of e⁻¹ being greater than 2 orders of magnitude faster than PDK-multivalent. The temperature-dependent data for G′ and G″ indicate a preservation of non-covalent and covalent crosslinking density in PDK-multivalent elastomers and an apparent lowering of the crosslinking density in PDK-monovalent elastomers. It follows that stress relaxation in PDK-monovalent elastomers is concomitant with a decrease in the non-covalent contribution to network crosslinking density, since covalent crosslinking density is constant. Moreover, this occurs only in PDK-monovalent elastomers because a substantial portion of linear segments within the network contain diketoenamine bonds and bond exchange therein can produce a less entangled a network of chains under the applied strain. Since the presence of physical entanglements in covalently crosslinked rubbery polymers can increase fracture toughness, the stress relaxation observations can couple with the results in FIG. 47, Panel D and indicate that permanent entanglements in PDK-monovalent are unlikely to persist on the timescale of these experiments.

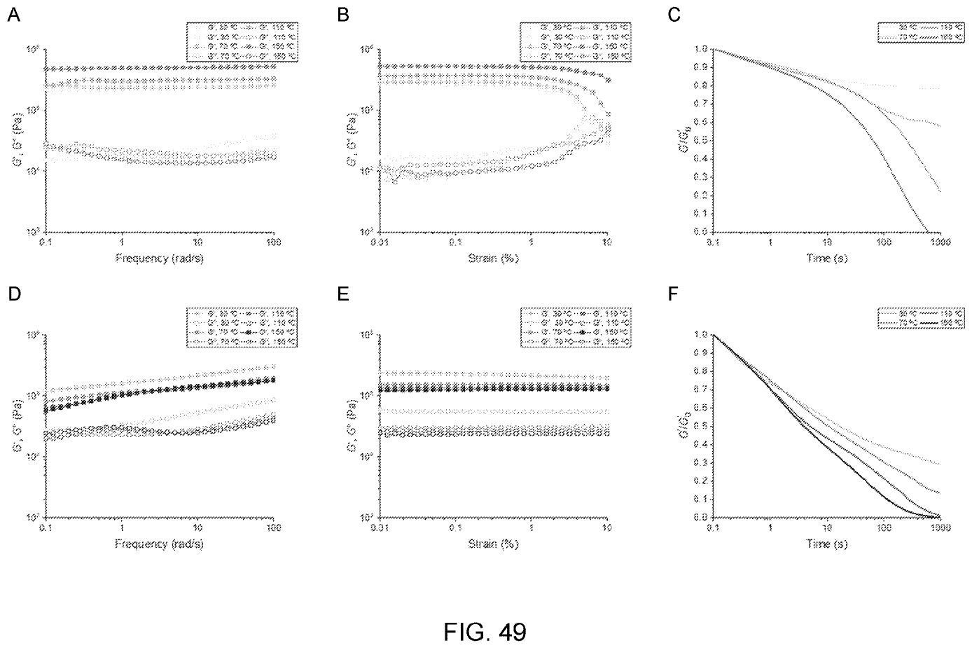

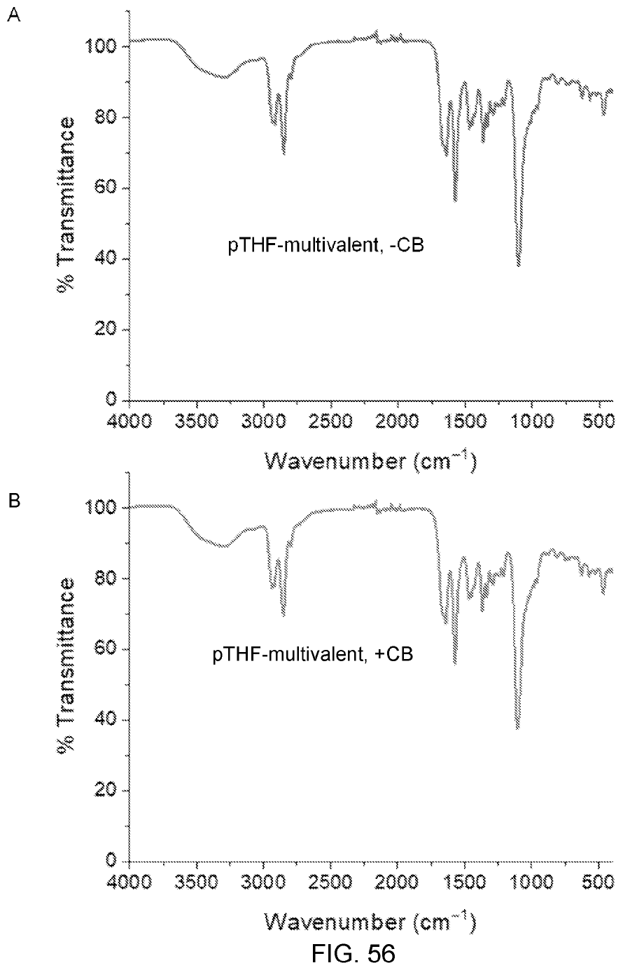

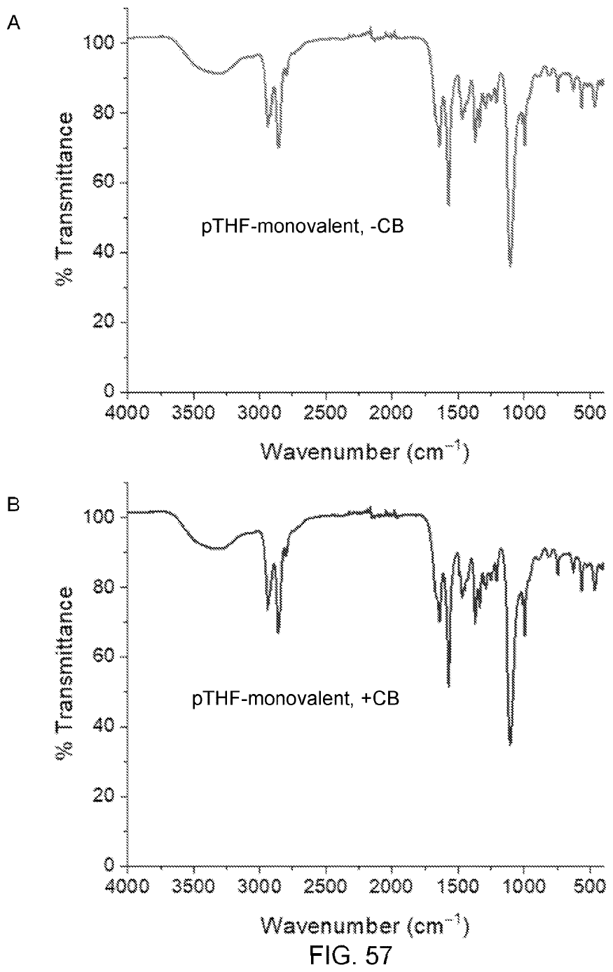

The use of how carbon black as a filler affects PDK elastomer rheology was also investigated. Carbon black is widely used to reinforce commercial rubbers and was easily dispersed into the PDK materials with no observable effect on polymerization. FTIR spectra for both PDK-multivalent and PDK-monovalent showed no changes in vibrational modes when carbon black was added (FIG. 56 and FIG. 57). Yet, a 40% increase in storage modulus was observed when incorporating only 0.5 wt % carbon black into PDK-multivalent elastomers (FIG. 49, Panels A-B), likely due to the formation of a bound rubber layer at the interface of the elastomer with carbon black. Because of the unique architecture of PDK-multivalent elastomers, the bound rubber layer is more likely to involve the pTHF segments, since potentially coordinating amine functionalities are most often found at sterically encumbered sites within the network. In stark contrast, coordinating amine functionality in PDK-monovalent elastomers may be found throughout the network thus promoting adsorption on that basis to a greater degree. Consequently, the reduction in chain mobility produced a larger reinforcing effect for PDK-monovalent elastomers, resulting in a reduced frequency dependence on modulus and an absence of G′/G″ crossover at any temperature over the range explored (FIG. 49, Panel D). The modulus for PDK-monovalent elastomers increased at all temperatures (FIG. 49, Panel E) compared to the unfilled formulation (FIG. 48, Panel E): for example, 250 kPa at 30° C. and 130 kPa at 150° C. in the carbon black-filled formulation compared to 190 kPa at 30° C. and 40 kPa at 150° C. in the unfilled formulation. While a decrease in modulus with temperature was again observed, the magnitude of the decrease was reduced, particularly at higher temperatures. Thus, microstructural attributes of PDK networks, particularly the manner in which excess amine functionality is presented throughout, strongly influence the reinforcing characteristics of carbon black fillers, tying back to differences in structure and dynamic properties of the bound rubber layer.

These differences in the bound rubber layers also impact stress relaxation behavior for both PDK-multivalent and PDK-monovalent carbon-reinforced rubbers (FIG. 49, Panel C and Panel F). The relaxation kinetics for PDK-multivalent rubbers did not change appreciably with the inclusion of carbon black, providing further evidence that excess amine functionality does not appreciably interact with the filler and that diketoenamine bond exchange permitting relaxation is highly localized. Conversely, relaxation in PDK-monovalent rubbers was substantially slower with the inclusion of carbon black, requiring >10-fold longer to relax to e⁻¹ compared to the unfilled sample. Thus, displaying amine functionality at sterically less hindered sites throughout the networks of PDK-monovalent elastomers permits their adsorption to filler surfaces and the relaxation behavior tied to that adsorption reflects slower chain dynamics of the bound rubber layer. When taken together, the results for unfilled and filled PDK elastomers validate the overall hypothesis by illustrating that multivalency in the flexible amine monomer is important to the creation of crosslinked PDK elastomers and rubbers that resist long-range viscous flow, yet retain capacity for short-range bond exchange to remain mechanically processable during thermoforming.

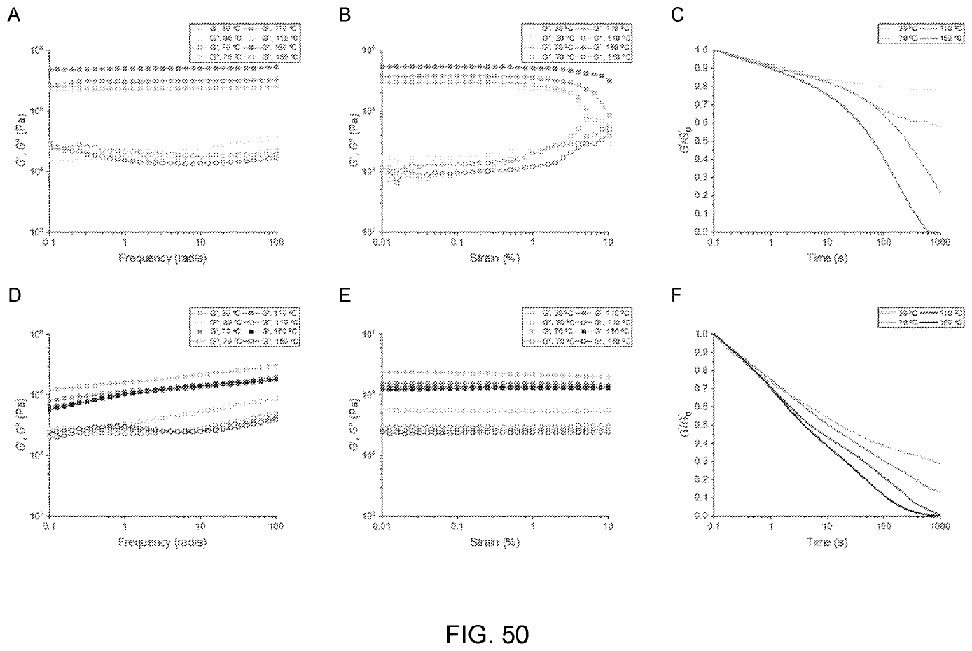

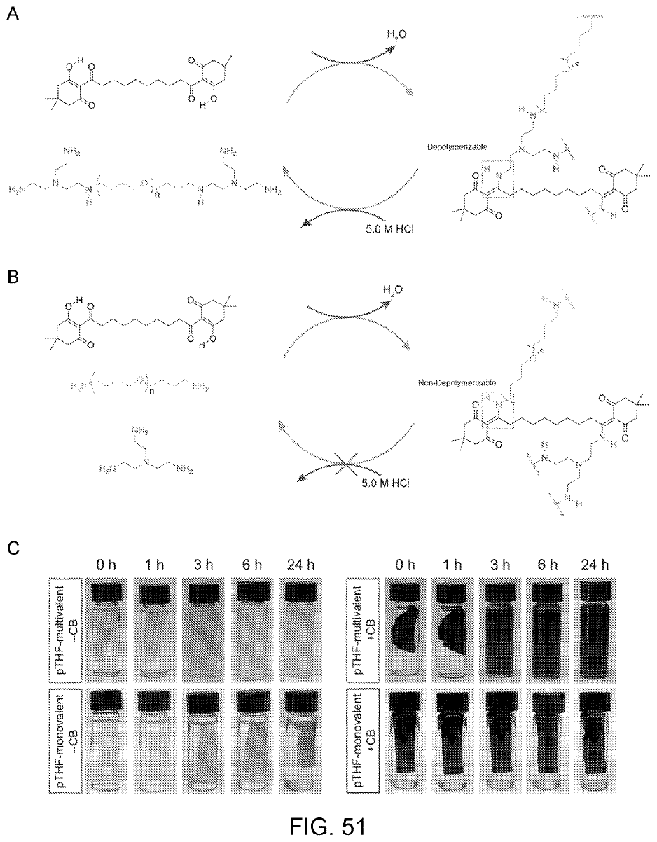

While dynamic covalent polymers are a promising platform for producing recyclable thermosets, bond exchange can lead to creep, which diminishes their use in applications that cannot tolerate material deformation under an applied load. Creep in both PDK-multivalent and PDK-monovalent elastomer networks was measured under 1 kPa stress and remarkably showed low creep for PDK-multivalent, with no sample reaching greater than 1% strain up to 150° C. (FIG. 50, Panel A). By contrast, PDK-monovalent flowed readily, due to high chain mobility, reaching >200% strain at 150° C. (FIG. 50, Panel B). The residual strain rate was calculated from a linear fit of the last 200 s of the strain vs time data; the strain rate was up to 2 orders of magnitude lower for PDK-multivalent elastomers than for PDK-monovalent elastomers, which reflects an increase in network viscosity. Adding carbon black to PDK-multivalent elastomers produced a small decrease in creep relative to unfilled materials (FIG. 51, Panel D), with all samples reaching a strain ≤0.7% up to 150° C. This behavior continued to stand out, even when carbon black was added to PDK-monovalent elastomers, which reduced creep by up to 13-fold relative to unfilled PDK-monovalent—although it was clear that continuous deformation could not be avoided at elevated temperature (FIG. 50, Panel E).

The residual strain rates for PDK-multivalent elastomers with and without carbon black were of comparable magnitude (FIG. 50, Panel F), and similar to the unfilled samples, PDK-multivalent elastomers had an approximately order of magnitude lower strain rate than PDK-monovalent elastomers when carbon black was added. Given that the modulus of PDK-multivalent elastomers had been observed to increase with the addition of carbon black, there is a clear effect in reducing chain mobility. However, carbon black had little effect on the rate of stress relaxation and the magnitude of creep in PDK-multivalent elastomers. Furthermore, PDK-multivalent elastomers with and without carbon black had little temperature-dependence on creep below 150° C. A plausible and non-limiting explanation is that the number of bonds that must simultaneously break and reform to allow long-range chain motion is sufficiently high in the unfilled material; carbon black simply increases the entropy penalty of deforming individual chain segments at the bound rubber layer, resulting in an increase in modulus. Introducing exchangeable bonds into linear segments in PDK-monovalent elastomers was further concluded to lead to a reduction in crosslink density at elevated temperature and production of undesirably high creep. From the earlier analysis, this reduction is due to reduced non-covalent entanglements enabled by bond exchange and network reorganization. Indeed, studies have demonstrated that increasing primary chain length in associative networks can reduce viscoelastic flow through maintaining molecular entanglements, and that reducing the number of exchangeable bonds in the linear segment is important to realizing this behavior.

Encouraged by the mechanical stability of pTHF-multivalent at high temperature, its thermal stability was further investigated to evaluate the potential for high service temperature applications without deleterious thermal degradation. <1% mass loss in pTHF-multivalent with or without carbon black was measured after 10,000 s at 150° C. by TGA, verifying excellent thermal stability at the highest temperature in the rheological experiments. The decomposition temperature at 50% mass loss for pTHF-multivalent was 419° C. without carbon black and 421° C. with carbon black, which was slightly higher but comparable to pTHF-monovalent (417 and 418° C. respectively), suggesting that the thermal stability arises from the network chemistry and not necessarily the crosslinking structure. To put these results in the context of thermal performance for conventional polymer formulations, the decomposition temperatures of pTHF-multivalent and pTHF-monovalent at 50% weight loss were compared with published data on polyurethanes that contained pTHF (Mₙ=2,000 g mol⁻¹) as a soft segment. It was assumed that 100% of the polyol content for the published formulations could be derived from biorenewable sources, and plotted decomposition temperature against the mass fraction of biorenewable content. The tested formulation shows a clear improvement in thermal stability with a relatively high biorenewable content, demonstrating the feasibility for deploying PDK-based elastomers in demanding environmental conditions.

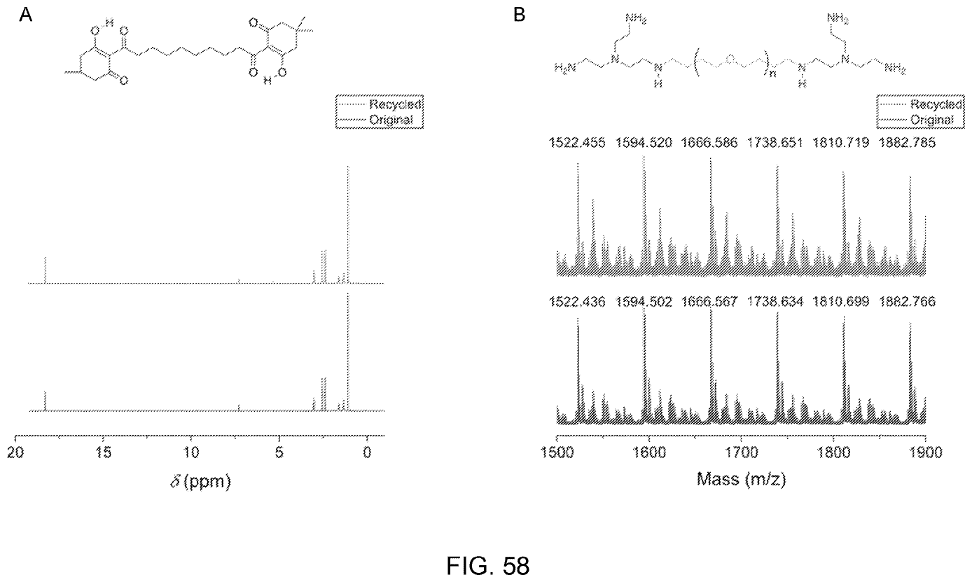

Having established a comprehensive understanding of how amine monomer valency in the PDK network affects the structure and dynamic properties in associated elastomers and carbon-reinforced rubbers, whether these architectural attributes were in any way influential on their deconstruction behavior were evaluated. It was hypothesized that the higher crosslinking density of PDK-multivalent elastomers might slow their deconstruction to monomer in strong acid. It was further hypothesized that achieving high materials efficiency in monomer recovery for PDK-monovalent elastomers might be compromised when the amine monomers are comprised of a mixture of compounds, in this case pTHF-diamine and TREN as the crosslinker. To evaluate the effects of PDK elastomer architecture on depolymerization rates, thermoformed samples were incubated in 5.0 M hydrochloric acid at ambient temperature for 24 h (FIG. 51, Panels A and B). Surprisingly (and invalidating these initial hypotheses), after only 6 h, PDK-multivalent samples both with or without carbon black had depolymerized to monomer, whereas PDK-monovalent samples swelled and softened, but remained intact after 24 h (FIG. 51, Panel C). TK-10 and pTHF-bis-TREN amine monomers were recovered from depolymerized PDK-multivalent elastomers in 90% yield for both components. Both were identical to pristine starting materials by NMR spectroscopy and MALDI-ToF mass spectrometry (FIG. 58). To understand the origins of this behavior, it was recognized that the end-group structure of pTHF diamine and TREN are inequivalent. Because TREN promotes facile PDK deconstruction, and does so expediently for PDK-multivalent elastomers when TREN end-caps pTHF-bis-TREN crosslinkers, it was hypothesized that the proximity of the tertiary amine to the diketoenamine bond may play an important role in acidolysis and therefore PDK depolymerization rates.

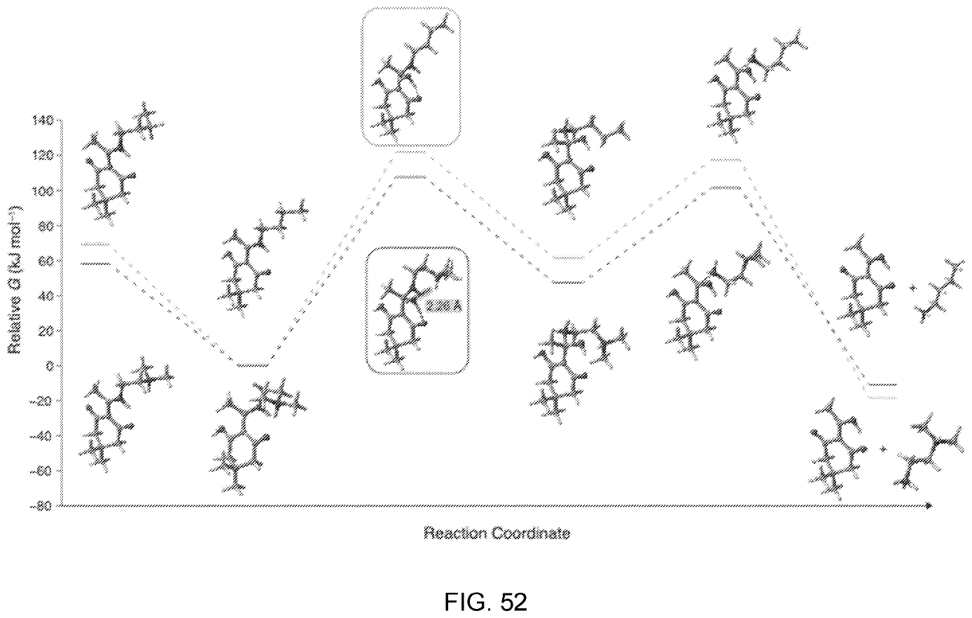

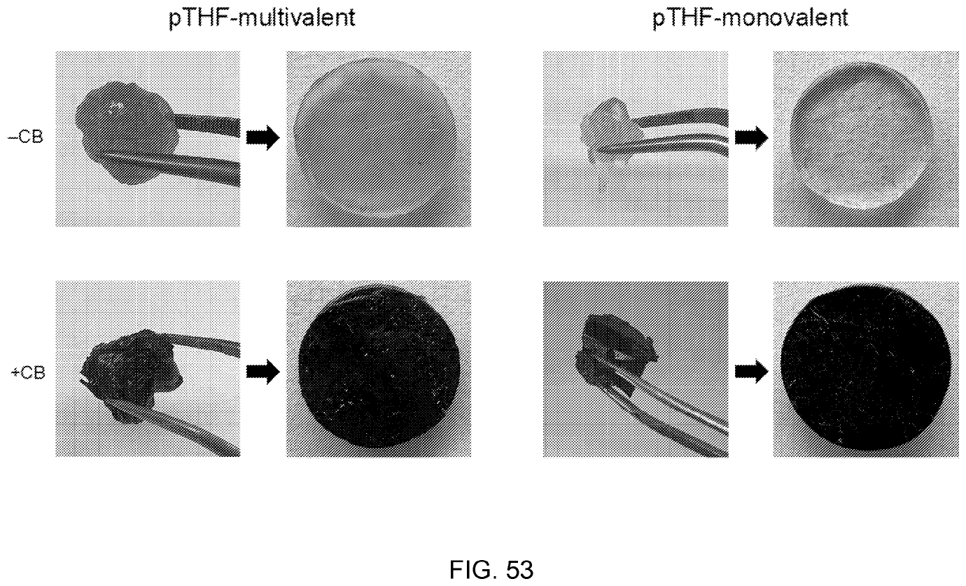

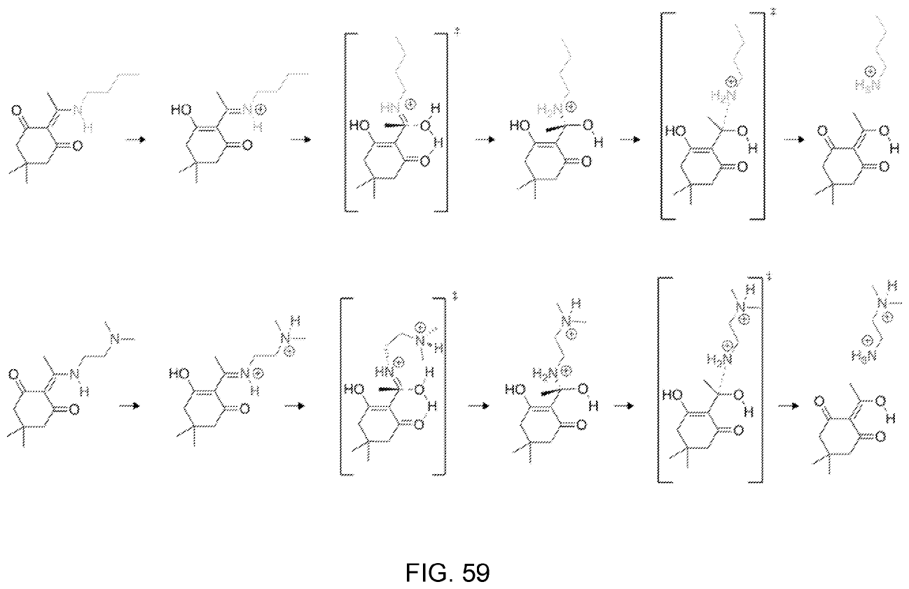

To test this revised hypothesis, a mechanistic understanding of how heteroatom proximity affects depolymerization energetics was sought. A computational simulation was carried out of acid-catalyzed diketoenamine hydrolysis using small-molecule surrogates for pTHF-bis-TREN and pTHF-diamine: specifically, diketoenamines featuring either a butyl group or an N,N-dimethylaminoethyl group (FIG. 52 and FIG. 59). The acidolysis of both diketoenamines is exergonic and is in fact more favorable for the pTHF-diamine surrogate than the pTHF-bis-TREN surrogate. However, the reaction kinetics can explain why pTHF-bis-TREN is depolymerizable while pTHF-diamine is not. In the rate-limiting step, water adds to a protonated iminium intermediate along the reaction coordinate. Here, it was found that the corresponding transition state for the butyl-functionalized diketoenamine has a standard free energy of activation approximately 13 kJ mol⁻¹ greater than that for the N,N-dimethylaminoethyl-functionalized diketoenamine. This difference in free energy of activation is due to the fact that the tertiary amine of the N,N-dimethylaminoethyl group stabilizes the transition state via a strong hydrogen bond (2.26 Å) with the incoming water, and thus decreases the free energy of activation for the N,N-dimethylaminoethyl-functionalized diketoenamine. Thus, the multivalent pTHF-bis-TREN end-group structure, in addition to providing for useful and advantaged PDK properties as elastomers and rubbers, is also essential for ensuring complete and rapid PDK depolymerization to triketone and amine monomers at ambient temperature in strong acid.

This example shows that PDK elastomers address ongoing challenges in the creation of highly recyclable yet mechanically stable rubbers that remain thermoformable, due to their dynamic covalent crosslinks. Elastomers prepared from commercially available pTHF-diamine soft segments contain exchangeable bonds that permit remolding, but their inability to undergo chemical recycling led to a discovery that heteroatom proximity to the diketoenamine bond can enable depolymerization to monomer. Covalently attaching TREN moieties to the soft segment restored the ability to undergo monomer-to-monomer chemical recycling, and also produced unexpected creep resistance that could not be achieved with the analogous PDK-monovalent formulation. This study advances fundamental insights into the macromolecular design of high-performance crosslinked elastomers that are amenable to circular manufacturing. This platform can be leveraged to broaden the range of properties exhibited by and to enable chemical recycling of more complex products featuring circular PDK elastomers alongside other materials.

Materials. Poly(tetrahydrofuran) (pTHF) (Mₙ≈2,000 g mol⁻¹), methanesulfonyl chloride (>99%), triethylamine (>99%), tris(2-aminoethyl)amine (TREN, 96%), 5,5-dimethyl-1,3-cyclohexanedione (dimedone, 95%), sebacic acid (99%), N,N′-dicyclohexylcarbodiimide (DCC, 99%), 4-(dimethylamino)pyridine (DMAP, >99%), sodium hydroxide (>97%), magnesium sulfate (anhydrous, >99.5%), Amberlyst A26 hydroxide form (basic) resin, and sodium trifluoroacetate (99%) were received from Sigma-Aldrich. Dichloromethane (>99.9%), chloroform (>99.8%), tetrahydrofuran (>99%), methanol (>99%) and concentrated hydrochloric acid were received from VWR. Carbon black (Super P conductive, >99%) was received from Alfa Aesar. Chloroform-d (99.8% D) was received from Cambridge Isotope Laboratories. Dithranol (99%) was received from MP Biomedicals. Poly(tetrahydrofuran) diamine (pTHF-diamine) was received from Huntsman Chemical.

Instrumentation. Nuclear Magnetic Resonance Spectroscopy (NMR). ¹H NMR spectra was recorded on a Bruker Avance II at 500 MHz. Chemical shifts are reported in δ (ppm) relative to CDCl₃ at 7.26 ppm.

Matrix-Assisted Laser Desorption/Ionization Time-of-Flight Mass Spectrometry (MALDI-ToF). MALDI mass spectra were recorded on a Bruker rapifleX spectrometer in positive reflector mode. A solution containing analyte (1 mg mL⁻¹) and dithranol (10 mg mL⁻¹) was prepared in THF, and 1 μL of this mixture was applied to a stainless-steel target plate and allowed to dry completely before analysis.

Fourier-Transform Infrared Spectroscopy (FT-IR). FT-IR spectra were recorded on a Thermo-Fisher Nicolet iS50 spectrometer in Attenuated Total Reflectance (ATR) mode.

Rheological Analysis. Amplitude sweep, frequency sweep, stress relaxation, and creep measurements were performed on a TA DHR-2 rheometer. Elastomer samples were cut into 8 mm discs with a biopsy punch and loaded onto a rheometer between 8-mm stainless steel parallel plates.

Differential Scanning Calorimetry (DSC). DSC measurements were performed on a TA Q200 from −80 to 100° C. with a temperature ramp of 10° C. min⁻¹. Data is reported for the second heating cycle for each sample.

Thermogravimetric Analysis (TGA). TGA measurements were performed on a TA 5500 from 150 to 800° C. with a 120 min isothermal hold at 150° C. and a 10° C. min⁻¹ temperature ramp. Isothermal measurements were performed at 150° C. for 10,000 s. All measurements were performed under nitrogen atmosphere.

Tensile testing. Tensile measurements were performed on an Instron 68TM-5 with 1 kN load cell at ambient temperature. Dog bone samples were prepared with width 4.5 mm, thickness 1 mm, and gauge length 25 mm. Samples were strained to failure at a tensile rate of 50 mm min⁻¹.

Methods. Synthesis of Ditopic Triketone Monomer, TK-10.

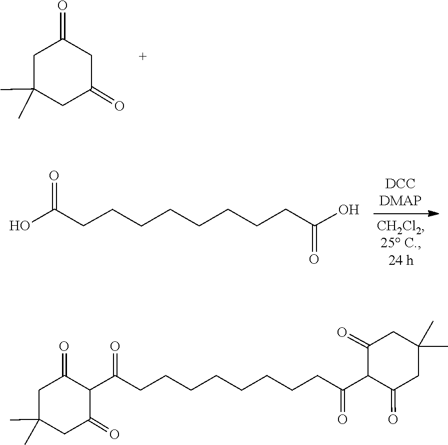

Briefly, the ditopic triketone monomer, TK-10, was synthesized as follows: a round bottom flask was charged with dimedone (20.0 g, 0.143 mol), DMAP (24.9 g, 0.204 mol), and dichloromethane (250 mL). A solution of DCC (33.7 g, 0.163 mol) in dichloromethane (80 mL) was added dropwise at room temperature, and the reaction was allowed to proceed for 18 h. The resulting solution was filtered to remove dicyclohexylurea, and the organic phase was washed three times with 10% HCl. The product was concentrated under reduced pressure to obtain a yellow-orange solid. The crude product was dissolved in 1.0 M NaOH, washed three times with dichloromethane, and acidified with 1.0 M HCl to precipitate an off-white solid. The product was collected by filtration and dried under reduced pressure.
Synthesis of pTHF-bis-mesylate.

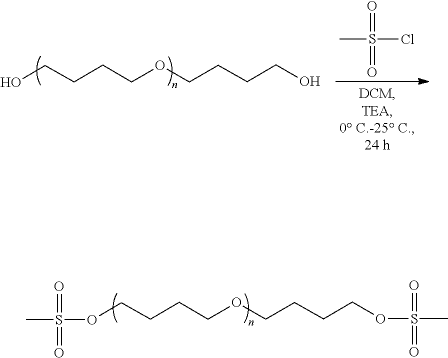

pTHF (30 g, 0.015 mol) was dissolved in dichloromethane (600 mL) in a round bottom flask with stirring. Triethylamine (10.6 g, 0.105 mol) was added, and the flask was transferred to an ice bath. Methanesulfonyl chloride (6.01 g, 0.053 mol) was dissolved in dichloromethane (20 mL) and added dropwise under nitrogen atmosphere. The reaction was stirred at 0° C. for 1 h, after which the ice bath was removed, and the reaction was stirred for 18 h at room temperature. The reaction mixture was concentrated to 200 mL DCM, combined with DI water (100 mL) and stirred for 30 min at room temperature. The solution was transferred to a separatory funnel and the organic phase was washed 3× with DI water. The organic phase was then dried over magnesium sulfate and the solvent was removed under reduced pressure to yield a waxy orange solid (25.6 g, 79%). ¹H NMR (500 MHz, CDCl₃, 25° C., TMS): δ 4.25 (t, J=6.51 Hz, 4H; CH₂—CH₂—O—SO₂—CH₃), 3.49-3.33 (m, 142H; (CH₂—CH₂—O)ₙ), 2.99 (s, 6H; CH₃), 1.70-1.52 (m, 143H; (CH₂—CH₂—O)ₙ).

Synthesis of pTHF-bis-TREN.

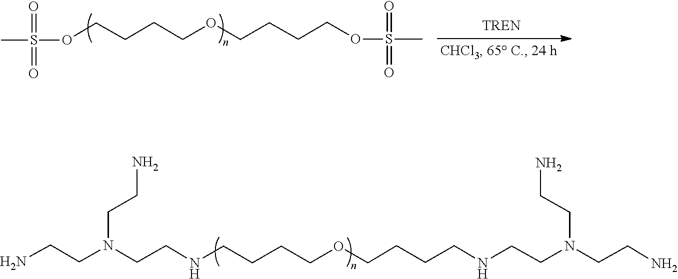

pTHF-bis-mesylate (20 g, 0.01 mol) was dissolved in chloroform (120 mL). A round bottom flask was charged with TREN (35.2 g, 0.24 mol) and heated to 65° C. in an oil bath. The pTHF-bis-mesylate solution was added dropwise at 0.2 mL min⁻¹ and stirred for an additional 12 h at 65° C. The crude reaction mixture was diluted with dichloromethane and filtered to remove TREN-sulfonate salts, and the soluble fraction was stirred for 18 h with Amberlyst A26 OH ion exchange resin. The resin was removed by filtration and the organic solvents were subsequently removed under reduced pressure to yield a heterogeneous yellow mixture. DI water (200 mL) was added and the mixture was divided into 50-mL centrifuge tubes and centrifuged at 10,000 rpm for 5 min. The recovered white solids were washed 2× with DI water and centrifuged after each wash step. The solids were collected in methanol and dried under reduced pressure to yield a waxy white solid (9.3 g, 47%). (500 MHz, CDCl₃, 25° C., TMS): δ 3.50-3.30 (s, 102H; (CH₂—CH₂—O)ₙ), 2.80-2.48 (m, 24H; N—CH₂—CH₂—NH₂), 1.76-1.47; (s, 115H; (CH₂—CH₂—O)ₙ).

Synthesis of PDK-multivalent Elastomers. In atypical synthesis, pTHF-bis-TREN (1.0 g, 0.50 mmol) was dissolved in THF (1.0 mL) in a glass vial and heated to 60° C. TK-10 (0.31 g, 0.69 mmol) was separately dissolved in THF (0.31 mL) in a glass vial and heated to 60° C. The TK-10 solution was rapidly added to the pTHF-bis-TREN solution and the mixture was stirred with a metal spatula. After approximately 30 s, a solid gel was obtained. The heat was increased to 75° C. and the gel was dried under vacuum to remove residual THF and water generated from the diketoenamine condensation. For samples containing carbon black, a solution of 0.5% w/v carbon black in THF was prepared by sonication, and combined with pTHF-bis-TREN and TK-10 as described. Elastomer samples were pressed in Teflon molds using a Stahls' Hotronix heat press at 150° C. and 60 psi for 5 min.

Synthesis of PDK-monovalent Elastomers. pTHF-diamine (4.0 g, 2.35 mmol) and TK-10 (1.25 g, 2.80 mmol) were combined in a glass vial and heated to 110° C. with stirring for 30 min until the mixture became homogeneous and evolution of bubbles ceased. The melt was cooled to 60° C., and TREN (0.1 g, 0.68 mmol) was added rapidly. The mixture was stirred with a metal spatula to obtain a viscous paste. The mixture was dried under vacuum at 70° C. to remove residual water. For samples containing carbon black, a solution of 0.5% w/v carbon black in THF was prepared by sonication, and combined with pTHF-diamine and TK-10 as described. Elastomer samples were pressed in Teflon molds using a Stahls' Hotronix heat press at 150° C. and 60 psi for 5 min.

Depolymerization of PDK elastomers. Elastomer samples were incubated in a solution of 5.0 M HCl at room temperature with stirring. Samples were centrifuged to pellet the precipitated TK-10, and the supernatant containing dissolved pTHF-bis-TREN·HCl was reserved. Solid TK-10 was washed with DI water and dried under vacuum. The aqueous solution containing pTHF-bis-TREN·HCl was stirred with Amberlyst A26 OH resin, and water was removed by distillation to yield pTHF-bis-TREN as the free base.

Computational Methods. All hybrid-DFT calculations were performed using Gaussian16. Input files were prepared and output files were parsed using Pymatgen. All free energies were calculated at the M062-X/6-311+G(d,p)//SMD level of theory using the Quasi-Rigid Rotor Harmonic Oscillator (Quasi-RRHO) method for calculating the vibrational entropy. To find the lowest-energy conformers contributing to the free energy of activation, a conformer search was performed on the ground state and transition state structures. For transition states, the optimization at the B97D level of theory was done with a fixed explicit water molecule involved in the transition state. Uniqueness was defined as structures with electronic energies greater than 0.1 kJ mol⁻¹ apart and RMSDs greater than 0.1 Å apart.

Note that in the conformer search for the ground state of the the N,N-dimethylaminoethyl-functionalized diketoenamine, the lowest-energy conformer does not match the configuration of the butyl-functionalized diketoenamine—the iminium does not form a hydrogen bond with the ketone. However, that conformation is similar in free energy and its use would not change the qualitative trend that the N,N-dimethylaminoethyl-functionalized diketoenamine has a significantly (>10 kJ mol⁻¹) lower free energy of activation than the butyl-functionalized diketoenamine.

For the diketoenamines featuring a butyl group, all other structures along the reaction coordinate were found by geometry optimization following substitution of the N,N-dimethylaminoethyl group (e.g., without an additional conformer search).

### Figure Captions for Example 25.

FIG. 47. Examples of commercial products that incorporate crosslinked elastomeric components that are challenging to recover and recycle (Panel A). Schematics of monomer and corresponding polymer network structure for PDK-multivalent and PDK-monovalent (Panel B). Monomer structures for multivalent soft segment: poly(tetrahydrofuran)-bis-tris-2(aminoethyl)amine (pTHF-bis-TREN); triketone: 2,2′-decanedioylbis(5,5-dimethylcyclohexane-1,3-dione) (TK-10); monovalent soft segment: poly(tetrahydrofuran)-diamine (pTHF-diamine); TREN: tris-2(aminoethyl)amine (Panel C). Stress-strain plots for PDK-multivalent (strain at break=104%, tensile strength at break=1.14 MPa, toughness=78.5 MJ m⁻³) and PDK-monovalent (strain at break=268%, tensile strength at break=0.257 MPa, toughness=49.9 MJ m⁻³) (Panel D).

FIG. 48. Frequency sweep (Panel A), amplitude sweep (Panel B), and stress relaxation measurements (Panel C) for PDK-multivalent elastomers. Frequency sweep (Panel D), amplitude sweep (Panel E), and stress relaxation measurements (Panel E) for PDK-monovalent elastomers.

FIG. 49. Frequency sweep (Panel A), amplitude sweep (Panel B), and stress relaxation measurements (Panel C) for PDK-multivalent containing 0.5 wt % carbon black. Frequency sweep (Panel D), amplitude sweep (Panel E), and stress relaxation measurements (Panel F) for PDK-monovalent containing 0.5 wt % carbon black.

FIG. 50. PDK-multivalent elastomer creep, showing exceptional creep resistance at all temperatures (Panel A). PDK-monovalent elastomer creep, showing high susceptibility to creep at all temperatures (Panel B). Strain rate (dγ/dt) vs temperature for PDK-multivalent and PDK-monovalent elastomers (Panel C). PDK-multivalent carbon-reinforced (0.5 wt %) rubber creep, showing exceptional creep resistance at all temperatures (Panel D). PDK-monovalent carbon-reinforced (0.5 wt %) creep, showing improved creep resistance at all temperatures (Panel E). Strain rate (dγ/dt) vs temperature for PDK-multivalent and PDK-monovalent carbon-reinforced (0.5 wt %) elastomers (Panel F). Strain rate was calculated from the last 200 s of the strain vs. time data.

FIG. 51 TK-10 and pTHF-bis-TREN form crosslinked elastomers through a condensation polymerization, and are depolymerized back to starting materials in the presence of aqueous HCl (Panel A). TK-10, pTHF-diamine, and TREN form crosslinked elastomers through a similar mechanism, but the diketoenamine bond formed between TK-10 and pTHF-diamine is non-depolymerizable in aqueous HCl (Panel B). Chemical depolymerization of PDK-multivalent and PDK-monovalent elastomers with and without carbon black (Panel C). Depolymerization experiments were performed in 5.0 M HCl at ambient temperature.

FIG. 52. Computational reaction coordinates for acid-catalyzed diketoenamine hydrolysis. Calculations were performed on small-molecule analogues of PDK-multivalent or PDK-monovalent using acyl dimedone and n-butylamine (top series in green) or N,N-dimethylaminoethylamine (bottom series in purple).

FIG. 53. Photographs of elastomer samples before and after reprocessing in a circular Teflon mold at 150° C. and 60 psi for 300 s.

FIG. 54. DSC traces of PDK-multivalent elastomers: without carbon black (Panel A), and with 0.5 wt % carbon black (Panel B).

FIG. 55. DSC traces of PDK-monovalent elastomers: without carbon black (Panel A), and with 0.5 wt % carbon black (Panel B).

FIG. 56. ATR-FTIR spectra of PDK-multivalent elastomers: without carbon black (Panel A), and with 0.5 wt % carbon black (Panel B).

FIG. 57. ATR-FTIR spectra of PDK-monovalent elastomers: without carbon black (Panel A), and with 0.5 wt % carbon black (Panel B).

FIG. 58 ¹H NMR spectra of recycled and pristine TK-10 (Panel A). MALDI mass spectra of recycled and pristine pTHF-bis-TREN (Panel B).

FIG. 59. Chemical structures of small-molecule analogues of PDK-multivalent or PDK-monovalent using acyl dimedone and n-butylamine (top series) or N,N-dimethylaminoethylamine (bottom series).

### Example 26—Variable Amine Spacing Determines Depolymerization Rate in Polydiketoenamines

The design of circular polymers has emerged as a necessity due to the lack of efficient recycling methods for many commodity plastics, particularly those used in durable products. Among the promising circular polymers, polydiketoenamines (PDKs) stand out for their ability to undergo highly selective depolymerization in strong acid, allowing monomers to be recovered from additives and fillers. Varying the triketone monomer in PDK variants is known to strongly affect the depolymerization rate; however, it remains unclear how the chemistry of the crosslinker, far from the reaction center, affects the depolymerization rate. Notably, a proximal amine in the crosslinker was found to dramatically accelerate PDK depolymerization when compared to crosslinkers obviating this functionality. Moreover, the spacing between this amine and the diketoenamine bond offers a previously unexplored opportunity to tune PDK depolymerization rates. In this way, the molecular basis for PDK circularity is revealed and further suggests new targets for the amine monomer design to diversify PDK properties, while ensuring circularity in chemical recycling.

Despite increasing attention paid to reducing plastic waste, plastics continue to accumulate in the environment and landfills, causing ecological harm and wasting non-renewable resources. Solving this problem is a multi-faceted issue, but one critical strategy towards a future with minimal waste is fundamentally a materials challenge: can we synthesize circular plastics—plastics that can be infinitely recycled back to their constituent monomers—that are recycled through green processes and provide significant value to society? Recent discoveries in circular polymers have demonstrated rapid depolymerization under mild conditions, e.g., through acidolysis, solvolysis, or catalytic ring-closing, showing a great potential to replace hard-to-recycle plastics. A new class of circular polymers, polydiketoenamines (PDKs), provides a promising step toward that goal. PDKs can be recycled back to monomers with high yield at room temperature in strong aqueous acid but remain stable in neutral, basic, and mildly acidic conditions. This controlled recycling is enabled through the presence of the hydrolyzable diketoenamine moiety in the polymer repeat unit. As long as the diketoenamine moiety is maintained, the chemistry of PDKs can in theory be tailored to access specific properties while maintaining their recyclability. For example, heteroatom substitutions on the triketone monomer enable circularity in mixed-plastic recycling by differentiating the hydrolysis rate of the diketoenamine. However, to access a wide range of properties it is often necessary to alter the chemistry of the crosslinker. To further expand the scope of properties that PDKs can access while maintaining circularity in chemical recycling, we can understand how the crosslinker affects the kinetics of the hydrolysis reaction.

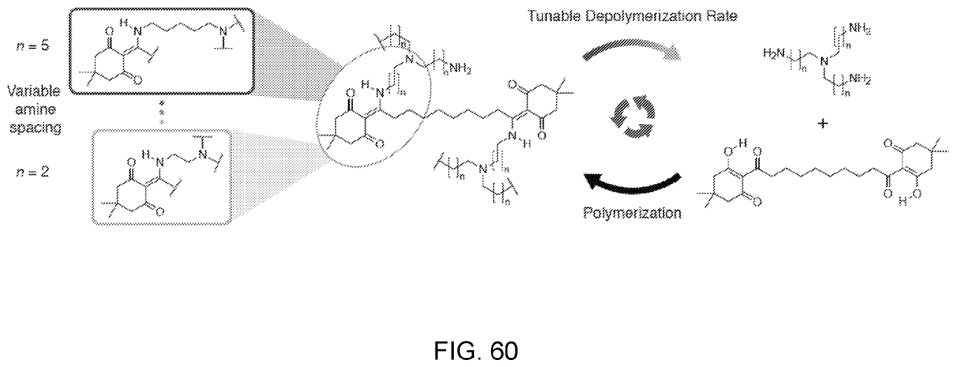

This example describes varying the amine spacing, e.g., the carbon spacing between the secondary and tertiary amine in a triamine crosslinker, to understand the role that the tertiary amine plays in the hydrolysis reaction (FIG. 60). Computational modeling and experiment are combined to gain mechanistic insight into the role of amine spacing in diketoenamine hydrolysis and how that translates to PDK depolymerization. This combined approach also allows for demonstrating that it is valuable to use Multi-Path Transition State Theory (MP-TST) to accurately connect simulations of diketoenamines with experiments, due to the large conformational freedom and strong non-covalent bonds in diketoenamines. Multi-path formulations of transition state theory have been shown to yield highly accurate rate constants for hydrogen shift reactions, especially when coupled with calculations of proton tunneling rates, and OH-reactions. This example demonstrates that MP-TST based on an ensemble of low-energy conformers yields highly accurate rate constants for diketoenamine hydrolysis when compared to experiment. Furthermore, analyzing the pathways that contribute to the reaction, the observed differences in reaction rate are found to be related to a highly variable stabilization of the transition state, which depends on an intramolecular hydrogen bond that forms during the addition of water. This example further indicates that a crosslinker heteroatom that is capable of forming a hydrogen bond while simultaneously minimizing strain energy is valuable for low-energy depolymerization of PDK resins. Indeed, this example highlights that stabilization due to intramolecular hydrogen bonds in a transition state can drastically change the rate of a reaction.

PDKs can be designed using the techniques described herein to access diverse properties while maintaining recyclability through low-temperature hydrolysis.

Results and discussion. First, hydrolysis rates of diketoenamines with varying amine spacing are discussed and those results are compared to measurements of hydrolysis kinetics of as-synthesized diketoenamines. Then, the calculated reaction pathways are analyzed to understand the chemical origins of the observed differences in rate constant and the advantages of using the MP-TST formalism are investigated for achieving high accuracy in comparison to experiment. Finally, two PDK materials are synthesized with varying amine spacing to verify that the small molecule study translates to polymer systems.

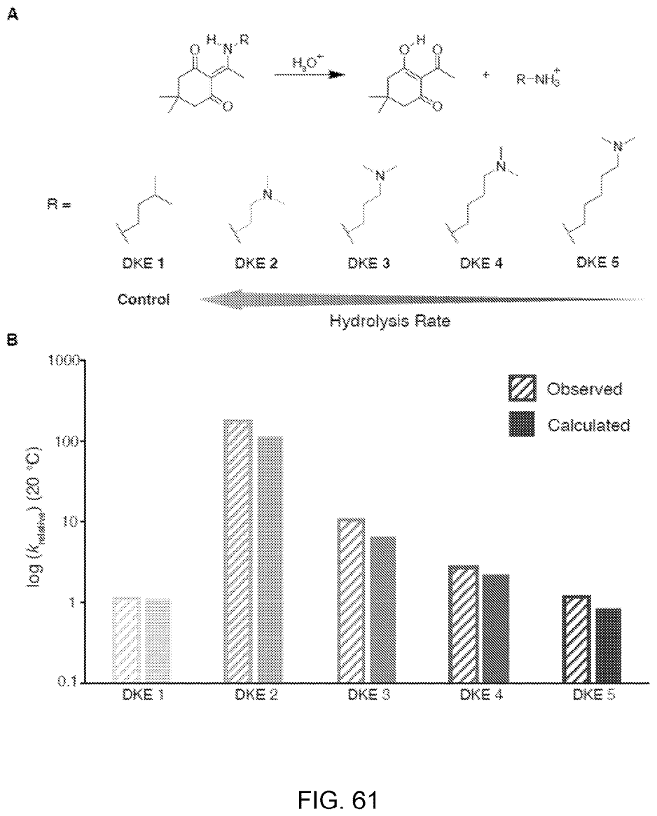

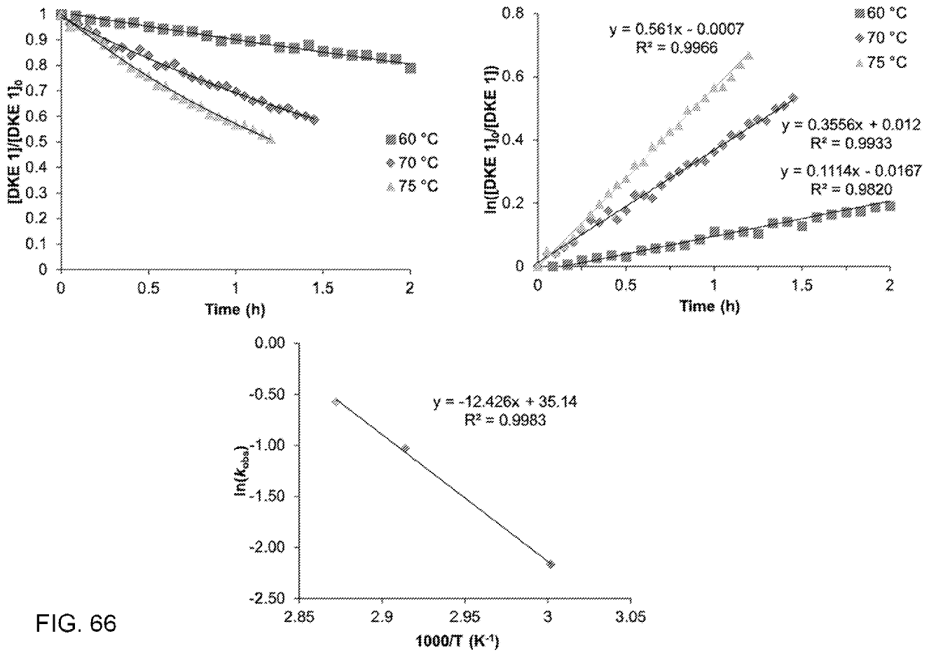

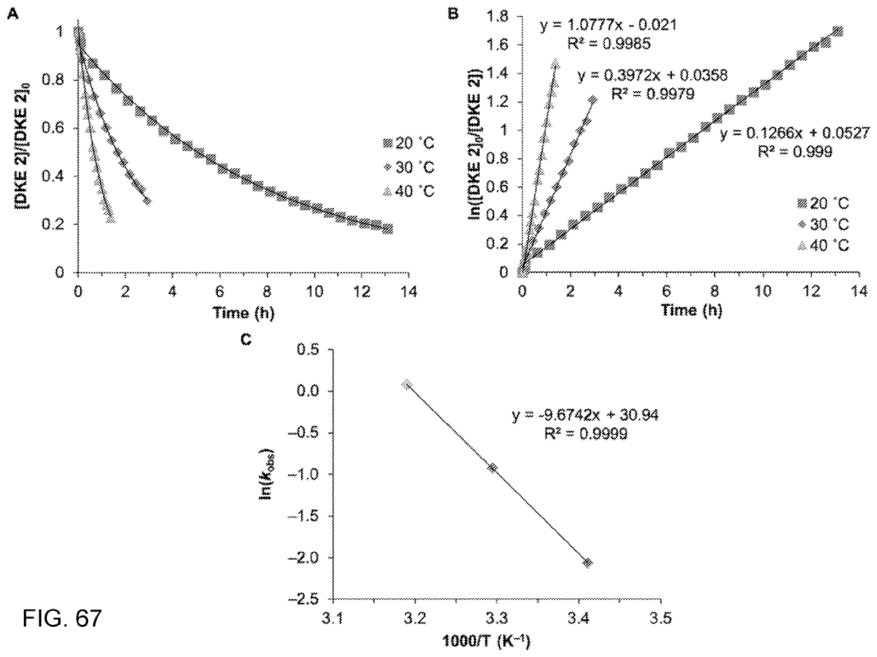

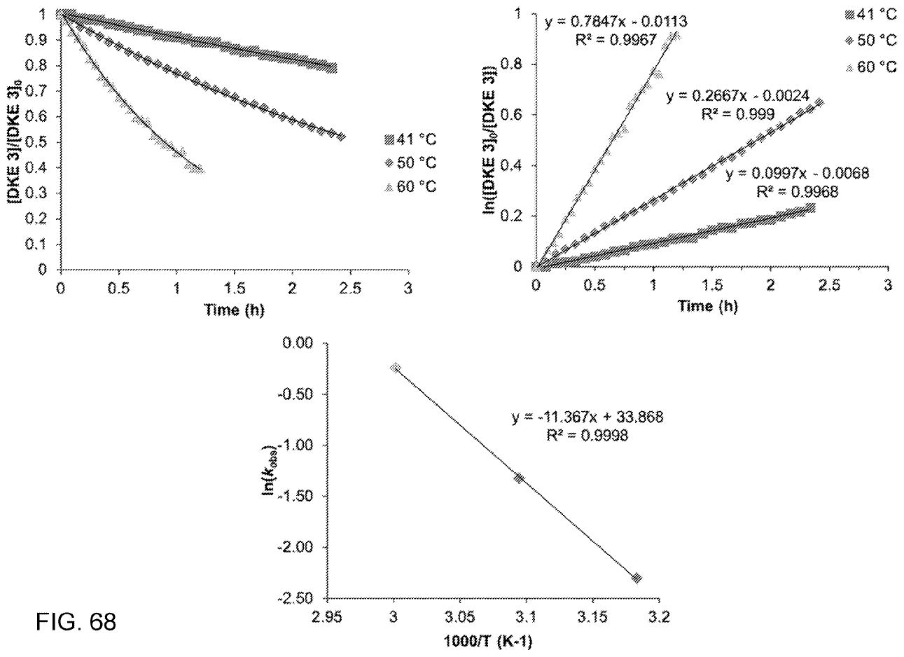

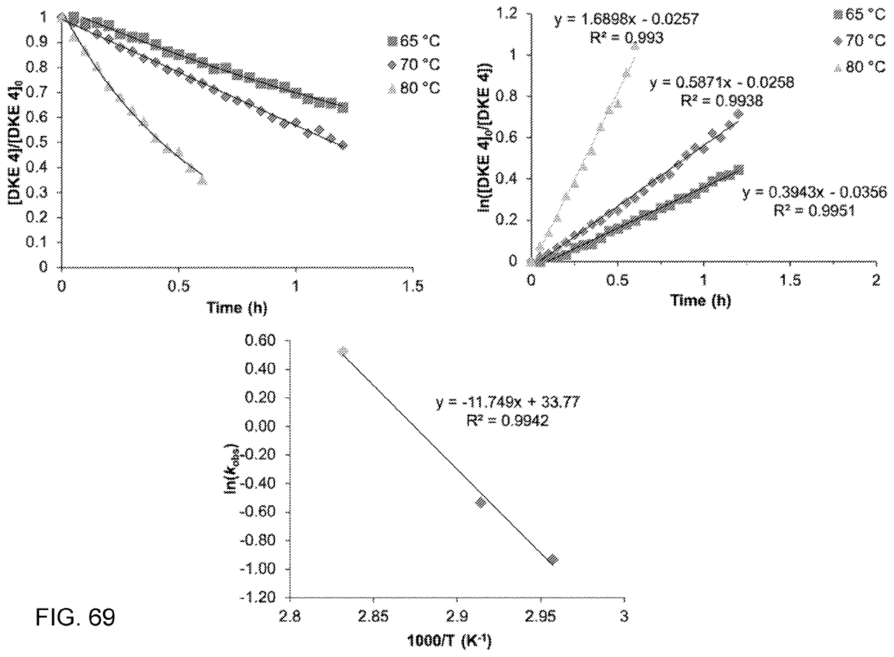

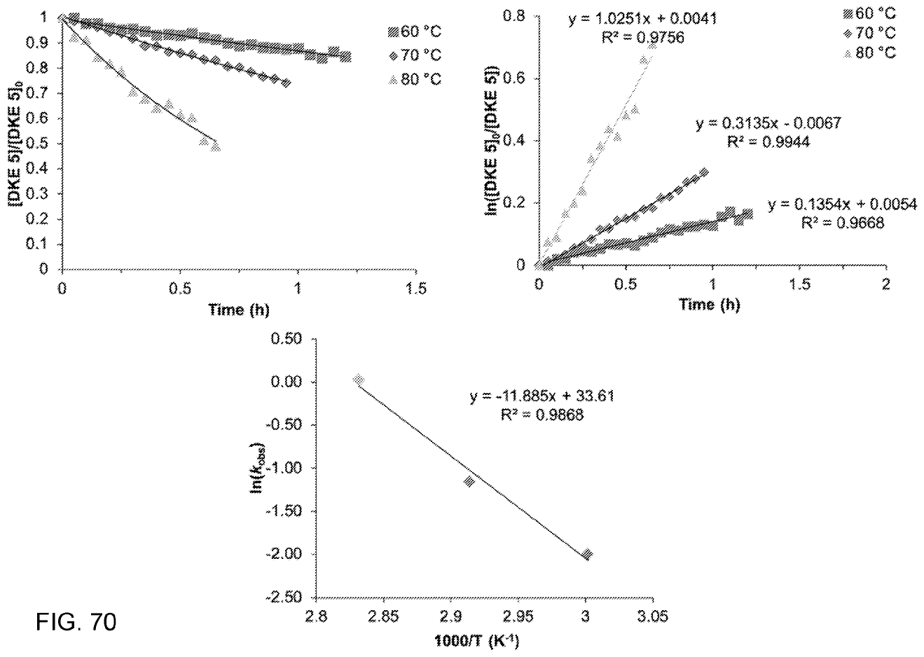

Diketoenamine hydrolysis rate. Five variations of the diketoenamine (DKE) chemistry were examined: DKE 1, which serves as the control with no tertiary amine, and DKEs 2, 3, 4, and 5, which have increasing carbon spacing from 2 to 5 carbons between the two nitrogen atoms (FIG. 61, Panel A). To calculate the hydrolysis rate constant, the addition of water in the two step addition-elimination hydrolysis reaction was a focus, as previous mechanistic studies of DKE 1 hydrolysis show the addition of H₂O to be rate-limiting. It was found that the MP-TST calculated hydrolysis rate constant for DKE 2 is 113 times larger than the control, DKE 1. The rate then dramatically decreases for DKE 3, to just 6 times greater than DKE 1, and then continues to decrease until DKE 5 shows no increase in rate compared to DKE 1 (Table 1). The experimentally observed rate constants closely follow this trend (FIG. 61, Panel B). These observed rate constants were determined via ¹H NMR kinetics at different temperatures (FIG. 66, FIG. 67, FIG. 68, FIG. 69, and FIG. 70). The only significant discrepancy between simulations and experimental observations is the relative rate constant of DKE 5. Experimentally, DKE 5 hydrolyzes slightly faster than DKE 1, at kᵣₑₗₐₜᵢᵥₑ=1.5, whereas the calculations predict that DKE 5 hydrolyzes slower than DKE 1, at kᵣₑₗₐₜᵢᵥₑ 0.76. While this result for DKE 5 leads to a qualitative error in the trend, the absolute difference is small and still indicates that the molecules hydrolyze at similar rates. In addition, values for the methods that are considered to be the useful for a system of this size are evaluated, but the quantitative rate constant calculation is determined to be sensitive to the method of approximating the vibrational entropy and the hybrid-DFT level of theory (Tables 2 and 3). Regardless, these data show a substantial difference in the hydrolysis rate for structures that only differ by substitutions far from the reaction center.

TABLE 1
MP-TST calculated and experimental relative
rate constants of diketoenamine hydrolysis.

| Molecule | Calculated kᵣₑₗₐₜᵢᵥₑ | Observed kᵣₑₗₐₜᵢᵥₑ |
|---|---|---|
| DKE 1 | 1.0 | 1.0 |
| DKE 2 | 112.7 | 178.9 |
| DKE 3 | 6.3 | 10.4 |
| DKE 4 | 2.1 | 2.6 |
| DKE 5 | 0.76 | 1.4 |

To further understand the rapid decrease in reaction rate with amine spacing, the dominant reaction pathways contributing to the calculated MP-TST rate are analyzed for each DKE. All DKEs with tertiary amines display similar features in the reactant and transition state. First, the most stable states of the reactant exhibit a planar conformation due to a six-membered hydrogen-bonded ring bearing the exchangeable proton and a slightly nonplanar conformation with both amines coordinating to the ketone. During the addition of water, the hydrogen bonds break to reach a tetrahedral intermediate where the tertiary amine coordinates with the incoming water to lower the energy of the transition state.

The variation in reaction rate can be qualitatively understood by analyzing the energetics of the single dominant pathway for each DKE. In all cases, the free energy barrier has significant enthalpic and entropic components at room temperature. However, the entropic contribution is similar for all 5 DKEs and does not trend with the overall free energy barrier, while the enthalpic contributions differ significantly and mirror the trend in the overall barrier (Table 4). Further analyzing the enthalpy barrier, it is seen that the variation in hydrolysis rate arises from an energetic balance in the transition state between the stabilization due to the formation of an intramolecular hydrogen bond and the strain due to reaching that conformation. This trend can be quantified by examining the distortion and interaction contributions to the energy barrier:

$$Δ {E}_{Total}^{‡} = Δ {E}_{Interaction}^{‡} + Δ {E}_{Distortion}^{‡} . \qquad ( 1 )$$

This energy decomposition separates

$$Δ {E}_{Interaction}^{‡} ,$$

the change in energy due to the mixing of electronic states in the two molecules, from

$$Δ {E}_{Distortion}^{‡} ,$$

the change in energy due to straining the geometries of each molecule individually. Analyzing the dominant mechanism for each DKE, there is observed a monotonic trend in the energy barrier that is consistent with the observed trend in hydrolysis rate and is reproduced in

$$Δ {E}_{Total}^{‡} and Δ {E}_{Distortion}^{‡}$$

but not

$$Δ {E}_{Interaction}^{‡}$$

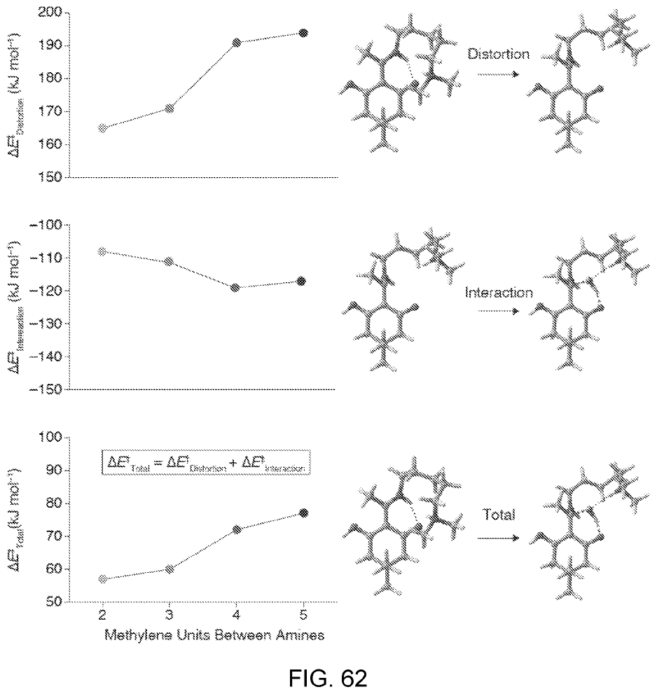

(FIG. 62). While larger amine spacings trend with increased stabilization of the transition state, the unfavorable distortion of a longer chain into the low-energy transition state ultimately dominates the energy barrier, leading to a steady increase in the kinetic barrier.

Origins of the MP-TST rate. While an energy decomposition based on a single pathway is useful for understanding the mechanistic origins of the decrease in rate with increasing amine spacing, the high accuracy of the rate predictions in comparison to experiment shown in FIG. 61 employ a method that takes into account the numerous reaction pathways available to this system. With two to five carbons between amines, there are up to seven rotatable bonds in a DKE, and the ketone, enol, iminium, tertiary amine, and water are all available to participate in hydrogen bonds. A standard approach to computational studies of the reaction rate for a bimolecular reaction with organic molecules of medium size and flexibility is to perform a conformer search of the transition state and calculate the energy barrier, ΔE^{‡}, as the difference in energy between the lowest-energy transition state and lowest-energy reactant, assuming facile interconversion among reactant conformers. Single-structure transition state theory (SS-TST) can then be used to obtain a reaction rate constant,

$${k}_{SS - TST} = κ \frac{{k}_{B} T}{h} \frac{{Q}_{TS}}{{Q}_{R}} {e}^{- \frac{Δ {E}^{‡}}{{k}_{B} T}} , \qquad ( 2 )$$

where Q_{TS} and Q_{R} are the partition functions of the transition state and reactants, k_{B} is the Boltzmann constant, h is the Planck constant, T is the temperature, and x is the tunneling coefficient.

To accurately represent systems with large conformational freedom, MP-TST allows many-to-many relationships between reactant and product phase space. Full MP-TST requires the identification of all distinct conformers of the transition state, and the SS-TST rate constant can be modified to be

$${k}_{MP - TST} = \frac{{k}_{B} T}{h} \frac{{\sum }_{i}^{TS conf .} κ {e}^{\frac{- Δ {E}_{j}}{{k}_{b} T}} {Q}_{TS , i}}{{\sum }_{j}^{R conf .} {e}^{\frac{- Δ {E}_{i}}{{k}_{b} T}} {Q}_{R , j}} {e}^{- \frac{{E}_{TS , 0} - {E}_{R , 0}}{{k}_{B} T}} . \qquad ( 3 )$$

Here, the single partition functions in equation 2 have been replaced by sums over all the partition functions of all conformers weighted by their relative energy to the lowest-energy conformer, and the energy barrier in the exponential term is the difference in the zero-point corrected electronic energy between the lowest-energy transition state conformer, E_{TS,0}, and the lowest-energy reactant conformer E_{R,0}. While MP-TST is formally true only for a sum over all conformers of the system, it has been shown that accurate rate constants can be calculated by only including the low-lying conformers. This approximation is important when considering systems as large as those studied here, where calculating the partition function of every conformer at a high level of electronic structure theory is currently not computationally feasible.

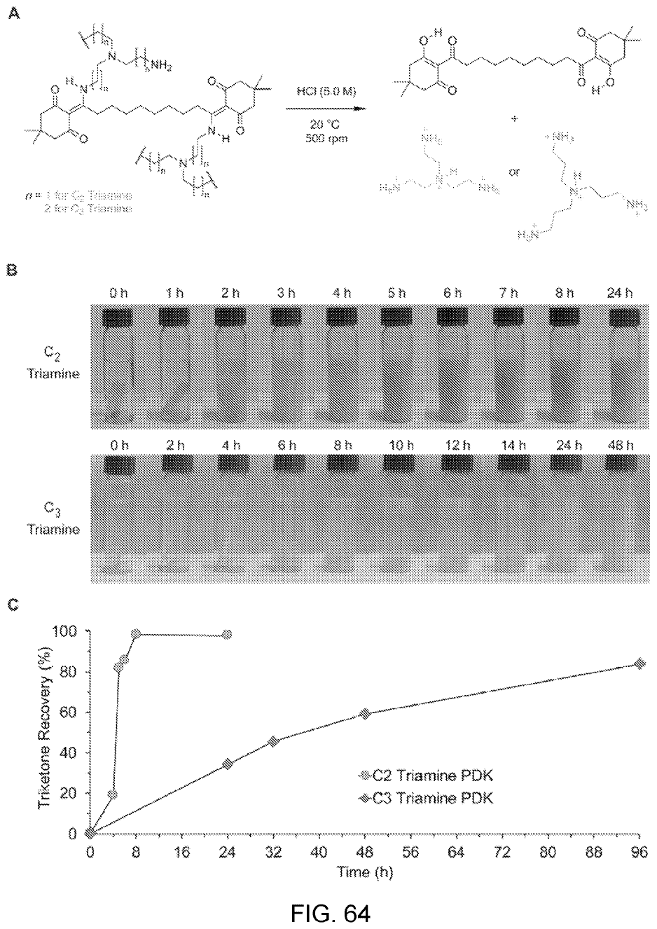

The impact of a multi-path model is perhaps best understood by examining the free energy barrier for the reaction. By analyzing the distribution of pathways, it is found that all DKEs contain reaction coordinates with a range of free energy barriers, but that tertiary amine-containing DKEs exhibit specific conformations with significant contributions, whereas DKE 1 has a roughly equal contribution from many pathways (FIG. 64, Panel A). Extracting one free energy barrier from that data is therefore not an obvious task. An energetic span approach,

$$Δ {G}^{‡} = {G}_{TS , 0} - {G}_{R , 0} , \qquad ( 4 )$$

shows a close match with the trends in

$$Δ {G}_{Observed}^{‡}$$

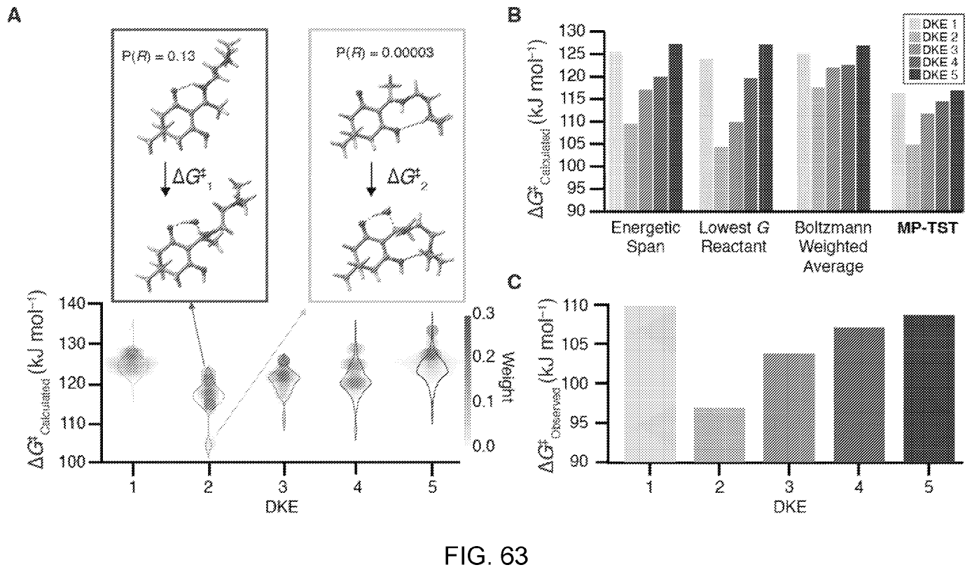

derived from an Eyring analysis of experimental rates (FIG. 63, Panels B and C). However, the energetic span approach also overestimates the relative ΔG^{‡} for DKE 5 compared to DKEs 2-4, and to a lesser extent the control, DKE 1. As the choice of hybrid-DFT level of theory can systematically impact the absolute free energy barriers, more importance is placed on the proper relative ordering of ΔG^{‡}. However, when comparing DKE 1 to DKE 5, the observed difference in ΔG^{‡} that is aimed to capture, +1.1 kJ mol⁻¹, is within the accuracy of the ωB97X-V functional for hydrolysis barrier heights: a mean absolute error (MAE) of 2.89 kJ mol⁻¹ when compared to highly accurate wavefunction methods. Still, one may expect the relative ΔG^{‡} of similar molecules to be more accurate than the MAE over a large dataset, and the error in ΔΔG^{‡} for DKE 1 and DKE 5 from the energetic span approach is 2.8 kJ mol⁻¹. Considering that errors are exponentiated when calculating the relative rate constant, higher accuracy is needed. Other reasonable approaches to calculating ΔG^{‡} encounter similar discrepancies. For example, one can calculate the free energy barrier of the reaction coordinate connecting the lowest free energy reactant with its corresponding transition state,

$$Δ {G}^{‡} = {G}_{TS , {R}^{*}} - {G}_{{R}^{*}} . \qquad ( 5 )$$

Or, one can use a Boltzmann weighted average,

$$Δ {G}^{‡} = {\sum }_{n = 1}^{∞} {e}^{- \frac{Δ {G}_{j}}{{k}_{B} T}} ( {G}_{TS , j} - {G}_{R , j} ) , \qquad ( 6 )$$

where ΔGⱼ is the free energy difference between the lowest energy reactant and the j^{th} reactant and G_{TS} is the free energy of the transition state connected to the j^{th} reactant. Both of these methods underestimate the relative barrier for DKE 1 compared to the tertiary amine containing DKEs. In addition, both methods improperly space DKEs 2-4. The lowest free energy reactant method results in a large gap between DKE 3 and DKE 4, rather than between DKE 2 and DKE 3, and the Boltzmann weighted average gives almost identical barriers for DKE 3 and DKE 4.

Instead, ΔG^{‡} is obtained from the MP-TST rate,

$$Δ {G}^{‡} = ln ( {k}_{MP - TST} \frac{h}{{k}_{B} T} ) RT , \qquad ( 7 )$$

and find excellent agreement with the trends in experimental free energy barriers. MP-TST reveals all the critical features in the experimental trend in hydrolysis rate constants: the sharp increase in barrier from DKE 2 to DKE 3, the plateau to DKE 5, and the similar barrier for DKE 1 and DKE 5. This agreement emphasizes the utility of a multi-path approach for calculating reaction rates in systems with a large number of reactant and transition state conformations, capturing the differences between systems that favor a few conformers to those with more flat conformer distributions.

Depolymerization of PDKs. To test the understanding and predictions of the calculated hydrolysis rates of DKE 2 and DKE 3 small molecules, two triamine monomers varying only in amine spacing were employed to experimentally study their recycling behavior at the macromolecular level. Two PDK resins were prepared via polycondensation of either tris(2-aminoethyl)amine or tris(3-aminopropyl)amine with a ditopic triketone monomer. These materials were then compression-molded into solid-bar samples prior to immersing them in in 5.0 M HCl at 20° C. to study their depolymerization over time (FIG. 64, Panel A). The deconstruction of the PDK networks gives rise to a precipitate comprising the triketone monomer; the liberated ammonium crosslinker (either C₂ or C₃) remains soluble in acid (FIG. 64, Panel B). The C₂ triamine PDK is faster to depolymerize compared to the C₃ triamine PDK: precipitation of the monomer starts at 2-3 h for C₂ triamine PDK, while the macroscopic cleavage of the solid C₃ triamine PDK into an intermediate swollen network appears at 8-10 h.

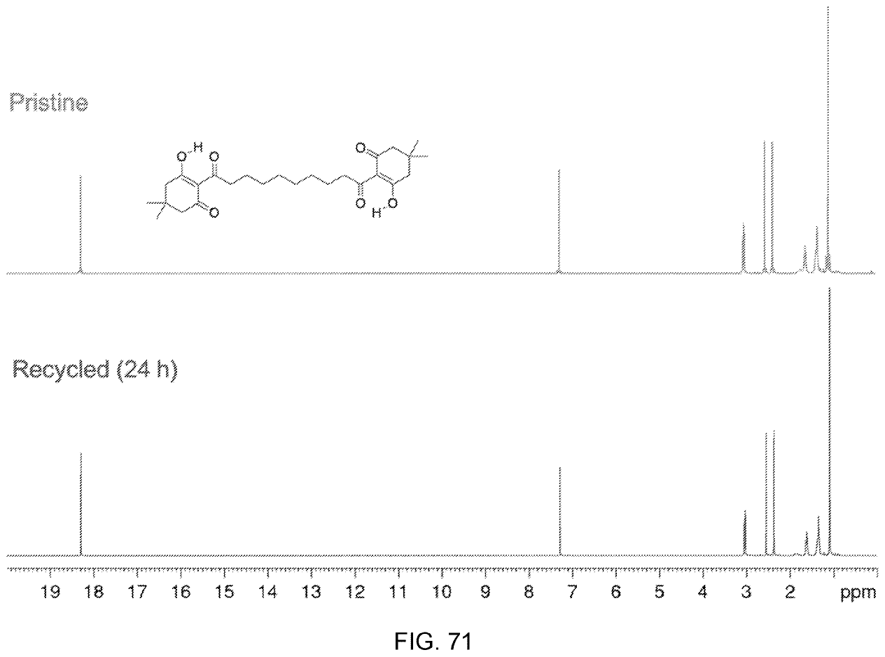

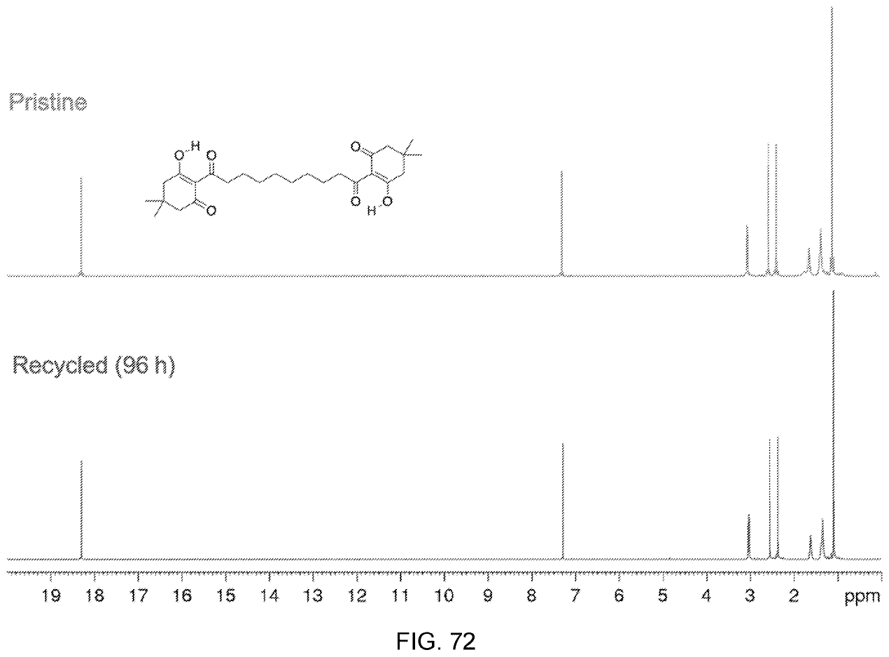

In order to anchor a visual interpretations of PDK acidolysis, the kinetics of the reaction were determined by mass recovery of the triketone monomer at different time points (FIG. 64, Panel C). Each experimental data point corresponds to a single sample that is filtered, basified, and precipitated in acid to ensure only the triketone monomer is recovered. The mass gravimetry difference between the starting and recovered materials allows us to determine the triketone recovery percentage shown in FIG. 64, Panel C. The C₂ triamine PDK reaches a maximum of depolymerization after 8 h and the C₃ triamine PDK takes about 96 h to reach 80% recovery. ¹H NMR spectra of the recovered monomer showed perfect overlap of the peaks with the spectra of the pristine triketone for both formulations (FIG. 71 and FIG. 72). These macromolecular findings regarding the recycling rates confirm what has been observed at the small molecule level, where DKE 2 hydrolyzes faster than DKE 3.

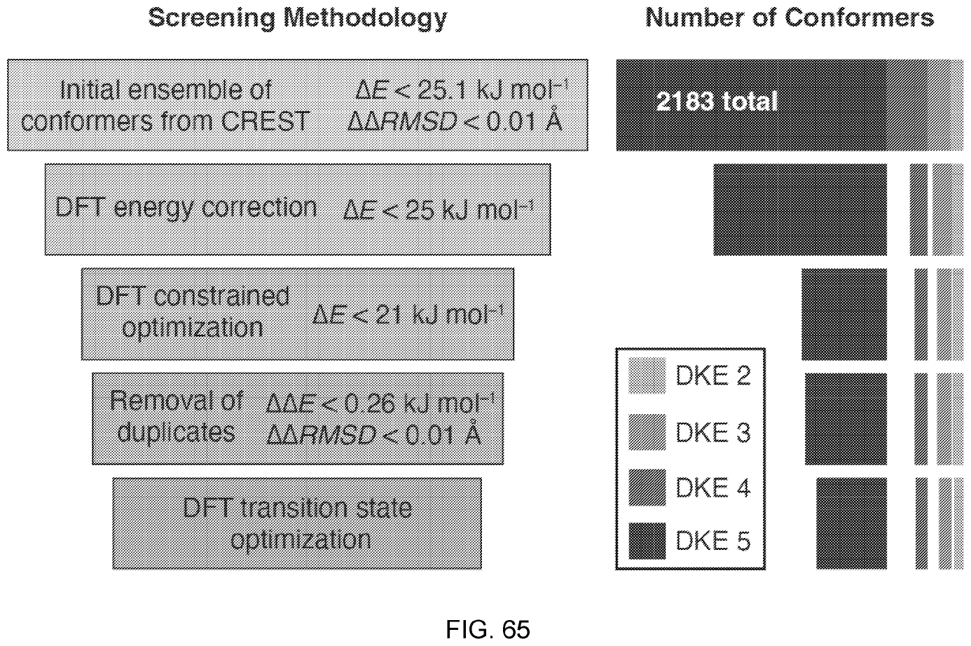

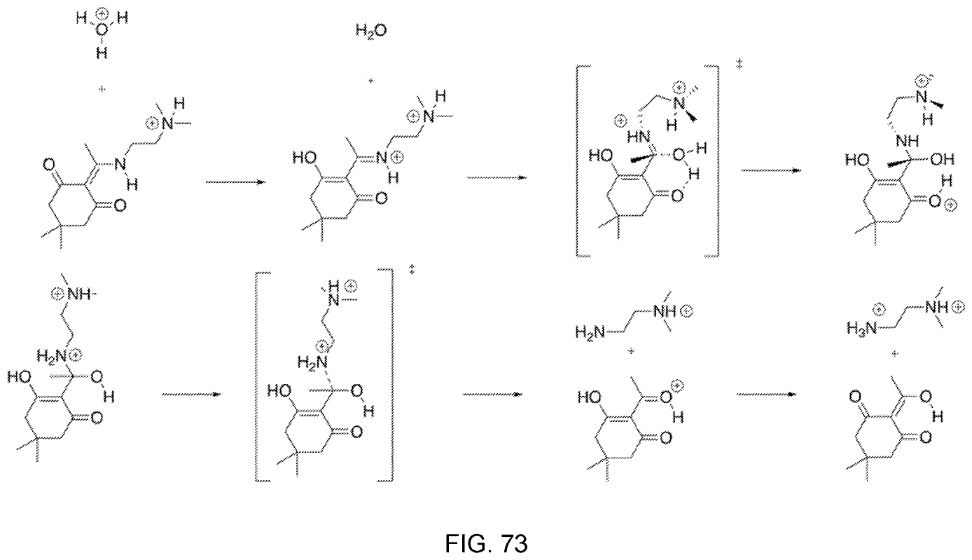

Experimental Section. A multi-stage conformer search method was used to identify the contributions to the MPTST rate calculation. First, a candidate transition state structure for the addition of water for each DKE was taken from the assumed hydrolysis reaction pathway (FIG. 73). The transition state was identified by optimization to a saddle point geometry using Gaussian16 at the ωb97XD/6-311+G(d,p)/SMD level of theory. An extensive conformer search was then performed using CREST to identify the ensemble of conformers with the lowest free energy (FIG. 65). Final transition state geometries were optimized at the ωb97XD/6-311+G(d,p)/SMD level of theory. Reactants were then generated by perturbing transition state structures along the vibrational mode corresponding to the single imaginary frequency in the Hessian to generate reactants. Reactants were then also optimized at the ωb97XD/6-311+G(d,p)/SMD level of theory. The free energy of the reactant was taken with the H₂O and DKE at infinite separation due to the variability in energies from small differences in the location of the single water molecule and the frequent failures in geometry optimization with one explicit water molecule. The final free energy includes a correction to the electronic energy with single-point calculations at the ωb97M-V/def2-TZVPD/SMD level of theory using Q-Chem 4.2. Single point corrections at ωb97XD/6-311++G(2df,2p)/SMD were also considered but overpredicted the acceleration of the hydrolysis in DKE 2. The distortion-interaction energy decomposition was performed at the ωb97M-V/def2-TZVPPD/SMD level of theory. Gaussian16 calculations were automated using QUACC29 and Q-Chem calculations were automated using Atomate.

For the MP-TST calculations described in eqn. 3, partition functions were calculated in the Quasi-Rigid Rotor Harmonic Oscillator approximation.

This example demonstrates how accurate simulations based on DFT calculations and MPTST modelling can help design circular polymer formulations where the placement of the amine can strongly influence the rate of depolymerization. The presence of a tertiary amine in proximity to the hydrolysis reaction center increases the hydrolysis rate by two orders of magnitude, but spacing this tertiary amine farther from the reaction center dramatically decreases the reaction rate to the point that there is no increase in rate over the control. Through computational analysis of the reaction pathway, it was found that this trend in reaction rate with amine spacing is due to the strength of the intramolecular hydrogen-bonded ring formed in the transition state of the addition of water to the iminium ion. Thus, it is expected that other hydrogen bond donors and acceptors can similarly coordinate with water to increase the reaction rate.

This example also showed the importance of using MP-TST based on the low-energy conformers of a system when calculating reaction kinetics for molecules with a large amount of conformational freedom and several non-covalent bonds accessible in select conformations. As polymer solvolysis is increasingly studied in plastics recycling, it is anticipated to see more computational studies for similar systems and emphasize that MP-TST with highly accurate conformer searching is beneficial.

Materials. 5,5-dimethyl-1,3-cyclohexanedione (dimedone, 95%), 2,2-dimethyl-1,3-dioxane-4,6-dione (Meldrum's acid, 95%), 4-(dimethylamino)pyridine (DMAP, 99%), N,N′-dicyclohexylcarbodiimide (DCC, 99%), 3-(dimethylamino)-1-propylamine (99%), 4-dimethylaminobutylamine, tris(2-aminoethyl)amine (TREN, 96%), tris(3-aminopropyl)amine (TAPA), potassium carbonate (K₂CO₃, 99%) and hydrochloric acid (HCl) were purchased from Sigma Aldrich and used as received. Acetic acid (>99.7%) was purchased from VWR and used as received. 5-(Dimethylamino)amylamine (98%) was purchased from Ambeed and used as received. All solvents—dichloromethane (DCM) (>99.9%), chloroform (CHCl₃) (>99.8%), ethyl acetate (>99.8%), methanol (>99.5%)—were purchased from VWR and used without further purification. 2-acetyl-5,5-dimethyl-1,3-cyclohexanedione and 1,10-bis(2-hydroxy-4,4-dimethyl-6-oxocyclohex-1-en-1-yl)decane-1,10-dione (Triketone monomer) were synthesized according to previously reported procedures¹.

Instrumentation. ¹H and ¹³C Nuclear Magnetic Resonance Spectroscopy. Spectra were acquired using a Bruker Avance II at 500 MHz and 125 MHz, respectively. Chemical shifts are reported in δ (ppm) relative to the residual solvent peak: 1) CDCl₃: 7.26 for ¹H, 77.16 for ¹³C or 2) D₂O: 4.8 for ¹H NMR for the hydrolysis kinetics in D₂O/DCl 5.0 M. Splitting patterns are designated as s (singlet), d (doublet), t (triplet), q (quartet), and m (multiplet).

Electrospray Ionization Mass Spectrometry (ESI-MS). Spectra were acquired using a Bruker microTOF-Q using acetonitrile containing either 0.1% trifluoroacetic acid as the ionization medium.

Fourier Transform Infrared Spectra (FTIR). Data were acquired using a Perkin Elmer Spectrum One spectrophotometer as an average of 32 scans over 400-4000 cm⁻¹.

Theoretical Methods. Reaction pathways were generated for each molecule according to the following steps:
1. Identification of a viable transition state for hydrolysis by optimizing to a saddle point and following the intrinsic reaction coordinate (IRC) to reactants and products.
2. Generation of an ensemble of transition state conformers with two constraints: one on the positions of the atoms in the H₂O and the second on the dihedral angle in the iminium, to prevent rotation about the double bond. Conformers were generated using CREST.
3. Calculation of single point energies on the GFN2-xtb geometries output by CREST at the ωb97XD/6-311+G(d,p)/SMD level of theory in Gaussian16.
4. Removal of structures 25 kJ/mol higher in energy than the lowest-energy structure.
5. Constrained optimization of GFN2-xtb geometries at the b97D/6-31+G(d) level of theory with density fitting.
6. Removal of duplicate structures. Duplicate structures were defined as those with an energy difference <0.1 kJ/mol and with an RMSD in the geometry <0.1 Å. RMSD was calculated using the Kabsch algorithm as implemented in Pymatgen.
7. Optimization to saddle points at the ωb97xd/6-311+G(d,p) level of theory
8. Perturbation of saddle point geometry towards reactant state.
9. Geometry optimization and frequency calculation of each reactant, separately, at ωb97XD/6-311+G(d,p)/SMD
10. Calculation of single point energy correction to final structures at ωb97M-V/def2-TZVPD/SMD using Q-Chem 4.2

These pathways were then used to calculate the MP-TST rate. Partition functions were calculated using the Quasi-RRHO approximation.

Permanent DOIs for computational data stored as jsons: 10.6084/m9.figshare.21809511, 10.6084/m9.figshare.21809490, 10.6084/m9.figshare.21809502, 10.6084/m9.figshare.21809508, 10.6084/m9.figshare.21809487, 10.6084/m9.figshare.21809499

Synthetic Methods. General Procedure for the Synthesis of Diketoenamines 1-5. To any of the acetyl-diones 1-5 (1.00 mmol) in CHCl₃ (4 mL) was added N,N-dimethylethylenediamine (1.00 mmol). The reaction mixture was stirred for 2 h at ambient temperature, after which the volatiles were removed under reduced pressure and the crude product was purified via a silica plug from 9:1 EtOAc:MeOH (v/v) to 100% MeOH to obtain the respective diketoenamines 1-5.

### Synthesis of 2-(1-(isopentylamino)ethylidene)-5,5-dimethylcyclohexane-1,3-dione (DKE 1)

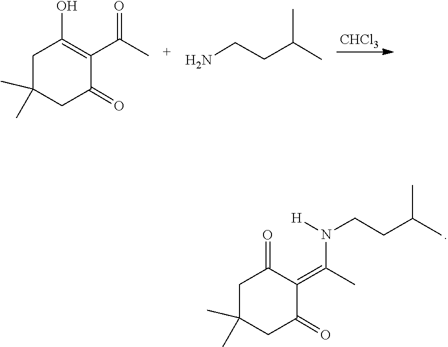

The crude product was purified via a silica plug from 3:1 Hex:EtOAc (v/v) to 1:1 Hex:EtOAc and was isolated as white crystalline solid (88% yield). Characterization data: ¹H NMR (500 MHz, CDCl₃): δ 13.43 (s, 1H), 3.44-3.40 (m, 2H), 2.58 (s, 3H), 2.38 (s, 4H), 1.80-1.73 (m, 1H), 1.62-1.57 (m, 2H), 1.05-0.97 (m, 12H) ppm; ¹³C NMR (125 MHz, CDCl₃): δ 197.9, 178.4, 107.8, 52.9, 41.6, 37.7, 30.1, 28.3, 25.7, 22.3, 17.9 ppm; ESI-MS: m/z for (C₁₅H₂₅NO₂)Na⁺ ([M+Na]⁺) calculated 274.1792, found 273.9408. FT-IR: 2957, 2928, 2871, 1634, 1568, 1452, 1419, 1384, 1366, 1335, 1322, 1308, 1289, 1271, 1248, 1202, 1174, 1139, 1126, 1087, 1036, 1017, 998, 973, 943, 904, 887, 822, 783, 708, 622, 597, 579, 567, 554, 472, 450 cm⁻¹.

### Synthesis of 2-(1-((2-(dimethylamino)ethyl)amino)ethylidene)-5,5-dimethylcyclohexane-1,3-dione (DKE 2)

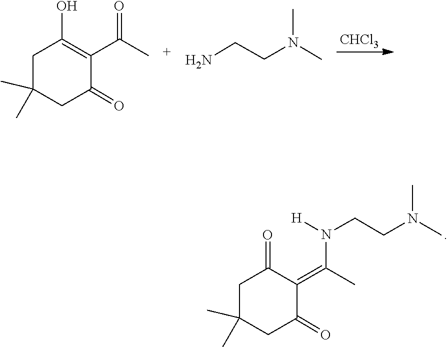

### Synthesis of 2-(1-((3-(dimethylamino)propyl)amino)ethylidene)-5,5-dimethylcyclohexane-1,3-dione (DKE 3)

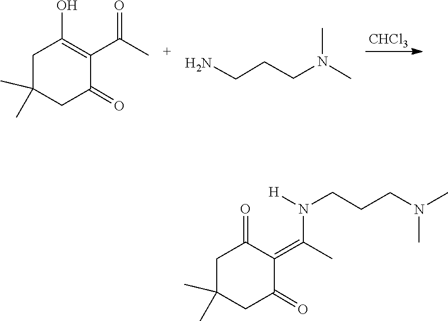

Isolated as white solid (86% yield). Characterization data: ¹H NMR (500 MHz, CDCl₃): δ 13.47 (s, 1H), 3.48-3.52 (m, 2H), 2.59 (s, 3H), 2.45-2.48 (t, ³J_{H-H}=6.9 Hz, 2H), 2.38 (s, 4H), 2.31 (s, 6H), 1.87-1.92 (m, 2H), 1.05 (s, 6H) ppm; ¹³C NMR (125 MHz, CDCl₃): δ 197.8, 173.5, 107.8, 56.3, 52.9, 45.3, 41.3, 30.1, 28.3, 27.0, 17.8 ppm; ESI-MS: m/z for (C₁₅H₂₆N₂O₂)Na⁺ ([M+Na]⁺) calculated 289.9892, found 288.9464. FT-IR: 2960, 2947, 2927, 2885, 2855, 2810, 2771, 2711, 1635, 1564, 1454, 1408, 1377, 1362, 1340, 1316, 1287, 1274, 1240, 1213, 1152, 1142, 1126, 1095, 1072, 1054, 1039, 1027, 1016, 1003, 962, 940, 911, 892, 828, 756, 709, 662, 628, 604, 582, 556, 467, 454 cm⁻¹.

### Synthesis of 2-(1-((4-(dimethylamino)butyl)amino)ethylidene)-5,5-dimethylcyclohexane-1,3-dione (DKE 4)

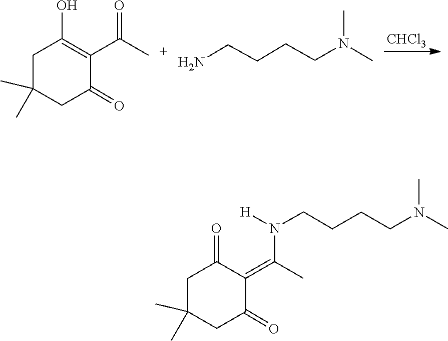

Isolated as white crystalline solid (36% yield). Characterization data: ¹H NMR (500 MHz, CDCl₃): δ 13.48 (s, 1H), 3.46-3.42 (m, 2H), 2.58 (s, 3H), 2.38-2.35 (m, 6H), 2.28 (s, 6H), 1.77-1.71 (m, ⁵J_{H-H}=6.85 Hz, 2H), 1.66-1.60 (m, ⁵J_{H-H}=6.85 Hz, 2H), 1.05 (s, 6H) ppm; ¹³C NMR (125 MHz, CDCl₃): δ 197.8, 173.4, 107.8, 58.9, 52.9, 45.4, 43.3, 30.1, 28.2, 27.0, 24.8, 17.9 ppm; ESI-MS: m/z for (C₁₆H₂₈N₂O₂)H⁺ ([M+H]⁺) calculated 281.2272, found 280.9836. FT-IR: 2964, 2940, 2889, 2866, 2811, 2779, 2758, 1626, 1571, 1455, 1421, 1366, 1344, 1326, 1290, 1270, 1256, 1227, 1204, 1174, 1167, 1141, 1128, 1109, 1098, 1083, 1068, 1055, 1040, 1028, 1008, 993, 919, 894, 844, 823, 796, 740, 702, 664, 642, 611, 584, 575, 545, 504, 462, 419 cm⁻¹.

### Synthesis of 2-(1-((5-(dimethylamino)pentyl)amino)ethylidene)-5,5-dimethylcyclohexane-1,3-dione (DKE 5)

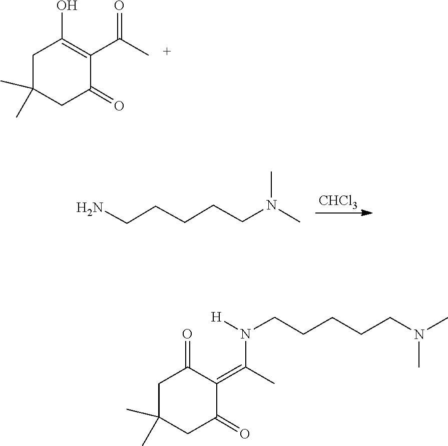

Isolated as white crystalline solid (63% yield). Characterization data: ¹H NMR (500 MHz, CDCl₃): 13.46 (s, 1H), 3.43-3.39 (m, 2H), 2.57 (s, 3H), 2.38-2.32 (m, 6H), 2.28 (s, 6H), 1.76-1.70 (m, ⁵J_{H-H}=7.35 Hz, 2H), 1.60-1.54 (m, ⁵J_{H-H}=7.05 Hz, 2H), 1.49-1.43 (m, 2H), 1.05 (s, 6H) ppm; ¹³C NMR (125 MHz, CDCl₃): δ 198.9, 198.4, 197.0, 107.8, 59.4, 53.3, 52.4, 45.4, 43.4, 30.1, 29.0, 27.2, 24.7, 17.9 ppm; ESI-MS: m/z for (C₁₇H₃₀N₂O₂)H⁺ ([M+H]⁺) calculated 295.2372, found 294.9969. FT-IR: 2939, 2863, 2812, 2760, 1627, 1571, 1457, 1382, 1365, 1337, 1289, 1266, 1241, 1201, 1173, 1154, 1141, 1127, 1111, 1099, 1086, 1062, 1042, 1020, 997, 962, 919, 896, 847, 823, 761, 731, 703, 664, 641, 584, 568, 546, 492, 462, 417 cm⁻¹.

Hydrolysis Studies of Diketoenamines 1-5. At a determined temperature, preliminary lock, tune, shim and run of the initial DKE spectra were realized before the kinetics runs. The DKE (5 mg) was solubilized in 600 μL of 5.0 M D₂O/DCl and transferred in a sealed-cap NMR tube. The NMR tube was introduced to the preheated NMR and spectra were subsequently acquired at different preset time intervals. Conversion values were calculated using the signal at 2.00 ppm (t, 2H, —NCH₂CH₂—) and the signal at 1.30 ppm (t, 2H, H₃N⁺—CH₂—CH₂—) of the released ammonium. The procedure was repeated at different temperatures.

Integration of the enamine peak in the ¹H NMR spectrum was used to calculate the disappearance of the diketoenamine starting material. The first-order rate law,

$$ln ( \frac{{[ diketoenamine ]}_{0}}{[ diketoenamine ]} ) = {k}_{obs} * t ,$$

was then used to calculate k_{obs}.

Based on this equation, k_{obs} could be calculated at 20° C. for each DKE molecule, which enables the calculation of relative k_{obs}.

ΔG^{‡} was determined using the Eyring equation (61):

$$ln ( \frac{{k}_{obs}}{T} ) = \frac{Δ {H}^{‡}}{RT} + ln \frac{{k}_{B}}{h} + \frac{Δ {S}^{‡}}{R}$$

From the extraction of ΔH^{‡} and ΔS^{‡}, ΔG^{‡} was calculated via ΔG^{‡}=ΔH^{‡}−TΔS^{‡}.

### C2-Triamine PDK Polymerization by Ball-Milling.

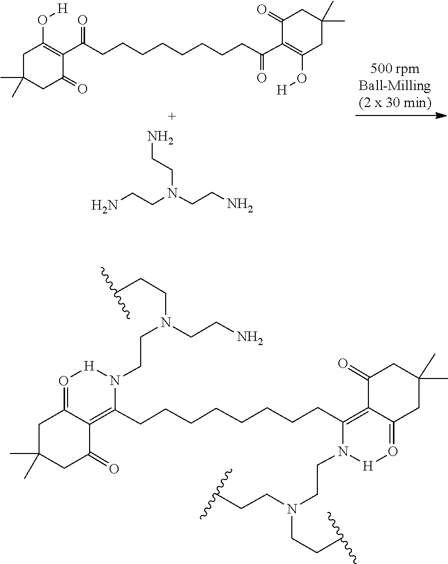

Ball-milling was performed using a Retsch Planetary Ball-Mill PM100. The container in which the reactions were carried out was a zirconium-coated cylinder either with an inner diameter of 4.5 cm and a height of 3.5 cm (reactor volume ˜50 mL) or with an inner diameter of 10 cm and a height of 7 cm (reactor volume ˜500 mL). All experiments reported herein used the same weight of zirconium oxide ball bearings (5 mm diameter) and triketone ratio, being 10 times the weight of triketone. The general procedure for all ball-milling reactions involved weighing out the appropriate amount of ditopic triketone monomer (2.0 or 10.0 g) and placing the powder at the bottom of the ball mill, along with the ball bearings (20 or 100 g). To the Triketone monomer (3.00 g, 6.72 mmol, 1 equivalent) was added tris(2-aminoethyl)amine (TREN) (0.739 mL, 4.93 mmol, 1.1 eq. amine) using a pre-calibrated micropipette, which was immediately followed by ball-milling the contents of the closed container for 30 min at a rotation of 500 rpm. The reactor was opened to air and the reactor walls were scraped to bring together the reactants homogeneously. Ball-milling was resumed for an additional 30 min under the same rotation speed. The powder was recovered from the reactor and the residual water removed under vacuum.

### C3-Triamine PDK Polymerization by Melt Polymerization.

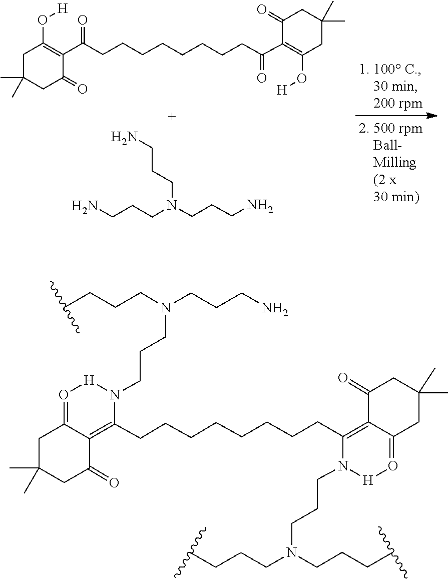

Triketone monomer (3.11 g, 6.96 mmol, 1 equivalent) was melted in a 20 mL vial at 100° C. in an oil bath for 20 min at 500 rpm. To the melt triketone monomer, tris(3-aminopropyl)amine (TAPA) (1.012 mL, 5.11 mmol, 1.1 eq. amine) was added using a pre-calibrated micropipette. The reaction was pursued for 30 min at 100° C. The crosslinked mixture was removed from the vial, broken down into centimeter-sized granules using a spatula and dried under vacuum overnight to remove the water formed during condensation reaction. The resulting solid was then reduced in size using a coffee grinder for 5 min in order to obtain a millimeter-sized yellow powder. The powder was recovered from the coffee grinder and used as it is for compression molding.

Processing of PDKs. Powdered PDK resins (˜1.2 g) were pressed using a stainless steel mold at 100° C. at 20 kpsi during 20 min to reach a homogeneous yellow rectangle. These rectangles were weighted and were shaped with dimensions of l=35 mm, w=12 mm, t=1 mm.

### Depolymerization Kinetics of C3-Triamine PDK.

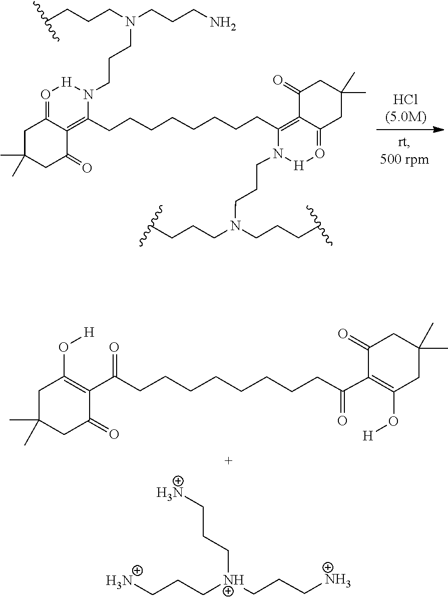

PDK solid bar samples (˜0.6 g) were depolymerized in aqueous 5.0 M HCl (15 mL) in a 40 mL vial during a 48 h 20° C. at 500 rpm.

A time lapse picture shot over one-hour intervals was recorded to visually determine the difference in depolymerization rates. At different time intervals, the liquid mixture containing triketone powder was separated from the solid polymer bar, centrifuged and rinsed twice with HCl (10 mL). Once the acid liquid removed, aqueous 2.0 M KOH (10 mL) was poured over the solid mixture to solubilize Triketone monomer. Basic Triketone solution was extracted and precipitated in HCl (2.0 M, 30 mL), the triketone precipitate was recovered by filtration and dried under vacuum for ¹H NMR analysis.

TABLE 2
Effect of the level of theory for single point energy corrections
on the rate constant calculated with MP-TST.

| | Calculated k_{Relative} (20° C.) | | | | |
|---|---|---|---|---|---|
| Level of Theory | DKE 1 | DKE 2 | DKE 3 | DKE 4 | DKE 5 |
| ωb97XD/6-311 ++ | 1.0 | 501.8 | 7.6 | 2.5 | 0.5 |
| G(2df, 2p) | | | | | |
| ωb97MV/def2 − TZVPD | 1.0 | 112.7 | 6.3 | 2.1 | 0.76 |

TABLE 3
Effect of the method used for calculating vibrational entropy
on the rate constant calculated with MP-TST. Vibrational Entropy
Approximation values for HO were taken directly from Gaussian16,
and Quasi-RRHO values were calculated with custom code.

| Vibrational Entropy | Calculated k_{Relative} (20° C.) | | | | |
|---|---|---|---|---|---|
| Approximation | DKE 1 | DKE 2 | DKE 3 | DKE 4 | DKE 5 |
| Quasi-RRHO | 1.0 | 112.7 | 6.3 | 2.1 | 0.76 |
| HO | 1.0 | 97.3 | 7.4 | 5.5 | 0.94 |

TABLE 4
Free energy barriers in the energetic span model. All
energies in kJ mol⁻¹. Free energies calculated with
the Quasi-RRHO approximation from structures optimized
at the ωb97XD/6-311 + G(d, p)/SMD level of theory
with ωB97MV/def2 − TZVPD/SMD electronic energy corrections.

| Molecule | ΔH^{‡} | TΔS^{‡} | ΔG^{‡} (298K) |
|---|---|---|---|
| DKE 1 | 66.73 | −58.68 | 125.40 |
| DKE 2 | 49.35 | −60.09 | 109.44 |
| DKE 3 | 60.50 | −56.49 | 116.99 |
| DKE 4 | 61.64 | −58.17 | 119.81 |
| DKE 5 | 65.86 | −61.27 | 127.12 |

### Figure Captions for Example 26

FIG. 60. Varying the amine spacing in circular polydiketoenamines tunes their depolymerization rate.

FIG. 61. Variation of the diketoenamine hydrolysis rate with increasing amine spacing. Structures of DKE 1, the control, and DKEs 2-5, with increasing amine spacing (Panel A). Calculated (MP-TST) and observed reaction rates for DKEs 1-5 show excellent agreement and a dramatic decrease in reaction rate with increasing amine spacing (Panel B).

FIG. 62. Decomposition of the energy barrier,

$$Δ {E}_{Total}^{‡} ,$$

in the distortion-interaction model. The distortion energy

$$Δ {E}_{Distortion}^{‡}$$

trends with the total energy barrier, while the interaction energy

$$Δ {E}_{Interaction}^{‡}$$

does not.

FIG. 63. Calculated and observed hydrolysis free energy barriers. Distribution of calculated free energy barriers contributing to the multi-path transition state theory rate calculation (Panel A). Points are shaded by the Boltzmann probability of the reactant for a given path. Comparison of methods for extracting the hydrolysis free energy barrier from the distribution of reaction paths (Panel B). Free energy barriers calculated from an Eyring analysis of the observed rates (Panel C).

FIG. 64. C₂ and C₃ PDK formulations hydrolysis. Hydrolysis reaction pathway of PDKs into the corresponding triketone and C₂ and C₃ ammonium monomers (Panel A). Visual deconstruction of the PDK networks over time (Panel B). Kinetics of triketone recovery over time (Panel C).

FIG. 65. Procedure for identifying the lowest energy conformers of the addition transition state. A Δ refers to values calculated with respect to the lowest-energy structure and A ΔΔ refers to values comparing each structure to all others.

FIG. 66. Hydrolysis kinetics of DKE 1 at 60, 70 and 75° C.

FIG. 67. Hydrolysis kinetics of DKE 2 at 20, 30, 40° C.

FIG. 68. Hydrolysis kinetics of DKE 3 at 41, 50, 60° C.

FIG. 69. Hydrolysis kinetics of DKE 4 at 65, 70 and 80° C.

FIG. 70. Hydrolysis kinetics of DKE 5 at 60, 70 and 80° C.

FIG. 71. ¹H NMR of pristine and chemically recycled triketone monomer from C₂ triamine PDK recycling after 24 h.

FIG. 72. ¹H NMR of pristine and chemically recycled triketone monomer from C₃ triamine PDK recycling after 96 h.

FIG. 73. Example reaction mechanism for the acid-catalyzed hydrolysis of DKE 2.

### Example 27—Biorenewable and Circular Polydiketoenamine Plastics

Amid growing concerns over the human health and environmental impacts of plastic waste, a valuable solution is to build a circular plastics economy where sustainability considerations dictate the full life cycle of plastics use including replacing petrochemicals with biorenewables. Here, it is shown that by incorporating the polyketide triacetic acid lactone (TAL) in polydiketoenamines (PDK), the working temperature of these circular plastics can be increased, opening the door wider to applications where circularity is urgently needed. By varying the number of carbons of TAL-derived monomers, both polymer properties and recycling efficiency are affected. Simply using glucose as the main carbon source, a process is engineered for producing bioTAL under fed-batch fermentation. A systems analysis of this bioprocess at different scenarios quantifies the environmental and economic benefits of PDK plastics and the risks when implemented at an industrial scale, providing opportunities in biorenewable circularity.

Bringing biorenewable circularity to plastics is critical to ensuring their sustainability. While bio-based monomers used to produce plastic resins are increasingly available from biomass and bioproducts, using them simply as drop-in replacements for commodity petrochemicals fails to deliver a bio-advantage in performance. Justifying their use in plastics production therefore remains difficult, as they are often produced at higher cost than the petrochemical they seek to replace. Furthermore, few existing plastics, even if produced from bio-monomers, are chemically recycled in closed loops, particularly monomer-to-monomer. Without biorenewable circularity, the dwindling supply of fossil resources may be consumed to meet the rapidly growing demand for plastics; moreover, there may be few incentives to recover plastic waste for recycling and reuse, failing to meet our goals for sustainable manufacturing. Future generations of plastics should emphasize bio-advantaged designs that achieve high efficiency in chemical recycling with respect to monomer recovery at end of life, so that the biorenewable content may be recirculated across the maximum possible number of manufacturing cycles. If this were realized, there could be a confluence of performance, manufacturing, and economic benefits to motivate the switch to new materials in the transition to a circular bio-plastics economy.

In support of this paradigm shift, circularity in emerging bio-plastics is demonstrated as achievable through co-design of polymers and chemical recycling processes (e.g., acidolysis, solvolysis, enzymolysis, and catalytic ring-closing depolymerization). Ring-chain equilibria have been exploited to enable circularity in polyesters and nylons from strained lactone and lactam monomers, some of which can be produced from bio-based raw materials. Complementing these efforts, solvolysis has been prioritized in the deconstruction of polyesters and polycarbonates from high molecular weight diols and carboxylic acids. Solvolysis and acidolysis are likewise applicable to bio-plastics featuring imine and diketoenamine bonds. Some of these bio-plastics have even shown properties similar to petroleum-derived plastics that remain difficult to recycle in closed-loops, such as polyethylene and polyurethane. Among them, bio-plastics based on polydiketoenamine (PDK) stand out for the concomitantly high efficiency and low cost required to chemically recycle them to the same monomers used in primary resin production. Yet, it remains a significant challenge to demonstrate circularity in bio-plastics, while deriving benefits from their constituent bio-monomers.

This example shows that biorenewable circularity in plastics concomitantly delivers a useful bio-advantage by incorporating the polyketide triacetic lactone (TAL) in polydiketoenamines (PDK), which are deconstructed to monomer at end of life with low carbon intensity in high yield and purity. The bio-advantage of TAL arises from its planarity, which promotes efficient stacking in the solid state and has the effect of densifying TAL-based PDKs (TAL-PDK). This densification raises the glass transition temperature (T_{g}) beyond a key threshold of 150° C., making possible the use of TAL-PDKs in broader applications (e.g., automotive), where structural integrity up to that temperature is of great importance. To understand the prospects for producing PDK resins from TAL, a fed-batch fermentation process is developed for bioTAL production using an engineered strain of E. coli that expresses a heterologous polyketoacyl-CoA thiolase, BktB, which converts acetyl-CoA into TAL in high titer. A detailed systems analysis of this process at different production volumes is also provided, where the environmental and economic benefits derived from biorenewability are quantified and delineated in the context of managing risk along the path to producing bioTAL and TAL-PDK resins at industrial scale.

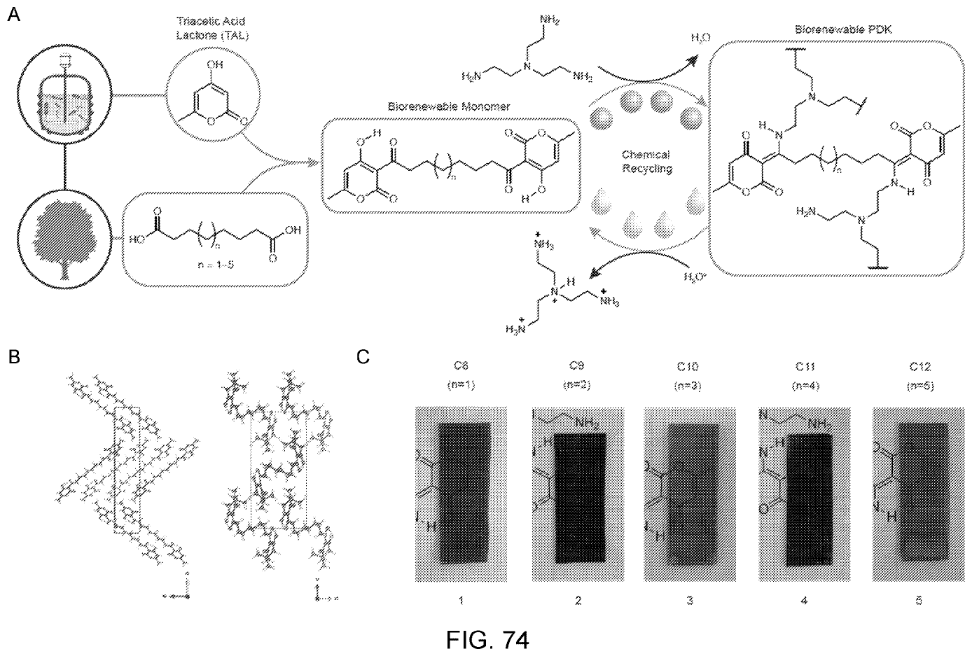

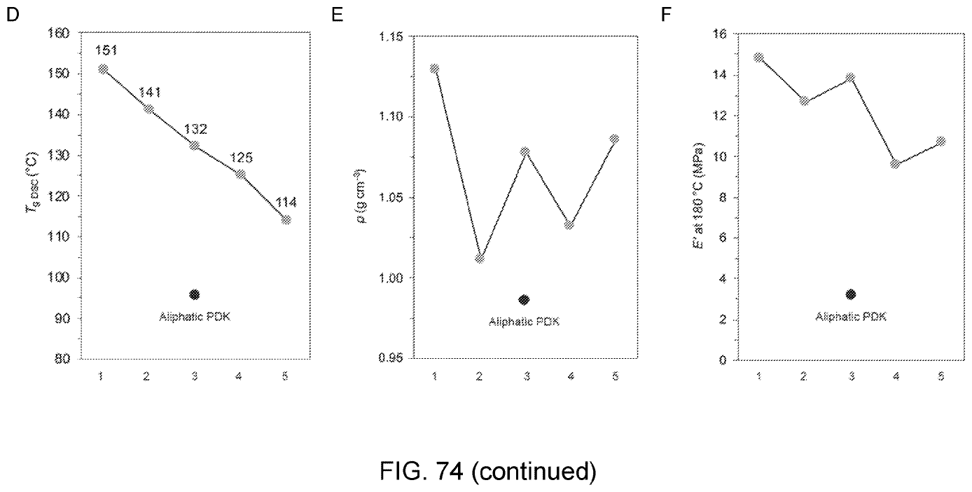

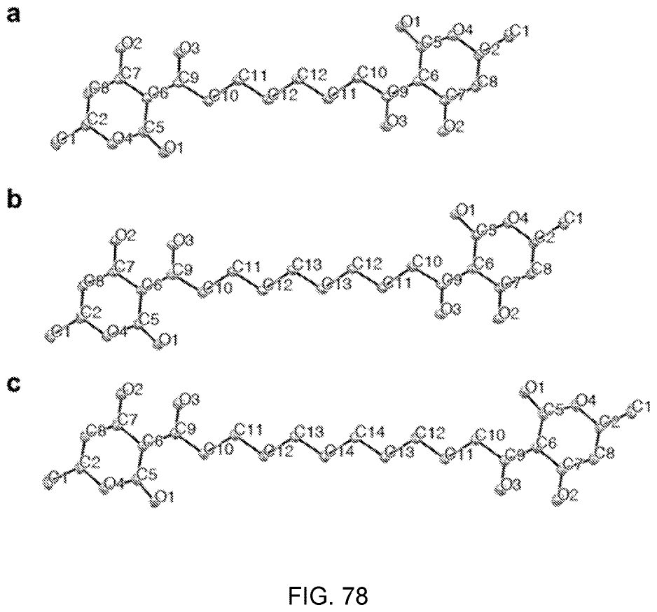

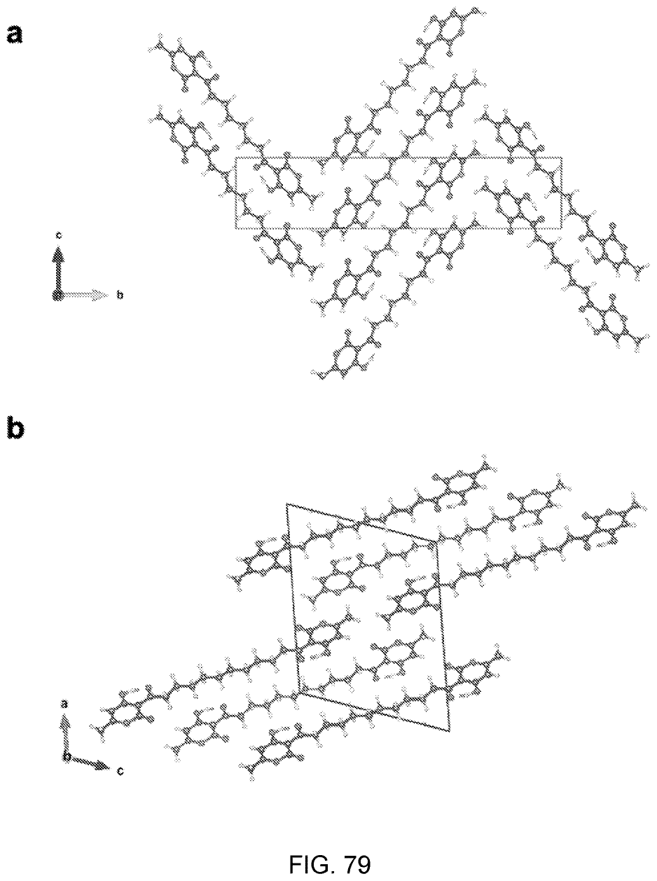

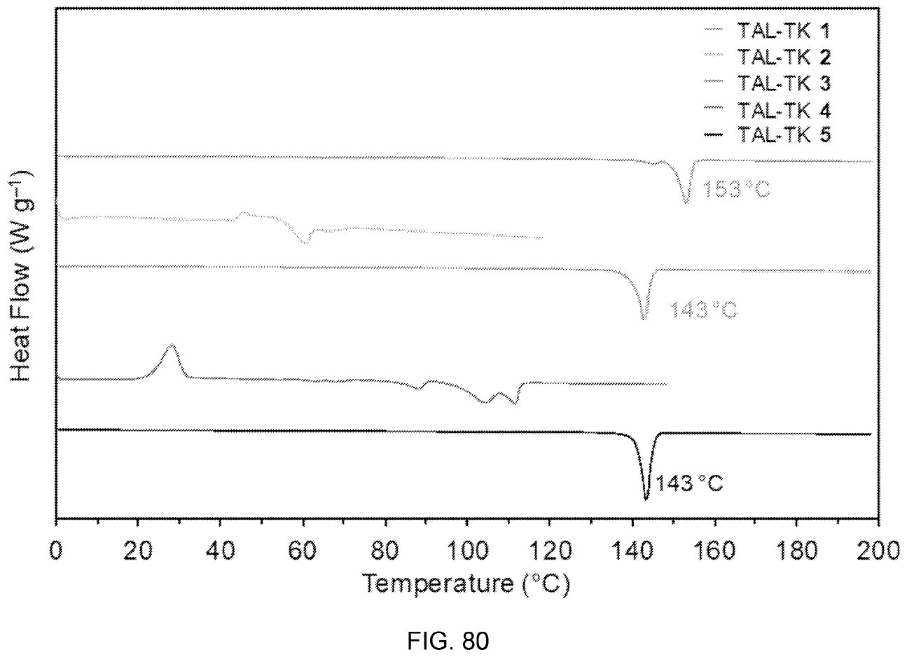

Results. Biorenewable Triacetic Acid Lactone-based PDK Resins. PDK resins are prepared via spontaneous “click” polycondensation reactions between polytopic triketone and amine monomers; no chemical condensation agent is required and water is the sole by-product of the reaction. Triketone monomers used in PDK production are synthesized from various 1,3-diketones and diacids. During synthesis, acylation of the 1,3-diketone typically occurs first at oxygen, which is then followed by an O- to C-acyl rearrangement catalyzed by 4-(dimethylamino)pyridine (DMAP). Owing to structural similarities between O-acyl intermediates and the likely reactivity of the preferred tautomer of TAL, it is hypothesized that TAL could stand in place of conventional petrochemical 1,3-diketones, such as dimedone, in monomer synthesis alongside common aliphatic C₈₋₁₂ dicarboxylic acids (FIG. 74, Panel A) to make available biorenewable triketone monomers (TAL-TK 1-5) and in turn PDK resins (TAL-PDK 1-5). Confirming this hypothesis, TAL-TK 1-5 were prepared in 40-63% yield (after recrystallization) using N,N′-dicyclohexylcarbodiimide (DCC) and DMAP. Single-crystal X-ray structures were obtained for TAL-TK 1, 3, and 5-all of which evidenced herring-bone packing of the monomers in the solid-state, due to stacking enabled by the planarity of TAL (FIG. 74, Panel B, FIG. 78, and FIG. 79). This propensity for stacking and densification is highly differentiated from that exhibited by a similar triketone monomer prepared from the petrochemical dimedone, which is not planar (Table 5). This difference in crystallinity is further exemplified in DSC analysis of TAL-TK monomers, showing sharp endothermic melting peaks for TAL-TKs 1, 3 and 5 (150, 143 and 143° C., respectively), while TAL-TKs 2 and 4 shows broader transitions at lower temperatures (60 and 100° C., respectively) (FIG. 80). It follows that the properties of PDK resins produced from these triketone monomers may likewise have different properties arising from the unique microstructures afforded to each.

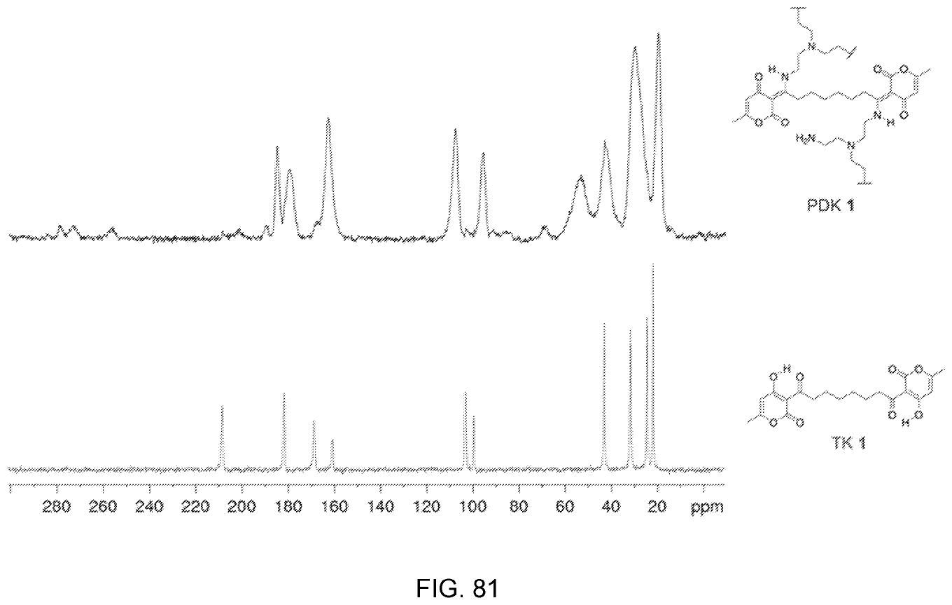

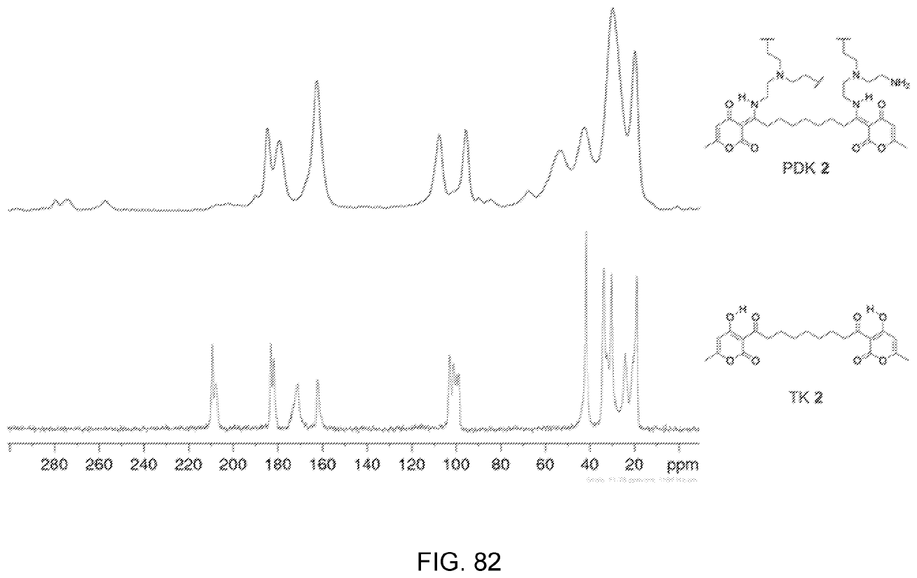

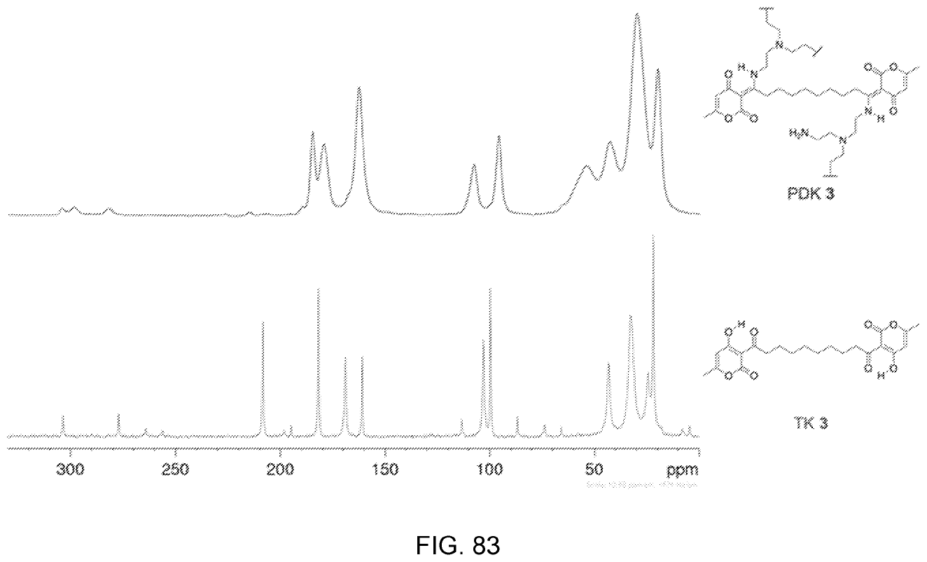

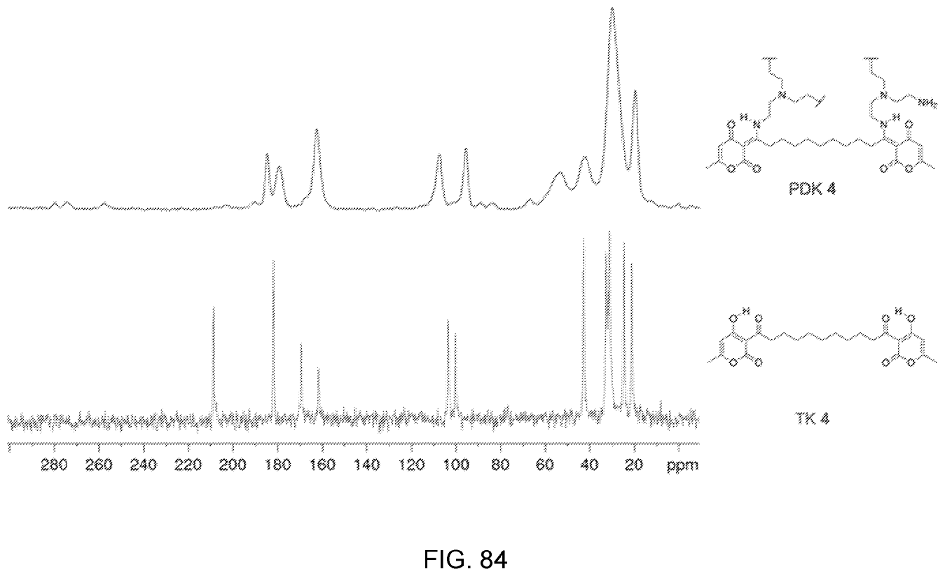

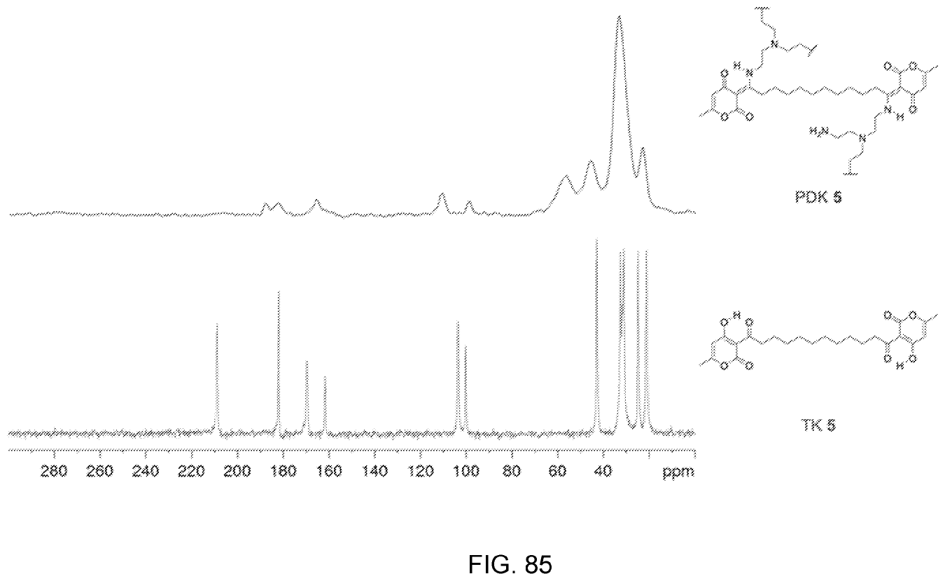

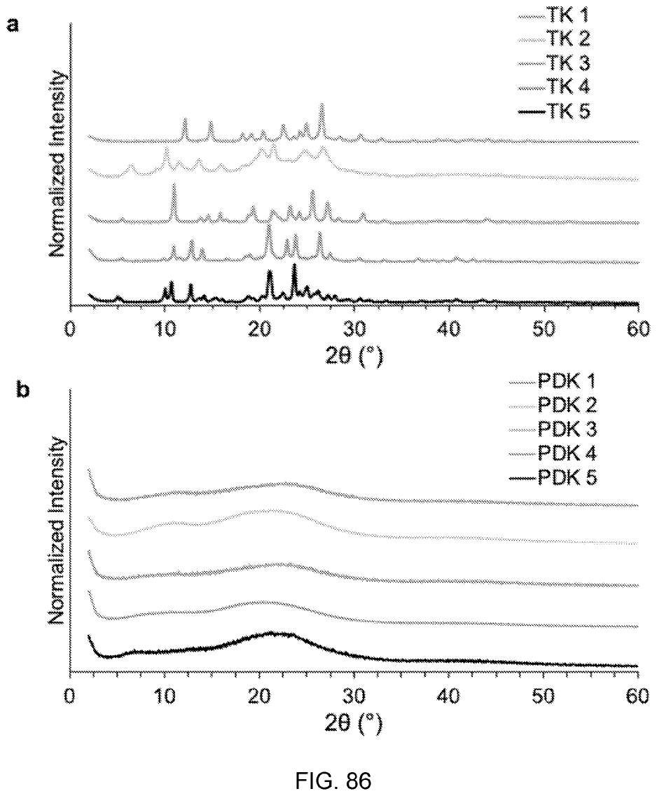

To understand these emergent microstructure-property relationships, TAL-PDK resins 1-5 were prepared from TAL-TK monomers 1-5 and tris(2-aminoethyl)amine (TREN); as a control, a PDK resin was also prepared from TREN and a triketone monomer derived from dimedone. To confirm that the polycondensation was complete, ¹³C solid-state nuclear magnetic resonance (¹³C SSNMR) spectroscopy was performed on powdered samples of TAL-PDK 1-5 (FIG. 81, FIG. 82, FIG. 83, FIG. 84, and FIG. 85). There is observed a disappearance of sharp peaks, otherwise corresponding to the crystalline triketone monomer, as well as a concomitant broadening of peaks corresponding to the polymer network. The extent of polymerization was further confirmed to be high by powder X-ray diffraction (PXRD), where sharp and intense peaks associated with the Bragg reflections of crystalline triketone monomers completely subsided to peaks exhibiting lower-intensity and significant broadening due to amorphization into a glassy vitrimer network (FIG. 86).

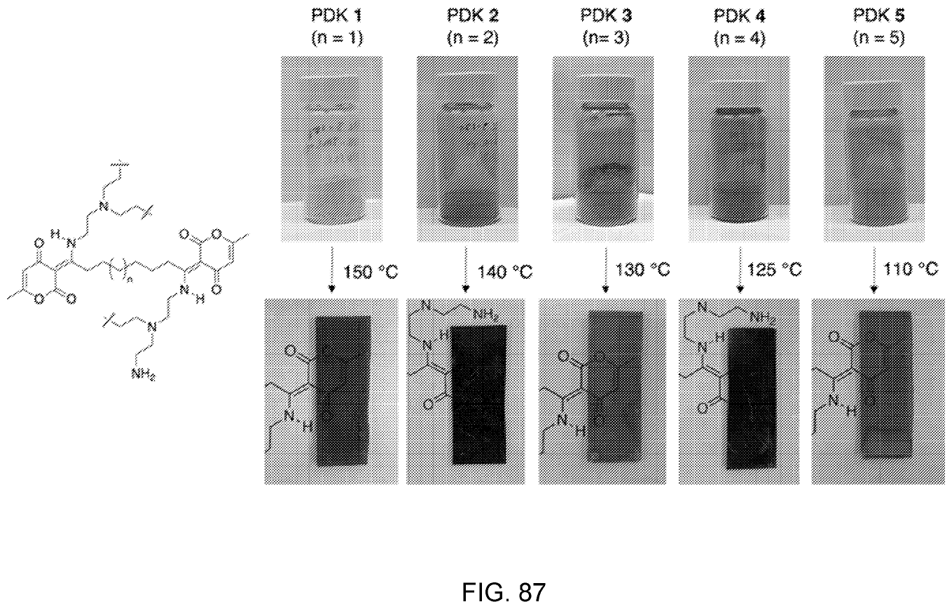

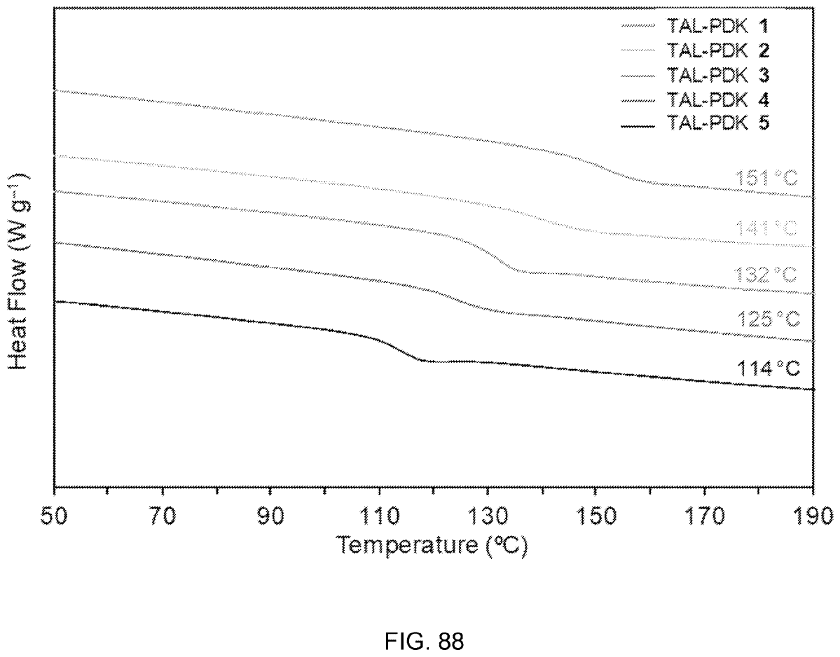

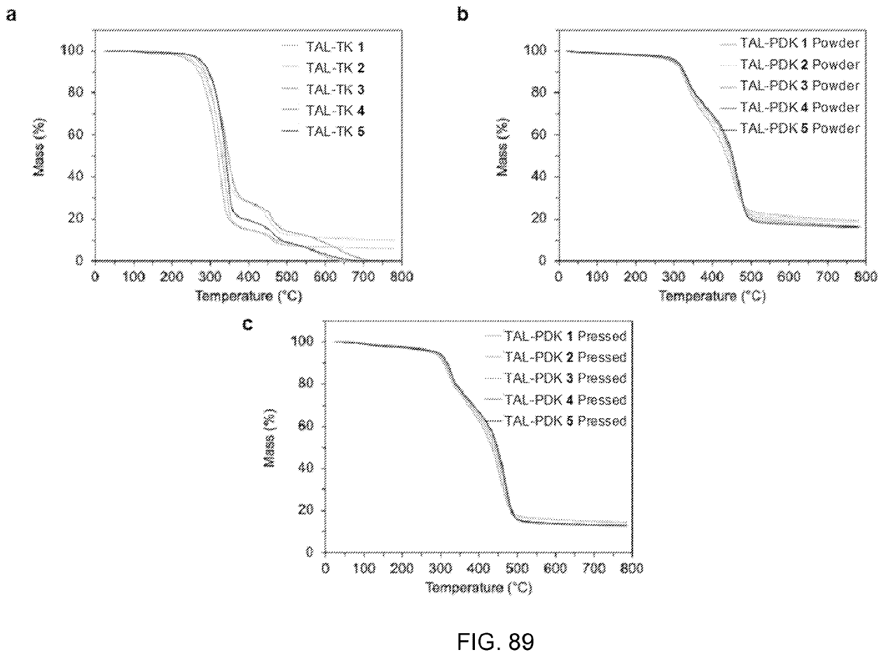

Enabled by the intrinsic dynamic covalent character of vitrimers, crosslinked PDK resin powders remain thermally processable, e.g., by compression molding. For TAL-PDK resins 1-5, compression molding at 20 kPsi pressure required temperatures of 150, 140, 130, 125, and 110° C., respectively, to fabricate samples (FIG. 74, Panel C and FIG. 87); thus, lowering vitrimer crosslinking density has the effect of lowering the energy requirement for PDK manufacturing. Whereas the expected color and high transparency of the vitrimers were readily apparent for TAL-PDK 1, 3, and 5 (as well as the control), colors were unexpectedly darker and hazier for TAL-PDK 2 and 4. The natural hue of TAL-PDK resins, which varies with crosslinking density and odd-even effects, would need to be accounted for in compounding with pigments to arrive at desired specifications. Nonetheless, it was observed a monotonic and well-behaved decrease in the glass transition temperature (T_{g}) with decreasing crosslinking density (FIG. 84, Panel D and FIG. 88). Importantly, in all cases, it was found that TAL dramatically raises T_{g} when incorporated into PDK resins: for example, the T_{g} of biorenewable TAL-PDK 3 is 36° C. higher than that of the related dimedone petrochemical control (T_{g}=96° C.), pointing to the key role of PDK microstructure on thermal properties. To provide context for thermal stability, thermal gravimetric analysis (TGA) data of the different formulations, with monomers (TAL-TK 1-5), powder and pressed-molded polymers (TAL-PDK 1-5) do not show any degradation below 200° C. (FIG. 89). Structural integrity of glassy polymer networks is critical for most commercial applications, from automotive to protective barriers and sporting gear; increasing T_{g} to meet product specifications remains an outstanding challenge, yet appears addressable with TAL-based PDK resins on account of their unique microstructure.

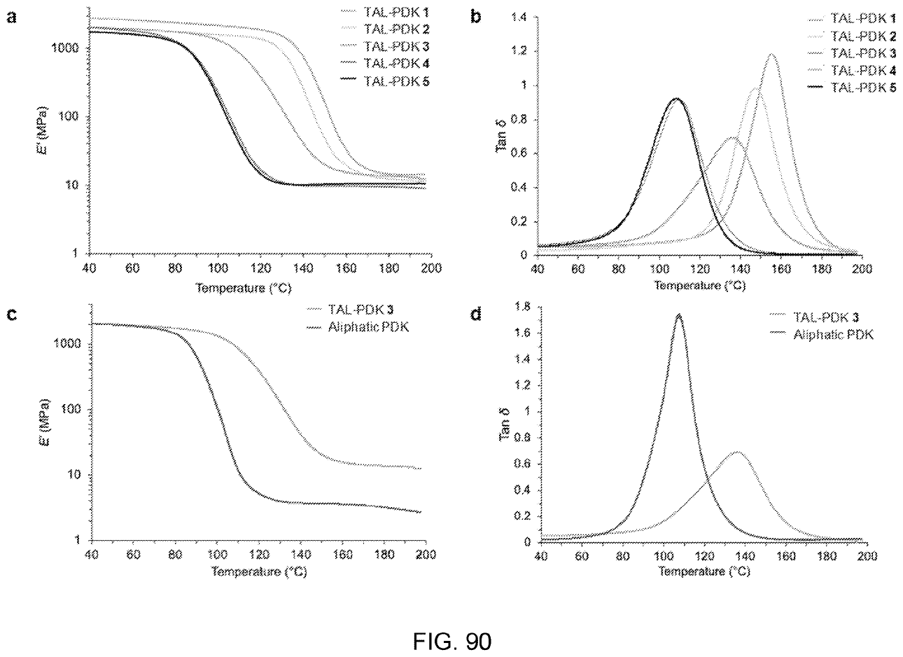

Motivated by this bio-advantage and its link to polymer microstructure, further studies of PDK properties were carried out, where microstructure often is relevant to or dictates outcomes, including density (p) and storage modulus (E) at temperatures above T_{g}. For elastic polymer networks, E′ is proportional top as well as the crosslinking density. Absent significant changes to ρ (which are rare), E′ should decrease monotonically with decreasing crosslinking density, as was observed for T_{g}; however, that is not what was observed experimentally. Instead, what was observed was odd-even effects in both ρ and E′ depending on the length of the diacid incorporated into the TAL-derived triketone monomer (FIG. 74, Panel E and F; FIG. 90). In all cases, ρ and E′ were higher for TAL-PDK materials than those of the related dimedone petrochemical control (Tables 6 and 7), consistent with the body of evidence presented herein, indicating more efficient packing in the solid-state and useful gains in elasticity and stiffness. For example, ρ is 1.078 g cm⁻³ and E′ is 13.5 MPa for TAL-PDK 3, whereas ρ is 0.987 g cm⁻³ and E′ is only 3.5 MPa for the control (i.e., 3.9-fold lower than TAL-PDK 3). The inventors are unaware of previous reports of odd-even effects in vitrimer microstructure-property relationships, yet they appear intrinsic and relevant to their design for function. Until now, odd-even effects in elastic polymer networks had only been explored theoretically, e.g., with respect to monomer topology, accounting for number of networking functionality in the monomers. Now, molecular configuration of constituent monomers and their influence on polymer chain conformation within the network architecture emerge as further points of interest and intrigue. Though unrelated, given the dissimilarity between linear and crosslinked polymer architectures, these observations are nonetheless reminiscent of odd-even effects in thermal properties of thermoplastics, e.g., the melting points of 1,n-nylons. Odd-Even effects, observed here in TAL-PDK materials, may be universal for polymer networks. In this way, the foundations of thermoplastic-like character of vitrimers, including biorenewable PDK resins produced from TAL can be unraveled.

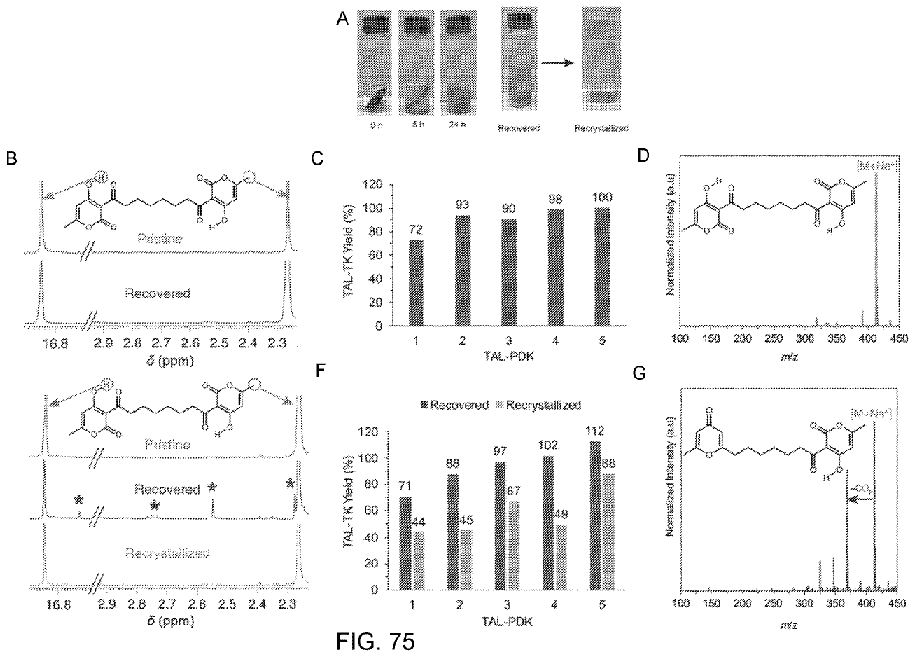

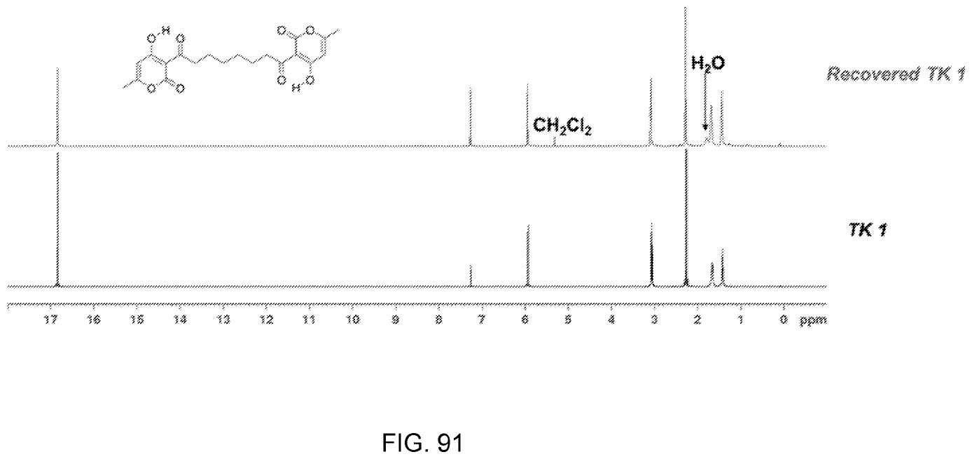

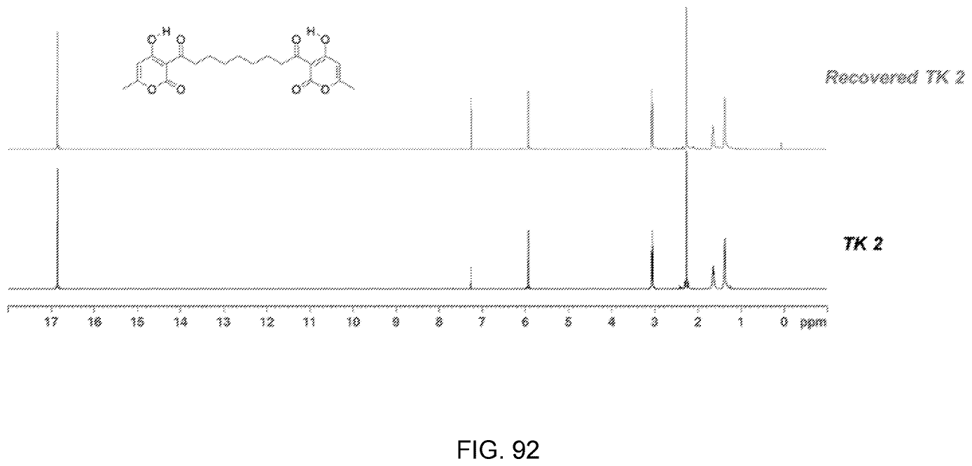

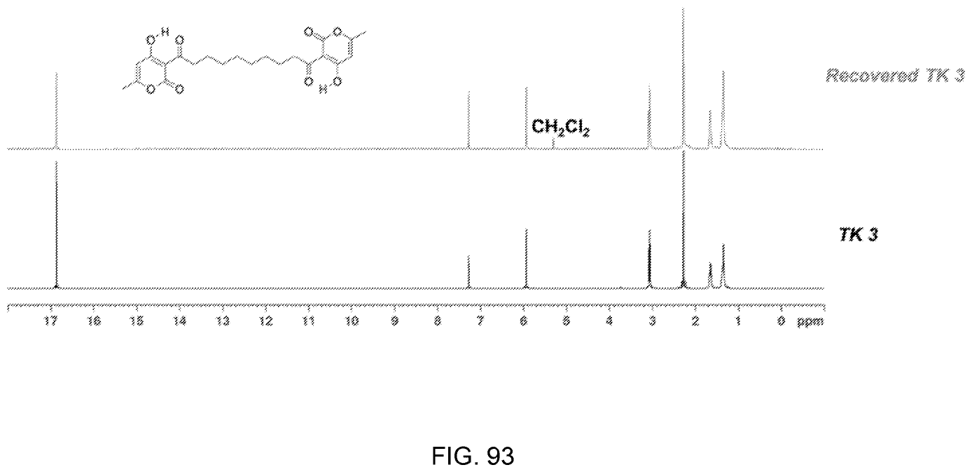

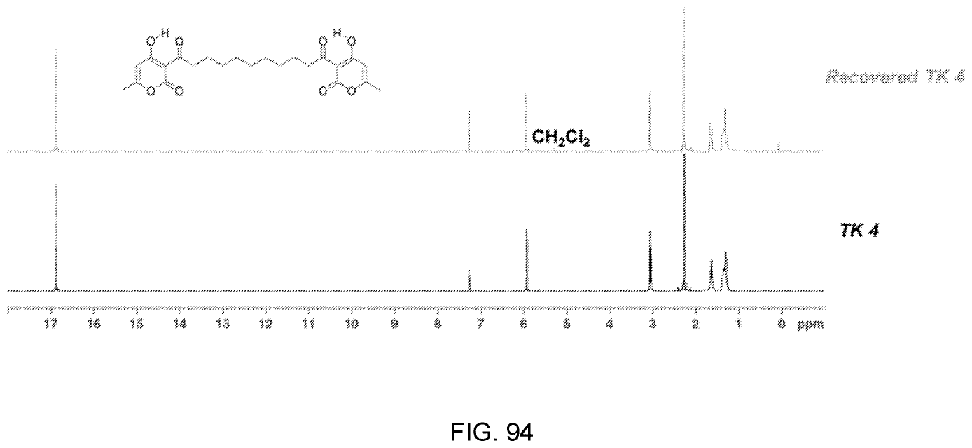

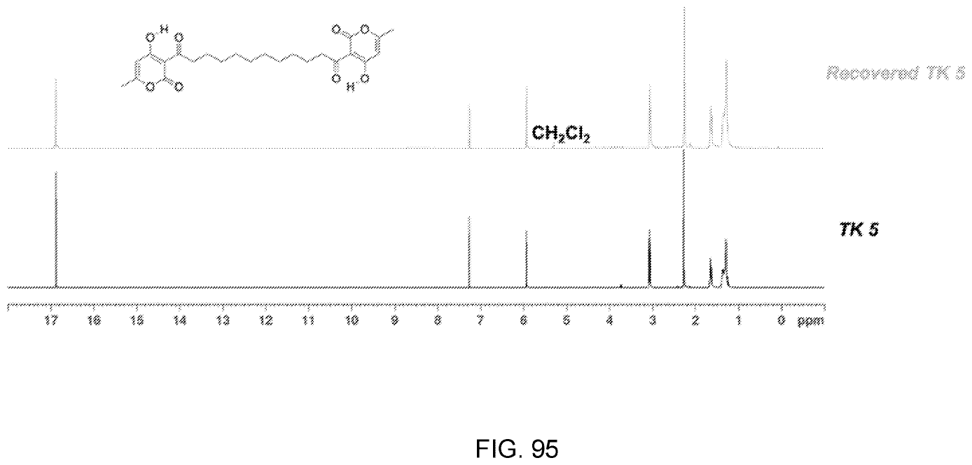

Molecular Basis for TAL-PDK Circularity in Recycling. PDK resins typically undergo deconstruction to triketone and amine monomers in strong acid at ambient temperature. Unlike triketone monomers derived from dimedone (i.e., the control), which have no cleavable linkages, those derived from TAL have motifs, such as the lactone, that may be susceptible to acidolysis. If lactone acidolysis is competitive with diketoenamine hydrolysis, then it could affect the materials efficiency of chemically recycling TAL-PDK resins back to monomer, e.g., if products other than TK 1-5 are also generated. To quantify the efficiency of circularity for TAL-PDK 1-5, we carried out their acidolysis in 5 M HCl for 24 h, after which all deconstructed to dispersed solids of TAL-TK 1-5, along with ionized TREN, which remained ionized in solution. We isolated TAL-TK 1-5 solids in 72, 93, 90, 98 and 100% yield, respectively (FIG. 75, Panel C); TREN can be recovered separately in high purity from the aqueous phase using a basic ion exchange resin. Recycled TAL-TK 1-5 were indistinguishable from the pristine monomers by ¹H NMR spectroscopy (FIG. 75, Panel B, FIG. 91, FIG. 92, FIG. 93, FIG. 94, and FIG. 95), indicating that the TAL-TK motif is remarkably stable under these conditions. Furthermore, when compared to yields and purity for monomer recovery for dimedone-based PDK resins (i.e., derived from petrochemicals), TAL-PDK circularity compares favorably, particularly for resins with lower crosslinking density.

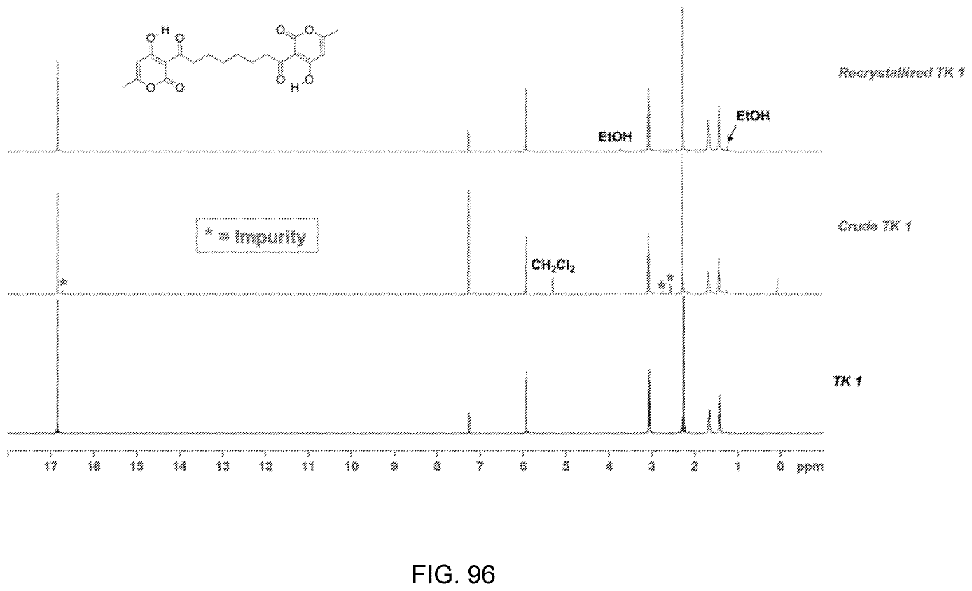

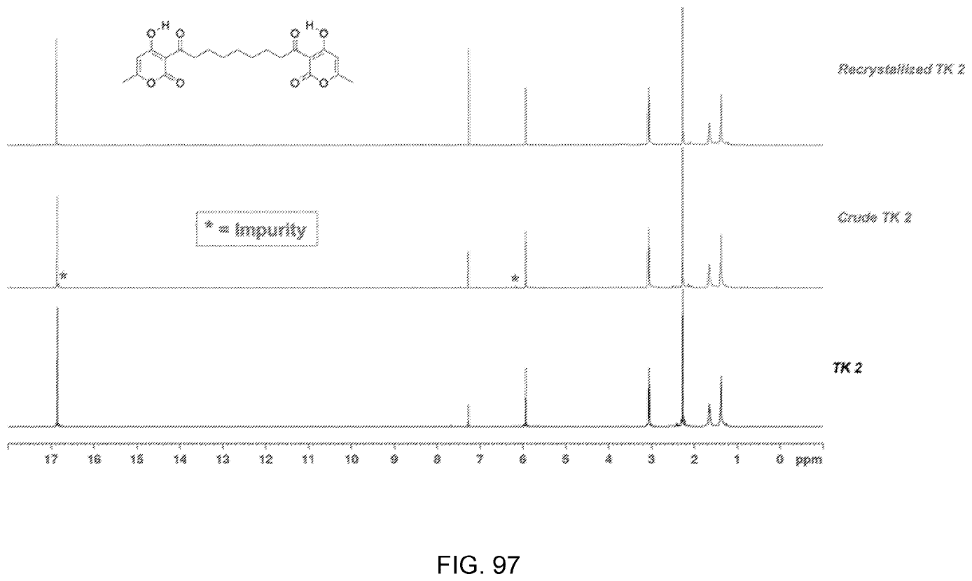

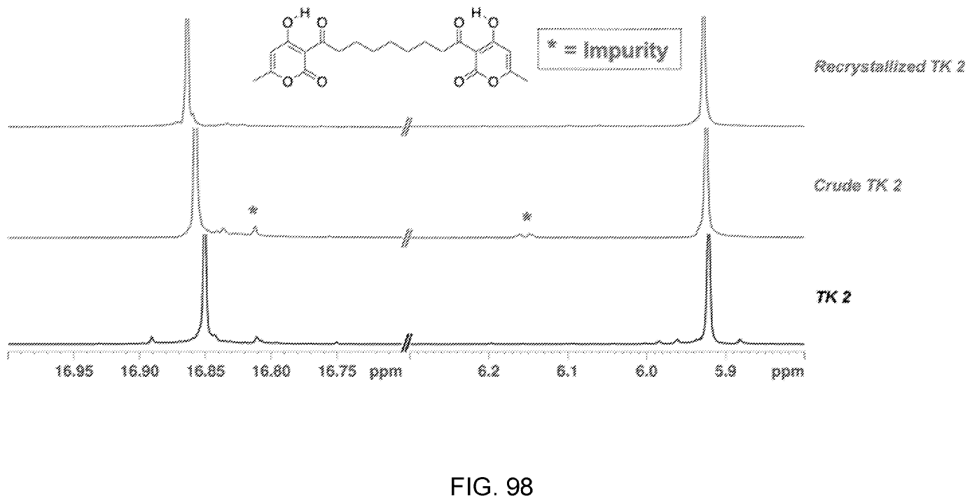

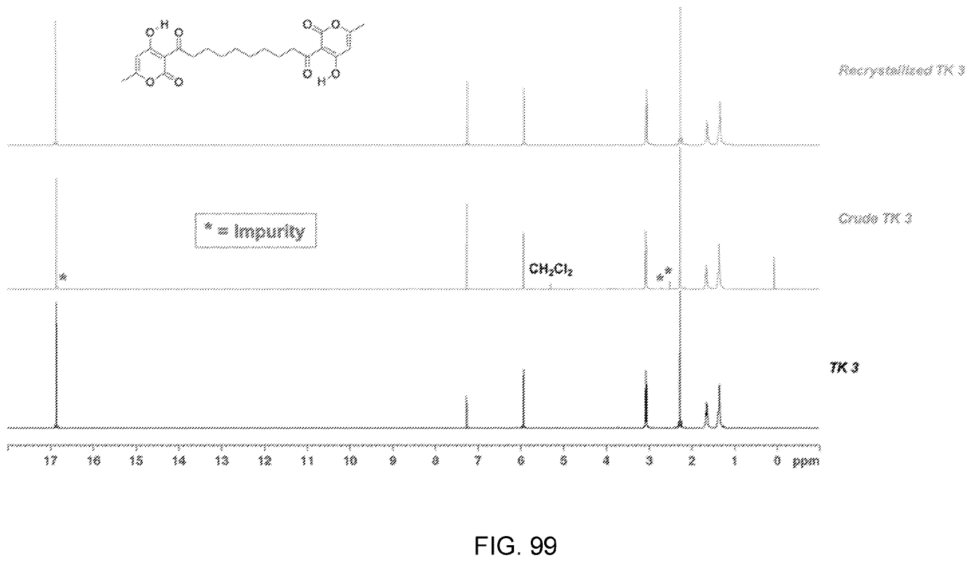

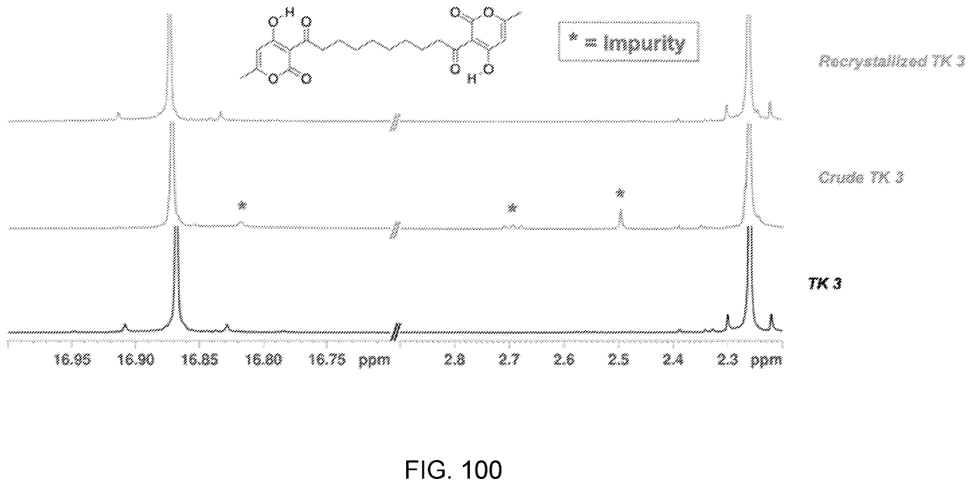

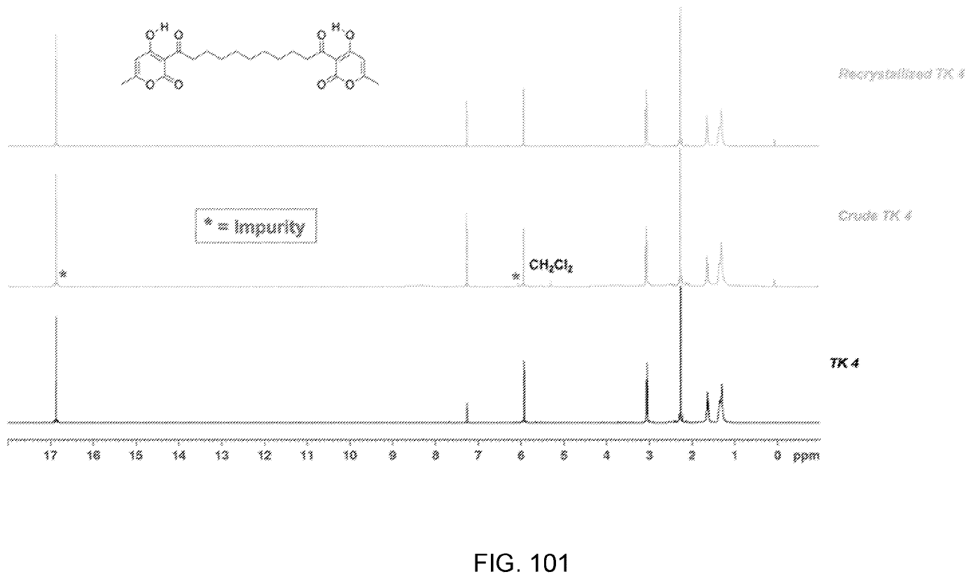

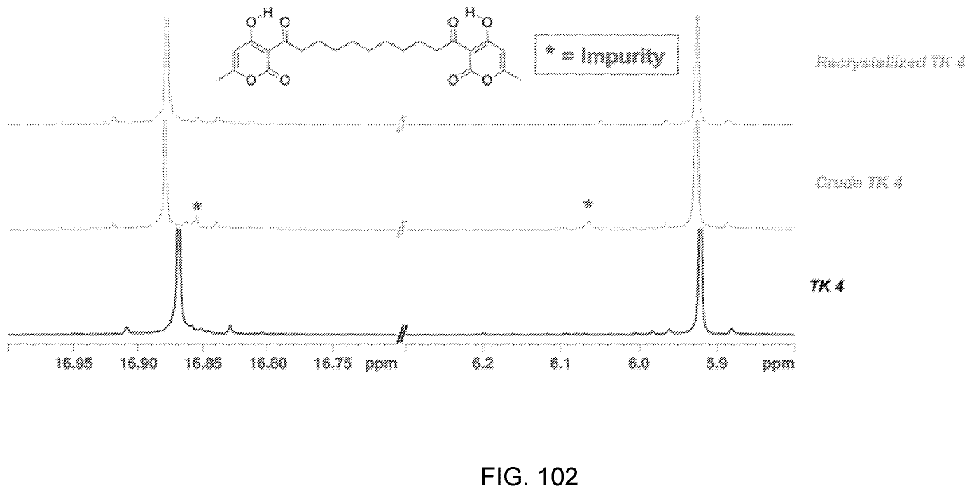

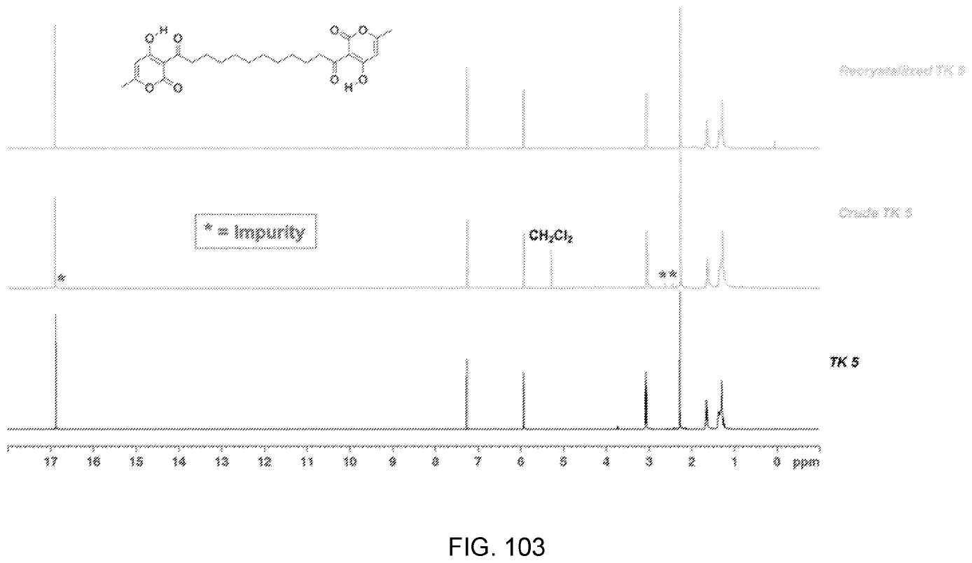

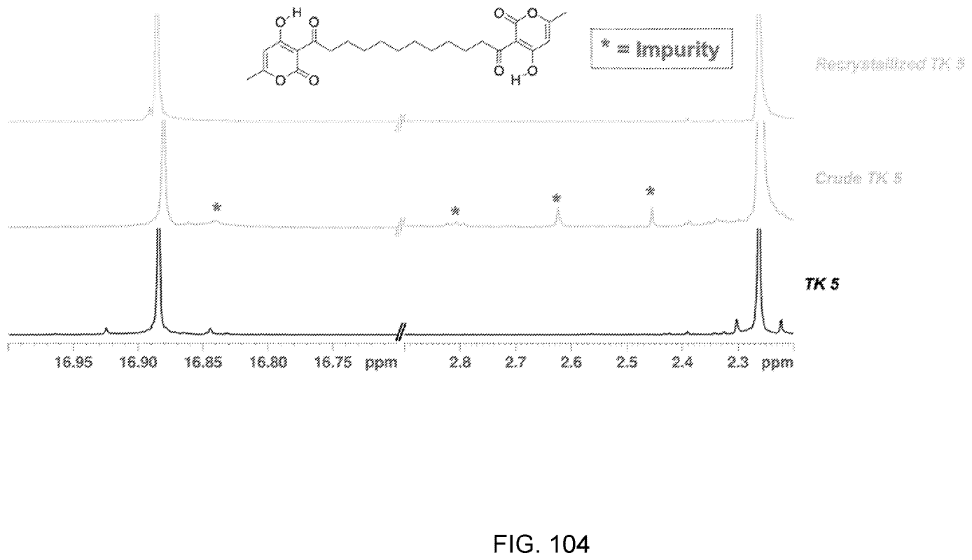

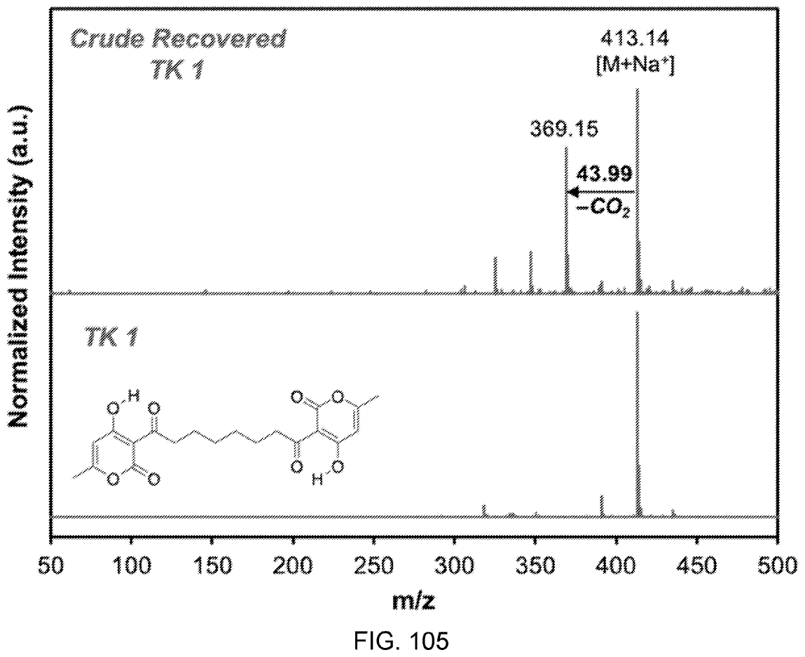

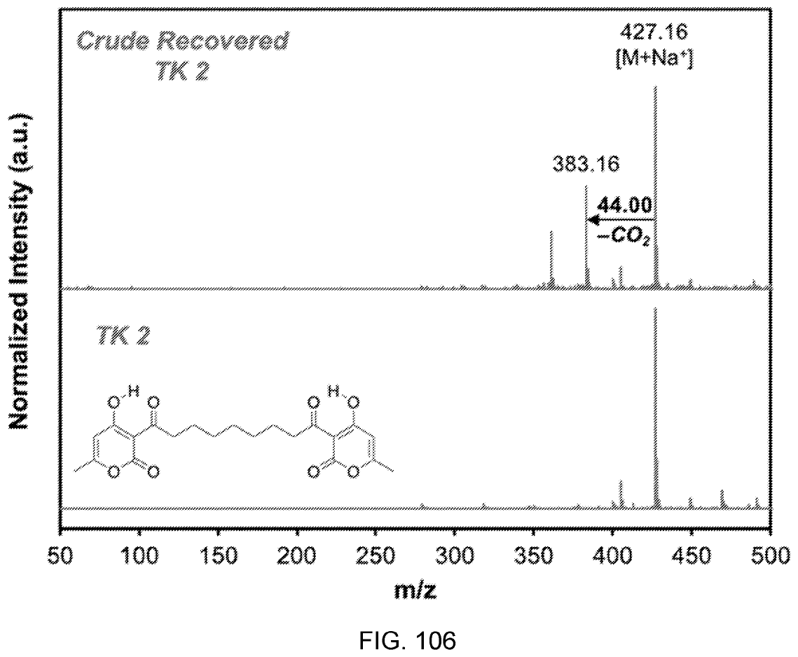

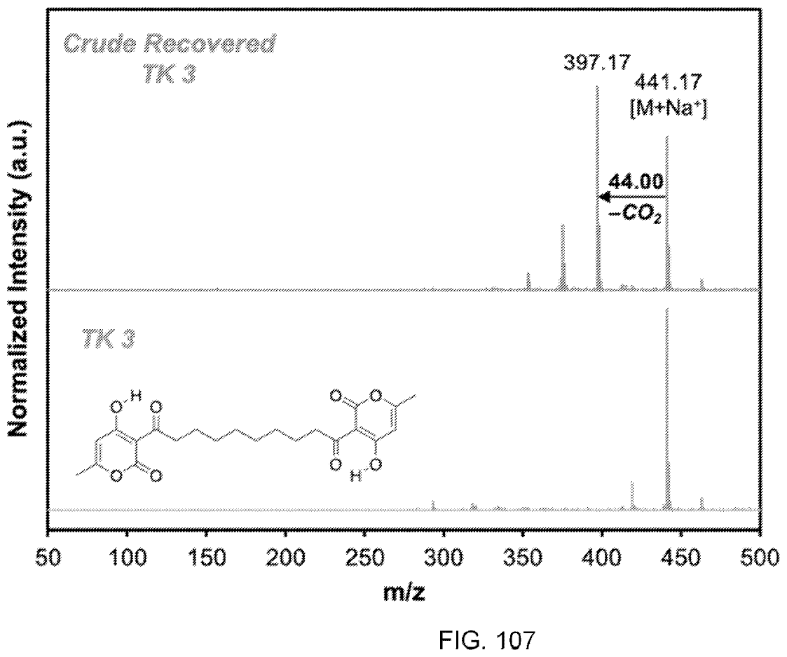

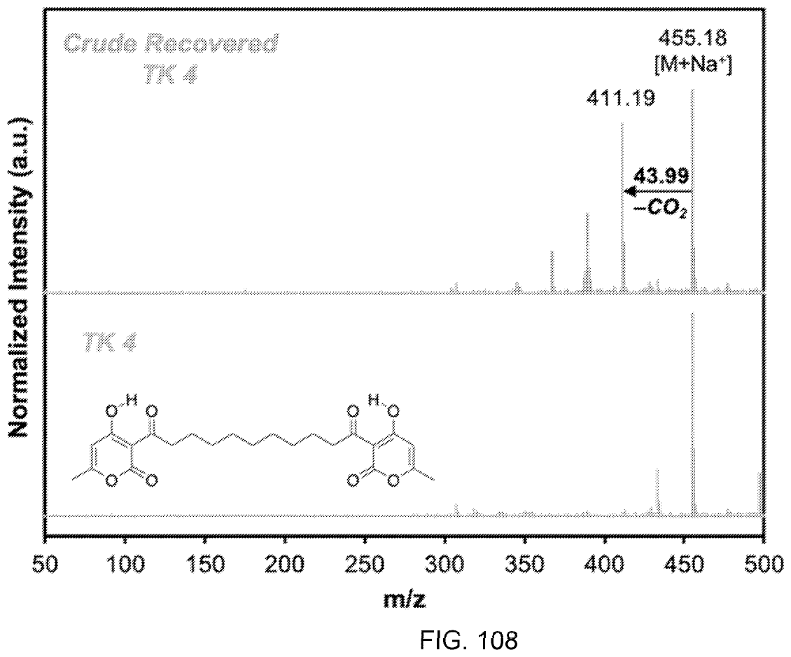

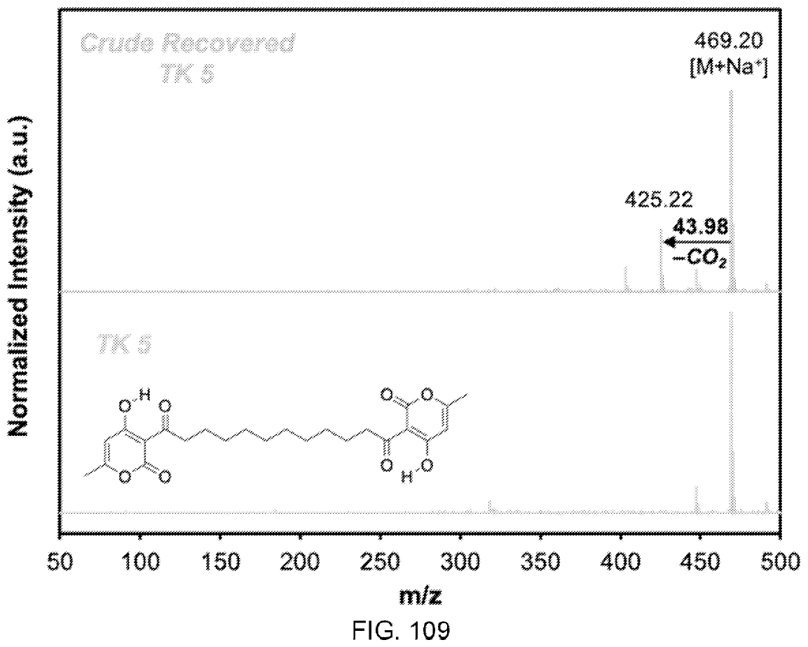

In quantifying further the efficiency of TAL-PDK circularity in compression-molded samples, we likewise found that lowering the crosslinking density was useful for ensuring high monomer recovery from TAL-PDK resins that had undergone conversion to various form-factors at high pressure and temperature (FIG. 75, Panel E and Panel F, FIG. 96, FIG. 97, FIG. 98, FIG. 99, FIG. 100, FIG. 101, FIG. 102, FIG. 103, FIG. 104, FIG. 105, FIG. 106, FIG. 107, FIG. 108, and FIG. 109). Polymer degradation during thermal processing during manufacturing or even mechanical recycling at end of life can be detrimental towards the development of circular plastics. An analysis of the degradation products formed after thermal processing can be informative towards an improved understanding of vulnerable structures and degradation pathways. These insights may provide new design criteria for constructing PDKs and other circular materials. To this end, we analyzed mass spectra for all crude triketones obtained after TAL-PDK deconstruction and compared these data with spectra obtained from the pristine monomers. In all cases, we observed a new and unique peak ˜44 mass units below the peak corresponding to a [TAL-TK+Na]⁺ ion (FIG. 75, Panel E and Panel G, FIG. 105, FIG. 106, FIG. 107, FIG. 108, and FIG. 109). This indicated a loss of carbon dioxide from some monomers recovered after depolymerization. In parallel, we observed new yet minor peaks (<15%) in the δ=16.7-17.0 ppm region of the ¹H NMR spectra for crude triketones. This indicated that while one triketone motif in the ditopic monomer remained intact in the minor byproduct of depolymerization reaction, the other had undergone decarboxylation during thermal processing (FIG. 96, FIG. 97, FIG. 98, FIG. 99, FIG. 100, FIG. 101, FIG. 102, FIG. 103, and FIG. 104). To remain consistent with the structural analysis afforded by mass spectrometry, it is likely this transformation generates a γ-pyrone. In cases where processing at high temperature and pressure led to materials degradation, we found triketone recrystallization from ethanol quite effective at removing unwanted γ-pyrone byproducts. Even with this additional purification process in place, TAL-TK 5 yields as high as 88% could be maintained, whereas in the absence of thermal processing and recrystallization, 100% yields were obtained. This understanding of the molecular basis for biorenewable circularity with TAL-PDK materials elevates future designs that benefit from lower crosslinking density to minimize mechanochemical activation of susceptible bonds within the TAL-PDK network.

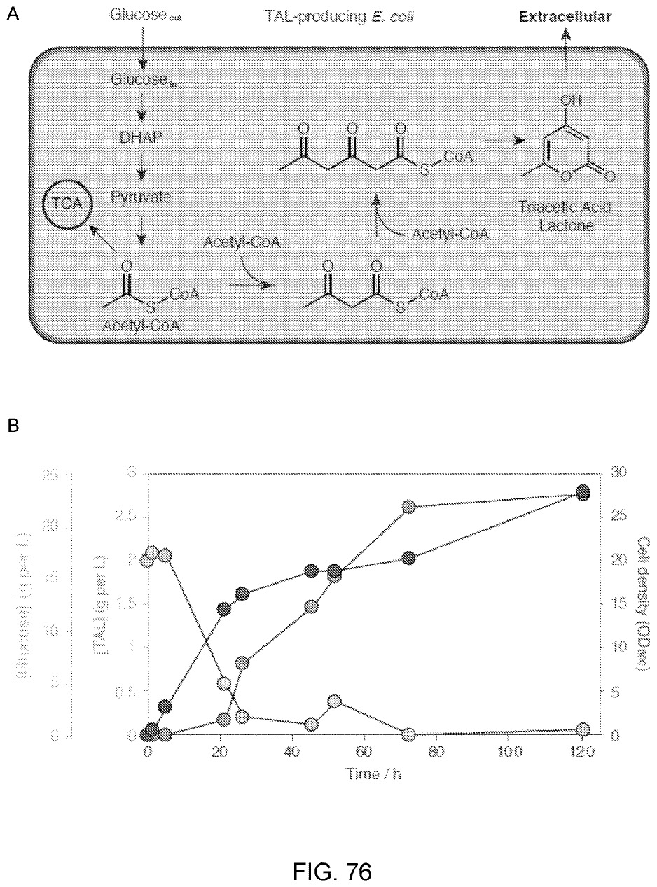

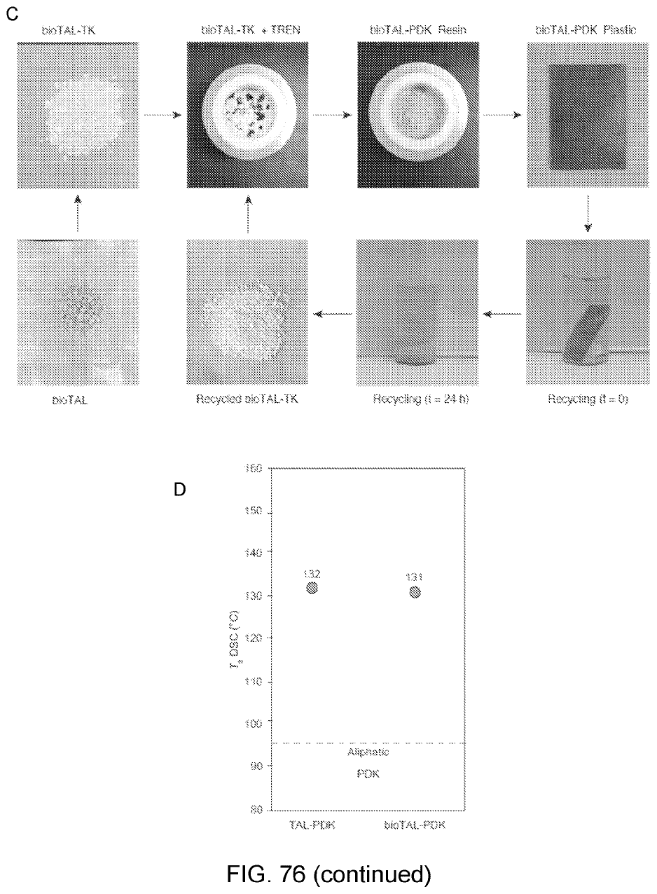

Bioproduction of TAL. Polyketide natural products are ubiquitous: some serve as important medicines, while others as useful chemicals or feedstocks for materials. TAL can be produced by the enzyme 2-pyrone synthase (2-PS), which catalyzes a succession of decarboxylative Claisen condensation reactions. TAL bioproduction with 2-PS has been achieved in Yarrowia lipolytica (36 g L⁻¹) and Rhodotorula toruloides (28 g L⁻¹), however overall yields from common carbon sources, such as glucose, could be improved if TAL synthases that perform non-decarboxylative Claisen condensation using acetyl-CoA as a substrate were used rather than those that perform decarboxylative condensation using malonyl-CoA as a substrate. Here, we used the non-decarboxylative polyketoacyl-CoA thiolase, BktB, for TAL bioproduction in an engineered strain, E. coli JBEI-3695 (FIG. 76, Panel A). If successful, the advance could open the door to directly converting sugars from plant biomass hydrolysates to this valuable bioproduct in high yield, closer to bringing biorenewable circularity to PDK resins.

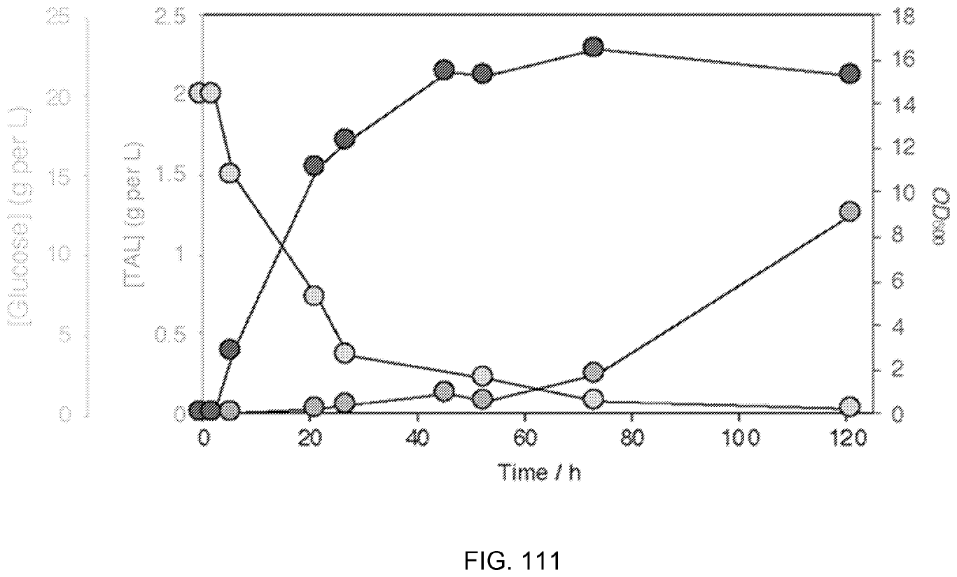

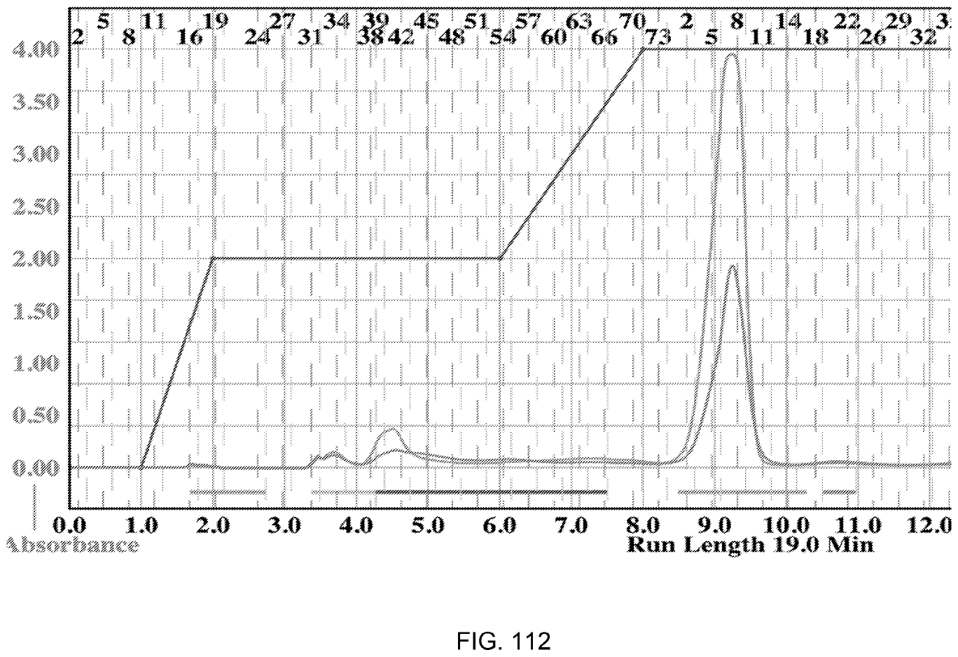

We synthesized the gene encoding BktB from Burkholderia sp. RF2-non_BP3 with codon-optimization for E. coli and cloned it into the vector pBbA5A, followed by transformation into E. coli JBEI-3695, which had some of its mixed-acid production enzyme genes deleted (ΔadhE ΔldhA ΔfrdBC Δpta) to enhance TAL production. We performed growth and production in a 1-L fed-batch bioreactor with optimized production media (Table 11), using glucose as the main carbon source. We monitored cell growth using optical density at 600 nm (OD₆₀₀) of 1-mL cell samples removed periodically during the production. The cells achieved a final OD₆₀₀ of 27.9 in 120 h and produced TAL to a final titer of 2.77 g L⁻¹ (FIG. 76, Panel B). The overall yield of TAL was ˜0.11 g TAL per g glucose, and the overall production rate was 0.035 g L⁻¹ h⁻¹. This experiment was repeated to produce additional TAL batches for bioTAL-PDK synthesis (FIG. 111). Though unoptimized, these results compare favorably to previous TAL yields from mixed carbon sources (glucose, fructose, sucrose, and acetate) in Rhodotorula toruloides by 2-PS (0.089 g TAL per g mixed carbon sources). After production and lyophilization of the broth, we extracted the mixture with ethyl acetate and isolated the desired TAL bioproduct (bioTAL) in high purity (FIG. 112). We then synthesized a biorenewable triketone monomer bioTAL-TK 3 from bioTAL and sebacic acid, which we obtained as a bioproduct from Arkema. These 100% biorenewable triketone monomers showed essentially identical properties, when used in the synthesis and chemical recycling of bioTAL-PDK 3 resins (FIG. 76, Panel D).

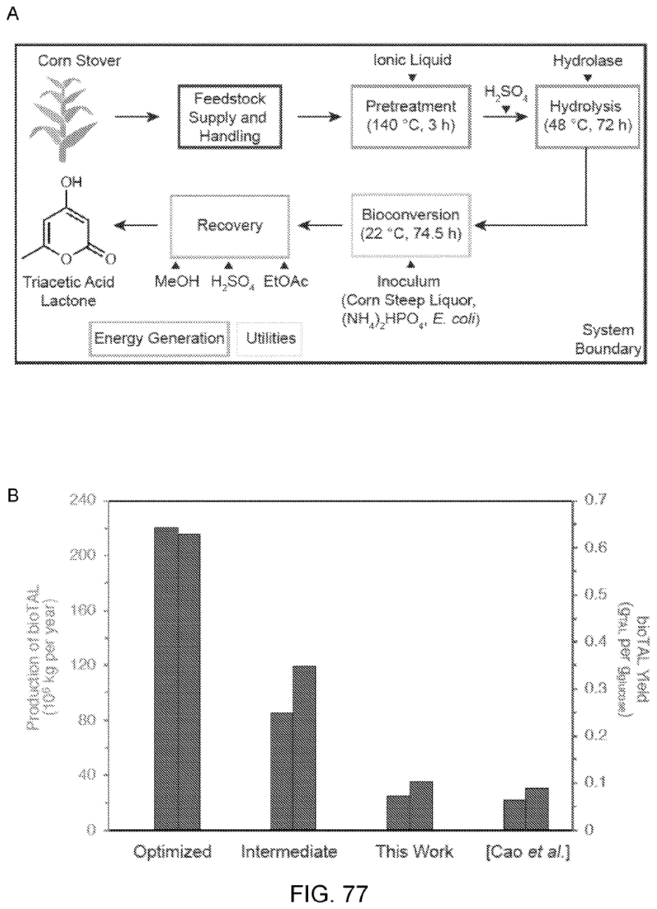

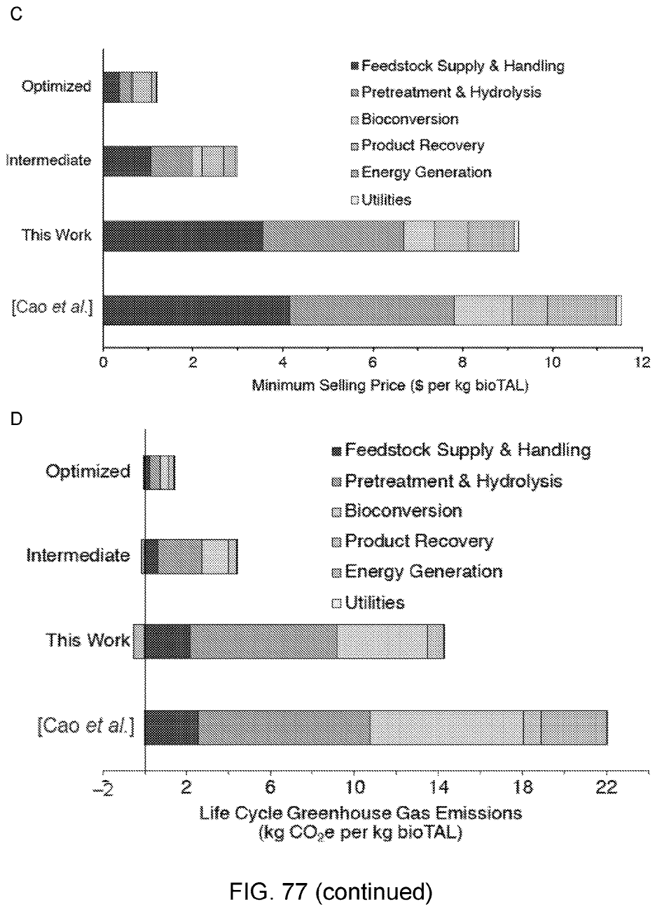

LCA and TEA of TAL bioproduction. To understand the key cost and GHG emissions drivers of bioTAL production, we carried out a system-level techno-economic analysis (TEA) and life-cycle GHG inventory. All costs and emissions estimates are based on the system summarized in FIG. 77, Panel A. We modeled a biorefinery where corn stover is pretreated with a bio-compatible ionic liquid (cholinium lysinate), followed by enzymatic hydrolysis, to generate a hydrolysate, which can be converted to bioTAL via bioconversion of both pentose and hexose sugars in engineered E. coli expressing the non-native BktB thiolase. The cost of production is captured by a single metric minimum selling price (MSP). MSP refers to the product selling price at which the net present value of the project equals zero, after incorporating an internal rate of return (10% for this study). In other words, the MSP is the minimum price a company must sell the product for to be as profitable as their next best investment option. To estimate the MSP and generate mass and energy balances, we used a combination of experimental data and chemical process modeling to design and simulate a hypothetical commercial-scale bioTAL production facility. All process simulation was conducted in a commercial process modeling software package (SuperPro Designer-V12). We translated outputs from the process model, including equipment sizing and costs, operating inputs, and waste processing costs, into a separate cash flow model to determine the MSP of bioTAL.

To understand the impact of bioTAL yield and other process parameters on MSP, we analyzed four biomass-derived bioTAL production scenarios based on the current demonstrated and future optimized yields. The scenario associated with “this work” reflects the bioTAL yield experimentally demonstrated in this study with glucose, extrapolated to xylose assuming a commercial-scale biorefinery would use a co-fermenting host. For comparison, we also considered a scenario built from the bioTAL yield in R. toruloides using 2-PS, as reported by Cao et al. Metabolic engineering of oleaginous yeast Rhodotorula toruloides for overproduction of triacetic acid lactone. Biotechnol. Bioeng. 119, 2529-2540 (2022). (FIG. 77, Panel B). The “intermediate” scenario extrapolates from this study with a moderate improvement upon the current bioTAL yield, reaching approximately 50% of the theoretical maximum (0.35 g bioTAL per g glucose, 0.315 g bioTAL per g xylose). Both “this work” and “intermediate” scenarios rely on a bioconversion residence time of approximately 75 h. An “optimized” scenario represents a mature facility in which the bioTAL yield reaches approximately 90% of theoretical maximum (0.63 g bioTAL per g glucose, 0.567 g bioTAL per g xylose) and all process parameters have been optimized to reach a practical minimum production cost, including a reduction in residence time to 48 h. Material balances for the intermediate scenario and major costs and revenues associated with bioTAL production are presented in Tables 12 & 13, respectively.

In biorenewable PDK production, bioTAL serves as a replacement to dimedone, a petrochemical priced at $10 per kg. As shown in FIG. 77, Panel C, all three scenarios tied to bioTAL production in E. coli using non-native BktB enzymes result in a lower MSP than the reported price for dimedone, whereas MSP is higher for the previously reported bioTAL production using 2-PS in R. toruloides. This encouraging result suggests that, provided the microbial host can be engineered to co-utilize both pentose and hexose sugars at comparable yields and rates, commercial-scale bioTAL production from corn stover can be cost-competitive with dimedone in the near term. The MSP results for the “optimized” scenario of approximately $2 per kg bioTAL represent a practical minimum and can be useful in screening for other applications where bioTAL may or may not compete with incumbent molecules. This optimized scenario can be viewed as something akin to a theoretical minimum; it is unlikely that costs could be reduced beyond that level. This exercise is useful because, if the optimized scenario were to result in higher costs than the petrochemical alternative (dimedone), this might suggest that bioTAL is not a viable replacement. For context, the price of HDPE, PU and PET is $2.3 per kg, $4 per kg, and $1.2 per kg, respectively. Using the optimized scenario, replacing dimedone with bioTAL would result in a cost lower than the previously published PDK cost. Improvements across all aspects of the production system, including lower-cost corn stover, improved sugar yields, higher ionic liquid recovery rates, and increases in titer, rate, and yield are needed to reach this ambitious target. In the near-term, further research can improve the bioTAL recovery process (e.g., bioTAL refinement via recrystallization, instead of chromatography), which will improve both the costs and energy use.

The life-cycle GHG assessment is based on a cradle-to-gate system boundary and the functional unit is defined as one kg of bioTAL produced. We obtained life-cycle inventory data and characterization factors for input materials and commodity polymers from peer-reviewed literature, and LCA databases including Ecoinvent, US Life Cycle Inventory (USLCI), GREET, and WARM models. The GHG emissions footprint for dimedone has been reported to be 0.7-15 kg CO₂e per kg dimedone in previous works. To contextualize our results, we use a median value of 8 kg CO₂e per kg of dimedone. The intermediate and optimized scenarios in FIG. 77, Panel D result in lower GHG emissions compared to dimedone. This result is encouraging, as it suggests that even with moderate improvement in current bioTAL yield for the intermediate scenario, keeping all other assumptions constant, commercial-scale bioTAL production from corn stover can result in lower GHG emissions when compared to dimedone in the near term. GHG emissions are higher for the previously reported bioTAL production using 2-PS in R. toruloides, when compared to dimedone. For “this work” scenario, we found that GHG emissions is 1.7 times higher than the median value, and approximately equal to the highest reported value of dimedone.

Lower product yields and long bioconversion residence times translate into higher energy use, which drives GHG emissions. If lignin recovered from biomass is sufficient to meet the facility's heat and electricity demands, no fossil fuels are directly required. Any excess electricity can be sold to the grid; we assume these exports offset the U.S. average grid mix. If lignin is not sufficient, supplemental natural gas is required for on-site combined heat and power generation. We found for the bioTAL production reported in “this work” scenario that the pretreatment process is the single largest contributor to life cycle GHG emissions (51%), followed by bioconversion (31%). Switching to non-fossil energy sources could drive down the GHG footprint. Another opportunity for GHG emissions reduction would be to improve the solvent recovery rate. We assume 95% solvent recovery (ethyl acetate, methanol), which is readily achieved at industrial scale.

Discussion Our summary findings show that biorenewable circularity with TAL-PDK is most promising when: bioprocesses for TAL production can be incorporated into lignocellulosic biorefineries that take in crop residues and other sustainable biomass feedstocks; engineered microorganisms metabolize both pentose and hexose sugars; and bioTAL yields are high. We also find that the high efficiency, low cost, and low-carbon intensity of PDK deconstruction and monomer recovery continues to stand out, even among emerging circular plastics. In particular, our use of TAL in place of petrochemicals in PDK production does not negatively impact PDK circularity. Instead, TAL provides an unexpected and useful bio-advantage with regard to the thermal behavior of TAL-PDK materials, which is exploited to expand the range of serviceable applications.

BioTAL production shows promise as a bio-advantaged alternative to dimedone in the formulation of biorenewable circular PDK resins. Even moderate improvements in yield can result in costs and life-cycle GHG emissions that are more competitive with the incumbent petrochemicals currently used in PDK production. However, large-scale production will require advancements along the entire supply chain to enable more efficient utilization of corn stover, including high sugar yields, the use of microbial hosts capable of metabolizing pentose and hexose sugars, and improvements in bioTAL yields. In the future, we anticipate that synthetic biology will play an increasingly important role in PDK development. PDK properties can be tailored by an interplay of structure and chirality in monomer designs. A wide variety of structurally diverse diacids and 1,3-diketones (i.e., beyond TAL) are, in principle, accessible as polyketide bioproducts, offering new targets for bioproduction (e.g., by engineered polyketide synthases). Depending on the process and the feedstock required, PDK sustainability may further benefit from these carbon-negative technologies.

Methods. Synthesis of Triketone Monomers. Triacetic acid lactone (2.1 eq), carboxylic diacid (1 eq), and dimethylaminopyridine (DMAP, 3 eq) were solubilized in tetrahydrofuran upon heating at 70° C. A separate solution of dicyclohexylcarbodiimide (DCC, 2.4 eq) in tetrahydrofuran was added slowly to the reaction mixture. The reaction mixture gradually turned yellow, accompanied by the formation of a white precipitate. After the complete addition of DCC, the reaction was allowed to cool to room temperature and pursued overnight (24 h). The mixture was filtered and washed with CH₂Cl₂ until the solid became colorless. The filtrate was concentrated and the recovered product, a dark red oil, was dissolved in CH₂Cl₂ and extracted twice with 2.0 M HCl. The organic phase was dried over MgSO₄ and concentrated, leaving the crude product as an orange paste. The crude product was recrystallized from ethanol/water to yield yellow/orange needles.

Synthesis of PDK Resins. The polymerization of PDK resins was realized using ball-milling. To the triketone monomer was added tris(2-aminoethyl)amine (TREN) using a pre-calibrated micropipette such that the molar ratio of amine to triketone functional groups is 1.1:1. This was immediately followed by ball-milling the contents of the closed container for 30 min at 500 rpm with changes in spinning direction in 1-min intervals. The reactor was opened to air and the reactor walls were scraped to bring together the reactants homogeneously. Ball-milling was resumed under identical conditions for an additional 30 min. The powders were recovered from the reactor and the residual water was removed under vacuum at 90° C.

Acid-Catalyzed Hydrolysis of PDK Samples. PDK materials were placed in separate 40 mL vials along with 5.0 M HCl (15 mL) and a magnetic stirrer. Depolymerization reactions were conducted over 24 h at room temperature while stirring at 500 rpm. Triketones were isolated by extraction with CH₂Cl₂ and evaporation of the organic phase.

Preparation of PDK Plastics for Hydrolysis. PDK resins obtained from ball-milling were compression-molded into sheets of ˜1 mm in thickness using a thermal press operating at 110° C. for TAL-PDK 5, 125° C. for TAL-PDK 4, 130° C. for TAL-PDK 3, 140° C. for TAL-PDK 2, and 150° C. for TAL-PDK 1 and 20,000 psi for 20 min. Small rectangular samples used for depolymerization studies were shaped with dimensions of l=20 mm, w=5 mm, t=1 mm.

Fed-batch fermentation for TAL production. The strain used for TAL production is E. coli JBEI-3695 harboring plasmid pBbA5a-bktB (jbei.org entry 20892). A single colony of the strain was inoculated into 10 ml of LB medium and grown overnight at 37° C. This seed culture (1 mL) was inoculated into LB (100 mL) in a 1-L shake flask and grown with shaking at 37° C. for 16 h, before inoculation into 1-L EZ-Rich medium (OD₆₀₀=0.05) in a 2-L bioreactor (Sartorius BIOSTAT B plus). Agitation, temperature, airflow, and pH were maintained constant at 300 rpm, 22° C., 0.5 vvm and pH 7.0, respectively. The culture was grown for 3-4 h at 37° C. to OD₆₀₀=0.6, at which point 0.1 mM IPTG was added to the culture to induce protein production. The temperature was adjusted to 22° C., and the culture was grown for 7 days. Fed-batch experiments employed a DO signal-triggered glucose feeding loop (ΔDO=15%, Flow rate=40 mL h⁻¹, Pump duration=5 min). 1 mL cell culture was removed every 24 h for cell density and TAL titer measurement. At the end of the 5-day culture, the cultures were harvested at 8,000 rpm, and TAL was extracted and purified from the supernatant.

System analysis of bioTAL production using SuperPro Model. The first step to conducting scenario analysis is establishing stoichiometrically maximum achievable yields. We calculated the stoichiometric maximum theoretical yield of bioTAL from glucose to be 0.7 g per g glucose. For all four scenarios considered in this study, we assumed xylose to bioTAL conversion to be 90% of that of glucose to bioTAL conversion. The scenario associated with “this work” reflects the bioTAL yield experimentally demonstrated in this study with glucose (14.9% of theoretical maximum yield), extrapolated to xylose assuming a commercial-scale biorefinery would use a co-fermenting host. A 2.6 g L⁻¹ titer was used to calculate the yield in glucose (0.104 g g⁻¹ glucose). Then, assuming xylose to bioTAL conversion to be 90% of that of glucose to bioTAL conversion, the yield from xylose to bioTAL conversion was also calculated. A 2.6 g L⁻¹ titer was used for the modeling purpose (instead of the highest reported titer of 2.77 g L⁻¹) as at 2.6 g L⁻¹, the temperature of the bioconversion units could be kept lower, which reduces the energy use of the system significantly, and thus lowers the MSP and the GHG emissions of the system. The intermediate and optimized scenario are based on the assumption of achieving 50% and 90% of theoretical yield based on glucose, respectively. The “Cao et al.” scenario is based on the bioTAL yield information obtained from article mentioned here (12.7% of theoretical maximum yield).

We conducted the process modeling in SuperPro Designer software. The biorefinery operates 330 days per year and 24 h per day (equivalent to 90% uptime). Capital cost accounts for equipment purchase cost, installation costs, warehouse, site development, permits, land, and other field expenses and project contingency costs. Annual operating cost accounts for materials, utilities, repair and maintenance, labor, and waste disposal costs. The assumptions for the model are consistent with Humbird et al. unless otherwise specified. The bulk prices for material costs were obtained from peer-reviewed literature, market price reports, and Alibaba. Equipment purchase prices were derived using built-in cost estimating function available in SuperPro. The process parameters and assumptions for “this work” and optimized scenarios are summarized in Table 13. With the exception of yield, all other process parameters remained the same for “this work” and intermediate scenarios. For the Cao et al. scenario, the same assumptions and parameters were used as that in “this work” scenario, except during bioconversion where we used a residence time and temperature of 120 h and 30° C., respectively, and during the recovery process, where we used crystallization to recover bioTAL instead of column chromatography.

Supplementary Methods. Materials. 4-Hydroxy-6-methyl-2-pyrone (Triacetic Acid Lactone, 98%), suberic acid (≥98%), azelaic acid (98%), dodecanedioic acid (99%), 4-dimethylaminopyridine (DMAP, ≥99%), N,N′-dicyclohexylcarbodiimide (DCC, 99%), and tris(2-aminoethyl)amine (TREN, 96%) were purchased from Sigma Aldrich. Bio-based sebacic acid (99%) was purchased from Arkema. 1,9-Nonanedicarboxylic acid (97%) was purchased from AmBeed. Anhydrous magnesium sulfate (MgSO₄, 99%) was purchased from Arcos Organics. Tetrahydrofuran (THF, ≥99.9%), dichloromethane (DCM, ≥99.9%), acetonitrile (ACN, >99.8%), ethanol (90%), hydrochloric acid (HCl, 36.5-38%), trifluoroacetic acid (>99.8%), and formic acid (98-100%) were purchased from VWR. Chloroform-d (CDCl₃, 99.8% D) was purchased from Cambridge Isotope Laboratories. All solvents and reagents were used without further purification.

LB broth (Miller), carbenicillin (100 mg mL⁻¹ in ethanol-water, 0.2-μm filtered), isopropyl β-D-1-thiogalactopyranoside (IPTG, >99%), dextrose (D-(+)-glucose), ethyl acetate (>99.7%), HPLC Water, sodium selenite (>98%) were purchased from Sigma Aldrich. MOPS EZ Rich Defined Medium Kit was purchased from Teknova, (M2105). The strain used for the fermentation is E. coli JBEI-3695 pBbA5a-bktB_Burk so.RF2-nonBP3 (registry.jbei.org/entry/148833).

Instrumentation. ¹H and ¹³C Solution State Nuclear Magnetic Resonance (NMR) Spectroscopy. ¹H and ¹³C solution state NMR spectroscopy was carried out using a Bruker Avance II at 500 and 125 MHz, respectively. Chemical shifts are reported relative to the residual solvent signal (¹H: δ=7.26 ppm (CDCl₃); ¹³C: δ=77.16 ppm (CDCl₃)). NMR data are reported as follows: chemical shift (multiplicity, coupling constants (where applicable), number of hydrogens). Splitting patterns are reported as s (singlet), d (doublet), t (triplet), and m (multiplet). All spectra were processed using Bruker TopSpin 4.1.1 software.

¹³C Solid-State Nuclear Magnetic Resonance (NMR) Spectroscopy. Solid-state ¹³C NMR spectra were carried out at 500.12 MHz (11.7 T) on a Bruker Avance spectrometer with a Bruker 4 mm narrow bore H/C/N magic angle spinning probe. Solid state ¹³C NMR spectra were in general acquired by cross-polarization from ¹H with a contact time of 5 ms at a spinning rate of 10 kHz. Adamantane was used as an external reference.

Electrospray Ionization Mass Spectrometry (ESI-MS). ESI-MS was carried out using a Bruker microTOF-Q mass spectrometer using an acetonitrile/H₂O (95:5 v/v) mixture containing 0.01% trifluoroacetic acid and 0.1% formic acid as the ionization medium.

Single Crystal X-Ray Diffraction (XRD). Single crystals for triketones TAL-TKs 1, 3, and 5 were selected, mounted on Mitegen loops with Paratone oil, and placed in an Oxford Cryosystems Cryostream 800 plus at T=100 K. Data were collected on beamline 12.2.1 at the Advanced Light Source (Berkeley, CA) with λ=0.7288 Å using a Bruker D8 diffractometer with a Bruker PHOTONII CPAD detector. Data reductions were performed and corrected for Lorentz and polarization effects using SAINT v8.40a and were corrected for absorption and other effects using TWINABS 2012/1. Structure solutions were performed by SHELXT using the direct method and were refined by least-square refinement against F2 by SHELXL.

Powder X-Ray Diffraction (PXRD). PXRD patterns were collected using a Rigaku MiniFlex 6G X-ray diffractometer with Cu K α radiation (λ=1.5418 Å). The voltage and current were set at 40 kV and 15 mA, respectively. The scanning rate was 10° min⁻¹ in the range of 2θ from 2°-60°.

Fourier Transform Infrared Spectroscopy (FT-IR). FT-IR data were collected on a Nicolet iS50 spectrophotometer using a built-in ATR. Data was reported as an average of 16 scans over an energy range of 400-4000 cm⁻¹.

Differential Scanning Calorimetry (DSC). DSC data were acquired using a TA Instruments Q200 Differential Scanning Calorimeter. TAL-PDK samples were heated over a temperature range of 0-200° C. at a rate of 10° C. min⁻¹ under a N₂ atmosphere. For each sample, data acquisition runs consisted of a heating step, a cooling step, and a second heating step. Glass transition temperatures (T_{g}) and melting temperatures (Tₘ) were interpreted and reported from the second heating curve.

Dynamic Mechanical Analysis (DMA). DMA data were acquired using a TA instruments DMA Q800 in tensile mode. All TAL-PDKs were fabricated as rectangular samples with dimensions of approximately l=20 mm, w=5 mm, t=1 mm. Each sample was tested at a frequency of 1 Hz with a displacement amplitude of 15 μm and a preload force of 0.01 N. Heating ramps of 3° C. min⁻¹ were applied from 40-200° C. The storage modulus and elastic modulus were reported at 40° C. and 180° C., respectively. The T_{g} was reported as the temperature at which the maximum value of tan δ was observed.

Thermogravimetric Analysis (TGA) was performed on a TA instruments TGA5500 Thermal Analyzer. Samples were heated under nitrogen at a rate of 10° C. min⁻¹ from 20 to 800° C.

### Synthesis of 1,10-bis(2-hydroxy-4,4-dimethyl-6-oxocyclohex-1-en-1-yl)decane-1,10-dione (Aliphatic Triketone)

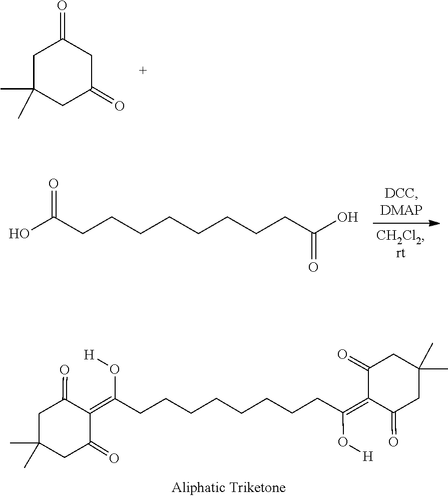

The synthesis of Aliphatic Triketone monomer was synthesized from dimedone and sebacic acid (C₁₀ diacid), according to a previously reported procedure.

### Synthesis of 1,8-bis(4-hydroxy-6-methyl-2-oxo-2H-pyran-3-yl)octane-1,8-dione (TAL-TK 1)

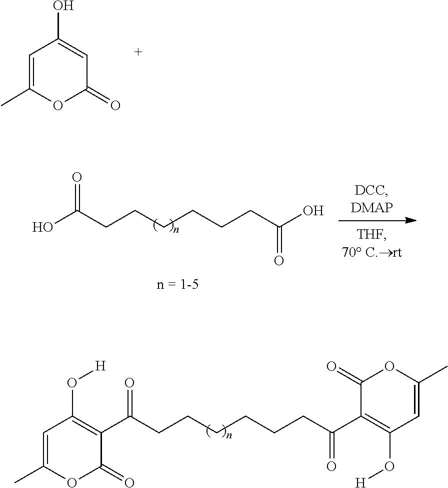

Triacetic acid lactone (10.10 g, 80.1 mmol), suberic acid (6.65 g, 38.2 mmol), and DMAP (14.04 g, 114.9 mmol) were solubilized in tetrahydrofuran (200 mL) upon heating at 70° C. A separate solution of DCC (18.65 g, 90.4 mmol) in tetrahydrofuran (50 mL) was added slowly to the reaction mixture. The reaction mixture gradually turned yellow, accompanied by the formation of a white precipitate. After the complete addition of DCC, the reaction was allowed to cool to room temperature and pursued overnight (24 h). The mixture was filtered and washed with CH₂Cl₂ until the solid became colorless. The filtrate was concentrated and the recovered product, a dark red oil, was dissolved in CH₂Cl₂ (400 mL) and extracted with 2 M HCl (2×150 mL). The organic phase was dried over MgSO₄ and concentrated, leaving the crude product as an orange paste. The crude product was recrystallized from ethanol to yield TAL-TK 1 as light orange needles (6.00 g, 15.4 mmol, 40.2%). ¹H NMR (500 MHz, CDCl₃): δ 16.84 (s, 2H), 5.92 (s, 2H), 3.05-3.08 (t, J=7.4 Hz, 4H), 2.26 (s, 6H), 1.65-1.67 (m, 4H), 1.40-1.43 (m, 4H) ppm; ¹³C NMR (125 MHz, CDCl₃): δ 207.99, 181.38, 168.94, 161.11, 101.66, 99.61, 41.70, 29.13, 23.88, 20.81 ppm; ESI-MS: m/z for (C₂₀H₂₂O₈)Na⁺ ([M+Na]⁺) calculated 413.1207, found 413.1350; FT-IR: 3076, 2951, 2860, 1711, 1643, 1601, 1540, 1451, 1421, 1377, 1354, 1313, 1237, 1164, 1027, 992, 972, 920, 855, 801, 781, 766, 711, 638, 582, 502, 484, 400 cm⁻¹. Crystallographic data for TAL-TK 1 is available free of charge from the Cambridge Crystallographic Date Centre under reference number 2223455.

Synthesis of 1,9-bis(4-hydroxy-6-methyl-2-oxo-2H-pyran-3-yl)nonane-1,9-dione (TAL-TK 2). Triacetic acid lactone (10.11 g, 80.2 mmol), azelaic acid (7.20 g, 38.3 mmol), and DMAP (14.01 g, 114.7 mmol) were solubilized in tetrahydrofuran (200 mL) upon heating at 70° C. A separate solution of DCC (18.63 g, 90.3 mmol) in tetrahydrofuran (50 mL) was added slowly to the reaction mixture. The reaction mixture gradually turned orange, accompanied by the formation of a white precipitate. After the complete addition of DCC, the reaction was allowed to cool to room temperature and pursued overnight (25 h). The mixture was filtered and washed with CH₂Cl₂ until the solid became colorless. The filtrate was concentrated and the recovered product, a dark red oil, was dissolved in CH₂Cl₂ (450 mL) and extracted with 2 M HCl (2×150 mL). The organic phase was dried over MgSO₄ and concentrated, leaving the crude product as a yellow paste. The crude product was co-recrystallized from ethanol and H₂O to yield TAL-TK 2 as a yellow powder (6.64 g, 16.4 mmol, 42.9%). ¹H NMR (500 MHz, CDCl₃): δ 16.85 (s, 2H), 5.92 (s, 2H), 3.04-3.07 (t, J=7.4 Hz, 4H), 2.26 (s, 6H), 1.63-1.65 (m, 4H), 1.36-1.39 (m, 6H) ppm; ¹³C NMR (125 MHz, CDCl₃): δ 208.08, 181.39, 168.92, 161.10, 101.66, 99.62, 41.73, 29.37, 29.16, 24.01, 20.78 ppm; ESI-MS: m/z for (C₂₁H₂₄O₈)Na⁺ ([M+Na]⁺) calculated 427.1364, found 427.1519; FT-IR: 3087, 2934, 2854, 1707, 1639, 1604, 1548, 1450, 1234, 992, 924, 773, 713, 639, 567, 502, 401 cm⁻¹.

Synthesis of 1,10-bis(4-hydroxy-6-methyl-2-oxo-2H-pyran-3-yl)decane-1,10-dione (TAL-TK 3). Triacetic acid lactone (10.05 g, 79.7 mmol), sebacic acid (7.71 g, 38.1 mmol), and DMAP (14.01 g, 114.7 mmol) were solubilized in tetrahydrofuran (200 mL) upon heating at 70° C. A separate solution of DCC (18.70 g, 90.6 mmol) in tetrahydrofuran (50 mL) was added slowly to the reaction mixture. The reaction mixture gradually turned orange, accompanied by the formation of a white precipitate. After the complete addition of DCC, the reaction was allowed to cool to room temperature and pursued overnight (24 h). The mixture was filtered and washed with CH₂Cl₂ until the solid became colorless. The filtrate was concentrated and the recovered product, a dark red oil, was dissolved in CH₂Cl₂ (450 mL) and extracted with 2 M HCl (2×150 mL). The organic phase was dried over MgSO₄ and concentrated, leaving the crude product as an orange paste. The crude product was recrystallized from ethanol to yield TAL-TK 3 as yellow-orange needles (6.48 g, 15.5 mmol, 40.6%). ¹H NMR (500 MHz, CDCl₃): δ 16.87 (s, 2H), 5.92 (s, 2H), 3.04-3.07 (t, J=7.4 Hz, 4H), 2.26 (s, 6H), 1.61-1.67 (m, 4H), 1.34-1.38 (m, 8H) ppm; ¹³C NMR (125 MHz, CDCl₃): δ 208.15, 181.40, 168.92, 161.13, 101.68, 99.63, 41.78, 29.41, 29.28, 24.04, 20.81 ppm; ESI-MS: m/z for (C₂₂H₂₆O₈)Na⁺ ([M+Na]⁺) calculated 441.1520, found 441.1350; FT-IR: 3076, 2930, 2856, 1710, 1641, 1603, 1543, 1455, 1421, 1335, 1285, 1234, 1165, 1024, 992, 918, 853, 776, 710, 637, 506, 431, 400 cm⁻¹. Crystallographic data for TAL-TK 3 is available free of charge from the Cambridge Crystallographic Date Centre under reference number 2223456.

Synthesis of 1,11-bis(4-hydroxy-6-methyl-2-oxo-2H-pyran-3-yl)undecane-1,11-dione (TAL-TK 4). Triacetic acid lactone (9.82 g, 77.9 mmol), 1,9-nonanedicarboxylic acid (8.03 g, 37.1 mmol), and DMAP (13.60 g, 111.3 mmol) were solubilized in tetrahydrofuran (200 mL) upon heating at 70° C. A separate solution of DCC (18.36 g, 89.0 mmol) in tetrahydrofuran (50 mL) was added slowly to the reaction mixture. The reaction mixture gradually turned orange, accompanied by the formation of a white precipitate. After the complete addition of DCC, the reaction was allowed to cool to room temperature and pursued overnight (24 h). The mixture was filtered and washed with CH₂Cl₂ until the solid became colorless. The filtrate was concentrated and the recovered product, a dark red oil, was dissolved in CH₂Cl₂ (600 mL) and extracted with 2 M HCl (2×150 mL). The organic phase was dried over MgSO₄ and concentrated, leaving the crude product as an orange paste. The crude product was co-recrystallized from ethanol and H₂O to yield TAL-TK 4 as a yellow granules (7.69 g, 17.8 mmol, 47.9%). ¹H NMR (500 MHz, CDCl₃): δ 16.87 (s, 2H), 5.92 (d, J=0.65 Hz, 2H), 3.04-3.07 (t, J=7.4 Hz, 4H), 2.26 (d, J=0.55 Hz, 6H), 1.60-1.66 (m, 4H), 1.30-1.35 (m, 10H) ppm; ¹³C NMR (125 MHz, CDCl₃): δ 208.17, 181.39, 168.91, 161.12, 101.67, 99.61, 41.78, 29.52, 29.43, 29.31, 24.04, 20.80 ppm; ESI-MS: m/z for (C₂₃H₂₈O₈)Na⁺ ([M+Na]⁺) calculated 455.1676, found 455.1831; FT-IR: 3094, 2926, 2857, 1711, 1643, 1600, 1549, 1456, 1423, 1388, 1385 1238, 1187, 1171, 1025, 994, 972, 919, 846, 775, 709, 640, 590, 506, 447 cm⁻¹.

Synthesis of 1,12-bis(4-hydroxy-6-methyl-2-oxo-2H-pyran-3-yl)dodecane-1,12-dione (TAL-TK 5). Triacetic acid lactone (10.15 g, 80.5 mmol), dodecanedioic acid (8.79 g, 38.2 mmol), and DMAP (13.94 g, 114.0 mmol) were dissolved in tetrahydrofuran (200 mL) under refluxing conditions at 70° C. A separate solution of DCC (17.78 g, 86.2 mmol) in tetrahydrofuran (100 mL) was added slowly to the reaction mixture. The reaction mixture gradually turned yellow, accompanied by the formation of a white precipitate. After the complete addition of DCC, the mixture was allowed to cool to room temperature and stirred overnight (23 h). The mixture was filtered and washed with CH₂Cl₂ until the solid became colorless. The filtrate was concentrated and the recovered product, an orange paste, was dissolved in CH₂Cl₂ (800 mL) and extracted with 2 M HCl (3×100 mL). The organic phase was dried over MgSO₄ and concentrated, leaving the crude product as an orange paste. The crude product was recrystallized from ethanol to yield TAL-TK 5 as yellow needles (10.58 g, 23.7 mmol, 62.1%). ¹H NMR (500 MHz, CDCl₃): δ 16.88 (s, 2H), 5.93 (d, J=0.65 Hz, 2H), 3.05-3.08 (t, J=7.4 Hz, 4H), 2.26 (d, J=0.5 Hz, 6H), 1.61-1.67 (m, 4H), 1.28-1.36 (m, 12H) ppm; ¹³C NMR (125 MHz, CDCl₃): δ 208.21, 181.41, 168.91, 161.14, 101.69, 99.63, 41.81, 29.57, 29.34, 24.07, 20.82 ppm; ESI-MS: m/z for (C24H30O8)Na⁺ ([M+Na]⁺) calculated 469.1833, found 469.1974; FT-IR: 2923, 2852, 1715, 1643, 1606, 1550, 1452, 1422, 1350, 1318, 1269, 1238, 1166, 1031, 993, 967, 919, 852, 768, 711, 640, 504, 416 cm⁻¹. Crystallographic data for TAL-TK 5 is available free of charge from the Cambridge Crystallographic Date Centre under reference number 2223457.

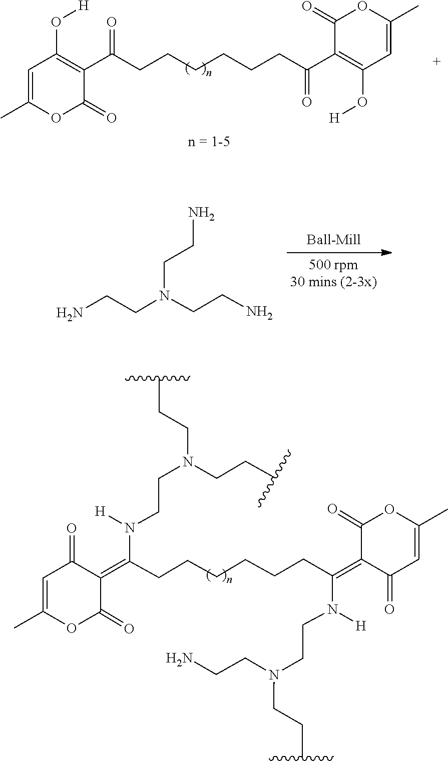

Synthesis of TAL-PDK Resins. Ball-milling was performed using a Retsch Planetary BallMill PM100. The container in which the reactions were carried out was a zirconium-coated cylinder either with an inner diameter of 4.5 cm and a height of 3.5 cm (reactor volume ˜50 mL) or with an inner diameter of 10 cm and a height of 7 cm (reactor volume ˜500 mL). All experiments reported herein used the same weight ratio of zirconium oxide ball bearings (5 mm diameter) to triketone monomer, with the ball bearings being 10 times the weight of triketone. The general procedure for all ball-milling reactions involved weighing out the appropriate amount of triketone monomer (2.0 or 10.0 g) and placing at the bottom of the ball mill, along with the ball bearings (20 or 100 g). To the triketone monomer was added tris(2-aminoethyl)amine (TREN) using a pre-calibrated micropipette such that the ratio of amine to triketone functional groups is 1.1 to 1. This was immediately followed by ball-milling the contents of the closed container for 30 min at 500 rpm with changes in spinning direction every 1 min. The reactor was opened to air and the reactor walls were scraped to bring together the reactants homogeneously. Ball-milling was resumed under identical conditions for an additional 30 min. For TAL-PDK 2, the walls of the reactor were scraped again, followed by one more round of ball-milling for 30 min. The powders were recovered from the reactor and the residual water was removed under vacuum at 90° C.

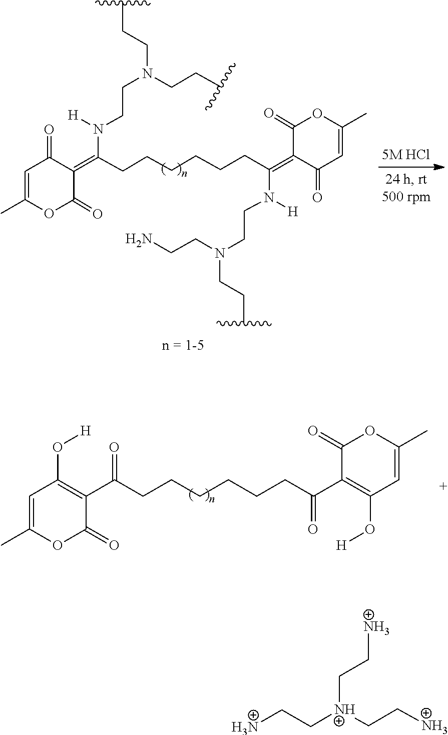

Acid-Catalyzed Hydrolysis of TAL-PDK Resins. TAL-PDK resins were placed in separate 40 mL vials along with 5.0 M HCl (15 mL) and a magnetic stirrer. Depolymerization reactions were conducted over 24 h at room temperature while stirring at 500 rpm. Triketones were isolated by extraction with CH₂Cl₂ and evaporation of the organic phase. Percent triketone recovery was calculated by the following equation:

$$Percent TK Recovery = \frac{TK ( g )}{\frac{PDK ( g )}{1 + x - \frac{2 ( {MW}_{H 2 O} )}{{MW}_{TK}}}} * 100 \%$$

where:
TK (g) is the mass of recovered triketone
PDK (g) is the mass of the TAL-PDK to be depolymerized
x is the mass ratio of TREN:Triketone used during TAL-PDK polymerization
MW_{H2O} is the molecular weight of H₂O
MW_{TK} is the molecular weight of the triketone

TAL-PDK 1 (472 mg) was completely depolymerized in aqueous 5.0 M HCl (15 mL) over 24 h at room temperature, yielding a light brown suspension. The mixture was extracted with CH₂Cl₂ (20 mL), and the organic layer was evaporated under vacuum to give an orange-brown paste. The product was dried under vacuum at 80° C. to yield TAL-TK 1 (yield=289 mg, 72.4%).

TAL-PDK 2 (320 mg) was completely depolymerized in aqueous 5.0 M HCl (15 mL) over 24 h at room temperature, yielding a light brown suspension. The mixture was extracted with CH₂Cl₂ (20 mL), and the organic layer was evaporated under vacuum to give a dark red solid. The product was dried under vacuum at 80° C. to yield TAL-TK 2 (yield=254 mg, 93.4%).

TAL-PDK 3 (487 mg) was completely depolymerized in aqueous 5.0 M HCl (15 mL) over 24 h at room temperature, yielding a dark yellow suspension. The mixture was extracted with CH₂Cl₂ (20 mL), and the organic layer was evaporated under vacuum to give a yellow-orange paste. The product was dried under vacuum at 80° C. to yield TAL-TK 3 (yield=376 mg, 90.3%).

TAL-PDK 4 (303 mg) was completely depolymerized in aqueous 5.0 M HCl (15 mL) over 24 h at room temperature, yielding a dark orange suspension. The mixture was extracted with CH₂Cl₂ (20 mL), and the organic layer was evaporated under vacuum to give a dark red solid. The product was dried under vacuum at 80° C. to yield TAL-TK 4 (yield=256 mg, 98.4%).

TAL-PDK 5 (527 mg) was completely depolymerized in aqueous 5.0 M HCl (15 mL) over 24 h at room temperature, yielding a brown suspension. The mixture was extracted with CH₂Cl₂ (20 mL), and the organic layer was evaporated under vacuum to give a yellow-brown paste. The product was dried under vacuum at 80° C. to yield TAL-TK 5 (yield=455 mg, 100%).

Preparation of TAL-PDK Plastics for Hydrolysis. TAL-PDK resins obtained from ball-milling were pressed into sheets of ˜1 mm in thickness using a thermal press operating at 110° C. for TAL-PDK 5, 125° C. for TAL-PDK 4, 130° C. for TAL-PDK 3, 140° C. for TAL-PDK 2, and 150° C. for TAL-PDK 1 and 20,000 psi for 20 min. Small rectangular samples used for depolymerization studies were shaped with dimensions of l=20 mm, w=5 mm, t=1 mm.

Acid-Catalyzed Hydrolysis of TAL-PDK Plastics. Plastic TAL-PDK samples were each placed in 40 mL vials containing 5.0 M HCl (15 mL) and a magnetic stirrer. Depolymerization reactions were conducted over 24 h at room temperature while stirring at 500 rpm. Crude triketones were isolated by extraction with CH₂Cl₂ and evaporation of the organic phase. Purified triketones were obtained by recrystallization in ethanol. Percent triketone recovery was calculated by the same equation as for TAL-PDK resin depolymerization.

TAL-PDK 1 (491 mg) was completely depolymerized in aqueous 5.0 M HCl (15 mL) over 24 h at room temperature, yielding a yellow-brown suspension. The mixture was extracted with CH₂Cl₂ (20 mL), and the organic layer was evaporated under vacuum to give a yellow-brown paste (crude yield=293 mg, 70.6%). The crude product was recrystallized from ethanol and dried under vacuum at 80° C. to yield pure TAL-TK 1 as a pale orange powder (yield=182 mg, 43.8%).

TAL-PDK 2 (539 mg) was completely depolymerized in aqueous 5.0 M HCl (15 mL) over 24 h at room temperature, yielding a brown suspension. The mixture was extracted with CH₂Cl₂ (20 mL), and the organic layer was evaporated under vacuum to give a dark red solid (crude yield=377 mg, 82.3%). The crude product was recrystallized from ethanol and dried under vacuum at 80° C. to yield pure TAL-TK 2 as brown granules (yield=206 mg, 45.0%).

TAL-PDK 3 (527 mg) was completely depolymerized in aqueous 5.0 M HCl (15 mL) over 24 h at room temperature, yielding a yellow-brown precipitate. The mixture was extracted with CH₂Cl₂ (20 mL), and the organic layer was evaporated under vacuum to give a yellow-brown paste (crude yield=437 mg, 97.1%). The crude product was recrystallized from ethanol and dried under vacuum at 80° C. to yield pure TAL-TK 3 as pale orange crystals (yield=302 mg, 67.0%).

TAL-PDK 4 (469 mg) was completely depolymerized in aqueous 5.0 M HCl (15 mL) over 24 h at room temperature, yielding a dark yellow precipitate. The mixture was extracted with CH₂Cl₂ (20 mL), and the organic layer was evaporated under vacuum to give a red-brown solid (crude yield=409 mg, 101.7%). The crude product was recrystallized from ethanol and dried under vacuum at 80° C. to yield pure TAL-TK 4 as a dark orange powder (yield=198 mg, 49.3%).

TAL-PDK 5 (564 mg) was completely depolymerized in aqueous 5.0 M HCl (15 mL) over 24 h at room temperature, yielding a yellow precipitate. The mixture was extracted with CH₂Cl₂ (20 mL), and the organic layer was evaporated under vacuum to give a dark yellow paste (crude yield=546 mg, 112.4%). The crude product was recrystallized from ethanol and dried under vacuum at 80° C. to yield TAL-TK 5 as dark yellow needles (yield=427 mg, 87.8%).

Quantification of TAL production. 100 μL cell culture was mixed with 100 μL acetonitrile before the analysis. This sample was filtered (AcroPrep Advance Filter Plates for Ultrafiltration—350 μL, Omega 3K MWCO) at 4,000×g for 1 h. 5 μL filtered sample was injected into the HPLC (Agilent Technologies) with a Diode-Array Detector (Agilent, G1315D) with a reverse phase column (Kinetex 5 μm EVO C18, 150×4.6 mm) for analysis. The starting mobile phase was 20% methanol, which increased to 72% in 6.5 min. It was further increased to 95% of methanol from 6.5 to 7.8 min. 95% methanol was maintained for 1 min to 8.8 min and decreased to 20% at 9 min. 20% methanol was maintained until 11.2 min. 0.1% formic acid was added in the mobile phase. TAL standards were prepared at 0.0625, 0.125, 0.25, 0.5, 1 g L⁻¹ under the same conditions for making the standard curve. The R square of the linear standard curve is >0.99.

Purification of TAL. The pH of the 1-L broth supernatant was adjusted to pH<2 with HCl and extracted with 3×2 L ethyl acetate. The organic phases were separated and combined to 2 L extraction. This extraction was concentrated to 20 mL in rotavapor. Column chromatography was applied on CombiFlash NEXTGEN 300+ with a silica column (RediSep Rf Gold Silica Gel Disposable columns, Cat. No. 69-2203-349) to purify TAL. All fractions containing TAL were combined and dried. NMR is used to determine the purity of TAL, which was >99%.

Techno-Economic Analysis and Life Cycle Assessment of bioTAL Production System. Biomass-to-TAL production process models used in this study were developed in a commercial process modeling software package—SuperPro Designer-V12. The process parameters and assumptions for “this work” and optimized scenarios are shown in Supplementary Table 11. Additionally, description and major assumptions for each of the major biomass-to-TAL production system are presented below. For the intermediate and Cao et al. scenarios, the same assumptions and parameters were used as that used for the “this work” scenario, unless otherwise noted in relevant sections below.

Feedstock handling. Corn stover was assumed as a representative biomass feedstock for the simulated biorefinery. The biorefinery utilizes 2000 bone-dry metric tons of corn stover per day. The feedstock handling process includes transportation from farm to refinery with shipping distance of 31 miles (50 km) and a handling dome. The milled biomass is routed to the biomass deconstruction unit for pretreatment and enzymatic hydrolysis. The greenhouse gas (GHG) emissions for the feedstock handling and supply of corn stover was assumed to be 83.8 kg CO₂e per metric ton of stover.

### Corn Stover Composition

| Corn stover composition | wt % |
|---|---|
| Acetate (bound) | 1.81 |
| Ash | 4.93 |
| Cellulose | 35.05 |
| Hemicellulose | 19.53 |
| Lignin | 15.76 |
| Other solids | 22.93 |

Biomass Deconstruction. The biomass undergoes pre-treatment to break the cell wall made of recalcitrant lignin and that aids in further enzymatic hydrolysis to convert glucose into cellulose. Here, bio-based ionic liquid (IL) cholinium lysinate [Ch][Lys] is chosen as preferred option for pre-treatment. The biomass deconstruction stage includes pretreatment, enzymatic hydrolysis, solid-liquid separation, and ionic liquid recovery units. The biomass solids loading rate is maintained at 30% by supplying additional water to the biomass and IL mixture. The water is added to ensure better heat and mass transfer when utilizing a high solid loading rate and IL mixture. For all scenarios except optimistic scenario, the IL loading rate is maintained at 0.29 kg per kg of bone-dry corn stover feedstock. High IL recovery of 97% (99% for optimistic scenario) is assumed, where IL is recovered post bioconversion through pervaporation technology. The lignin fraction on cellulose can inhibit enzyme accessible areas and overall sugar yields. Treatment with [Ch][Lys] rectifies this and results in dissolution of 31% (17% for optimistic scenario) of lignin fraction. The pretreated biomass is sent to the enzymatic hydrolysis unit after pH adjustment using sulfuric acid.

Enzymatic Hydrolysis. Enzymatic hydrolysis releases fermentable sugars, including glucose from cellulose and xylose from xylan. Cellulose to glucose conversion is modeled at 84% and xylan to xylose conversion is considered to be 80%. The enzyme loading rate is maintained at 20 mg-protein/g-celluose. Initial solids loading is at 20 wt %. Enzymatic hydrolysis is operated at a temperature of 48° C. for 72 h. The liquid fraction consisting of glucose and xylose is sent to the bioconversion unit. The solid fraction primarily consisting of lignin is sent to on-site combustor for energy generation. The slurry is cooled to 32° C. for bioconversion with a heat exchanger.

Bioconversion. The currently modeled bioconversion process uses E. coli. The bioconversion time is assumed to be 74.5 h. Xylose to bioTAL conversion is assumed to be 90% of that of glucose to bioTAL conversion. The fermenter requires 10 vol % of inoculum, and the ratio is maintained by sending 10% of the slurry from enzymatic hydrolysis to the seed fermenters, and the rest to the main bioconversion tank. The seed bioconversion consists of five reactors and designed with the modeling assumptions. For both seed bioconversion and main bioconversion unit, the nutrient source is assumed to be corn steep liquor (CSL) and diammonium phosphate (DAP). CSL and DAP are used as a placeholder for a low-cost source of providing nitrogen, phosphorus, and other trace minerals and are not actually used in laboratory experiments. However, for production at scale, an alternate source of nutrients could be explored, which may be separate from both modeled source and media used in experiments. Therefore, in future it is possible to lower the cost of media and obtain consistent yields. For the Cao et al. scenario, the bioconversion time and temperature are assumed to be 120 h and 30° C., respectively.

Product Separation and Recovery. BioTAL production is extracellular, and therefore the first step in product separation and recovery is separation through solids (cell biomass) from liquid (supernatant). Here, separation is modeled through a centrifuge, and the cell biomass solids are sent to the boiler for co-firing for heat and electricity generation. The supernatant stream undergoes solvent extraction using ethyl acetate. The stream is subsequently treated with sulfuric acid to reduce the pH to 2. The acidification is followed by second round of solvent extraction with ethyl acetate to extract bioTAL with limited contaminants. The ethyl acetate is recovered through distillation. The stream with bioTAL product undergoes drying using a drum (to simulate vacuum evaporation) and then column chromatography, to obtain a product with purity greater than 95%. Methanol used for column chromatography is also recovered through distillation. For both ethyl acetate and methanol used, a 95% of solvent recovery rate is assumed. For the scenario using yield data from Cao et al., crystallization is used to recover bioTAL instead of column chromatography.

Onsite Energy Generation. The onsite energy (heat and power) generation relies on a combination of unutilized biomass and supplemental natural gas (when needed). Any excess energy is sold to the grid for a credit based on average U.S. electricity prices. While solvent recovery and recycling increases the facility's on-site energy consumption, the cost associated with hazardous waste management is reduced by maximizing solvent recycling. The process model accounts for waste heat recovery.

### Figure Captions for Example 27.

FIG. 74. Biorenewable circularity in PDK plastics derived from triacetic acid lactone (TAL). a, Synthesis and chemical recycling of biorenewable PDK resins derived from TAL (TAL-PDKs 1-5). b, Single-crystal X-ray structures of triketone TAL-TK 3 (top) and a related aliphatic triketone prepared from the petrochemical dimedone in place of TAL (bottom). c, Compression-molded samples of TAL-PDKs 1-5. d, Glass transition temperatures (T_{g}) measured by DSC for TAL-PDK 1-5 and a related aliphatic PDK resin prepared from dimedone. e, Density and f, storage modulus (at rubbery state, 180° C.) of compression-molded TAL-PDK 1-5 and a related aliphatic PDK prepared from dimedone.

FIG. 75. Recycling of TAL-PDK formulations. a, Acid-catalyzed depolymerization of TAL-PDK 1 plastic and recovery of TAL-TK 1 monomer. b, ¹H NMR spectra of pristine TAL-TK 1 (top) along with crude TAL-TK 1 recovered from chemically-recycled TAL-PDK 1 resin (bottom). c, TAK-TK yields after acidolysis of TAL-PDK resins. d, ESI-MS spectrum of TAL-TK 1. e, ¹H NMR spectra of pristine TAL-TK 1 (top), crude TAL-TK 1 recovered after acidolysis of thermally-processed TAL-PDK 1 (middle), and TAL-TK 1 recovered from the crude after recrystallization in EtOH (bottom). Characteristic peaks in the ¹H NMR spectra for important structural motifs are highlighted with red arrows, while impurities are identified by using purple asterisks (*). f, TAL-TK yields for both crude (purple) and recrystallized (green) monomers after acidolysis of TAL-PDK plastics. g, ESI-MS spectrum of crude TAL-TK 1 recovered after acidolysis of thermally-processed TAK-PDK 1.

FIG. 76. Biosynthesis of triacetic acid lactone (bioTAL) and biorenewable TAL-PDK characterization. a, Metabolic pathway for TAL biosynthesis in engineered E. coli JBEI-3695. b, Cell growth (OD₆₀₀), TAL titer, and glucose concentration in the 1-L fed-batch fermentation using E. coli JBEI-3695 pBbA5A-BktB. c, Closed-loop production and chemical recycling of biorenewable TAL-PDK 3. d, Comparison of glass transition temperatures for petrochemical and biorenewable TAL-PDK 3, as well as a similar aliphatic PDK material produced from dimedone in place of BioTAL. Abbreviations: TCA, tricarboxylic acid cycle; DHAP, dihydroxyacetone phosphate.

FIG. 77. Systems analysis of the production of bioTAL. a, Simplified schematic system boundary. b, bioTAL annual production and yield under four different scenarios, adjusting for factors such as the product yield (Cao et al.: 12.7% of theoretical maximum, This Work: 14.9% of theoretical maximum, Intermediate: 50% of theoretical maximum, and Optimized: 90% of theoretical maximum) and projected efficiencies gained at industrial scale. c, Minimum selling price (MSP) and d, Life cycle GHG emissions for bioTAL production across all four scenarios.

FIG. 78. Single-crystal XRD. a, TAL-TK 1. b, TAL-TK 3. c, TAL-TK 5. Crystallographic data for compounds TAL-TK 1, TAL-TK 3 and TAL-TK 5 are available free of charge from the Cambridge Crystallographic Date Centre under reference numbers 2223455, 2223456 and 2223457, respectively.

FIG. 79. Single-crystal XRD. a, TAL-TK 1. b, TAL-TK 5. Crystallographic data for compounds TAL-TK 1 and TAL-TK 5 are available free of charge from the Cambridge Crystallographic Date Centre under reference numbers 2223455 and 2223457, respectively.

FIG. 80. DSC of TAL-TK 1-5.

FIG. 81. Solid-state ¹³C NMR spectra of TAL-TK 1 and TAL-PDK 1.

FIG. 82. Solid-state ¹³C NMR spectra of TAL-TK 2 and TAL-PDK 2.

FIG. 83. Solid-state ¹³C NMR spectra of TAL-TK 3 and TAL-PDK 3.

FIG. 84. Solid-state ¹³C NMR spectra of TAL-TK 4 and TAL-PDK 4.

FIG. 85. Solid-state ¹³C NMR spectra of TAL-TK 5 and TAL-PDK 5.

FIG. 86. PXRD of TAL-TK 1-5 and TAL-PDK 1-5.

FIG. 87. Processing of TAL-PDK resins into solid bar samples.

FIG. 88. DSC of TAL-PDK 1-5.

FIG. 89. TGA of TAL-TK 1-5 and powder and pressed TAL-PDK 1-5.

FIG. 90. DMA of TAL-PDK 1-5 and Aliphatic PDK.

FIG. 91. ¹H NMR of TAL-TK 1 recovered from depolymerized TAL-PDK 1 resin (top) and original TAL-TK 1 monomer (bottom).

FIG. 92. ¹H NMR of TAL-TK 2 recovered from depolymerized TAL-PDK 2 resin (top) and original TAL-TK 2 monomer (bottom).

FIG. 93. ¹H NMR of TAL-TK 3 recovered from depolymerized TAL-PDK 3 resin (top) and original TAL-TK 3 monomer (bottom).

FIG. 94. ¹H NMR of TAL-TK 4 recovered from depolymerized TAL-PDK 4 resin (top) and original TAL-TK 4 monomer (bottom).

FIG. 95. ¹H NMR of TAL-TK 5 recovered from depolymerized TAL-PDK 5 resin (top) and original TAL-TK 5 monomer (bottom).

FIG. 96. ¹H NMR spectra of purified TAL-TK 1 recovered from depolymerized TAL-PDK 1 compression-molded plastics (top), crude TAL-TK 1 recovered from depolymerized TAL-PDK 1 compression-molded plastics (middle), and original TAL-TK 1 monomer (bottom). Notable impurities caused by parasitic side reactions are identified by asterisks (*).

FIG. 97. ¹H NMR spectra of purified TAL-TK 2 recovered from depolymerized TAL-PDK 2 compression-molded plastics (top), crude TAL-TK 2 recovered from depolymerized TAL-PDK 2 compression-molded plastics (middle), and original TAL-TK 2 monomer (bottom). Notable impurities caused by parasitic side reactions are identified by asterisks (*).

FIG. 98. ¹H NMR spectra of purified TAL-TK 2 recovered from depolymerized TAL-PDK 2 compression-molded plastics (top), crude TAL-TK 2 recovered from depolymerized TAL-PDK 2 compression-molded plastics (middle), and original TAL-TK 2 monomer (bottom) zoomed in on 5.8-6.3 ppm and 16.7-17.0 ppm. Notable impurities caused by parasitic side reactions are identified by asterisks (*).

FIG. 99. ¹H NMR spectra of purified TAL-TK 3 recovered from depolymerized TAL-PDK 3 compression-molded plastics (top), crude TAL-TK 3 recovered from depolymerized TAL-PDK 3 compression-molded plastics (middle), and original TAL-TK 3 monomer (bottom). Notable impurities caused by parasitic side reactions are identified by asterisks (*).

FIG. 100. ¹H NMR spectra of purified TAL-TK 3 recovered from depolymerized TAL-PDK 3 compression-molded plastics (top), crude TAL-TK 3 recovered from depolymerized TAL-PDK 3 compression-molded plastics (middle), and original TAL-TK 3 monomer (bottom) zoomed in on 2.2-2.9 ppm and 16.7-17.0 ppm. Notable impurities caused by parasitic side reactions are identified by asterisks (*).

FIG. 101. ¹H NMR spectra of purified TAL-TK 4 recovered from depolymerized TAL-PDK 4 compression-molded plastics (top), crude TAL-TK 4 recovered from depolymerized TAL-PDK 4 compression-molded plastics (middle), and original TAL-TK 4 monomer (bottom). Notable impurities caused by parasitic side reactions are identified by asterisks (*).

FIG. 102. ¹H NMR spectra of purified TAL-TK 4 recovered from depolymerized TAL-PDK 4 compression-molded plastics (top), crude TAL-TK 4 recovered from depolymerized TAL-PDK 4 compression-molded plastics (middle), and original TAL-TK 4 monomer (bottom) zoomed in on 5.8-6.3 ppm and 16.7-17.0 ppm. Notable impurities caused by parasitic side reactions are identified by asterisks (*).

FIG. 103. ¹H NMR spectra of purified TAL-TK 5 recovered from depolymerized TAL-PDK 5 compression-molded plastics (top), crude TAL-TK 5 recovered from depolymerized TAL-PDK 5 compression-molded plastics (middle), and original TAL-TK 5 monomer (bottom). Notable impurities caused by parasitic side reactions are identified by asterisks (*).

FIG. 104. ¹H NMR spectra of purified TAL-TK 5 recovered from depolymerized TAL-PDK 5 plastic (top), crude TAL-TK 5 recovered from depolymerized TAL-PDK 5 plastic (middle), and original TAL-TK 5 monomer (bottom) zoomed in on 2.2-2.9 ppm and 16.7-17.0 ppm. Notable impurities caused by parasitic side reactions are identified by asterisks (*).

FIG. 105. ESI-MS spectra of crude recovered TAL-TK 1 from depolymerized TAL-PDK 1 compression-molded plastics (top) and original TAL-TK 1 monomer (bottom). The prevalent peak corresponding to a decarboxylated subproduct is identified.

FIG. 106. ESI-MS spectra of crude recovered TAL-TK 2 from depolymerized TAL-PDK 2 compression-molded plastics (top) and original TAL-TK 2 monomer (bottom). The prevalent peak corresponding to a decarboxylated subproduct is identified.

FIG. 107. ESI-MS spectra of crude recovered TAL-TK 3 from depolymerized TAL-PDK 3 compression-molded plastics (top) and original TAL-TK 3 monomer (bottom). The prevalent peak corresponding to a decarboxylated subproduct is identified.

FIG. 108. ESI-MS spectra of crude recovered TAL-TK 4 from depolymerized TAL-PDK 4 compression-molded plastics (top) and original TAL-TK 4 monomer (bottom). The prevalent peak corresponding to a decarboxylated subproduct is identified.

FIG. 109. ESI-MS spectra of crude recovered TAL-TK 5 from depolymerized TAL-PDK 5 compression-molded plastics (top) and original TAL-TK 5 monomer (bottom). The prevalent peak corresponding to a decarboxylated subproduct is identified.

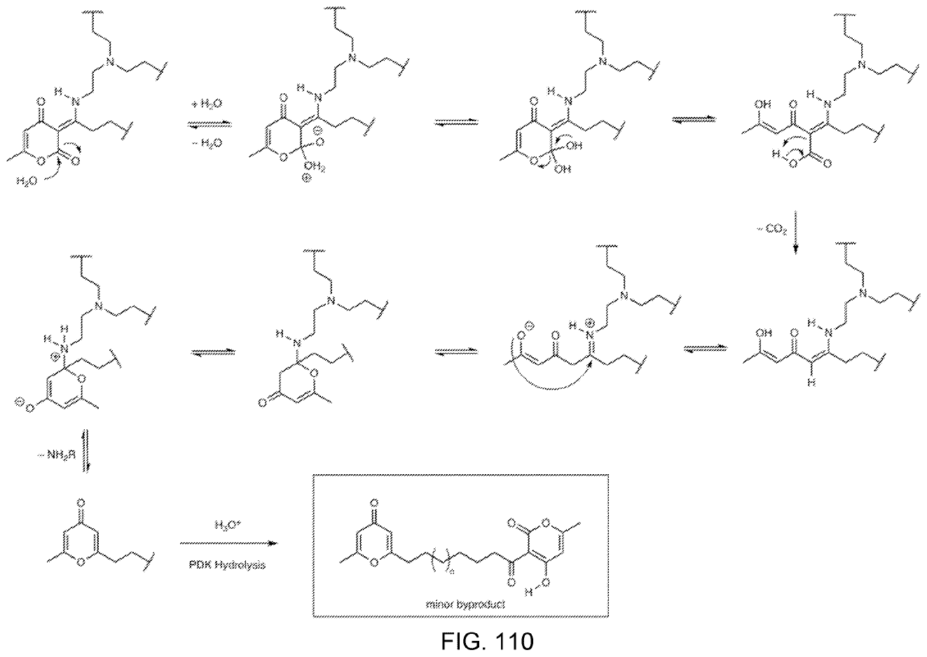

FIG. 110. Mechanistic hypothesis of TAL-PDK degradation leading to pyrone-triketone monomer.

FIG. 111. 1-L fed batch fermentation of TAL production with E. coli JBEI-3695 pBbA5A-BktB. Cell growth (OD₆₀₀), TAL titer and glucose concentration during the 120-h production run.

FIG. 112. Dual-wavelength optical densities of eluent acquired during TAL purification by column chromatography. TAL was eluted at 8.5-9.7 min (i.e., the major component of the extracted material post-fermentation). Flow Rate: 110 mL min⁻¹, Solvent A: Hexane, Solvent B: Ethyl Acetate, Wavelength 1 (red): 254 nm, Wavelength 2 (purple): 280 nm.

TABLE 5
Selected O—H bond distances and calculated crystal density extracted from X-
ray crystallography data for triketones TAL-TK 1, 3, and 5 as well as a dimedone-derived TAL-
TK reference

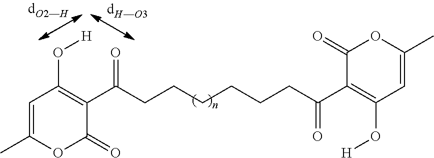

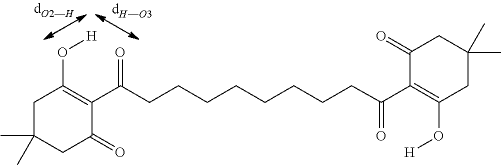

| Triketone | d_{O2—H}(Å) | d_{H—O3} (Å) | Crystal ρ (mg m⁻³) |
|---|---|---|---|
| TAL-TK 1 (n = 1) | 1.03 | 1.47 | 1.476 |
| TAL-TK 3 (n = 3) | 0.99 | 1.50 | 1.426 |
| TAL-TK 5 (n = 5) | 1.03 | 1.45 | 1.354 |
| Dimedone-derived TK | 1.08 | 1.38 | 1.270 |

TABLE 6
Densities of TAL-PDK 1-5 and a dimedone-derived PDK reference were
determined by displacement via immersion of solid samples (~250
mg) in 5-mL volumetric flasks filled with water at 18° C.

| | ρ (g mL⁻¹) at 18° C. |
|---|---|
| TAL-PDK 1 | 1.129 |
| TAL-PDK 2 | 1.011 |
| TAL-PDK 3 | 1.078 |
| TAL-PDK 4 | 1.032 |
| TAL-PDK 5 | 1.086 |
| Dimedone-derived PDK | 0.987 |

TABLE 7
Summary of thermo-mechanical data for PDK plastics
collected by DMA. T_{g} measured with tan δ (DMA) is
also compared with the T_{g} measured by DSC.

| | Tensile Storage | Tensile Elastic | | |
|---|---|---|---|---|
| | Modulus | Modulus | T_{g} | T_{g} |
| | at 40° C. | at 180° C. | (DMA) | (DSC) |
| | (GPa) | (MPa) | (° C.) | (° C.) |
| TAL-PDK 1 | 2.78 | 14.8 | 155 | 151 |
| TAL-PDK 2 | 2.02 | 12.7 | 147 | 141 |
| TAL-PDK 3 | 2.06 | 13.8 | 136 | 132 |
| TAL-PDK 4 | 2.04 | 9.6 | 110 | 125 |
| TAL-PDK 5 | 1.76 | 10.7 | 108 | 114 |
| Dimedone- | 2.08 | 3.21 | 108 | 96 |
| derived PDK | | | | |

TABLE 8
Crystal data and structure refinement for TAL-TK 1.

| Identification code | TAL-TK 1 | |
|---|---|---|
| Empirical formula | C20 H22 O8 | |
| Formula weight | 390.37 | |
| Temperature | 100(2) K | |
| Wavelength | 0.7288 Å | |
| Crystal system | Monoclinic | |
| Space group | P2₁/n | |
| Unit cell dimensions | a = 4.7046(3) Å | a = 90°. |
| | b = 29.1012(15) Å | b = 97.232(2)°. |
| | c = 6.4669(3) Å | g = 90°. |

| | |
|---|---|
| Volume | 878.34(8) Å³ |
| Z | 2 |
| Density (calculated) | 1.476 Mg/m³ |
| Absorption coefficient | 0.120 mm⁻¹ |
| F(000) | 412 |
| Crystal size | 0.140 × 0.080 × 0.010 mm³ |
| Theta range for data collection | 2.871 to 33.558°. |
| Index ranges | −7 <= h <= 7, −44 <= k <= 44, −9 <= |
| | l <= 9 |
| Reflections collected | 30567 |
| Independent reflections | 3200 [R(int) = 0.0348] |
| Completeness to theta = 25.930° | 99.9% |
| Absorption correction | Semi-empirical from equivalents |
| Max. and min. transmission | 0.999 and 0.913 |
| Refinement method | Full-matrix least-squares on F² |
| Data/restraints/parameters | 3200/0/132 |
| Goodness-of-fit on F² | 1.055 |
| Final R indices [I > 2sigma(I)] | R1 = 0.0392, wR2 = 0.1075 |
| R indices (all data) | R1 = 0.0479, wR2 = 0.1141 |
| Extinction coefficient | n/a |
| Largest diff. peak and hole | 0.395 and −0.194 e · Å⁻³ |

TABLE 9
Crystal data and structure refinement for TAL-TK 3.

| Identification code | TAL-TK 3 | |
|---|---|---|
| Empirical formula | C₂₂ H₂₆ O₈ | |
| Formula weight | 418.43 | |
| Temperature | 100(2) K | |
| Wavelength | 0.7288 Å | |
| Crystal system | Monoclinic | |
| Space group | P2₁/n | |
| Unit cell dimensions | a = 4.7597(2) Å | a = 90°. |
| | b = 32.0911(14) Å | b = 97.147(2)°. |
| | c = 6.4284(3) Å | g = 90°. |

| | |
|---|---|
| Volume | 974.27(7) Å³ |
| Z | 2 |
| Density (calculated) | 1.426 Mg/m³ |
| Absorption coefficient | 0.114 mm⁻¹ |
| F(000) | 444 |
| Crystal size | 0.100 × 0.090 × 0.010 mm³ |
| Theta range for data collection | 2.603 to 29.974°. |
| Index ranges | −6 <= h <= 6, −43 <= k <= 43, −8 <= |
| | l <= 8 |
| Reflections collected | 15447 |
| Independent reflections | 2640 [R(int) = 0.0363] |
| Completeness to theta = 25.930° | 99.9% |
| Absorption correction | Semi-empirical from equivalents |
| Max. and min. transmission | 0.999 and 0.909 |
| Refinement method | Full-matrix least-squares on F² |
| Data/restraints/parameters | 2640/0/141 |
| Goodness-of-fit on F² | 1.077 |
| Final R indices [I > 2sigma(I)] | R1 = 0.0365, wR2 = 0.0945 |
| R indices (all data) | R1 = 0.0447, wR2 = 0.1002 |
| Extinction coefficient | n/a |
| Largest diff. peak and hole | 0.351 and −0.192 e · Å⁻³ |

TABLE 10
Crystal data and structure refinement for TAL-TK 5.

| Identification code | TAL-TK 5 | |
|---|---|---|
| Empirical formula | C₂₄ H₃₀ O₈ | |
| Formula weight | 446.48 | |
| Temperature | 100(2) K | |
| Wavelength | 0.7288 Å | |
| Crystal system | Monoclinic | |
| Space group | P2₁/c | |
| Unit cell dimensions | a = 17.1106(11) Å | a = 90°. |
| | b = 4.8661(3) Å | b = 108.440(2)°. |
| | c = 13.8696(9) Å | g = 90°. |

| | |
|---|---|
| Volume | 1095.52(12) Å³ |
| Z | 2 |
| Density (calculated) | 1.354 Mg/m³ |
| Absorption coefficient | 0.106 mm⁻¹ |
| F(000) | 476 |
| Crystal size | 0.310 × 0.020 × 0.010 mm³ |
| Theta range for data collection | 2.573 to 31.397° |
| Index ranges | −24 <= h <= 24, −6 <= k <= 6, −19 <= |
| | l <= 19 |
| Reflections collected | 35253 |
| Independent reflections | 3335 [R(int) = 0.0416] |
| Completeness to theta = 25.930° | 99.8% |
| Absorption correction | Semi-empirical from equivalents |
| Max. and min. transmission | 0.999 and 0.907 |
| Refinement method | Full-matrix least-squares on F² |
| Data/restraints/parameters | 3335/0/150 |
| Goodness-of-fit on F² | 1.057 |
| Final R indices [I > 2sigma(I)] | R1 = 0.0374, wR2 = 0.1098 |
| R indices (all data) | R1 = 0.0441, wR2 = 0.1152 |
| Extinction coefficient | n/a |
| Largest diff. peak and hole | 0.356 and −0.188 e · Å⁻³ |

TABLE 11
Recipe of the EZ-rich media.

| Composition | Volume/μL |
|---|---|
| 10x MOPS | 1000 |
| 0.132M K₂HPO₄ | 100 |
| 10x AGCU | 1000 |
| 5x Supplement EZ | 2000 |
| 100 g L⁻¹ tryptone + 50 g L⁻¹ yeast extract, autoclaved | 1000 |
| 600 g L⁻¹ glucose | 330 |
| Autoclaved water | 4500 |
| 1.0M Calcium pantothenate | 50 |
| 100 mg mL⁻¹ carbenicilln | 10 |
| 5.0 mM sodium selenite | 10 |
| Total | 10000 |

TABLE 12
Material mass-balance for bioTAL production
for the intermediate scenario.

| Material | Input (kg per year) | Output (kg per year) |
|---|---|---|
| Acetate (bound) | 11,962,500 | 21,463 |
| Ash | 32,505,000 | 33,889,661 |
| bioTAL | 0 | 85,126,585 |
| Cellulose | 231,330,000 | 31,083,796 |
| Ionic liquid | 3,473,684 | 3,181,788 |
| Corn steep liquor | 2,591,164 | 0 |
| Diammonium phosphate | 324,507 | 0 |
| E. coli | 6 | 81,413,189 |
| Ethyl acetate | 8,595,738 | 8,595,738 |
| Glucose | 0 | 35,438,391 |
| Hemicellulose | 128,865,000 | 21,737,943 |
| Hydrolase | 4,626,600 | 752,604 |
| Lactic acid | 0 | 8,382,764 |
| Lignin | 104,032,500 | 128,793 |
| Methane | 1,188,000 | 0 |
| Methanol | 4,343 | 4,343 |
| Other solids | 151,305,000 | 1,340,004 |
| Return cooling water | 8,395,200,000 | 0 |
| Soluble lignin | 0 | 285,617 |
| Sulfuric acid | 132,387,595 | 128,416,213 |
| Water | 1,582,686,415 | 10,129,267,443 |
| Xylose | 0 | 19,316,246 |
| TOTAL | 10,791,078,052 | 10,588,382,581 |

*Only make-up amount is shown in the mass-balance stream. Solvent recycling is assumed to be 95% for the intermediate case.

TABLE 13
Process parameters and assumptions

| | Units | Value | Optimized |
|---|---|---|---|
| Process Parameters | | | |
| Corn stover | bone-dry | 2000 | 2000 |
| | metric | | |
| | ton(bdt)/day | | |
| Cost of corn stover | $/bdt | 87.83 | 71.26 |
| Cellulose | wt % | 28.04 | 35.1 |
| Hemicellulose | wt % | 15.62 | 19.5 |
| Ionic liquid (IL) | | | |
| pre-treatment | | | |
| Solids loading rate | wt % | 30 | 30 |
| Ionic liquid | kg/kg-dry | 0.29 | 0.125 |
| ([Ch][Lys]) loading | biomass | | |
| rate | | | |
| Ionic liquid | $/kg | 2 | 0.5 |
| ([Ch][Lys]) cost | | | |
| Sulfuric acid loading | kg/kg- IL | 0.2 | 0.07 |
| Sulfuric acid price | $/kg | 0.14 | 0.14 |
| Lignin to soluble lignin | wt % | 31 | 17 |
| Pretreatment time | h | 3 | 1 |
| IL recovery rate | wt % | 97 | 99 |
| Enzymatic hydrolysis | | | |
| Solids loading rate | wt % | 25 | 25 |
| Enzyme loading rate | mg/g-glucan | 20 | 10 |
| Cellulose to glucose | wt % | 84 | 95 |
| Xylan to xylose | wt % | 80 | 90 |
| Temperature | ° C. | 48 | 48 |
| Hydrolysis time | h | 72 | 48 |
| Enzyme (hydrolase) | $/kg-protein | 4.29 | 4.29 |
| price | | | |
| Bioconversion | | | |
| Cost of corn steep | $/kg | 0.1 | 0.1 |
| liquor | | | |
| Cost of diammonium | $/kg | 1 | 1 |
| phosphate | | | |
| Aeration rate | vvm | 0.5 | 0.5 |
| Power consumption | kW/m³ | 0.5 | 0.5 |
| Power dissipation to | % | 40 | 40 |
| heat | | | |
| Temperature | ° C. | 22 | 22 |
| Bioconversion time | h | 74.5* | 48^{†} |
| Glucose to bioTAL | wt % | 14.9 | 90 |
| Xylose to bioTAL | g bioTAL/g | 0.094 | 0.567 |
| | xylose | | |
| Onsite energy | | | |
| generation | | | |
| Boiler heat loss | | 5% | 5% |

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2 and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by examples, embodiments, and optional features, modification and variation of the concepts herein disclosed may be resorted to by those skilled in the art, and that such modifications and variations are considered to be within the scope of this invention as defined by the appended claims.

1. A polydiketoenamine according to Formula IA: 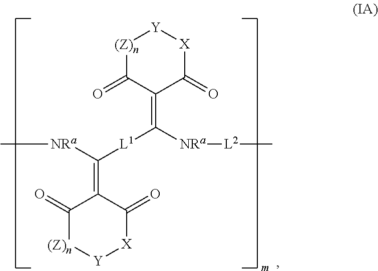
or a tautomer thereof, wherein:
subscript n is an integer ranging from 0 to 2;
L¹ is a linear, divalent linker moiety, and
L² is a linear, divalent linker moiety or a branched, trivalent linker moiety,
provided that at least one of L¹ and L² comprises one or more heteroatoms selected from the group consisting of O, S, Se, and P;
each subscript n is independently 0 or 1;
each X is independently selected from the group consisting of O, CR^{1a}R^{1b}, SiR^{1a}R^{1b}, NR^{1c}S, Se, and PR^{1d};
each Z is independently selected from the group consisting of O, CR^{3a}R^{3b}, SiR^{3a}R^{3b}, NR^{3c}, S, Se, and PR^{3d};
each Y is independently selected from the group consisting of O, CR^{2a}R^{2b}, SiR^{2a}R^{2b}, NR^{2c}, S, Se, and PR^{2d}, or Y is CR^{2a}R^{2b} when X is O, SiR^{1a}R^{1b}, NR^{1c}, S, Se, and PR^{1d}, or Y is CR^{2a}R^{2b} when Z is O, SiR^{3a}R^{3b}, NR^{3c}, S, Se, and PR^{3d};
each R^{1a}, R^{1b}, R^{2a}, R^{2b}, R^{3a}, and R^{3b} is independently selected from the group consisting of hydrogen, C₁₋₂₀ alkyl, C₂₋₂₀ alkenyl, C₂₋₂₀ alkynyl, C₃₋₈ cycloalkyl, C₆₋₁₄ aryl, 3- to 12-membered heterocyclyl, and 5- to 12-membered heteroaryl, wherein each alkyl is optionally and independently substituted with C₃₋₈ cycloalkyl, C₆₋₁₄ aryl, 3- to 12-membered heterocyclyl, or 5- to 12-membered heteroaryl;
each R^{1c}, R^{2c}, and R^{3c} is independently selected from the group consisting of hydrogen and C₁₋₆ alkyl, wherein each alkyl is optionally substituted with C₃₋₈ cycloalkyl, C₆₋₁₄ aryl, 3- to 12-membered heterocyclyl, or 5- to 12-membered heteroaryl;
each R^{1d}, R^{2d}, and R^{3d} is independently selected from the group consisting of C₁₋₆ alkyl, C₃₋₈ cycloalkyl, and C₆₋₁₄ aryl, wherein each alkyl is optionally substituted with C₃₋₈ cycloalkyl, C₆₋₁₄ aryl, 3- to 12-membered heterocyclyl, or 5- to 12-membered heteroaryl; and
R^{2a} is optionally taken together with R^{1a} or R^{3a} to form a carbon-carbon double bond.

2. The polydiketoenamine of claim 1, wherein:
L¹ is a divalent hydrocarbon linker, optionally containing one or more heteroatoms selected from the group consisting of O, N, S, Se, and P; and
L² is a divalent hydrocarbon linker, optionally containing one or more heteroatoms selected from the group consisting of O, N, S, Se, and P, or
L² is a trivalent hydrocarbon linker containing one or more heteroatoms selected from the group consisting of O, N, S, Se, and P.

3. The polydiketoenamine of claim 1, wherein L¹ is —CH₂OCH₂— or —CH₂OCH₂CH₂OCH₂—.

4. The polydiketoenamine of claim 3, wherein:
L² is C₁₋₂₀ alkylene, 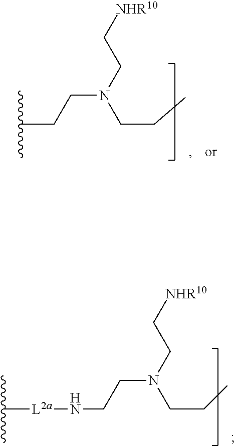
R¹⁰ is H or a branching polydiketoenamine moiety; and
L^{2a} is an oxygen-containing divalent polymer.

5-7. (canceled)

8. The polydiketoenamine of claim 1, wherein L¹ is C₁₋₂₀ alkylene.

9. The polydiketoenamine of claim 8, wherein:
L² is —(CH₂CH₂O)ₚCH₂CH₂—, —(CH₂)₃O(CH₂)₄O(CH₂)₃—, or 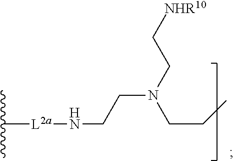
and
L^{2a} is an oxygen-containing divalent polymer.

10-11. (canceled)

12. The polydiketoenamine of claim 1, wherein:
X is O,
Y is CR^{2a}R^{2b},
subscript n is 1, and
Z is CR^{3a}R^{3b}.

13. The polydiketoenamine of claim 12, wherein R^{2a} and R^{3a} are taken together to form a carbon-carbon double bond, R^{2b} is C₁₋₆ alkyl, and R^{3b} is hydrogen.

14. The polydiketoenamine of claim 12, wherein:
(a) R^{2a} and R^{3a} are hydrogen and R^{2b} and R^{3b} are each independently C₁₋₆ alkyl; or
(b) R^{2a} and R^{2b} are each independently C₁₋₆ alkyl and R^{3a} and R^{3b} are hydrogen.

15. (canceled)

16. A composition comprising a polydiketoenamine according to claim 1, and one or more materials selected from the group consisting of an additional polymer, a filler material, a substrate material, a flame-retardant, and a pigment.

17-22. (canceled)

23. A polydiketoenamine according to claim 1, which is provided as a population of fibers having an average diameter, width, or thickness ranging from about 0.5 nm to about 1.0 mm and an average length ranging from about 5 nm to about 5000 meters.

24. A porous material comprising a polydiketoenamine according to claim 1, the porous material having pore sizes ranging from about 0.5 nm to about 5000 nm.

25-26. (canceled)

27. A foam comprising a polydiketoenamine according to claim 1, the foam having a density ranging from about 0.1 pounds per cubic foot to about 10 pounds per cubic foot.

28. An emulsion comprising a suspension of a polydiketoenamine according to claim 1 in a solvent.

29. (canceled)

30. A conductive material comprising a polydiketoenamine according to claim 1.

31. An insulating material comprising a polydiketoenamine according to claim 1.

32. A method for recycling a polydiketoenamine, the method comprising combining a polydiketoenamine according to claim 1 with an acid or a base or a combination thereof to depolymerize the polydiketoenamine.

33-35. (canceled)

36. A compound according to Formula IIA: 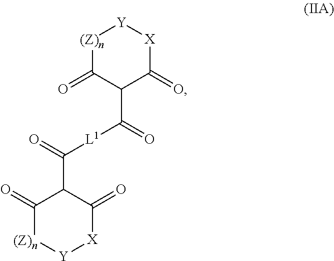
or a tautomer thereof, wherein:
L¹ is a linear, divalent linker moiety comprising one or more heteroatoms selected from the group consisting of O, N, S, Se, and P;
each subscript n is independently 0 or 1;
each X is independently selected from the group consisting of O, CR^{1a}R^{1b}, SiR^{1a}R^{1b}, NR^{1c}S, Se, and PR^{1d};
each Z is independently selected from the group consisting of O, CR^{3a}R^{3b}, SiR^{3a}R^{3b}, NR^{3c}, S, Se, and PR^{3d};
each Y is independently selected from the group consisting of O, CR^{2a}R^{2b}, SiR^{2a}R^{2b}, NR^{2c}, S, Se, and PR^{2d}, or Y is CR^{2a}R^{2b} when X is O, SiR^{1a}R^{1b}, NR^{1c}, S, Se, or PR^{1d}, or Y is CR^{2a}R^{2b} when Z is O, SiR^{3a}R^{3b}, NR^{3c}, S, Se, or PR^{3d};
each R^{1a}, R^{1b}, R^{2a}, R^{2b}, R^{3a}, and R^{3b} is independently selected from the group consisting of hydrogen, C₁₋₂₀ alkyl, C₂₋₂₀ alkenyl, C₂₋₂₀ alkynyl, C₃₋₈ cycloalkyl, C₆₋₁₄ aryl, 3- to 12-membered heterocyclyl, and 5- to 12-membered heteroaryl, wherein each alkyl is optionally and independently substituted with C₃₋₈ cycloalkyl, C₆₋₁₄ aryl, 3- to 12-membered heterocyclyl, or 5- to 12-membered heteroaryl;
each R^{1c}, R^{2c}, and R^{3c} is independently selected from the group consisting of hydrogen and C₁₋₆ alkyl, wherein each alkyl is optionally substituted with C₃₋₈ cycloalkyl, C₆₋₁₄ aryl, 3- to 12-membered heterocyclyl, or 5- to 12-membered heteroaryl;
each R^{1d}, R^{2d}, and R^{3d} is independently selected from the group consisting of C₁₋₆ alkyl, C₃₋₈ cycloalkyl, and C₆₋₁₄ aryl, wherein each alkyl is optionally substituted with C₃₋₈ cycloalkyl, C₆₋₁₄ aryl, 3- to 12-membered heterocyclyl, or 5- to 12-membered heteroaryl; and
R^{2a} is optionally taken together with R^{1a} or R^{3a} to form a carbon-carbon double bond.

37. The compound of claim 36, wherein L¹ is —CH₂OCH₂— or —CH₂OCH₂CH₂OCH₂—.

38. The compound of claim 36, wherein:
(a) R^{2a} and R^{3a} are taken together to form a carbon-carbon double bond, R^{2b} is C₁₋₆ alkyl, and R^{3b} is hydrogen;
(b) R^{2a} and R^{3a} are hydrogen and R^{2b} and R^{3b} are independently C₁₋₆ alkyl; or
(c) R^{2a} and R^{2b} are independently C₁₋₆ alkyl and R^{3a} and R^{3b} are hydrogen.

39-40. (canceled)
